SEQUENCE LISTING (1197 sequences)

SEQ ID NO: 1 - DNA, 6414 bp, Homo sapiens
  source 1..6414: mol_type = unassigned DNA
ctgggattgggacacactttctgggcactgctggccagtcccaaaatggaacataaggaagtggttcttctacttcttttatttctgaaatcagcagcacctgagcaaagccatgtggtccaggattgctaccatggtgatggacagagttatcgaggcacgtactccaccactgtcacaggaaggacctgccaagcttggtcatctatgacaccacatcaacataataggaccacagaaaactacccaaatgctggcttgatcatgaactactgcaggaatccagatgctgtggcagctccttattgttatacgagggatcccggtgtcaggtgggagtactgcaacctgacgcaatgctcagacgcagaagggactgccgtcgcgcctccgactgttaccccggttccaagcctagaggctccttccgaacaagcaccgactgagcaaaggcctggggtgcaggagtgctaccatggtaatggacagagttatcgaggcacatactccaccactgtcacaggaagaacctgccaagcttggtcatctatgacaccacactcgcatagtcggaccccagaatactacccaaatgctggcttgatcatgaactactgcaggaatccagatgctgtggcagctccttattgttatacgagggatcccggtgtcaggtgggagtactgcaacctgacgcaatgctcagacgcagaagggactgccgtcgcgcctccgactgttaccccggttccaagcctagaggctccttccgaacaagcaccgactgagcaaaggcctggggtgcaggagtgctaccatggtaatggacagagttatcgaggcacatactccaccactgtcacaggaagaacctgccaagcttggtcatctatgacaccacactcgcatagtcggaccccagaatactacccaaatgctggcttgatcatgaactactgcaggaatccagatgctgtggcagctccttattgttatacgagggatcccggtgtcaggtgggagtactgcaacctgacgcaatgctcagacgcagaagggactgccgtcgcgcctccgactgttaccccggttccaagcctagaggctccttccgaacaagcaccgactgagcagaggcctggggtgcaggagtgctaccacggtaatggacagagttatcgaggcacatactccaccactgtcactggaagaacctgccaagcttggtcatctatgacaccacactcgcatagtcggaccccagaatactacccaaatgctggcttgatcatgaactactgcaggaatccagatgctgtggcagctccttattgttatacgagggatcccggtgtcaggtgggagtactgcaacctgacgcaatgctcagacgcagaagggactgccgtcgcgcctccgactgttaccccggttccaagcctagaggctccttccgaacaagcaccgactgagcaaaggcctggggtgcaggagtgctaccatggtaatggacagagttatcgaggcacatactccaccactgtcacaggaagaacctgccaagcttggtcatctatgacaccacactcgcatagtcggaccccagaatactacccaaatgctggcttgatcatgaactactgcaggaatccagatgctgtggcagctccttattgttatacgagggatcccggtgtcaggtgggagtactgcaacctgacgcaatgctcagacgcagaagggactgccgtcgcgcctccgactgttaccccggttccaagcctagaggctccttccgaacaagcaccgactgagcaaaggcctggggtgcaggagtgctaccatggtaatggacagagttatcgaggcacatactccaccactgtcacaggaagaacctgccaagcttggtcatctatgacaccacactcgcatagtcggaccccagaatactacccaaatgctggcttgatcatgaactactgcaggaatccagatgctgtggcagctccttattgttatacgagggatcccggtgtcaggtgggagtactgcaacctgacgcaatgctcagacgcagaagggactgccgtcgcgcctccgactgttaccccggttccaagcctagaggctccttccgaacaagcaccgactgagcaaaggcctggggtgcaggagtgctaccatggtaatggacagagttatcgaggcacatactccaccactgtcacaggaagaacctgccaagcttggtcatctatgacaccacactcgcatagtcggaccccagaatactacccaaatgctggcttgatcatgaactactgcaggaatccagatgctgtggcagctccttattgttatacgagggatcccggtgtcaggtgggagtactgcaacctgacgcaatgctcagacgcagaagggactgccgtcgcgcctccgactgttaccccggttccaagcctagaggctccttccgaacaagcaccgactgagcagaggcctggggtgcaggagtgctaccacggtaatggacagagttatcgaggcacatactccaccactgtcactggaagaacctgccaagcttggtcatctatgacaccacactcgcatagtcggaccccagaatactacccaaatgctggcttgatcatgaactactgcaggaatccagatcctgtggcagccccttattgttatacgagggatcccagtgtcaggtgggagtactgcaacctgacacaatgctcagacgcagaagggactgccgtcgcgcctccaactattaccccgattccaagcctagaggctccttctgaacaagcaccaactgagcaaaggcctggggtgcaggagtgctaccacggaaatggacagagttatcaaggcacatacttcattactgtcacaggaagaacctgccaagcttggtcatctatgacaccacactcgcatagtcggaccccagcatactacccaaatgctggcttgatcaagaactactgccgaaatccagatcctgtggcagccccttggtgttatacaacagatcccagtgtcaggtgggagtactgcaacctgacacgatgctcagatgcagaatggactgccttcgtccctccgaatgttattctggctccaagcctagaggctttttttgaacaagcactgactgaggaaacccccggggtacaggactgctactaccattatggacagagttaccgaggcacatactccaccactgtcacaggaagaacttgccaagcttggtcatctatgacaccacaccagcatagtcggaccccagaaaactacccaaatgctggcctgaccaggaactactgcaggaatccagatgctgagattcgcccttggtgttacaccatggatcccagtgtcaggtgggagtactgcaacctgacacaatgcctggtgacagaatcaagtgtccttgcaactctcacggtggtcccagatccaagcacagaggcttcttctgaagaagcaccaacggagcaaagccccggggtccaggattgctaccatggtgatggacagagttatcgaggctcattctctaccactgtcacaggaaggacatgtcagtcttggtcctctatgacaccacactggcatcagaggacaacagaatattatccaaatggtggcctgaccaggaactactgcaggaatccagatgctgagattagtccttggtgttataccatggatcccaatgtcagatgggagtactgcaacctgacacaatgtccagtgacagaatcaagtgtccttgcgacgtccacggctgtttctgaacaagcaccaacggagcaaagccccacagtccaggactgctaccatggtgatggacagagttatcgaggctcattctccaccactgttacaggaaggacatgtcagtcttggtcctctatgacaccacactggcatcagagaaccacagaatactacccaaatggtggcctgaccaggaactactgcaggaatccagatgctgagattcgcccttggtgttataccatggatcccagtgtcagatgggagtactgcaacctgacgcaatgtccagtgatggaatcaactctcctcacaactcccacggtggtcccagttccaagcacagagcttccttctgaagaagcaccaactgaaaacagcactggggtccaggactgctaccgaggtgatggacagagttatcgaggcacactctccaccactatcacaggaagaacatgtcagtcttggtcgtctatgacaccacattggcatcggaggatcccattatactatccaaatgctggcctgaccaggaactactgcaggaatccagatgctgagattcgcccttggtgttacaccatggatcccagtgtcaggtgggagtactgcaacctgacacgatgtccagtgacagaatcgagtgtcctcacaactcccacagtggccccggttccaagcacagaggctccttctgaacaagcaccacctgagaaaagccctgtggtccaggattgctaccatggtgatggacggagttatcgaggcatatcctccaccactgtcacaggaaggacctgtcaatcttggtcatctatgataccacactggcatcagaggaccccagaaaactacccaaatgctggcctgaccgagaactactgcaggaatccagattctgggaaacaaccctggtgttacacaaccgatccgtgtgtgaggtgggagtactgcaatctgacacaatgctcagaaacagaatcaggtgtcctagagactcccactgttgttccagttccaagcatggaggctcattctgaagcagcaccaactgagcaaacccctgtggtccggcagtgctaccatggtaatggccagagttatcgaggcacattctccaccactgtcacaggaaggacatgtcaatcttggtcatccatgacaccacaccggcatcagaggaccccagaaaactacccaaatgatggcctgacaatgaactactgcaggaatccagatgccgatacaggcccttggtgttttaccatggaccccagcatcaggtgggagtactgcaacctgacgcgatgctcagacacagaagggactgtggtcgctcctccgactgtcatccaggttccaagcctagggcctccttctgaacaagactgtatgtttgggaatgggaaaggataccggggcaagaaggcaaccactgttactgggacgccatgccaggaatgggctgcccaggagccccatagacacagcacgttcattccagggacaaataaatgggcaggtctggaaaaaaattactgccgtaaccctgatggtgacatcaatggtccctggtgctacacaatgaatccaagaaaactttttgactactgtgatatccctctctgtgcatcctcttcatttgattgtgggaagcctcaagtggagccgaagaaatgtcctggaagcattgtaggggggtgtgtggcccacccacattcctggccctggcaagtcagtctcagaacaaggtttggaaagcacttctgtggaggcaccttaatatccccagagtgggtgctgactgctgctcactgcttgaagaagtcctcaaggccttcatcctacaaggtcatcctgggtgcacaccaagaagtgaacctcgaatctcatgttcaggaaatagaagtgtctaggctgttcttggagcccacacaagcagatattgccttgctaaagctaagcaggcctgccgtcatcactgacaaagtaatgccagcttgtctgccatccccagactacatggtcaccgccaggactgaatgttacatcactggctggggagaaacccaaggtacctttgggactggccttctcaaggaagcccagctccttgttattgagaatgaagtgtgcaatcactataagtatatttgtgctgagcatttggccagaggcactgacagttgccagggtgacagtggagggcctctggtttgcttcgagaaggacaaatacattttacaaggagtcacttcttggggtcttggctgtgcacgccccaataagcctggtgtctatgctcgtgtttcaaggtttgttacttggattgagggaatgatgagaaataattaattggacgggagacagagtgaagcatcaacctacttagaagctgaaacgtgggtaaggatttagcatgctggaaataatagacagcaatcaaacgaagacactgttcccagctaccagctatgccaaaccttggcatttttggtatttttgtgtataagcttttaaggtctgactgacaaattctgtattaaggtgtcatagctatgacatttgttaaaaataaactctgcacttattttgatttga

SEQ ID NO: 2 - DNA, 5387 bp, Macaca fascicularis
  source 1..5387: mol_type = unassigned DNA
gatgctgcatacttaatgtcgaaaggttgcttcatccaagagcctggagttttcagagacactgtcctgaaactatgtcctgaaactatgtcattgaaactgaaacattgtcctgaagctggtattgggcaataccagcgcctgcaggcaacagctcggatgcacttaagatttaaatattacccacagaagttctggcttgtctgggaaaaccttttgctaaacagaagagcaacatttttttttttttcttttctggaatttgtaaacagcatttattctcagccttaccttccaaacgttgcacttggaacattgctgggccccgtggaaacagaagcgaacgtcagccaggccggcagggggcggcagaccccacacttcgccgggcgccctcacctccctgggagggagtgtgcagctgccaaaatcttcggcggggttcagtccaagcgacttcagccagcagatggtcattctcctgtgaccgtgtgtactacagactgtttcaaaaccgggcaggcaattaacaatgggaattctgccatcatcgctgacaaagtcatcccagtttgtctgccatccccaaattatgtggtcgccaaccagactgaatgttatgtcactggctggggagaaacccaagcactacctgagcaaagccatgtggtccaggattgctaccatggtgatggacagagttatcaaggcacatcctccaccactgtcacaggaaggacctgccaagcttggtcatctatggaaccacatcagcataatagaaccacagaaaactacccaaatgctggcttgatcaggaactactgcaggaatccagatcctgtggcagccccttattgttatacgatggatcccaatgtcaggtgggagtactgcaacctgacacaatgctcagacgcagaagggactgccgtcgcacctccgaatgtcaccccggttccaagcctagaggctccttccgaacaagcaccgactgagcaaaggcctggggtgcaggagtgctaccacggtaatggacagagttatcgaggcacatacttcaccactgtgacaggaagaacctgccaagcttggtcatctatgacaccgcactctcatagtcggaccccggaaaactacccaaatggtggcttgatcaggaactactgcaggaatccagatcctgtggcagccccttattgttataccatggatcccaatgtcaggtgggagtactgcaacctgacacaatgctcagacgcagaagggattgccgtcacacctctgactgttaccccggttccaagcctagaggctccttccaagcaagcaccaactgagcaaaggcctggtgtccaggagtgctaccacggtaatggacagagttatcgaggcacatacttcaccactgtgacaggaagaacctgccaagcttggtcatctatgacaccacattctcatagtcgtaccccagaaaactacccaaatggtggcttgatcaggaactactgcaggaatccagatcctgtggcagccccttattgttataccatggatcccaatgtcaggtgggagtactgcaacctgacacaatgctcagacgcagaagggactgccgtcgcacctccgactgtcaccccggttccaagcctagaggctccttccgaacaagcaccgactgagcaaaggcctggggtgcaggagtgctaccacggtaatggacagagttatcgaggcacatacttcaccactgtgacaggaagaacctgccaagcttggtcatctatgacaccgcactctcatagtcggaccccggaaaactacccaaatggtggcttgatcaggaactactgcaggaatccagatcctgtggcagccccttattgttataccatggatcccaatgtcaggtgggagtactgcaacctgacacaatgctcagacgcagaagggactgccgtcgcacctccgaatgtcaccccggttccaagcctagaggctccttctgagcaagcaccaactgagcaaaggcttggggtgcaggagtgctaccacggtaatggacagagttatcgaggcacatacttcaccactgtgacaggaagaacctgccaagcttggtcatctatgacaccacactctcatagtcggaccccagaaaactacccaaatgctggcttggtcaagaactactgccgaaatccagatcctgtggcagccccttggtgttatacaacggatcccagtgtcaggtgggagtactgcaacctgacacgatgctcagatgcagaagggactgctgttgtgcctccaaatattattccggttccaagcctagaggcttttcttgaacaagaaccgactgaggaaacccccggggtacaggagtgctactaccattatggacagagttatagaggcacatactccaccactgttacaggaagaacttgccaagcttggtcatctatgacaccacaccagcatagtcggaccccaaaaaactatccaaatgctggcctgaccaggaactactgcaggaatccagatgctgagattcgcccttggtgttataccatggatcccagtgtcaggtgggagtactgcaacctgacacaatgtctggtgacagaatcaagtgtccttgaaactctcacagtggtcccagatccaagcacacaggcttcttctgaagaagcaccaacggagcaaagtcccgaggtccaggactgctaccatggtgatggacagagttatcgaggctcattctccaccactgtcacaggaaggacatgtcagtcttggtcctctatgacaccacactggcatcagaggacaacagaatattatccagatggtggcctgaccaggaactactgcaggaatccagatgctgagattcgcccttggtgttataccatggatcccagtgtcaggtgggagtactgcaacctgacacaatgtccagtgacagaatcaagtgtcctcgcaacgtccatggctgtttctgaacaagcaccaatggagcaaagccccggggtccaggactgctaccatggtgatggacagagttatcgaggttcattctccaccactgtcacaggaaggacatgtcagtcttggtcctctatgacaccacactggcatcagaggaccatagaatactacccaaatggtggcctgaccaagaactactgcaggaatccagatgctgagattcgcccttggtgttataccatggatcccagagtcagatgggagtactgcaacctgacacaatgtgtggtgatggaatcaagtgtccttgcaactcccatggtggtcccagttccaagcagagaggttccttctgaagaagcaccaactgaaaacagccctggggtccaggactgctaccaaggtgatggacagagttatcgaggcacattctccaccactatcacaggaagaacatgtcagtcttggttgtctatgacaccacatcggcatcggaggatcccattacgctatccaaatgctggcctgaccaggaactattgcagaaatccagatgctgagattcgcccttggtgttacaccatggatcccagtgtcaggtgggagtactgcaacctgacacaatgtccagtgacagaatcaagtgtcctcacaactcccacggtggtcccggttccaagcacagaggctccttctgaacaagcaccacctgagaaaagccctgtggtccaggattgctaccatggtgatggacagagttatcgaggcacatcctccaccactgtcacaggaaggaactgtcagtcttggtcatctatgataccacactggcatcagaggaccccagaaaactacccaaatgctggcctgaccaggaactactgcaggaatccagattctgggaaacaaccctggtgttacacgactgatccatgtgtgaggtgggagtactgcaacctgacacaatgctcagaaacagaatcaggtgtcctagagactcccactgttgttccggttccaagcatggaagctcattctgaagcagcaccaactgagcaaactcctgtggtccagcagtgctaccatggtaatggacagagttatcgaggcacattctccaccactgtcacaggaaggacatgtcaatcttggtcatccatgacaccacaccagcataagaggaccccggaaaaccacccaaatgatgacttgacaatgaactactgcaggaatccagatgctgacacaggcccttggtgttttaccatggaccccagcgtcaggcgggagtactgcaacctgacgcgatgctcagacacagaagggactgtggtcacacctccgactgttatcccggttccaagcctagaggctccttctgaacaagcatcctcttcatttgattgtgggaagcctcaagtggagccaaagaaatgtcctggaagcattgtaggtgggtgtgtggcccacccacattcctggccctggcaagtcagtcttagaacaaggtttggaaagcacttctgtggaggcaccttaatatccccagagtgggtgctgactgctgcttgctgcttggagacgttctcaaggccttccttctacaaggtcatcctgggtgcacaccaagaagtgaatctcgaatctcacgttcaagaaatagaagtgtctaggttgttcttggagcccataggagcagatattgccttgctaaagctaagcaggcctgccatcatcactgacaaagtaatcccagcctgtctgccgtctccaaattacgtgatcaccgtctggactgaatgttacatcactggctggggagaaacccaaggtacctttggggctggccttctcaaggaagcccagcttcatgtgattgagaatacagtgtgcaatcactacgagtttctgaatggaagagtcaaatccaccgagctctgtgctgggcatttggccggaggcactgacagatgccagggtgacagtggagggcctgtggtttgcttcgacaaggacaaatacattttacgaggaataacttcttggggtcctggctgtgcatgccccaataagcctggtgtctatgttcgtgtttcaagctttgtcacttggattgagggagtgatgagaaataattaattgaacaagagacagagtgaagcattgactcacctagaggctagaatgggggtagggatttagcacgctggaaataacggacagtaatcaaacgaagacactgtccccagctaccaactatgccaaacctcagcatttttggtattattgtgtataagcttttcccgtctgactgctgggttctccaataaggtgacatagctatgccatttgttaaaaataaactctgtacttattttgatttgagtaaa

SEQ ID NO: 3 - DNA, 6428 bp, Macaca mulatta
  source 1..6428: mol_type = unassigned DNA
  misc_difference 2053: note = misc_feature - a, c, g, or t
  misc_difference 2272: note = misc_feature - a, c, g, or t
  misc_difference 2395: note = misc_feature - a, c, g, or t
agccttgcctttgaaatgttccagttggaacattgctgggcagcgtgcaaacaggagcgaacgtcagccggggcggcagggggcagcagaccccacactttgtccatgcctcaggtgggaggaagtgtccggctccagaaacctgccgcgggctttatcccaagcgacttcagccagcagacggttcatgtcctgaggctgcaaaatacgagttctgccatcatcgctgacaaagtcatcccagtttgtctgccatccccaaattatgcgatcgccaaccagactgaatgttatgtcactggctggggagaaacccaagcactacctgagcaaagccatgtggtccaggattgctaccatggtgatggacagagttatcaaggcacatcctccaccactgtcacaggaaggacctgccaagcttggtcatctatggaaccacatcagcataatagaaccacagaaaactacccaaatgctggcttgatcaggaactactgcaggaatccagatcctgtggcagccccttattgttatacgatggatcccaatgtcaggtgggagtactgcaacctgacacaatgctcagacgcagaagggactgccgtcgcacctccgaatgtcaccccggttccaagcctagaggctctttccgaacaagcaccgactgagcaaaggcctggggtgcaggagtgctaccacggtaatggacagagttatcgaggcacatacttcaccactgtgacaggaagaacctgccaagcttggtcatctatgacaccacattctcatagtcgtaccccagaaaactacccaaatggtggcttgatcaggaactactgcaggaatccagatcctgtggcagccccttattgttataccatggatcccaatgtcaggtgggagtactgcaacctgacacaatgctcagacgcagaagggactgccgtcgcacctccgaatgtcaccccggttccaagcctagaggctccttccgaacaagcaccgactgagcaaaggcctggggtgcaggagtgctaccacggtaatggacagagttatcgaggcacatacttcaccactgtgacaggaagaacctgccaagcttggtcatctatgacaccacattctcatagtcgtaccccagaaaactacccaaatggtggcttgatcaggaactactgcaggaatccagatcctgtggcagccccttattgttataccatggatcccaatgtcaggtgggagtactgcaacctgacacaatgctcagacgcagaggggactgccgtcgcacctccgactgtcaccccggttccaagcctagaggctccttctgagcaagcaccgactgagcaaaggcctggggtgcaggagtgctaccacggtaatggacagagttatcgaggcacatacttcaccactgtgacaggaagaacctgccaagcttggtcatctatgacaccgcactctcatagtcggaccccggaaaactacccaaatggtggcttgatcaggaactactgcaggaatccagatcctgtggcagccccttattgttatacgatggatcccaatgtcaggtgggagtactgcaacctgacacaatgctcagacgcagaagggactgccgtcgcacctccgaatgtcaccccggttccaagcctagaggctccttccgaacaagcaccgactgagcaaaggcctggggtgcaggagtgctaccacggtaatggacagagttatcgaggcacatacttcaccactgtgacaggaagaacctgccaagcttggtcatctatgacaccgcactctcatagtcggaccccggaaaactacccaaatggtggcttgatcaggaactactgcaggaatccagatcctgtggcagccccttattgttatacgatggatcccaatgtcaggtgggagtactgcaacctgacacaatgctcagacgcagaagggactgccgtcgcacctccgaatgtcaccccggttccaagcctagaggctccttccgaacaagcaccaactgagcaaaggcctgggntgcaggagtgctaccatggtaatggacagagttatcgaggcacatacttcaccactgtgacaggaagaacctgccaagcttggtcatctatgacaccgcactctcatagtcggaccccggaaaactacccaaatggtggcttgatcaggaactactgcaggaatccagatcctgtggcagccccttattgttataccatggatcccaatgtcaggtggnagtactgcaacctgacacaatgctcagacgcagaagggactgccgtcgcacctccgactgtcaccccggttccaagcctagaggctccttcgagcaaggcaccgactgagcaaaggcctgggntgcaggagtgctaccacggtaatggacagagttatcgaggcacatacttcaccactgtgacaggaagaacctgccaagcttggtcatctatgacaccgcactctcatagtcggaccccggaaaactacccaaatggtggcttgatcaggaactactgcaggaatccagatcctgtggcagccccttattgttatacgatggatcccaatgtcaggtgggagtactgcaacctgacacaatgctcagacgcagaagggactgccgtcgcacctccgaatgtcaccccggttccaagcctagaggctccttccgaacaagcaccgactgagcaaaggcctggggtgcaggagtgctaccacggtaatggacagagttatcgaggcacatacttcaccactgtgacaggaagaacctgccaagcttggtcatctatgacaccgcactctcatagtcggaccccggaaaactacccaaatggtggcttgatcaggaactactgcaggaatccagatcctgtggcagccccttattgttataccatggatcccaatgtcaggtgggagtactgcaacctgacacaatgctcagacgcagaagggactgccgtcgcacctccgaatgtcaccccggttccaagcctagaggctccttctgagcaagcaccaactgagcaaaggcttggggtgcaggagtgctaccacagtaatggacagagttatcgaggcacatacttcaccactgtgacaggaagaacctgccaagcttggtcatctatgacaccacactctcatagtcggaccccagaaaactacccaaatgctggcttggtcaagaactactgccgaaatccagatcctgtggcagccccttggtgttatacaacggatcccagtgtcaggtgggagtactgcaacctgacacgatgctcagatgcagaagggactgctgtcatgcctccaaatattattccggttccaagcctagaggcttttcttgaacaagaacctactgaggaaacccccggggtacaggagtgctactaccattatggacagagttatcgaggcacatactccaccactgttacaggaagaacttgccaagcttggtcatctatgacaccacaccagcatagtcggaccccaaaaaactatccaaatgctggcctgaccaggaactactgcaggaatccagatgctgagattcgcccttggtgttataccatggatcccagtgtcaggtgggagtactgcaacctgacacaatgtctggtgacagaatcaagtgtccttgaaactctcacagtggtcccagatccaagcacacaggcttcttctgaagaagcaccaacggagcaaagtcccgaggtccaggactgctaccatggtgatggacagagttatcgaggctcattctccaccactgtcacaggaaggacatgtcagtcttggtcctctatgacaccacactggcatcagaggacaacagaatattatccagatggtggcctgaccaggaactactgcaggaatccagatgctgagattcgcccttggtgttataccatggatcccagtgtcaggtgggagtactgcaacctgacacaatgtccagtgacagaatcaagtgtcctcgcaacgtccatggctgtttctgaacaagcaccaatggagcaaagccccggggtccaggactgctaccatggtgatggacagagttatcgaggttcattctccaccactgtcacaggaaggacatgtcagtcttggtcctctatgacaccacactggcatcagaggaccatagaatactacccaaatggtggcctgaccaagaactactgcaggaatccagatgctgagattcgcccttggtgttataccatggatcccagagtcagatgggagtactgcaacctgacacaatgtgtggtgatggaatcaagtgtccttgcaactcccatggtggtcccagttccaagcagagaggttccttctgaagaagcaccaactgaaaacagccctggggtccaggactgctaccaaggtgatggacagagttatcgaggcacattctccaccactatcacaggaagaacatgtcagtcttggttgtctatgacaccacatcggcatcggaggatcccattacgctatccaaatgctggcctgaccaggaactattgcagaaatccagatgctgagattcgcccttggtgttacaccatggatcccagtgtcaggtgggagtactgcaacctgacacaatgtccagtgacagaatcaagtgtcctcacaactcccacggtggtcccggttccaagcacagaggctccttctgaacaagcaccacctgagaaaagccctgtggtccaggattgctaccatggtgatggacagagttatcgaggcacatcctccaccactgtcacaggaaggaactgtcaatcttggtcatctatgataccacactggcatcagaggaccccagaaaactacccaaatgctggcctgaccaggaactactgcaggaatccagattctgggaaacaaccctggtgttacacgactgatccatgtgtgaggtgggagtactgcaacctgacacaatgctcagaaacagaatcaggtgtcctagagactcccactgttgttccggttccaagcatggaagctcattctgaagcagcaccaactgagcaaacccctgtggtccagcagtgctaccatggtaatggacagagttatcgaggcacattctccaccactgtcacaggaaggacatgtcaatcttggtcatccatgacaccacaccagcataagaggaccccggaaaaccacccaaatgatgacttgacaatgaactactgcaggaatccagatgctgacacaggcccttggtgttttaccatggaccccagcgtcaggcgggagtactgcaacctgacgcgatgctcagacacagaagggactgtggtcacacctccgactgttatcccggttccaagcctagaggctccttctgaacaagcatcctcttcatttgattgtgggaagcctcaagtggagccaaagaaatgtcctggaagcattgtaggtgggtgtgtggcccacccacattcctggccctggcaagtcagtcttagaacaaggtttggaaagcacttctgtggaggcaccttaatatccccagagtgggtgctgactgctgcttgctgcttggagacgttctcaaggccttccttctacaaggtcatcctgggtgcacaccaagaagtgaatctcgaatctcatgttcaagaaatagaagtgtctaggttgttcttggagcccataggagcagatattgccttgctaaagctaagcaggcctgccatcatcactgacaaagtaatcccagcctgtctgccgtctccaaattacgtgatcaccgcctggactgaatgttacatcactggctggggagaaacccaaggtacctttggggctggccttctcaaggaagcccagcttcatgtgattgagaatacagtgtgcaatcactacgagtttctgaatggaagagtcaaatccactgagctctgtgctgggcatttggccggaggcactgacagatgccagggtgacaatggagggcctgtggtttgcttcgacaaggacaaatacattttacgaggaataacttcttggggtcctggctgtgcatgccccaataagcctggtgtctatgttcgtgtttcaagctttgtcacttggattgagggagtgatgagaaataattaattgaacaagagacagagtgaagcattgactcacctagaggctagaatgggggtagggatttagcacgctggaaataacggacagtaatcaaacgaagacactgtccccagctaccaactatgccaaacctcagcatttttggtattattgtgtataagcttttcctgtctgactgctgggttctccaataaggtgacatagctatgccatttgttaaaaataaactctgtacttattttgatttgagtaaa

SEQ ID NO: 4 - RNA, 25 nt, synthetic construct
  source 1..25: mol_type = other RNA; note = Synthetic
ctgagcaaagccatgtggtacagga

SEQ ID NO: 5 - DNA, 25 bp, synthetic construct
  source 1..25: mol_type = other DNA; note = Synthetic
  modified_base 24: mod_base = OTHER; note = thymine
agcaaagccatgtggtccaagattg

SEQ ID NO: 6 - RNA, 25 nt, synthetic construct
  source 1..25: mol_type = other RNA; note = Synthetic
aagccatgtggtccaggatagctac

SEQ ID NO: 7 - RNA, 25 nt, synthetic construct
  source 1..25: mol_type = other RNA; note = Synthetic
agccatgtggtccaggattactacc

SEQ ID NO: 8 - RNA, 25 nt, synthetic construct
  source 1..25: mol_type = other RNA; note = Synthetic
gccatgtggtccaggattgatacca

SEQ ID NO: 9 - DNA, 25 bp, synthetic construct
  source 1..25: mol_type = other DNA; note = Synthetic
  modified_base 25: mod_base = OTHER; note = thymine
ccatgtggtccaggattgcaaccat

SEQ ID NO: 10 - DNA, 25 bp, synthetic construct
  source 1..25: mol_type = other DNA; note = Synthetic
  modified_base 24: mod_base = OTHER; note = thymine
catgtggtccaggattgctaccatg

SEQ ID NO: 11 - RNA, 25 nt, synthetic construct
  source 1..25: mol_type = other RNA; note = Synthetic
atgtggtccaggattgctaacatgg

SEQ ID NO: 12 - DNA, 25 bp, synthetic construct
  source 1..25: mol_type = other DNA; note = Synthetic
  modified_base 25: mod_base = OTHER; note = thymine
tgtggtccaggattgctacaatggt

SEQ ID NO: 13 - RNA, 25 nt, synthetic construct
  source 1..25: mol_type = other RNA; note = Synthetic
ggtgatggacagagttatcaaggca

SEQ ID NO: 14 - DNA, 25 bp, synthetic construct
  source 1..25: mol_type = other DNA; note = Synthetic
  modified_base 25: mod_base = OTHER; note = thymine
tccaccactgtcacaggaaagacct

SEQ ID NO: 15 - DNA, 25 bp, synthetic construct
  source 1..25: mol_type = other DNA; note = Synthetic
  modified_base 24: mod_base = OTHER; note = thymine
ccaccactgtcacaggaagaacctg

SEQ ID NO: 16 - RNA, 25 nt, synthetic construct
  source 1..25: mol_type = other RNA; note = Synthetic
ctgtcacaggaaggacctgacaagc

SEQ ID NO: 17 - DNA, 25 bp, synthetic construct
  source 1..25: mol_type = other DNA; note = Synthetic
  modified_base 25: mod_base = OTHER; note = thymine
ggaaggacctgccaagcttagtcat

SEQ ID NO: 18 - DNA, 25 bp, synthetic construct
  source 1..25: mol_type = other DNA; note = Synthetic
  modified_base 24: mod_base = OTHER; note = thymine
gaaggacctgccaagcttgatcatc

SEQ ID NO: 19 - DNA, 25 bp, synthetic construct
  source 1..25: mol_type = other DNA; note = Synthetic
  modified_base 24: mod_base = OTHER; note = thymine
aggacctgccaagcttggtaatcta

SEQ ID NO: 20 - DNA, 25 bp, synthetic construct
  source 1..25: mol_type = other DNA; note = Synthetic
  modified_base 25: mod_base = OTHER; note = thymine
ggacctgccaagcttggtcatctat

SEQ ID NO: 21 - DNA, 25 bp, synthetic construct
  source 1..25: mol_type = other DNA; note = Synthetic
  modified_base 24: mod_base = OTHER; note = thymine
gacctgccaagcttggtcaactatg

SEQ ID NO: 22 - RNA, 25 nt, synthetic construct
  source 1..25: mol_type = other RNA; note = Synthetic
acctgccaagcttggtcatatatga

SEQ ID NO: 23 - RNA, 25 nt, synthetic construct
  source 1..25: mol_type = other RNA; note = Synthetic
cctgccaagcttggtcatcaatgac

SEQ ID NO: 24 - DNA, 25 bp, synthetic construct
  source 1..25: mol_type = other DNA; note = Synthetic
  modified_base 25: mod_base = OTHER; note = thymine
agcttggtcatctatgacaacacat

SEQ ID NO: 25 - DNA, 25 bp, synthetic construct
  source 1..25: mol_type = other DNA; note = Synthetic
  modified_base 25: mod_base = OTHER; note = thymine
gtcatctatgacaccacataaacat

SEQ ID NO: 26 - RNA, 25 nt, synthetic construct
  source 1..25: mol_type = other RNA; note = Synthetic
cacagaaaactacccaaatactggc

SEQ ID NO: 27 - DNA, 25 bp, synthetic construct
  source 1..25: mol_type = other DNA; note = Synthetic
  modified_base 24: mod_base = OTHER; note = thymine
agaaaactacccaaatgctagcttg

SEQ ID NO: 28 - DNA, 25 bp, synthetic construct
  source 1..25: mol_type = other DNA; note = Synthetic
  modified_base 25: mod_base = OTHER; note = thymine
aaaactacccaaatgctggattgat

SEQ ID NO: 29 - RNA, 25 nt, synthetic construct
  source 1..25: mol_type = other RNA; note = Synthetic
acccaaatgctggcttgataatgaa

SEQ ID NO: 30 - RNA, 25 nt, synthetic construct
  source 1..25: mol_type = other RNA; note = Synthetic
cccaaatgctggcttgatcatgaac

SEQ ID NO: 31 - DNA, 25 bp, synthetic construct
  source 1..25: mol_type = other DNA; note = Synthetic
  modified_base 25: mod_base = OTHER; note = thymine
gaactactgcaggaatccaaatgct

SEQ ID NO: 32 - RNA, 25 nt, synthetic construct
  source 1..25: mol_type = other RNA; note = Synthetic
tactgcaggaatccagatgatgtgg

SEQ ID NO: 33 - RNA, 25 nt, synthetic construct
  source 1..25: mol_type = other RNA; note = Synthetic
actgcaggaatccagatgcagtggc

SEQ ID NO: 34 - RNA, 25 nt, synthetic construct
  source 1..25: mol_type = other RNA; note = Synthetic
tgcaggaatccagatgctgaggcag

SEQ ID NO: 35 - RNA, 25 nt, synthetic construct
  source 1..25: mol_type = other RNA; note = Synthetic
aggtgggagtactgcaaccagacgc

SEQ ID NO: 36 - RNA, 25 nt, synthetic construct
  source 1..25: mol_type = other RNA; note = Synthetic
aatgctcagacgcagaaggaactgc

SEQ ID NO: 37 - DNA, 25 bp, synthetic construct
  source 1..25: mol_type = other DNA; note = Synthetic
  modified_base 24: mod_base = OTHER; note = thymine
gactgttaccccggttccaagccta

SEQ ID NO: 38 - RNA, 25 nt, synthetic construct
  source 1..25: mol_type = other RNA; note = Synthetic
actgttaccccggttccaaacctag

SEQ ID NO: 39 - RNA, 25 nt, synthetic construct
  source 1..25: mol_type = other RNA; note = Synthetic
ctgttaccccggttccaagactaga

SEQ ID NO: 40 - RNA, 25 nt, synthetic construct
  source 1..25: mol_type = other RNA; note = Synthetic
tgttaccccggttccaagcatagag

SEQ ID NO: 41 - RNA, 25 nt, synthetic construct
  source 1..25: mol_type = other RNA; note = Synthetic
gttaccccggttccaagccaagagg

SEQ ID NO: 42 - RNA, 25 nt, synthetic construct
  source 1..25: mol_type = other RNA; note = Synthetic
ttaccccggttccaagcctagaggc

SEQ ID NO: 43 - DNA, 25 bp, synthetic construct
  source 1..25: mol_type = other DNA; note = Synthetic
  modified_base 25: mod_base = OTHER; note = thymine
taccccggttccaagcctaaaggct

SEQ ID NO: 44 - DNA, 25 bp, synthetic construct
  source 1..25: mol_type = other DNA; note = Synthetic
  modified_base 25: mod_base = OTHER; note = thymine
gtgctaccatggtaatggaaagagt

SEQ ID NO: 45 - DNA, 25 bp, synthetic construct
  source 1..25: mol_type = other DNA; note = Synthetic
  modified_base 24: mod_base = OTHER; note = thymine
  modified_base 25: mod_base = OTHER; note = thymine
tgctaccatggtaatggacagagtt

SEQ ID NO: 46 - DNA, 25 bp, synthetic construct
  source 1..25: mol_type = other DNA; note = Synthetic
  modified_base 24: mod_base = OTHER; note = thymine
gctaccatggtaatggacaaagtta

SEQ ID NO: 47 - DNA, 25 bp, synthetic construct
  source 1..25: mol_type = other DNA; note = Synthetic
  modified_base 25: mod_base = OTHER; note = thymine
ctaccatggtaatggacagagttat

SEQ ID NO: 48 - DNA, 25 bp, synthetic construct
  source 1..25: mol_type = other DNA; note = Synthetic
  modified_base 24: mod_base = OTHER; note = thymine
taccatggtaatggacagaattatc

SEQ ID NO: 49 - RNA, 25 nt, synthetic construct
  source 1..25: mol_type = other RNA; note = Synthetic
accatggtaatggacagagatatcg

SEQ ID NO: 50 - RNA, 25 nt, synthetic construct
  source 1..25: mol_type = other RNA; note = Synthetic
ccatggtaatggacagagtaatcga

SEQ ID NO: 51 - RNA, 25 nt, synthetic construct
  source 1..25: mol_type = other RNA; note = Synthetic
catggtaatggacagagttatcgag

SEQ ID NO: 52 - RNA, 25 nt, synthetic construct
  source 1..25: mol_type = other RNA; note = Synthetic
atggtaatggacagagttaacgagg

SEQ ID NO: 53 - RNA, 25 nt, synthetic construct
  source 1..25: mol_type = other RNA; note = Synthetic
tggtaatggacagagttatagaggc

SEQ ID NO: 54 - RNA, 25 nt, synthetic construct
  source 1..25: mol_type = other RNA; note = Synthetic
ggtaatggacagagttatcaaggca

SEQ ID NO: 55 - RNA, 25 nt, synthetic construct
  source 1..25: mol_type = other RNA; note = Synthetic
gtaatggacagagttatcgaggcac

SEQ ID NO: 56 - RNA, 25 nt, synthetic construct
  source 1..25: mol_type = other RNA; note = Synthetic
ggcacatactccaccactgacacag

SEQ ID NO: 57 - DNA, 25 bp, synthetic construct
  source 1..25: mol_type = other DNA; note = Synthetic
  modified_base 24: mod_base = OTHER; note = thymine
cttggtcatctatgacaccacactc

SEQ ID NO: 58 - DNA, 25 bp, synthetic construct
  source 1..25: mol_type = other DNA; note = Synthetic
  modified_base 25: mod_base = OTHER; note = thymine
gtcatctatgacaccacacacgcat

SEQ ID NO: 59 - DNA, 25 bp, synthetic construct
  source 1..25: mol_type = other DNA; note = Synthetic
  modified_base 24: mod_base = OTHER; note = thymine
tcatctatgacaccacactagcata

SEQ ID NO: 60 - RNA, 25 nt, synthetic construct
  source 1..25: mol_type = other RNA; note = Synthetic
catctatgacaccacactcacatag

SEQ ID NO: 61 - RNA, 25 nt, synthetic construct
  source 1..25: mol_type = other RNA; note = Synthetic
gcacatactccaccactgtaactgg

SEQ ID NO: 62 - DNA, 25 bp, synthetic construct
  source 1..25: mol_type = other DNA; note = Synthetic
  modified_base 25: mod_base = OTHER; note = thymine
agccccttattgttatacgagggat

SEQ ID NO: 63 - DNA, 25 bp, synthetic construct
  source 1..25: mol_type = other DNA; note = Synthetic
  modified_base 24: mod_base = OTHER; note = thymine
gccccttattgttatacgaaggatc

SEQ ID NO: 64 - RNA, 25 nt, synthetic construct
  source 1..25: mol_type = other RNA; note = Synthetic
ccaagcctagaggctccttatgaac

SEQ ID NO: 65 - RNA, 25 nt, synthetic construct
  source 1..25: mol_type = other RNA; note = Synthetic
aggctccttctgaacaagcaccaac

SEQ ID NO: 66 - DNA, 25 bp, synthetic construct
  source 1..25: mol_type = other DNA; note = Synthetic
  modified_base 24: mod_base = OTHER; note = thymine
atggacagagttatcaaggaacata

SEQ ID NO: 67 - RNA, 25 nt, synthetic construct
  source 1..25: mol_type = other RNA; note = Synthetic
tggacagagttatcaaggcacatac

SEQ ID NO: 68 - DNA, 25 bp, synthetic construct
  source 1..25: mol_type = other DNA; note = Synthetic
  modified_base 25: mod_base = OTHER; note = thymine
ggacagagttatcaaggcaaatact

SEQ ID NO: 69 - DNA, 25 bp, synthetic construct
  source 1..25: mol_type = other DNA; note = Synthetic
  modified_base 24: mod_base = OTHER; note = thymine
  modified_base 25: mod_base = OTHER; note = thymine
gacagagttatcaaggcacatactt

SEQ ID NO: 70 - DNA, 25 bp, synthetic construct
  source 1..25: mol_type = other DNA; note = Synthetic
  modified_base 24: mod_base = OTHER; note = thymine
acagagttatcaaggcacaaacttc

SEQ ID NO: 71 - RNA, 25 nt, synthetic construct
  source 1..25: mol_type = other RNA; note = Synthetic
cagagttatcaaggcacatacttca

SEQ ID NO: 72 - RNA, 25 nt, synthetic construct
  source 1..25: mol_type = other RNA; note = Synthetic
tacccaaatgctggcttgaacaaga

SEQ ID NO: 73 - DNA, 25 bp, synthetic construct
  source 1..25: mol_type = other DNA; note = Synthetic
  modified_base 25: mod_base = OTHER; note = thymine
ccaaatgctggcttgatcaagaact

SEQ ID NO: 74 - RNA, 25 nt, synthetic construct
  source 1..25: mol_type = other RNA; note = Synthetic
tcaagaactactgccgaaaaccaga

SEQ ID NO: 75 - DNA, 25 bp, synthetic construct
  source 1..25: mol_type = other DNA; note = Synthetic
  modified_base 25: mod_base = OTHER; note = thymine
caagaactactgccgaaatacagat

SEQ ID NO: 76 - DNA, 25 bp, synthetic construct
  source 1..25: mol_type = other DNA; note = Synthetic
  modified_base 24: mod_base = OTHER; note = thymine
aagaactactgccgaaatcaagatc

SEQ ID NO: 77 - DNA, 25 bp, synthetic construct
  source 1..25: mol_type = other DNA; note = Synthetic
  modified_base 25: mod_base = OTHER; note = thymine
gaactactgccgaaatccaaatcct

SEQ ID NO: 78 - DNA, 25 bp, synthetic construct
  source 1..25: mol_type = other DNA; note = Synthetic
  modified_base 24: mod_base = OTHER; note = thymine
ctactgccgaaatccagatactgtg

SEQ ID NO: 79 - RNA, 25 nt, synthetic construct
  source 1..25: mol_type = other RNA; note = Synthetic
tgtggcagccccttggtgtaataca

SEQ ID NO: 80 - RNA, 25 nt, synthetic construct
  source 1..25: mol_type = other RNA; note = Synthetic
gtggcagccccttggtgttatacaa

SEQ ID NO: 81 - RNA, 25 nt, synthetic construct
  source 1..25: mol_type = other RNA; note = Synthetic
tggcagccccttggtgttaaacaac

SEQ ID NO: 82 - RNA, 25 nt, synthetic construct
  source 1..25: mol_type = other RNA; note = Synthetic
ggcagccccttggtgttatacaaca

SEQ ID NO: 83 - RNA, 25 nt, synthetic construct
  source 1..25: mol_type = other RNA; note = Synthetic
gcagccccttggtgttataaaacag

SEQ ID NO: 84 - RNA, 25 nt, synthetic construct
  source 1..25: mol_type = other RNA; note = Synthetic
cagccccttggtgttatacaacaga

SEQ ID NO: 85 - DNA, 25 bp, synthetic construct
  source 1..25: mol_type = other DNA; note = Synthetic
  modified_base 25: mod_base = OTHER; note = thymine
agccccttggtgttatacaacagat

SEQ ID NO: 86 - DNA, 25 bp, synthetic construct
  source 1..25: mol_type = other DNA; note = Synthetic
  modified_base 24: mod_base = OTHER; note = thymine
gccccttggtgttatacaaaagatc

SEQ ID NO: 87 - RNA, 25 nt, synthetic construct
  source 1..25: mol_type = other RNA; note = Synthetic
ccccttggtgttatacaacagatcc

SEQ ID NO: 88 - RNA, 25 nt, synthetic construct
  source 1..25: mol_type = other RNA; note = Synthetic
ggtgggagtactgcaacctaacacg

SEQ ID NO: 89 - RNA, 25 nt, synthetic construct
  source 1..25: mol_type = other RNA; note = Synthetic
gtgggagtactgcaacctgacacga

SEQ ID NO: 90 - RNA, 25 nt, synthetic construct
  source 1..25: mol_type = other RNA; note = Synthetic
ggagtactgcaacctgacaagatgc

SEQ ID NO: 91 - DNA, 25 bp, synthetic construct
  source 1..25: mol_type = other DNA; note = Synthetic
  modified_base 25: mod_base = OTHER; note = thymine
gagtactgcaacctgacacaatgct

SEQ ID NO: 92 - RNA, 25 nt, synthetic construct
  source 1..25: mol_type = other RNA; note = Synthetic
gtactgcaacctgacacgaagctca

SEQ ID NO: 93 - RNA, 25 nt, synthetic construct
  source 1..25: mol_type = other RNA; note = Synthetic
tactgcaacctgacacgatactcag

SEQ ID NO: 94 - RNA, 25 nt, synthetic construct
  source 1..25: mol_type = other RNA; note = Synthetic
actgcaacctgacacgatgatcaga

SEQ ID NO: 95 - DNA, 25 bp, synthetic construct
  source 1..25: mol_type = other DNA; note = Synthetic
  modified_base 25: mod_base = OTHER; note = thymine
ctgcaacctgacacgatgcacagat

SEQ ID NO: 96 - DNA, 25 bp, synthetic construct
  source 1..25: mol_type = other DNA; note = Synthetic
  modified_base 24: mod_base = OTHER; note = thymine
tgcaacctgacacgatgctaagatg

SEQ ID NO: 97 - RNA, 25 nt, synthetic construct
  source 1..25: mol_type = other RNA; note = Synthetic
caacctgacacgatgctcaaatgca

SEQ ID NO: 98 - RNA, 25 nt, synthetic construct
  source 1..25: mol_type = other RNA; note = Synthetic
acctgacacgatgctcagaagcaga

SEQ ID NO: 99 - RNA, 25 nt, synthetic construct
  source 1..25: mol_type = other RNA; note = Synthetic
cctgacacgatgctcagatacagaa

SEQ ID NO: 100 - DNA, 25 bp, synthetic construct
  source 1..25: mol_type = other DNA; note = Synthetic
  modified_base 25: mod_base = OTHER; note = thymine
ctgacacgatgctcagatgaagaat

SEQ ID NO: 101 - DNA, 25 bp, synthetic construct
  source 1..25: mol_type = other DNA; note = Synthetic
  modified_base 24: mod_base = OTHER; note = thymine
tgacacgatgctcagatgcagaatg

SEQ ID NO: 102 - RNA, 25 nt, synthetic construct
  source 1..25: mol_type = other RNA; note = Synthetic
gacacgatgctcagatgcaaaatgg

SEQ ID NO: 103 - RNA, 25 nt, synthetic construct
  source 1..25: mol_type = other RNA; note = Synthetic
acacgatgctcagatgcagaatgga

SEQ ID NO: 104 - RNA, 25 nt, synthetic construct
  source 1..25: mol_type = other RNA; note = Synthetic
cacgatgctcagatgcagaatggac

SEQ ID NO: 105 - DNA, 25 bp, synthetic construct
  source 1..25: mol_type = other DNA; note = Synthetic
  modified_base 24: mod_base = OTHER; note = thymine
  modified_base 25: mod_base = OTHER; note = thymine
tgctactaccattatggacagagtt

SEQ ID NO: 106 - DNA, 25 bp, synthetic construct
  source 1..25: mol_type = other DNA; note = Synthetic
  modified_base 24: mod_base = OTHER; note = thymine
gctactaccattatggacaaagtta

SEQ ID NO: 107 - RNA, 25 nt, synthetic construct
  source 1..25: mol_type = other RNA; note = Synthetic
ctactaccattatggacagagttac

SEQ ID NO: 108 - RNA, 25 nt, synthetic construct
  source 1..25: mol_type = other RNA; note = Synthetic
tactaccattatggacagaattacc

SEQ ID NO: 109 - RNA, 25 nt, synthetic construct
  source 1..25: mol_type = other RNA; note = Synthetic
actaccattatggacagagataccg

SEQ ID NO: 110 - RNA, 25 nt, synthetic construct
  source 1..25: mol_type = other RNA; note = Synthetic
ctaccattatggacagagtaaccga

SEQ ID NO: 111 - RNA, 25 nt, synthetic construct
  source 1..25: mol_type = other RNA; note = Synthetic
taccattatggacagagttaccgag

SEQ ID NO: 112 - RNA, 25 nt, synthetic construct
  source 1..25: mol_type = other RNA; note = Synthetic
accattatggacagagttaacgagg

SEQ ID NO: 113 - RNA, 25 nt, synthetic construct
  source 1..25: mol_type = other RNA; note = Synthetic
gaggcacatactccaccacagtcac

SEQ ID NO: 114 - DNA, 25 bp, synthetic construct
  source 1..25: mol_type = other DNA; note = Synthetic
  modified_base 25: mod_base = OTHER; note = thymine
ctccaccactgtcacaggaagaact

SEQ ID NO: 115 - DNA, 25 bp, synthetic construct
  source 1..25: mol_type = other DNA; note = Synthetic
  modified_base 25: mod_base = OTHER; note = thymine
acaggaagaacttgccaagattggt

SEQ ID NO: 116 - DNA, 25 bp, synthetic construct
  source 1..25: mol_type = other DNA; note = Synthetic
  modified_base 24: mod_base = OTHER; note = thymine
caggaagaacttgccaagcatggtc

SEQ ID NO: 117 - RNA, 25 nt, synthetic construct
  source 1..25: mol_type = other RNA; note = Synthetic
aggaagaacttgccaagctaggtca

SEQ ID NO: 118 - DNA, 25 bp, synthetic construct
  source 1..25: mol_type = other DNA; note = Synthetic
  modified_base 25: mod_base = OTHER; note = thymine
ggaagaacttgccaagcttagtcat

SEQ ID NO: 119 - DNA, 25 bp, synthetic construct
  source 1..25: mol_type = other DNA; note = Synthetic
  modified_base 24: mod_base = OTHER; note = thymine
gaagaacttgccaagcttgatcatc

SEQ ID NO: 120 - DNA, 25 bp, synthetic construct
  source 1..25: mol_type = other DNA; note = Synthetic
  modified_base 25: mod_base = OTHER; note = thymine
aagaacttgccaagcttggacatct

SEQ ID NO: 121 - DNA, 25 bp, synthetic construct
  source 1..25: mol_type = other DNA; note = Synthetic
  modified_base 24: mod_base = OTHER; note = thymine
agaacttgccaagcttggtaatcta

SEQ ID NO: 122 - DNA, 25 bp, synthetic construct
  source 1..25: mol_type = other DNA; note = Synthetic
  modified_base 25: mod_base = OTHER; note = thymine
gaacttgccaagcttggtcatctat

SEQ ID NO: 123 - DNA, 25 bp, synthetic construct
  source 1..25: mol_type = other DNA; note = Synthetic
  modified_base 24: mod_base = OTHER; note = thymine
aacttgccaagcttggtcaactatg

SEQ ID NO: 124 - RNA, 25 nt, synthetic construct
  source 1..25: mol_type = other RNA; note = Synthetic
acttgccaagcttggtcatatatga

SEQ ID NO: 125 - RNA, 25 nt, synthetic construct
  source 1..25: mol_type = other RNA; note = Synthetic
cttgccaagcttggtcatcaatgac

SEQ ID NO: 126 - RNA, 25 nt, synthetic construct
  source 1..25: mol_type = other RNA; note = Synthetic
ttgccaagcttggtcatctatgaca

SEQ ID NO: 127 - RNA, 25 nt, synthetic construct
  source 1..25: mol_type = other RNA; note = Synthetic
tgccaagcttggtcatctaagacac

SEQ ID NO: 128 - RNA, 25 nt, synthetic construct
  source 1..25: mol_type = other RNA; note = Synthetic
gcttggtcatctatgacacaacacc

SEQ ID NO: 129 - RNA, 25 nt, synthetic construct
  source 1..25: mol_type = other RNA; note = Synthetic
ttggtcatctatgacaccaaaccag

SEQ ID NO: 130 - RNA, 25 nt, synthetic construct
  source 1..25: mol_type = other RNA; note = Synthetic
tggtcatctatgacaccacaccagc

SEQ ID NO: 131 - DNA, 25 bp, synthetic construct
  source 1..25: mol_type = other DNA; note = Synthetic
  modified_base 25: mod_base = OTHER; note = thymine
gtcatctatgacaccacacaagcat

SEQ ID NO: 132 - RNA, 25 nt, synthetic construct
  source 1..25: mol_type = other RNA; note = Synthetic
catctatgacaccacaccaacatag

SEQ ID NO: 133 - DNA, 25 bp, synthetic construct
  source 1..25: mol_type = other DNA; note = Synthetic
  modified_base 25: mod_base = OTHER; note = thymine
atctatgacaccacaccagaatagt

SEQ ID NO: 134 - DNA, 25 bp, synthetic construct
  source 1..25: mol_type = other DNA; note = Synthetic
ctatgacaccacaccagcaaagtcg

SEQ ID NO: 135 - RNA, 25 nt, synthetic construct
  source 1..25: mol_type = other RNA; note = Synthetic
gtcggaccccagaaaactaaccaaa

SEQ ID NO: 136 - DNA, 25 bp, synthetic construct
  source 1..25: mol_type = other DNA; note = Synthetic
  modified_base 25: mod_base = OTHER; note = thymine
tcggaccccagaaaactacacaaat

SEQ ID NO: 137 - RNA, 25 nt, synthetic construct
  source 1..25: mol_type = other RNA; note = Synthetic
gaaaactacccaaatgctgacctga

SEQ ID NO: 138 - RNA, 25 nt, synthetic construct
  source 1..25: mol_type = other RNA; note = Synthetic
gctgagattcgcccttggtattaca

SEQ ID NO: 139 - RNA, 25 nt, synthetic construct
  source 1..25: mol_type = other RNA; note = Synthetic
ctgagattcgcccttggtgatacac

SEQ ID NO: 140 - RNA, 25 nt, synthetic construct
  source 1..25: mol_type = other RNA; note = Synthetic
gagattcgcccttggtgttacacca

SEQ ID NO: 141 - DNA, 25 bp, synthetic construct
  source 1..25: mol_type = other DNA; note = Synthetic
  modified_base 25: mod_base = OTHER; note = thymine
agattcgcccttggtgttaaaccat

SEQ ID NO: 142 - RNA, 25 nt, synthetic construct
  source 1..25: mol_type = other RNA; note = Synthetic
ttcgcccttggtgttacacaatgga

SEQ ID NO: 143 - RNA, 25 nt, synthetic construct
  source 1..25: mol_type = other RNA; note = Synthetic
cttggtgttacaccatggaacccag

SEQ ID NO: 144 - DNA, 25 bp, synthetic construct
  source 1..25: mol_type = other DNA; note = Synthetic
  modified_base 25: mod_base = OTHER; note = thymine
ttggtgttacaccatggataccagt

SEQ ID NO: 145 - DNA, 25 bp, synthetic construct
  source 1..25: mol_type = other DNA; note = Synthetic
  modified_base 24: mod_base = OTHER; note = thymine
tggtgttacaccatggatcacagtg

SEQ ID NO: 146 - DNA, 25 bp, synthetic construct
  source 1..25: mol_type = other DNA; note = Synthetic
  modified_base 25: mod_base = OTHER; note = thymine
ggtgttacaccatggatccaagtgt

SEQ ID NO: 147 - RNA, 25 nt, synthetic construct
  source 1..25: mol_type = other RNA; note = Synthetic
tgttacaccatggatcccaatgtca

SEQ ID NO: 148 - RNA, 25 nt, synthetic construct
  source 1..25: mol_type = other RNA; note = Synthetic
gaatcaagtgtccttgcaaatctca

SEQ ID NO: 149 - RNA, 25 nt, synthetic construct
  source 1..25: mol_type = other RNA; note = Synthetic
aatcaagtgtccttgcaacactcac

SEQ ID NO: 150 - RNA, 25 nt, synthetic construct
  source 1..25: mol_type = other RNA; note = Synthetic
atcaagtgtccttgcaactatcacg

SEQ ID NO: 151 - DNA, 25 bp, synthetic construct
  source 1..25: mol_type = other DNA; note = Synthetic
  modified_base 24: mod_base = OTHER; note = thymine
  modified_base 25: mod_base = OTHER; note = thymine
atggacagagttatcgaggatcatt

SEQ ID NO: 152 - DNA, 25 bp, synthetic construct
  source 1..25: mol_type = other DNA; note = Synthetic
  modified_base 24: mod_base = OTHER; note = thymine
tggacagagttatcgaggcacattc

SEQ ID NO: 153 - DNA, 25 bp, synthetic construct
  source 1..25: mol_type = other DNA; note = Synthetic
acaccacactggcatcagaagacaa

SEQ ID NO: 154 - DNA, 25 bp, synthetic construct
  source 1..25: mol_type = other DNA; note = Synthetic
  modified_base 25: mod_base = OTHER; note = thymine
ttggtgttataccatggataccaat

SEQ ID NO: 155 - DNA, 25 bp, synthetic construct
  source 1..25: mol_type = other DNA; note = Synthetic
  modified_base 24: mod_base = OTHER; note = thymine
tggtgttataccatggatcacaatg

SEQ ID NO: 156 - DNA, 25 bp, synthetic construct
  source 1..25: mol_type = other DNA; note = Synthetic
  modified_base 25: mod_base = OTHER; note = thymine
ggtgttataccatggatccaaatgt

SEQ ID NO: 157 - DNA, 25 bp, synthetic construct
  source 1..25: mol_type = other DNA; note = Synthetic
  modified_base 24: mod_base = OTHER; note = thymine
gtgttataccatggatcccaatgtc

SEQ ID NO: 158 - RNA, 25 nt, synthetic construct
  source 1..25: mol_type = other RNA; note = Synthetic
tcagatgggagtactgcaaactgac

SEQ ID NO: 159 - RNA, 25 nt, synthetic construct
  source 1..25: mol_type = other RNA; note = Synthetic
gatgggagtactgcaacctaacaca

SEQ ID NO: 160 - RNA, 25 nt, synthetic construct
  source 1..25: mol_type = other RNA; note = Synthetic
atgggagtactgcaacctgacacaa

SEQ ID NO: 161 - DNA, 25 bp, synthetic construct
  source 1..25: mol_type = other DNA; note = Synthetic
  modified_base 25: mod_base = OTHER; note = thymine
tgggagtactgcaacctgaaacaat

SEQ ID NO: 162 - DNA, 25 bp, synthetic construct
  source 1..25: mol_type = other DNA; note = Synthetic
  modified_base 24: mod_base = OTHER; note = thymine
gggagtactgcaacctgacacaatg

SEQ ID NO: 163 - RNA, 25 nt, synthetic construct
  source 1..25: mol_type = other RNA; note = Synthetic
ggctgtttctgaacaagcaacaacg

SEQ ID NO: 164 - RNA, 25 nt, synthetic construct
  source 1..25: mol_type = other RNA; note = Synthetic
gtttctgaacaagcaccaaaggagc

SEQ ID NO: 165 - RNA, 25 nt, synthetic construct
  source 1..25: mol_type = other RNA; note = Synthetic
ctccaccactgttacaggaaggaca

SEQ ID NO: 166 - RNA, 24 nt, synthetic construct
  source 1..24: mol_type = other RNA; note = Synthetic
tccaccactgttacaggaaagaca

SEQ ID NO: 167 - RNA, 24 nt, synthetic construct
  source 1..24: mol_type = other RNA; note = Synthetic
ccaccactgttacaggaagaacag

SEQ ID NO: 168 - RNA, 25 nt, synthetic construct
  source 1..25: mol_type = other RNA; note = Synthetic
gacaccacactggcatcagagaacc

SEQ ID NO: 169 - RNA, 25 nt, synthetic construct
  source 1..25: mol_type = other RNA; note = Synthetic
acaccacactggcatcagaaaacca

SEQ ID NO: 170 - RNA, 24 nt, synthetic construct
  source 1..24: mol_type = other RNA; note = Synthetic
agaatactacccaaatggtagccg

SEQ ID NO: 171 - RNA, 25 nt, synthetic construct
  source 1..25: mol_type = other RNA; note = Synthetic
gaatactacccaaatggtgacctga

SEQ ID NO: 172 - RNA, 25 nt, synthetic construct
  source 1..25: mol_type = other RNA; note = Synthetic
aatactacccaaatggtggactgac

SEQ ID NO: 173 - RNA, 25 nt, synthetic construct
  source 1..25: mol_type = other RNA; note = Synthetic
tccttctgaagaagcaccaactgaa

SEQ ID NO: 174 - RNA, 25 nt, synthetic construct
  source 1..25: mol_type = other RNA; note = Synthetic
ccttctgaagaagcaccaaatgaaa

SEQ ID NO: 175 - RNA, 25 nt, synthetic construct
  source 1..25: mol_type = other RNA; note = Synthetic
cttctgaagaagcaccaacagaaaa

SEQ ID NO: 176 - RNA, 25 nt, synthetic construct
  source 1..25: mol_type = other RNA; note = Synthetic
ttctgaagaagcaccaactaaaaac

SEQ ID NO: 177 - RNA, 25 nt, synthetic construct
  source 1..25: mol_type = other RNA; note = Synthetic
tctgaagaagcaccaactgaaaaca

SEQ ID NO: 178 - RNA, 25 nt, synthetic construct
  source 1..25: mol_type = other RNA; note = Synthetic
ctgaagaagcaccaactgaaaacag

SEQ ID NO: 179 - RNA, 25 nt, synthetic construct
  source 1..25: mol_type = other RNA; note = Synthetic
tgaagaagcaccaactgaaaacagc

SEQ ID NO: 180 - RNA, 25 nt, synthetic construct
  source 1..25: mol_type = other RNA; note = Synthetic
gaagaagcaccaactgaaaacagca

SEQ ID NO: 181 - RNA, 25 nt, synthetic construct
  source 1..25: mol_type = other RNA; note = Synthetic
aagaagcaccaactgaaaaaagcac

SEQ ID NO: 182 - RNA, 24 nt, synthetic construct
  source 1..24: mol_type = other RNA; note = Synthetic
agaagcaccaactgaaaacagcac

SEQ ID NO: 183 - RNA, 24 nt, synthetic construct
  source 1..24: mol_type = other RNA; note = Synthetic
gaagcaccaactgaaaacaacacg

SEQ ID NO: 184 - RNA, 25 nt, synthetic construct
  source 1..25: mol_type = other RNA; note = Synthetic
aagcaccaactgaaaacagaactgg

SEQ ID NO: 185 - RNA, 25 nt, synthetic construct
  source 1..25: mol_type = other RNA; note = Synthetic
aggtgatggacagagttatagaggc

SEQ ID NO: 186 - RNA, 25 nt, synthetic construct
  source 1..25: mol_type = other RNA; note = Synthetic
ctccaccactatcacaggaagaaca

SEQ ID NO: 187 - RNA, 24 nt, synthetic construct
  source 1..24: mol_type = other RNA; note = Synthetic
tccaccactatcacaggaaaaaca

SEQ ID NO: 188 - RNA, 24 nt, synthetic construct
  source 1..24: mol_type = other RNA; note = Synthetic
ccaccactatcacaggaagaacag

SEQ ID NO: 189 - RNA, 24 nt, synthetic construct
  source 1..24: mol_type = other RNA; note = Synthetic
caccactatcacaggaagaacatg

SEQ ID NO: 190 - RNA, 24 nt, synthetic construct
  source 1..24: mol_type = other RNA; note = Synthetic
accactatcacaggaagaaaatgc

SEQ ID NO: 191 - RNA, 25 nt, synthetic construct
  source 1..25: mol_type = other RNA; note = Synthetic
ccactatcacaggaagaacatgtca

SEQ ID NO: 192 - RNA, 25 nt, synthetic construct
  source 1..25: mol_type = other RNA; note = Synthetic
cactatcacaggaagaacaagtcag

SEQ ID NO: 193 - RNA, 24 nt, synthetic construct
  source 1..24: mol_type = other RNA; note = Synthetic
actatcacaggaagaacatatcag

SEQ ID NO: 194 - RNA, 24 nt, synthetic construct
  source 1..24: mol_type = other RNA; note = Synthetic
ctatcacaggaagaacatgacagc

SEQ ID NO: 195 - RNA, 24 nt, synthetic construct
  source 1..24: mol_type = other RNA; note = Synthetic
tatcacaggaagaacatgtaagtc

SEQ ID NO: 196 - RNA, 23 nt, synthetic construct
  source 1..23: mol_type = other RNA; note = Synthetic
atcacaggaagaacatgtcagtc

SEQ ID NO: 197 - RNA, 24 nt, synthetic construct
  source 1..24: mol_type = other RNA; note = Synthetic
tcacaggaagaacatgtcaatctg

SEQ ID NO: 198 - RNA, 25 nt, synthetic construct
  source 1..25: mol_type = other RNA; note = Synthetic
cacaggaagaacatgtcagacttgg

SEQ ID NO: 199 - RNA, 24 nt, synthetic construct
  source 1..24: mol_type = other RNA; note = Synthetic
acaggaagaacatgtcagtattgg

SEQ ID NO: 200 - RNA, 24 nt, synthetic construct
  source 1..24: mol_type = other RNA; note = Synthetic
ggaagaacatgtcagtcttagtcg

SEQ ID NO: 201 - RNA, 24 nt, synthetic construct
  source 1..24: mol_type = other RNA; note = Synthetic
gaagaacatgtcagtcttgatcgc

SEQ ID NO: 202 - RNA, 24 nt, synthetic construct
  source 1..24: mol_type = other RNA; note = Synthetic
aagaacatgtcagtcttggacgtc

SEQ ID NO: 203 - RNA, 24 nt, synthetic construct
  source 1..24: mol_type = other RNA; note = Synthetic
agaacatgtcagtcttggtagtca

SEQ ID NO: 204 - RNA, 24 nt, synthetic construct
  source 1..24: mol_type = other RNA; note = Synthetic
ggcatcggaggatcccattataca

SEQ ID NO: 205 - RNA, 25 nt, synthetic construct
  source 1..25: mol_type = other RNA; note = Synthetic
actatccaaatgctggcctaaccag

SEQ ID NO: 206 - RNA, 25 nt, synthetic construct
  source 1..25: mol_type = other RNA; note = Synthetic
gcacagaggctccttctgaacaagc

SEQ ID NO: 207 - RNA, 25 nt, synthetic construct
  source 1..25: mol_type = other RNA; note = Synthetic
tccttctgaacaagcaccaactgag

SEQ ID NO: 208 - RNA, 25 nt, synthetic construct
  source 1..25: mol_type = other RNA; note = Synthetic
ccttctgaacaagcaccacatgaga

SEQ ID NO: 209 - RNA, 25 nt, synthetic construct
  source 1..25: mol_type = other RNA; note = Synthetic
cttctgaacaagcaccaccagagaa

SEQ ID NO: 210 - RNA, 25 nt, synthetic construct
  source 1..25: mol_type = other RNA; note = Synthetic
ttctgaacaagcaccacctaagaaa

SEQ ID NO: 211 - RNA, 25 nt, synthetic construct
  source 1..25: mol_type = other RNA; note = Synthetic
tctgaacaagcaccacctgagaaaa

SEQ ID NO: 212 - RNA, 25 nt, synthetic construct
  source 1..25: mol_type = other RNA; note = Synthetic
ctgaacaagcaccacctgaaaaaag

SEQ ID NO: 213 - RNA, 25 nt, synthetic construct
  source 1..25: mol_type = other RNA; note = Synthetic
tgaacaagcaccacctgagaaaagc

SEQ ID NO: 214 - RNA, 25 nt, synthetic construct
  source 1..25: mol_type = other RNA; note = Synthetic
gaacaagcaccacctgagaaaagcc

SEQ ID NO: 215 - RNA, 25 nt, synthetic construct
  source 1..25: mol_type = other RNA; note = Synthetic
aacaagcaccacctgagaaaagccc

SEQ ID NO: 216 - RNA, 24 nt, synthetic construct
  source 1..24: mol_type = other RNA; note = Synthetic
caagcaccacctgagaaaaacccg

SEQ ID NO: 217 - RNA, 24 nt, synthetic construct
  source 1..24: mol_type = other RNA; note = Synthetic
aagcaccacctgagaaaagacctg

SEQ ID NO: 218 - RNA, 24 nt, synthetic construct
  source 1..24: mol_type = other RNA; note = Synthetic
agcaccacctgagaaaagcactgg

SEQ ID NO: 219 - RNA, 25 nt, synthetic construct
  source 1..25: mol_type = other RNA; note = Synthetic
ctgagaaaagccctgtggtacagga

SEQ ID NO: 220 - RNA, 25 nt, synthetic construct
  source 1..25: mol_type = other RNA; note = Synthetic
gccctgtggtccaggattgatacca

SEQ ID NO: 221 - RNA, 25 nt, synthetic construct
  source 1..25: mol_type = other RNA; note = Synthetic
ctgtggtccaggattgctaacatgg

SEQ ID NO: 222 - RNA, 25 nt, synthetic construct
  source 1..25: mol_type = other RNA; note = Synthetic
ctccaccactgtcacaggaaggacc

SEQ ID NO: 223 - RNA, 24 nt, synthetic construct
  source 1..24: mol_type = other RNA; note = Synthetic
tccaccactgtcacaggaaagacc

SEQ ID NO: 224 - RNA, 24 nt, synthetic construct
  source 1..24: mol_type = other RNA; note = Synthetic
tcttggtcatctatgatacaacac

SEQ ID NO: 225 - RNA, 24 nt, synthetic construct
  source 1..24: mol_type = other RNA; note = Synthetic
cttggtcatctatgataccacacg

SEQ ID NO: 226 - RNA, 25 nt, synthetic construct
  source 1..25: mol_type = other RNA; note = Synthetic
ttggtcatctatgataccaaactgg

SEQ ID NO: 227 - RNA, 25 nt, synthetic construct
  source 1..25: mol_type = other RNA; note = Synthetic
tggtcatctatgataccacactggc

SEQ ID NO: 228 - RNA, 25 nt, synthetic construct
  source 1..25: mol_type = other RNA; note = Synthetic
ggtcatctatgataccacaatggca

SEQ ID NO: 229 - RNA, 24 nt, synthetic construct
  source 1..24: mol_type = other RNA; note = Synthetic
gtcatctatgataccacacaggca

SEQ ID NO: 230 - RNA, 24 nt, synthetic construct
  source 1..24: mol_type = other RNA; note = Synthetic
tcatctatgataccacactagcac

SEQ ID NO: 231 - RNA, 25 nt, synthetic construct
  source 1..25: mol_type = other RNA; note = Synthetic
catctatgataccacactgacatca

SEQ ID NO: 232 - RNA, 25 nt, synthetic construct
  source 1..25: mol_type = other RNA; note = Synthetic
atctatgataccacactggaatcag

SEQ ID NO: 233 - RNA, 25 nt, synthetic construct
  source 1..25: mol_type = other RNA; note = Synthetic
tctatgataccacactggcatcaga

SEQ ID NO: 234 - RNA, 25 nt, synthetic construct
  source 1..25: mol_type = other RNA; note = Synthetic
ctatgataccacactggcaacagag

SEQ ID NO: 235 - RNA, 25 nt, synthetic construct
  source 1..25: mol_type = other RNA; note = Synthetic
tgataccacactggcatcaaaggac

SEQ ID NO: 236 - RNA, 25 nt, synthetic construct
  source 1..25: mol_type = other RNA; note = Synthetic
ataccacactggcatcagaagaccc

SEQ ID NO: 237 - RNA, 25 nt, synthetic construct
  source 1..25: mol_type = other RNA; note = Synthetic
agaggaccccagaaaactaaccaaa

SEQ ID NO: 238 - RNA, 24 nt, synthetic construct
  source 1..24: mol_type = other RNA; note = Synthetic
gaggaccccagaaaactacacaaa

SEQ ID NO: 239 - RNA, 24 nt, synthetic construct
  source 1..24: mol_type = other RNA; note = Synthetic
agaactactgcaggaatccagatc

SEQ ID NO: 240 - RNA, 25 nt, synthetic construct
  source 1..25: mol_type = other RNA; note = Synthetic
actactgcaggaatccagaatctgg

SEQ ID NO: 241 - RNA, 25 nt, synthetic construct
  source 1..25: mol_type = other RNA; note = Synthetic
tactgcaggaatccagattatggga

SEQ ID NO: 242 - RNA, 25 nt, synthetic construct
  source 1..25: mol_type = other RNA; note = Synthetic
actgcaggaatccagattcagggaa

SEQ ID NO: 243 - RNA, 25 nt, synthetic construct
  source 1..25: mol_type = other RNA; note = Synthetic
ctgcaggaatccagattctaggaaa

SEQ ID NO: 244 - RNA, 25 nt, synthetic construct
  source 1..25: mol_type = other RNA; note = Synthetic
tgcaggaatccagattctgagaaac

SEQ ID NO: 245 - RNA, 25 nt, synthetic construct
  source 1..25: mol_type = other RNA; note = Synthetic
gcaggaatccagattctggaaaaca

SEQ ID NO: 246 - RNA, 25 nt, synthetic construct
  source 1..25: mol_type = other RNA; note = Synthetic
ggaatccagattctgggaaacaacc

SEQ ID NO: 247 - RNA, 25 nt, synthetic construct
  source 1..25: mol_type = other RNA; note = Synthetic
gggaaacaaccctggtgttacacaa

SEQ ID NO: 248 - RNA, 25 nt, synthetic construct
  source 1..25: mol_type = other RNA; note = Synthetic
ggaaacaaccctggtgttaaacaac

SEQ ID NO: 249 - RNA, 24 nt, synthetic construct
  source 1..24: mol_type = other RNA; note = Synthetic
tgtgtgaggtgggagtactacaac

SEQ ID NO: 250 - RNA, 24 nt, synthetic construct
  source 1..24: mol_type = other RNA; note = Synthetic
gtgtgaggtgggagtactgaaatc

SEQ ID NO: 251 - RNA, 25 nt, synthetic construct
  source 1..25: mol_type = other RNA; note = Synthetic
gtgaggtgggagtactgcaatctga

SEQ ID NO: 252 - RNA, 25 nt, synthetic construct
  source 1..25: mol_type = other RNA; note = Synthetic
tgaggtgggagtactgcaaactgac

SEQ ID NO: 253 - RNA, 24 nt, synthetic construct
  source 1..24: mol_type = other RNA; note = Synthetic
ctgacacaatgctcagaaaaagaa

SEQ ID NO: 254 - RNA, 24 nt, synthetic construct
  source 1..24: mol_type = other RNA; note = Synthetic
tgacacaatgctcagaaacagaac

SEQ ID NO: 255 - RNA, 25 nt, synthetic construct
  source 1..25: mol_type = other RNA; note = Synthetic
gacacaatgctcagaaacaaaatca

SEQ ID NO: 256 - RNA, 25 nt, synthetic construct
  source 1..25: mol_type = other RNA; note = Synthetic
acacaatgctcagaaacagaatcag

SEQ ID NO: 257 - RNA, 25 nt, synthetic construct
  source 1..25: mol_type = other RNA; note = Synthetic
cacaatgctcagaaacagaatcagg

SEQ ID NO: 258 - RNA, 24 nt, synthetic construct
  source 1..24: mol_type = other RNA; note = Synthetic
acaatgctcagaaacagaaacagg

SEQ ID NO: 259 - RNA, 24 nt, synthetic construct
  source 1..24: mol_type = other RNA; note = Synthetic
caatgctcagaaacagaataaggg

SEQ ID NO: 260 - RNA, 24 nt, synthetic construct
  source 1..24: mol_type = other RNA; note = Synthetic
aatgctcagaaacagaatcaggtg

SEQ ID NO: 261 - RNA, 24 nt, synthetic construct
  source 1..24: mol_type = other RNA; note = Synthetic
atgctcagaaacagaatcaagtgc

SEQ ID NO: 262 - RNA, 25 nt, synthetic construct
  source 1..25: mol_type = other RNA; note = Synthetic
tgctcagaaacagaatcagatgtcc

SEQ ID NO: 263 - RNA, 24 nt, synthetic construct
  source 1..24: mol_type = other RNA; note = Synthetic
ctcagaaacagaatcaggtatcca

SEQ ID NO: 264 - RNA, 25 nt, synthetic construct
  source 1..25: mol_type = other RNA; note = Synthetic
cagaaacagaatcaggtgtactaga

SEQ ID NO: 265 - RNA, 25 nt, synthetic construct
  source 1..25: mol_type = other RNA; note = Synthetic
agaaacagaatcaggtgtcatagag

SEQ ID NO: 266 - RNA, 25 nt, synthetic construct
  source 1..25: mol_type = other RNA; note = Synthetic
gaaacagaatcaggtgtccaagaga

SEQ ID NO: 267 - RNA, 25 nt, synthetic construct
  source 1..25: mol_type = other RNA; note = Synthetic
aaacagaatcaggtgtcctagagac

SEQ ID NO: 268 - RNA, 24 nt, synthetic construct
  source 1..24: mol_type = other RNA; note = Synthetic
aacagaatcaggtgtcctaaagac

SEQ ID NO: 269 - RNA, 25 nt, synthetic construct
  source 1..25: mol_type = other RNA; note = Synthetic
cagaatcaggtgtcctagaaactcc

SEQ ID NO: 270 - RNA, 25 nt, synthetic construct
  source 1..25: mol_type = other RNA; note = Synthetic
gaatcaggtgtcctagagaatccca

SEQ ID NO: 271 - RNA, 24 nt, synthetic construct
  source 1..24: mol_type = other RNA; note = Synthetic
atcaggtgtcctagagactaccac

SEQ ID NO: 272 - RNA, 24 nt, synthetic construct
  source 1..24: mol_type = other RNA; note = Synthetic
ggtgtcctagagactcccaatgtg

SEQ ID NO: 273 - RNA, 25 nt, synthetic construct
  source 1..25: mol_type = other RNA; note = Synthetic
cctagagactcccactgttattcca

SEQ ID NO: 274 - RNA, 25 nt, synthetic construct
  source 1..25: mol_type = other RNA; note = Synthetic
ctagagactcccactgttgatccag

SEQ ID NO: 275 - RNA, 24 nt, synthetic construct
  source 1..24: mol_type = other RNA; note = Synthetic
tagagactcccactgttgtaccag

SEQ ID NO: 276 - RNA, 23 nt, synthetic construct
  source 1..23: mol_type = other RNA; note = Synthetic
agagactcccactgttgttacag

SEQ ID NO: 277 - RNA, 24 nt, synthetic construct
  source 1..24: mol_type = other RNA; note = Synthetic
gagactcccactgttgttcaagtc

SEQ ID NO: 278 - RNA, 25 nt, synthetic construct
  source 1..25: mol_type = other RNA; note = Synthetic
agactcccactgttgttccagttcc

SEQ ID NO: 279 - RNA, 24 nt, synthetic construct
  source 1..24: mol_type = other RNA; note = Synthetic
gctcattctgaagcagcacaaacg

SEQ ID NO: 280 - RNA, 25 nt, synthetic construct
  source 1..25: mol_type = other RNA; note = Synthetic
ctcattctgaagcagcaccaactga

SEQ ID NO: 281 - RNA, 25 nt, synthetic construct
  source 1..25: mol_type = other RNA; note = Synthetic
tcattctgaagcagcaccaactgag

SEQ ID NO: 282 - RNA, 25 nt, synthetic construct
  source 1..25: mol_type = other RNA; note = Synthetic
cattctgaagcagcaccaaatgagc

SEQ ID NO: 283 - RNA, 25 nt, synthetic construct
  source 1..25: mol_type = other RNA; note = Synthetic
attctgaagcagcaccaacagagca

SEQ ID NO: 284 - RNA, 25 nt, synthetic construct
  source 1..25: mol_type = other RNA; note = Synthetic
ttctgaagcagcaccaactaagcaa

SEQ ID NO: 285 - RNA, 25 nt, synthetic construct
  source 1..25: mol_type = other RNA; note = Synthetic
tctgaagcagcaccaactgagcaaa

SEQ ID NO: 286 - RNA, 25 nt, synthetic construct
  source 1..25: mol_type = other RNA; note = Synthetic
ctgaagcagcaccaactgaacaaac

SEQ ID NO: 287 - RNA, 25 nt, synthetic construct
  source 1..25: mol_type = other RNA; note = Synthetic
tgaagcagcaccaactgagaaaacc

SEQ ID NO: 288 - RNA, 25 nt, synthetic construct
  source 1..25: mol_type = other RNA; note = Synthetic
gaagcagcaccaactgagcaaaccc

SEQ ID NO: 289 - RNA, 25 nt, synthetic construct
  source 1..25: mol_type = other RNA; note = Synthetic
aagcagcaccaactgagcaaacccc

SEQ ID NO: 290 - RNA, 25 nt, synthetic construct
  source 1..25: mol_type = other RNA; note = Synthetic
cagtgctaccatggtaatgaccaga

SEQ ID NO: 291 - RNA, 25 nt, synthetic construct
  source 1..25: mol_type = other RNA; note = Synthetic
agtgctaccatggtaatggacagag

SEQ ID NO: 292 - RNA, 25 nt, synthetic construct
  source 1..25: mol_type = other RNA; note = Synthetic
acattctccaccactgtcaaaggaa

SEQ ID NO: 293 - RNA, 25 nt, synthetic construct
  source 1..25: mol_type = other RNA; note = Synthetic
cactgtcacaggaaggacaagtcaa

SEQ ID NO: 294 - RNA, 24 nt, synthetic construct
  source 1..24: mol_type = other RNA; note = Synthetic
actgtcacaggaaggacatatcaa

SEQ ID NO: 295 - RNA, 24 nt, synthetic construct
  source 1..24: mol_type = other RNA; note = Synthetic
ctgtcacaggaaggacatgacaac

SEQ ID NO: 296 - RNA, 24 nt, synthetic construct
  source 1..24: mol_type = other RNA; note = Synthetic
tgtcacaggaaggacatgtaaatc

SEQ ID NO: 297 - RNA, 23 nt, synthetic construct
  source 1..23: mol_type = other RNA; note = Synthetic
gtcacaggaaggacatgtcaatc

SEQ ID NO: 298 - RNA, 24 nt, synthetic construct
  source 1..24: mol_type = other RNA; note = Synthetic
tcacaggaaggacatgtcaatctg

SEQ ID NO: 299 - RNA, 24 nt, synthetic construct
  source 1..24: mol_type = other RNA; note = Synthetic
acaggaaggacatgtcaatattgg

SEQ ID NO: 300 - RNA, 24 nt, synthetic construct
  source 1..24: mol_type = other RNA; note = Synthetic
caggaaggacatgtcaatcatggc

SEQ ID NO: 301 - RNA, 25 nt, synthetic construct
  source 1..25: mol_type = other RNA; note = Synthetic
aggaaggacatgtcaatctaggtca

SEQ ID NO: 302 - RNA, 24 nt, synthetic construct
  source 1..24: mol_type = other RNA; note = Synthetic
ggaaggacatgtcaatcttagtca

SEQ ID NO: 303 - RNA, 24 nt, synthetic construct
  source 1..24: mol_type = other RNA; note = Synthetic
gaaggacatgtcaatcttgatcac

SEQ ID NO: 304 - RNA, 25 nt, synthetic construct
  source 1..25: mol_type = other RNA; note = Synthetic
aaggacatgtcaatcttggacatcc

SEQ ID NO: 305 - RNA, 25 nt, synthetic construct
  source 1..25: mol_type = other RNA; note = Synthetic
aggacatgtcaatcttggtaatcca

SEQ ID NO: 306 - RNA, 24 nt, synthetic construct
  source 1..24: mol_type = other RNA; note = Synthetic
ggacatgtcaatcttggtcatcca

SEQ ID NO: 307 - RNA, 24 nt, synthetic construct
  source 1..24: mol_type = other RNA; note = Synthetic
gacatgtcaatcttggtcaaccag

SEQ ID NO: 308 - RNA, 25 nt, synthetic construct
  source 1..25: mol_type = other RNA; note = Synthetic
acatgtcaatcttggtcatacatga

SEQ ID NO: 309 - RNA, 25 nt, synthetic construct
  source 1..25: mol_type = other RNA; note = Synthetic
catgtcaatcttggtcatcaatgac

SEQ ID NO: 310 - RNA, 25 nt, synthetic construct
  source 1..25: mol_type = other RNA; note = Synthetic
atgtcaatcttggtcatccatgaca

SEQ ID NO: 311 - RNA, 25 nt, synthetic construct
  source 1..25: mol_type = other RNA; note = Synthetic
tgtcaatcttggtcatccaagacac

SEQ ID NO: 312 - RNA, 25 nt, synthetic construct
  source 1..25: mol_type = other RNA; note = Synthetic
gtcaatcttggtcatccataacacc

SEQ ID NO: 313 - RNA, 25 nt, synthetic construct
  source 1..25: mol_type = other RNA; note = Synthetic
tcaatcttggtcatccatgacacca

SEQ ID NO: 314 - RNA, 25 nt, synthetic construct
  source 1..25: mol_type = other RNA; note = Synthetic
caatcttggtcatccatgaaaccac

SEQ ID NO: 315 - RNA, 25 nt, synthetic construct
  source 1..25: mol_type = other RNA; note = Synthetic
aatcttggtcatccatgacaccaca

SEQ ID NO: 316 - RNA, 25 nt, synthetic construct
  source 1..25: mol_type = other RNA; note = Synthetic
atcttggtcatccatgacaacacac

SEQ ID NO: 317 - RNA, 25 nt, synthetic construct
  source 1..25: mol_type = other RNA; note = Synthetic
tgacaatgaactactgcagaaatcc

SEQ ID NO: 318 - RNA, 25 nt, synthetic construct
  source 1..25: mol_type = other RNA; note = Synthetic
gacaatgaactactgcaggaatcca

SEQ ID NO: 319 - RNA, 25 nt, synthetic construct
  source 1..25: mol_type = other RNA; note = Synthetic
acaatgaactactgcaggaatccag

SEQ ID NO: 320 - RNA, 25 nt, synthetic construct
  source 1..25: mol_type = other RNA; note = Synthetic
caatgaactactgcaggaaaccaga

SEQ ID NO: 321 - RNA, 24 nt, synthetic construct
  source 1..24: mol_type = other RNA; note = Synthetic
aatgaactactgcaggaatacaga

SEQ ID NO: 322 - RNA, 24 nt, synthetic construct
  source 1..24: mol_type = other RNA; note = Synthetic
atgaactactgcaggaatcaagag

SEQ ID NO: 323 - RNA, 25 nt, synthetic construct
  source 1..25: mol_type = other RNA; note = Synthetic
tgaactactgcaggaatccagatgc

SEQ ID NO: 324 - RNA, 24 nt, synthetic construct
  source 1..24: mol_type = other RNA; note = Synthetic
ctactgcaggaatccagataccga

SEQ ID NO: 325 - RNA, 25 nt, synthetic construct
  source 1..25: mol_type = other RNA; note = Synthetic
caggcccttggtgttttacaatgga

SEQ ID NO: 326 - RNA, 25 nt, synthetic construct
  source 1..25: mol_type = other RNA; note = Synthetic
cttggtgttttaccatggaacccag

SEQ ID NO: 327 - RNA, 25 nt, synthetic construct
  source 1..25: mol_type = other RNA; note = Synthetic
ttggtgttttaccatggacaccagc

SEQ ID NO: 328 - RNA, 25 nt, synthetic construct
  source 1..25: mol_type = other RNA; note = Synthetic
tggtgttttaccatggaccacagca

SEQ ID NO: 329 - RNA, 24 nt, synthetic construct
  source 1..24: mol_type = other RNA; note = Synthetic
ggtgttttaccatggacccaagca

SEQ ID NO: 330 - RNA, 24 nt, synthetic construct
  source 1..24: mol_type = other RNA; note = Synthetic
gtgttttaccatggaccccagcac

SEQ ID NO: 331 - RNA, 25 nt, synthetic construct
  source 1..25: mol_type = other RNA; note = Synthetic
gttttaccatggaccccagaatcag

SEQ ID NO: 332 - RNA, 25 nt, synthetic construct
  source 1..25: mol_type = other RNA; note = Synthetic
ggagtactgcaacctgacgagatgc

SEQ ID NO: 333 - RNA, 24 nt, synthetic construct
  source 1..24: mol_type = other RNA; note = Synthetic
gagtactgcaacctgacgcaatgc

SEQ ID NO: 334 - RNA, 25 nt, synthetic construct
  source 1..25: mol_type = other RNA; note = Synthetic
gtactgcaacctgacgcgaagctca

SEQ ID NO: 335 - RNA, 25 nt, synthetic construct
  source 1..25: mol_type = other RNA; note = Synthetic
tactgcaacctgacgcgatactcag

SEQ ID NO: 336 - RNA, 25 nt, synthetic construct
  source 1..25: mol_type = other RNA; note = Synthetic
tgcaacctgacgcgatgctaagaca

SEQ ID NO: 337 - RNA, 25 nt, synthetic construct
  source 1..25: mol_type = other RNA; note = Synthetic
cctgacgcgatgctcagacacagaa

SEQ ID NO: 338 - RNA, 25 nt, synthetic construct
  source 1..25: mol_type = other RNA; note = Synthetic
ctgacgcgatgctcagacaaagaag

SEQ ID NO: 339 - RNA, 24 nt, synthetic construct
  source 1..24: mol_type = other RNA; note = Synthetic
gatgctcagacacagaaggaactg

SEQ ID NO: 340 - RNA, 24 nt, synthetic construct
  source 1..24: mol_type = other RNA; note = Synthetic
atgctcagacacagaagggactgg

SEQ ID NO: 341 - RNA, 24 nt, synthetic construct
  source 1..24: mol_type = other RNA; note = Synthetic
agacacagaagggactgtgatcgc

SEQ ID NO: 342 - RNA, 25 nt, synthetic construct
  source 1..25: mol_type = other RNA; note = Synthetic
gcatcctcttcatttgattatggga

SEQ ID NO: 343 - RNA, 25 nt, synthetic construct
  source 1..25: mol_type = other RNA; note = Synthetic
catcctcttcatttgattgagggaa

SEQ ID NO: 344 - RNA, 25 nt, synthetic construct
  source 1..25: mol_type = other RNA; note = Synthetic
atcctcttcatttgattgtaggaag

SEQ ID NO: 345 - RNA, 25 nt, synthetic construct
  source 1..25: mol_type = other RNA; note = Synthetic
tcctcttcatttgattgtgagaagc

SEQ ID NO: 346 - RNA, 25 nt, synthetic construct
  source 1..25: mol_type = other RNA; note = Synthetic
cctcttcatttgattgtggaaagcc

SEQ ID NO: 347 - RNA, 25 nt, synthetic construct
  source 1..25: mol_type = other RNA; note = Synthetic
cttcatttgattgtgggaaacctca

SEQ ID NO: 348 - RNA, 25 nt, synthetic construct
  source 1..25: mol_type = other RNA; note = Synthetic
ttcatttgattgtgggaagactcaa

SEQ ID NO: 349 - RNA, 25 nt, synthetic construct
  source 1..25: mol_type = other RNA; note = Synthetic
tcatttgattgtgggaagcatcaag

SEQ ID NO: 350 - RNA, 24 nt, synthetic construct
  source 1..24: mol_type = other RNA; note = Synthetic
catttgattgtgggaagccacaag

SEQ ID NO: 351 - RNA, 24 nt, synthetic construct
  source 1..24: mol_type = other RNA; note = Synthetic
atttgattgtgggaagcctaaagg

SEQ ID NO: 352 - RNA, 25 nt, synthetic construct
  source 1..25: mol_type = other RNA; note = Synthetic
gtgggaagcctcaagtggaaccgaa

SEQ ID NO: 353 - RNA, 25 nt, synthetic construct
  source 1..25: mol_type = other RNA; note = Synthetic
aagaaatgtcctggaagcaatgtag

SEQ ID NO: 354 - RNA, 25 nt, synthetic construct
  source 1..25: mol_type = other RNA; note = Synthetic
agaaatgtcctggaagcatagtagg

SEQ ID NO: 355 - RNA, 25 nt, synthetic construct
  source 1..25: mol_type = other RNA; note = Synthetic
gaaatgtcctggaagcattataggg

SEQ ID NO: 356 - RNA, 25 nt, synthetic construct
  source 1..25: mol_type = other RNA; note = Synthetic
aatgtcctggaagcattgtaggggg

SEQ ID NO: 357 - RNA, 25 nt, synthetic construct
  source 1..25: mol_type = other RNA; note = Synthetic
atgtcctggaagcattgtaaggggg

SEQ ID NO: 358 - RNA, 24 nt, synthetic construct
  source 1..24: mol_type = other RNA; note = Synthetic
agaacaaggtttggaaagcacttc

SEQ ID NO: 359 - RNA, 24 nt, synthetic construct
  source 1..24: mol_type = other RNA; note = Synthetic
gaacaaggtttggaaagcaattcg

SEQ ID NO: 360 - RNA, 24 nt, synthetic construct
  source 1..24: mol_type = other RNA; note = Synthetic
aacaaggtttggaaagcacatctg

SEQ ID NO: 361 - RNA, 24 nt, synthetic construct
  source 1..24: mol_type = other RNA; note = Synthetic
acaaggtttggaaagcactactgg

SEQ ID NO: 362 - RNA, 25 nt, synthetic construct
  source 1..25: mol_type = other RNA; note = Synthetic
caaggtttggaaagcacttatgtgg

SEQ ID NO: 363 - RNA, 25 nt, synthetic construct
  source 1..25: mol_type = other RNA; note = Synthetic
aaggtttggaaagcacttcagtgga

SEQ ID NO: 364 - RNA, 25 nt, synthetic construct
  source 1..25: mol_type = other RNA; note = Synthetic
aggtttggaaagcacttctatggag

SEQ ID NO: 365 - RNA, 25 nt, synthetic construct
  source 1..25: mol_type = other RNA; note = Synthetic
tggaaagcacttctgtggaagcacc

SEQ ID NO: 366 - RNA, 25 nt, synthetic construct
  source 1..25: mol_type = other RNA; note = Synthetic
gtggaggcaccttaatatcaccaga

SEQ ID NO: 367 - RNA, 24 nt, synthetic construct
  source 1..24: mol_type = other RNA; note = Synthetic
cttaatatccccagagtggatgcg

SEQ ID NO: 368 - RNA, 25 nt, synthetic construct
  source 1..25: mol_type = other RNA; note = Synthetic
taatatccccagagtgggtactgac

SEQ ID NO: 369 - RNA, 25 nt, synthetic construct
  source 1..25: mol_type = other RNA; note = Synthetic
agagtgggtgctgactgctactcac

SEQ ID NO: 370 - RNA, 25 nt, synthetic construct
  source 1..25: mol_type = other RNA; note = Synthetic
caaggtcatcctgggtgcaaaccaa

SEQ ID NO: 371 - RNA, 25 nt, synthetic construct
  source 1..25: mol_type = other RNA; note = Synthetic
cctgggtgcacaccaagaaatgaac

SEQ ID NO: 372 - RNA, 25 nt, synthetic construct
  source 1..25: mol_type = other RNA; note = Synthetic
gtgcacaccaagaagtgaaactcga

SEQ ID NO: 373 - RNA, 24 nt, synthetic construct
  source 1..24: mol_type = other RNA; note = Synthetic
agcagatattgccttgctaaagca

SEQ ID NO: 374 - RNA, 25 nt, synthetic construct
  source 1..25: mol_type = other RNA; note = Synthetic
gcagatattgccttgctaaagctaa

SEQ ID NO: 375 - RNA, 25 nt, synthetic construct
  source 1..25: mol_type = other RNA; note = Synthetic
cagatattgccttgctaaaactaag

SEQ ID NO: 376 - RNA, 25 nt, synthetic construct
  source 1..25: mol_type = other RNA; note = Synthetic
agatattgccttgctaaagataagc

SEQ ID NO: 377 - RNA, 25 nt, synthetic construct
  source 1..25: mol_type = other RNA; note = Synthetic
gatattgccttgctaaagcaaagca

SEQ ID NO: 378 - RNA, 25 nt, synthetic construct
  source 1..25: mol_type = other RNA; note = Synthetic
atattgccttgctaaagctaagcag

SEQ ID NO: 379 - RNA, 25 nt, synthetic construct
  source 1..25: mol_type = other RNA; note = Synthetic
tattgccttgctaaagctaagcagg

SEQ ID NO: 380 - RNA, 25 nt, synthetic construct
  source 1..25: mol_type = other RNA; note = Synthetic
tcatcactgacaaagtaataccagc

SEQ ID NO: 381 - RNA, 24 nt, synthetic construct
  source 1..24: mol_type = other RNA; note = Synthetic
ggactgaatgttacatcacaggcg

SEQ ID NO: 382 - RNA, 25 nt, synthetic construct
  source 1..25: mol_type = other RNA; note = Synthetic
gactgaatgttacatcactagctgg

SEQ ID NO: 383 - RNA, 25 nt, synthetic construct
  source 1..25: mol_type = other RNA; note = Synthetic
actgaatgttacatcactgactggg

SEQ ID NO: 384 - RNA, 25 nt, synthetic construct
  source 1..25: mol_type = other RNA; note = Synthetic
ctgaatgttacatcactggatgggg

SEQ ID NO: 385 - RNA, 25 nt, synthetic construct
  source 1..25: mol_type = other RNA; note = Synthetic
tgaatgttacatcactggcagggga

SEQ ID NO: 386 - RNA, 25 nt, synthetic construct
  source 1..25: mol_type = other RNA; note = Synthetic
aatgttacatcactggctgaggaga

SEQ ID NO: 387 - RNA, 24 nt, synthetic construct
  source 1..24: mol_type = other RNA; note = Synthetic
gaaacccaaggtacctttgagacg

SEQ ID NO: 388 - RNA, 36 nt, synthetic construct
  source 1..36: mol_type = other RNA; note = Synthetic
tccaccactgtcacaggaaagcagccgaaaggctgc

SEQ ID NO: 389 - RNA, 36 nt, synthetic construct
  source 1..36: mol_type = other RNA; note = Synthetic
tggtaatggacagagttatagcagccgaaaggctgc

SEQ ID NO: 390 - RNA, 36 nt, synthetic construct
  source 1..36: mol_type = other RNA; note = Synthetic
tactgcaacctgacacgatagcagccgaaaggctgc

SEQ ID NO: 391 - RNA, 36 nt, synthetic construct
  source 1..36: mol_type = other RNA; note = Synthetic
agaacttgccaagcttggtagcagccgaaaggctgc

SEQ ID NO: 392 - RNA, 36 nt, synthetic construct
  source 1..36: mol_type = other RNA; note = Synthetic
aacttgccaagcttggtcaagcagccgaaaggctgc

SEQ ID NO: 393 - RNA, 36 nt, synthetic construct
  source 1..36: mol_type = other RNA; note = Synthetic
ttgccaagcttggtcatctagcagccgaaaggctgc

SEQ ID NO: 394 - RNA, 36 nt, synthetic construct
  source 1..36: mol_type = other RNA; note = Synthetic
atggacagagttatcgaggagcagccgaaaggctgc

SEQ ID NO: 395 - RNA, 36 nt, synthetic construct
  source 1..36: mol_type = other RNA; note = Synthetic
tggacagagttatcgaggcagcagccgaaaggctgc

SEQ ID NO: 396 - RNA, 36 nt, synthetic construct
  source 1..36: mol_type = other RNA; note = Synthetic
tggtcatctatgataccacagcagccgaaaggctgc

SEQ ID NO: 397 - RNA, 36 nt, synthetic construct
  source 1..36: mol_type = other RNA; note = Synthetic
tactgcaggaatccagattagcagccgaaaggctgc

SEQ ID NO: 398 - RNA, 36 nt, synthetic construct
  source 1..36: mol_type = other RNA; note = Synthetic
agaaatgtcctggaagcatagcagccgaaaggctgc

SEQ ID NO: 399 - RNA, 36 nt, synthetic construct
  source 1..36: mol_type = other RNA; note = Synthetic
gacaacagaatattatccaagcagccgaaaggctgc

SEQ ID NO: 400 - RNA, 36 nt, synthetic construct
  source 1..36: mol_type = other RNA; note = Synthetic
atggacagagttatcaaggagcagccgaaaggctgc

SEQ ID NO: 401 - RNA, 36 nt, synthetic construct; identical to SEQ ID NO: 399
  source 1..36: mol_type = other RNA; note = Synthetic

SEQ ID NO: 402 - RNA, 36 nt, synthetic construct; identical to SEQ ID NO: 400
  source 1..36: mol_type = other RNA; note = Synthetic

SEQ ID NO: 403 - RNA, 36 nt, synthetic construct; identical to SEQ ID NO: 399
  source 1..36: mol_type = other RNA; note = Synthetic

SEQ ID NO: 404 - RNA, 27 nt, synthetic construct
  source 1..27: mol_type = other RNA; note = Synthetic
tcctgtaccacatggctttgctcaggt

SEQ ID NO: 405 - RNA, 27 nt, synthetic construct
  source 1..27: mol_type = other RNA; note = Synthetic
caatcttggaccacatggctttgctca

SEQ ID NO: 406 - RNA, 27 nt, synthetic construct
  source 1..27: mol_type = other RNA; note = Synthetic
gtagctatcctggaccacatggctttg

SEQ ID NO: 407 - RNA, 27 nt, synthetic construct
  source 1..27: mol_type = other RNA; note = Synthetic
ggtagtaatcctggaccacatggcttt

SEQ ID NO: 408 - RNA, 27 nt, synthetic construct
  source 1..27: mol_type = other RNA; note = Synthetic
tggtatcaatcctggaccacatggctt

SEQ ID NO: 409 - RNA, 27 nt, synthetic construct
  source 1..27: mol_type = other RNA; note = Synthetic
atggttgcaatcctggaccacatggct

SEQ ID NO: 410 - RNA, 27 nt, synthetic construct
  source 1..27: mol_type = other RNA; note = Synthetic
catggtagcaatcctggaccacatggc

SEQ ID NO: 411 - RNA, 27 nt, synthetic construct
  source 1..27: mol_type = other RNA; note = Synthetic
ccatgttagcaatcctggaccacatgg

SEQ ID NO: 412 - RNA, 27 nt, synthetic construct
  source 1..27: mol_type = other RNA; note = Synthetic
accattgtagcaatcctggaccacatg

SEQ ID NO: 413 - RNA, 27 nt, synthetic construct
  source 1..27: mol_type = other RNA; note = Synthetic
tgccttgataactctgtccatcaccat

SEQ ID NO: 414 - RNA, 27 nt, synthetic construct
  source 1..27: mol_type = other RNA; note = Synthetic
aggtctttcctgtgacagtggtggagt

SEQ ID NO: 415 - RNA, 27 nt, synthetic construct
  source 1..27: mol_type = other RNA; note = Synthetic
caggttcttcctgtgacagtggtggag

SEQ ID NO: 416 - RNA, 27 nt, synthetic construct
  source 1..27: mol_type = other RNA; note = Synthetic
gcttgtcaggtccttcctgtgacagtg

SEQ ID NO: 417 - RNA, 27 nt, synthetic construct
  source 1..27: mol_type = other RNA; note = Synthetic
atgactaagcttggcaggtccttcctg

SEQ ID NO: 418 - RNA, 27 nt, synthetic construct
  source 1..27: mol_type = other RNA; note = Synthetic
gatgatcaagcttggcaggtccttcct

SEQ ID NO: 419 - RNA, 27 nt, synthetic construct
  source 1..27: mol_type = other RNA; note = Synthetic
tagattaccaagcttggcaggtccttc

SEQ ID NO: 420 - RNA, 27 nt, synthetic construct
  source 1..27: mol_type = other RNA; note = Synthetic
atagatgaccaagcttggcaggtcctt

SEQ ID NO: 421 - RNA, 27 nt, synthetic construct
  source 1..27: mol_type = other RNA; note = Synthetic
catagttgaccaagcttggcaggtcct

SEQ ID NO: 422 - RNA, 27 nt, synthetic construct
  source 1..27: mol_type = other RNA; note = Synthetic
tcatatatgaccaagcttggcaggtcc

SEQ ID NO: 423 - RNA, 27 nt, synthetic construct
  source 1..27: mol_type = other RNA; note = Synthetic
gtcattgatgaccaagcttggcaggtc

SEQ ID NO: 424 - RNA, 27 nt, synthetic construct
  source 1..27: mol_type = other RNA; note = Synthetic
atgtgttgtcatagatgaccaagcttg

SEQ ID NO: 425 - RNA, 27 nt, synthetic construct
  source 1..27: mol_type = other RNA; note = Synthetic
atgtttatgtggtgtcatagatgacca

SEQ ID NO: 426 - RNA, 27 nt, synthetic construct
  source 1..27: mol_type = other RNA; note = Synthetic
gccagtatttgggtagttttctgtggt

SEQ ID NO: 427 - RNA, 27 nt, synthetic construct
  source 1..27: mol_type = other RNA; note = Synthetic
caagctagcatttgggtagttttctgt

SEQ ID NO: 428 - RNA, 27 nt, synthetic construct
  source 1..27: mol_type = other RNA; note = Synthetic
atcaatccagcatttgggtagttttct

SEQ ID NO: 429 - RNA, 27 nt, synthetic construct
  source 1..27: mol_type = other RNA; note = Synthetic
ttcattatcaagccagcatttgggtag

SEQ ID NO: 430 - RNA, 27 nt, synthetic construct
  source 1..27: mol_type = other RNA; note = Synthetic
gttcatgatcaagccagcatttgggta

SEQ ID NO: 431 - RNA, 27 nt, synthetic construct
  source 1..27: mol_type = other RNA; note = Synthetic
agcatttggattcctgcagtagttcat

SEQ ID NO: 432 - RNA, 27 nt, synthetic construct
  source 1..27: mol_type = other RNA; note = Synthetic
ccacatcatctggattcctgcagtagt

SEQ ID NO: 433 - RNA, 27 nt, synthetic construct
  source 1..27: mol_type = other RNA; note = Synthetic
gccactgcatctggattcctgcagtag

SEQ ID NO: 434 - RNA, 27 nt, synthetic construct
  source 1..27: mol_type = other RNA; note = Synthetic
ctgcctcagcatctggattcctgcagt

SEQ ID NO: 435 - RNA, 27 nt, synthetic construct
  source 1..27: mol_type = other RNA; note = Synthetic
gcgtctggttgcagtactcccacctga

SEQ ID NO: 436 - RNA, 27 nt, synthetic construct
  source 1..27: mol_type = other RNA; note = Synthetic
gcagttccttctgcgtctgagcattgc

SEQ ID NO: 437 - RNA, 27 nt, synthetic construct
  source 1..27: mol_type = other RNA; note = Synthetic
taggcttggaaccggggtaacagtcgg

SEQ ID NO: 438 - RNA, 27 nt, synthetic construct
  source 1..27: mol_type = other RNA; note = Synthetic
ctaggtttggaaccggggtaacagtcg

SEQ ID NO: 439 - RNA, 27 nt, synthetic construct
  source 1..27: mol_type = other RNA; note = Synthetic
tctagtcttggaaccggggtaacagtc

SEQ ID NO: 440 - RNA, 27 nt, synthetic construct
  source 1..27: mol_type = other RNA; note = Synthetic
ctctatgcttggaaccggggtaacagt

SEQ ID NO: 441 - RNA, 27 nt, synthetic construct
  source 1..27: mol_type = other RNA; note = Synthetic
cctcttggcttggaaccggggtaacag

SEQ ID NO: 442 - RNA, 27 nt, synthetic construct
  source 1..27: mol_type = other RNA; note = Synthetic
gcctctaggcttggaaccggggtaaca

SEQ ID NO: 443 - RNA, 27 nt, synthetic construct
  source 1..27: mol_type = other RNA; note = Synthetic
agcctttaggcttggaaccggggtaac

SEQ ID NO: 444 - RNA, 27 nt, synthetic construct
  source 1..27: mol_type = other RNA; note = Synthetic
actctttccattaccatggtagcactc

SEQ ID NO: 445 - RNA, 27 nt, synthetic construct
  source 1..27: mol_type = other RNA; note = Synthetic
aactctgtccattaccatggtagcact

SEQ ID NO: 446 - RNA, 27 nt, synthetic construct
  source 1..27: mol_type = other RNA; note = Synthetic
taactttgtccattaccatggtagcac

SEQ ID NO: 447 - RNA, 27 nt, synthetic construct
  source 1..27: mol_type = other RNA; note = Synthetic
ataactctgtccattaccatggtagca

SEQ ID NO: 448 - RNA, 27 nt, synthetic construct
  source 1..27: mol_type = other RNA; note = Synthetic
gataattctgtccattaccatggtagc

SEQ ID NO: 449 - RNA, 27 nt, synthetic construct
  source 1..27: mol_type = other RNA; note = Synthetic
cgatatctctgtccattaccatggtag

SEQ ID NO: 450 - RNA, 27 nt, synthetic construct
  source 1..27: mol_type = other RNA; note = Synthetic
tcgattactctgtccattaccatggta

SEQ ID NO: 451 - RNA, 27 nt, synthetic construct
  source 1..27: mol_type = other RNA; note = Synthetic
ctcgataactctgtccattaccatggt

SEQ ID NO: 452 - RNA, 27 nt, synthetic construct
  source 1..27: mol_type = other RNA; note = Synthetic
cctcgttaactctgtccattaccatgg

SEQ ID NO: 453 - RNA, 27 nt, synthetic construct
  source 1..27: mol_type = other RNA; note = Synthetic
gcctctataactctgtccattaccatg

SEQ ID NO: 454 - RNA, 27 nt, synthetic construct
  source 1..27: mol_type = other RNA; note = Synthetic
tgccttgataactctgtccattaccat

SEQ ID NO: 455 - RNA, 27 nt, synthetic construct
  source 1..27: mol_type = other RNA; note = Synthetic
gtgcctcgataactctgtccattacca

SEQ ID NO: 456 - RNA, 27 nt, synthetic construct
  source 1..27: mol_type = other RNA; note = Synthetic
ctgtgtcagtggtggagtatgtgcctc

SEQ ID NO: 457 - RNA, 27 nt, synthetic construct
  source 1..27: mol_type = other RNA; note = Synthetic
gagtgtggtgtcatagatgaccaagct

SEQ ID NO: 458 - RNA, 27 nt, synthetic construct
  source 1..27: mol_type = other RNA; note = Synthetic
atgcgtgtgtggtgtcatagatgacca

SEQ ID NO: 459 - RNA, 27 nt, synthetic construct
  source 1..27: mol_type = other RNA; note = Synthetic
tatgctagtgtggtgtcatagatgacc

SEQ ID NO: 460 - RNA, 27 nt, synthetic construct
  source 1..27: mol_type = other RNA; note = Synthetic
ctatgtgagtgtggtgtcatagatgac

SEQ ID NO: 461 - RNA, 27 nt, synthetic construct
  source 1..27: mol_type = other RNA; note = Synthetic
ccagttacagtggtggagtatgtgcct

SEQ ID NO: 462 - RNA, 27 nt, synthetic construct
  source 1..27: mol_type = other RNA; note = Synthetic
atccctcgtataacaataaggggctgc

SEQ ID NO: 463 - RNA, 27 nt, synthetic construct
  source 1..27: mol_type = other RNA; note = Synthetic
gatccttcgtataacaataaggggctg

SEQ ID NO: 464 - RNA, 27 nt, synthetic construct
  source 1..27: mol_type = other RNA; note = Synthetic
gttcataaggagcctctaggcttggaa

SEQ ID NO: 465 - RNA, 27 nt, synthetic construct
  source 1..27: mol_type = other RNA; note = Synthetic
gttggtgcttgttcagaaggagcctct

SEQ ID NO: 466 - RNA, 27 nt, synthetic construct
  source 1..27: mol_type = other RNA; note = Synthetic
tatgttccttgataactctgtccattt

SEQ ID NO: 467 - RNA, 27 nt, synthetic construct
  source 1..27: mol_type = other RNA; note = Synthetic
gtatgtgccttgataactctgtccatt

SEQ ID NO: 468 - RNA, 27 nt, synthetic construct
  source 1..27: mol_type = other RNA; note = Synthetic
agtatttgccttgataactctgtccat

SEQ ID NO: 469 - RNA, 27 nt, synthetic construct
  source 1..27: mol_type = other RNA; note = Synthetic
aagtatgtgccttgataactctgtcca

SEQ ID NO: 470 - RNA, 27 nt, synthetic construct
  source 1..27: mol_type = other RNA; note = Synthetic
gaagtttgtgccttgataactctgtcc

SEQ ID NO: 471 - RNA, 27 nt, synthetic construct
  source 1..27: mol_type = other RNA; note = Synthetic
tgaagtatgtgccttgataactctgtc

SEQ ID NO: 472 - RNA, 27 nt, synthetic construct
  source 1..27: mol_type = other RNA; note = Synthetic
tcttgttcaagccagcatttgggtagt

SEQ ID NO: 473 - RNA, 27 nt, synthetic construct
  source 1..27: mol_type = other RNA; note = Synthetic
agttcttgatcaagccagcatttgggt

SEQ ID NO: 474 - RNA, 27 nt, synthetic construct
  source 1..27: mol_type = other RNA; note = Synthetic
tctggttttcggcagtagttcttgatc

SEQ ID NO: 475 - RNA, 27 nt, synthetic construct
  source 1..27: mol_type = other RNA; note = Synthetic
atctgtatttcggcagtagttcttgat

SEQ ID NO: 476 - RNA, 27 nt, synthetic construct
  source 1..27: mol_type = other RNA; note = Synthetic
gatcttgatttcggcagtagttcttga

SEQ ID NO: 477 - RNA, 27 nt, synthetic construct
  source 1..27: mol_type = other RNA; note = Synthetic
aggatttggatttcggcagtagttctt

SEQ ID NO: 478 - RNA, 27 nt, synthetic construct
  source 1..27: mol_type = other RNA; note = Synthetic
cacagtatctggatttcggcagtagtt

SEQ ID NO: 479 - RNA, 27 nt, synthetic construct
  source 1..27: mol_type = other RNA; note = Synthetic
tgtattacaccaaggggctgccacagg

SEQ ID NO: 480 - RNA, 27 nt, synthetic construct
  source 1..27: mol_type = other RNA; note = Synthetic
ttgtataacaccaaggggctgccacag

SEQ ID NO: 481 - RNA, 27 nt, synthetic construct
  source 1..27: mol_type = other RNA; note = Synthetic
gttgtttaacaccaaggggctgccaca

SEQ ID NO: 482 - RNA, 27 nt, synthetic construct
  source 1..27: mol_type = other RNA; note = Synthetic
tgttgtataacaccaaggggctgccac

SEQ ID NO: 483 - RNA, 27 nt, synthetic construct
  source 1..27: mol_type = other RNA; note = Synthetic
ctgttttataacaccaaggggctgcca

SEQ ID NO: 484 - RNA, 27 nt, synthetic construct
  source 1..27: mol_type = other RNA; note = Synthetic
tctgttgtataacaccaaggggctgcc

SEQ ID NO: 485 - RNA, 27 nt, synthetic construct
  source 1..27: mol_type = other RNA; note = Synthetic
atctgttgtataacaccaaggggctgc

SEQ ID NO: 486 - RNA, 27 nt, synthetic construct
  source 1..27: mol_type = other RNA; note = Synthetic
gatcttttgtataacaccaaggggctg

SEQ ID NO: 487 - RNA, 27 nt, synthetic construct
  source 1..27: mol_type = other RNA; note = Synthetic
ggatctgttgtataacaccaaggggct

SEQ ID NO: 488 - RNA, 27 nt, synthetic construct
  source 1..27: mol_type = other RNA; note = Synthetic
cgtgttaggttgcagtactcccacctg

SEQ ID NO: 489 - RNA, 27 nt, synthetic construct
  source 1..27: mol_type = other RNA; note = Synthetic
tcgtgtcaggttgcagtactcccacct

SEQ ID NO: 490 - RNA, 27 nt, synthetic construct
  source 1..27: mol_type = other RNA; note = Synthetic
gcatcttgtcaggttgcagtactccca

SEQ ID NO: 491 - RNA, 27 nt, synthetic construct
  source 1..27: mol_type = other RNA; note = Synthetic
agcattgtgtcaggttgcagtactccc

SEQ ID NO: 492 - RNA, 27 nt, synthetic construct
  source 1..27: mol_type = other RNA; note = Synthetic
tgagcttcgtgtcaggttgcagtactc

SEQ ID NO: 493 - RNA, 27 nt, synthetic construct
  source 1..27: mol_type = other RNA; note = Synthetic
ctgagtatcgtgtcaggttgcagtact

SEQ ID NO: 494 - RNA, 27 nt, synthetic construct
  source 1..27: mol_type = other RNA; note = Synthetic
tctgatcatcgtgtcaggttgcagtac

SEQ ID NO: 495 - RNA, 27 nt, synthetic construct
  source 1..27: mol_type = other RNA; note = Synthetic
atctgtgcatcgtgtcaggttgcagta

SEQ ID NO: 496 - RNA, 27 nt, synthetic construct
  source 1..27: mol_type = other RNA; note = Synthetic
catcttagcatcgtgtcaggttgcagt

SEQ ID NO: 497 - RNA, 27 nt, synthetic construct
  source 1..27: mol_type = other RNA; note = Synthetic
tgcatttgagcatcgtgtcaggttgca

SEQ ID NO: 498 - RNA, 27 nt, synthetic construct
  source 1..27: mol_type = other RNA; note = Synthetic
tctgcttctgagcatcgtgtcaggttg

SEQ ID NO: 499 - RNA, 27 nt, synthetic construct
  source 1..27: mol_type = other RNA; note = Synthetic
ttctgtatctgagcatcgtgtcaggtt

SEQ ID NO: 500 - RNA, 27 nt, synthetic construct
  source 1..27: mol_type = other RNA; note = Synthetic
attcttcatctgagcatcgtgtcaggt

SEQ ID NO: 501 - RNA, 27 nt, synthetic construct
  source 1..27: mol_type = other RNA; note = Synthetic
cattctgcatctgagcatcgtgtcagg

SEQ ID NO: 502 - RNA, 27 nt, synthetic construct
  source 1..27: mol_type = other RNA; note = Synthetic
ccattttgcatctgagcatcgtgtcag

SEQ ID NO: 503 - RNA, 27 nt, synthetic construct
  source 1..27: mol_type = other RNA; note = Synthetic
tccattctgcatctgagcatcgtgtca

SEQ ID NO: 504 - RNA, 27 nt, synthetic construct
  source 1..27: mol_type = other RNA; note = Synthetic
gtccattctgcatctgagcatcgtgtc

SEQ ID NO: 505 - RNA, 27 nt, synthetic construct
  source 1..27: mol_type = other RNA; note = Synthetic
aactctgtccataatggtagtagcagt

SEQ ID NO: 506 - RNA, 27 nt, synthetic construct
  source 1..27: mol_type = other RNA; note = Synthetic
taactttgtccataatggtagtagcag

SEQ ID NO: 507 - RNA, 27 nt, synthetic construct
  source 1..27: mol_type = other RNA; note = Synthetic
gtaactctgtccataatggtagtagca

SEQ ID NO: 508 - RNA, 27 nt, synthetic construct
  source 1..27: mol_type = other RNA; note = Synthetic
ggtaattctgtccataatggtagtagc

SEQ ID NO: 509 - RNA, 27 nt, synthetic construct
  source 1..27: mol_type = other RNA; note = Synthetic
cggtatctctgtccataatggtagtag

SEQ ID NO: 510 - RNA, 27 nt, synthetic construct
  source 1..27: mol_type = other RNA; note = Synthetic
tcggttactctgtccataatggtagta

SEQ ID NO: 511 - RNA, 27 nt, synthetic construct
  source 1..27: mol_type = other RNA; note = Synthetic
ctcggtaactctgtccataatggtagt

SEQ ID NO: 512 - RNA, 27 nt, synthetic construct
  source 1..27: mol_type = other RNA; note = Synthetic
cctcgttaactctgtccataatggtag

SEQ ID NO: 513 - RNA, 27 nt, synthetic construct
  source 1..27: mol_type = other RNA; note = Synthetic
gtgactgtggtggagtatgtgcctcgg

SEQ ID NO: 514 - RNA, 27 nt, synthetic construct
  source 1..27: mol_type = other RNA; note = Synthetic
agttcttcctgtgacagtggtggagta

SEQ ID NO: 515 - RNA, 27 nt, synthetic construct
  source 1..27: mol_type = other RNA; note = Synthetic
accaatcttggcaagttcttcctgtga

SEQ ID NO: 516 - RNA, 27 nt, synthetic construct
  source 1..27: mol_type = other RNA; note = Synthetic
gaccatgcttggcaagttcttcctgtg

SEQ ID NO: 517 - RNA, 27 nt, synthetic construct
  source 1..27: mol_type = other RNA; note = Synthetic
tgacctagcttggcaagttcttcctgt

SEQ ID NO: 518 - RNA, 27 nt, synthetic construct
  source 1..27: mol_type = other RNA; note = Synthetic
atgactaagcttggcaagttcttcctg

SEQ ID NO: 519 - RNA, 27 nt, synthetic construct
  source 1..27: mol_type = other RNA; note = Synthetic
gatgatcaagcttggcaagttcttcct

SEQ ID NO: 520 - RNA, 27 nt, synthetic construct
  source 1..27: mol_type = other RNA; note = Synthetic
agatgtccaagcttggcaagttcttcc

SEQ ID NO: 521 - RNA, 27 nt, synthetic construct
  source 1..27: mol_type = other RNA; note = Synthetic
tagattaccaagcttggcaagttcttc

SEQ ID NO: 522 - RNA, 27 nt, synthetic construct
  source 1..27: mol_type = other RNA; note = Synthetic
atagatgaccaagcttggcaagttctt

SEQ ID NO: 523 - RNA, 27 nt, synthetic construct
  source 1..27: mol_type = other RNA; note = Synthetic
catagttgaccaagcttggcaagttct

SEQ ID NO: 524 - RNA, 27 nt, synthetic construct
  source 1..27: mol_type = other RNA; note = Synthetic
tcatatatgaccaagcttggcaagttc

SEQ ID NO: 525 - RNA, 27 nt, synthetic construct
  source 1..27: mol_type = other RNA; note = Synthetic
gtcattgatgaccaagcttggcaagtt

SEQ ID NO: 526 - RNA, 27 nt, synthetic construct
  source 1..27: mol_type = other RNA; note = Synthetic
tgtcatagatgaccaagcttggcaagt

SEQ ID NO: 527 - RNA, 27 nt, synthetic construct
  source 1..27: mol_type = other RNA; note = Synthetic
gtgtcttagatgaccaagcttggcaag

SEQ ID NO: 528 - RNA, 27 nt, synthetic construct
  source 1..27: mol_type = other RNA; note = Synthetic
ggtgttgtgtcatagatgaccaagctt

SEQ ID NO: 529 - RNA, 27 nt, synthetic construct
  source 1..27: mol_type = other RNA; note = Synthetic
ctggtttggtgtcatagatgaccaagc

SEQ ID NO: 530 - RNA, 27 nt, synthetic construct
  source 1..27: mol_type = other RNA; note = Synthetic
gctggtgtggtgtcatagatgaccaag

SEQ ID NO: 531 - RNA, 27 nt, synthetic construct
  source 1..27: mol_type = other RNA; note = Synthetic
atgcttgtgtggtgtcatagatgacca

SEQ ID NO: 532 - RNA, 27 nt, synthetic construct
  source 1..27: mol_type = other RNA; note = Synthetic
ctatgttggtgtggtgtcatagatgac

SEQ ID NO: 533 - RNA, 27 nt, synthetic construct
  source 1..27: mol_type = other RNA; note = Synthetic
actattctggtgtggtgtcatagatga

SEQ ID NO: 534 - RNA, 27 nt, synthetic construct
  source 1..27: mol_type = other RNA; note = Synthetic
cgactttgctggtgtggtgtcatagat

SEQ ID NO: 535 - RNA, 27 nt, synthetic construct
  source 1..27: mol_type = other RNA; note = Synthetic
tttggttagttttctggggtccgacta

SEQ ID NO: 536 - RNA, 27 nt, synthetic construct
  source 1..27: mol_type = other RNA; note = Synthetic
atttgtgtagttttctggggtccgact

SEQ ID NO: 537 - RNA, 27 nt, synthetic construct
  source 1..27: mol_type = other RNA; note = Synthetic
tcaggtcagcatttgggtagttttctg

SEQ ID NO: 538 - RNA, 27 nt, synthetic construct
  source 1..27: mol_type = other RNA; note = Synthetic
tgtaataccaagggcgaatctcagcat

SEQ ID NO: 539 - RNA, 27 nt, synthetic construct
  source 1..27: mol_type = other RNA; note = Synthetic
gtgtatcaccaagggcgaatctcagca

SEQ ID NO: 540 - RNA, 27 nt, synthetic construct
  source 1..27: mol_type = other RNA; note = Synthetic
tggtgtaacaccaagggcgaatctcag

SEQ ID NO: 541 - RNA, 27 nt, synthetic construct
  source 1..27: mol_type = other RNA; note = Synthetic
atggtttaacaccaagggcgaatctca

SEQ ID NO: 542 - RNA, 27 nt, synthetic construct
  source 1..27: mol_type = other RNA; note = Synthetic
tccattgtgtaacaccaagggcgaatc

SEQ ID NO: 543 - RNA, 27 nt, synthetic construct
  source 1..27: mol_type = other RNA; note = Synthetic
ctgggttccatggtgtaacaccaaggg

SEQ ID NO: 544 - RNA, 27 nt, synthetic construct
  source 1..27: mol_type = other RNA; note = Synthetic
actggtatccatggtgtaacaccaagg

SEQ ID NO: 545 - RNA, 27 nt, synthetic construct
  source 1..27: mol_type = other RNA; note = Synthetic
cactgtgatccatggtgtaacaccaag

SEQ ID NO: 546 - RNA, 27 nt, synthetic construct
  source 1..27: mol_type = other RNA; note = Synthetic
acacttggatccatggtgtaacaccaa

SEQ ID NO: 547 - RNA, 27 nt, synthetic construct
  source 1..27: mol_type = other RNA; note = Synthetic
tgacattgggatccatggtgtaacacc

SEQ ID NO: 548 - RNA, 27 nt, synthetic construct
  source 1..27: mol_type = other RNA; note = Synthetic
tgagatttgcaaggacacttgattctg

SEQ ID NO: 549 - RNA, 27 nt, synthetic construct
  source 1..27: mol_type = other RNA; note = Synthetic
gtgagtgttgcaaggacacttgattct

SEQ ID NO: 550 - RNA, 27 nt, synthetic construct
  source 1..27: mol_type = other RNA; note = Synthetic
cgtgatagttgcaaggacacttgattc

SEQ ID NO: 551 - RNA, 27 nt, synthetic construct
  source 1..27: mol_type = other RNA; note = Synthetic
aatgatcctcgataactctgtccatca

SEQ ID NO: 552 - RNA, 27 nt, synthetic construct
  source 1..27: mol_type = other RNA; note = Synthetic
gaatgtgcctcgataactctgtccatc

SEQ ID NO: 553 - RNA, 27 nt, synthetic construct
  source 1..27: mol_type = other RNA; note = Synthetic
ttgtcttctgatgccagtgtggtgtca

SEQ ID NO: 554 - RNA, 27 nt, synthetic construct
  source 1..27: mol_type = other RNA; note = Synthetic
attggtatccatggtataacaccaagg

SEQ ID NO: 555 - RNA, 27 nt, synthetic construct
  source 1..27: mol_type = other RNA; note = Synthetic
cattgtgatccatggtataacaccaag

SEQ ID NO: 556 - RNA, 27 nt, synthetic construct
  source 1..27: mol_type = other RNA; note = Synthetic
acatttggatccatggtataacaccaa

SEQ ID NO: 557 - RNA, 27 nt, synthetic construct
  source 1..27: mol_type = other RNA; note = Synthetic
gacattgggatccatggtataacacca

SEQ ID NO: 558 - RNA, 27 nt, synthetic construct
  source 1..27: mol_type = other RNA; note = Synthetic
gtcagtttgcagtactcccatctgaca

SEQ ID NO: 559 - RNA, 27 nt, synthetic construct
  source 1..27: mol_type = other RNA; note = Synthetic
tgtgttaggttgcagtactcccatctg

SEQ ID NO: 560 - RNA, 27 nt, synthetic construct
  source 1..27: mol_type = other RNA; note = Synthetic
ttgtgtcaggttgcagtactcccatct

SEQ ID NO: 561 - RNA, 27 nt, synthetic construct
  source 1..27: mol_type = other RNA; note = Synthetic
attgtttcaggttgcagtactcccatc

SEQ ID NO: 562 - RNA, 27 nt, synthetic construct
  source 1..27: mol_type = other RNA; note = Synthetic
cattgtgtcaggttgcagtactcccat

SEQ ID NO: 563 - RNA, 27 nt, synthetic construct
  source 1..27: mol_type = other RNA; note = Synthetic
cgttgttgcttgttcagaaacagccgt

SEQ ID NO: 564 - RNA, 27 nt, synthetic construct
  source 1..27: mol_type = other RNA; note = Synthetic
gctcctttggtgcttgttcagaaacag

SEQ ID NO: 565 - RNA, 27 nt, synthetic construct
  source 1..27: mol_type = other RNA; note = Synthetic
tgtccttcctgtaacagtggtggagaa

SEQ ID NO: 566 - RNA, 27 nt, synthetic construct
  source 1..27: mol_type = other RNA; note = Synthetic
atgtctttcctgtaacagtggtggaga

SEQ ID NO: 567 - RNA, 27 nt, synthetic construct
  source 1..27: mol_type = other RNA; note = Synthetic
catgttcttcctgtaacagtggtggag

SEQ ID NO: 568 - RNA, 27 nt, synthetic construct
  source 1..27: mol_type = other RNA; note = Synthetic
ggttctctgatgccagtgtggtgtcat

SEQ ID NO: 569 - RNA, 27 nt, synthetic construct
  source 1..27: mol_type = other RNA; note = Synthetic
tggttttctgatgccagtgtggtgtca

SEQ ID NO: 570 - RNA, 27 nt, synthetic construct
  source 1..27: mol_type = other RNA; note = Synthetic
caggctaccatttgggtagtattctgt

SEQ ID NO: 571 - RNA, 27 nt, synthetic construct
  source 1..27: mol_type = other RNA; note = Synthetic
tcaggtcaccatttgggtagtattctg

SEQ ID NO: 572 - RNA, 27 nt, synthetic construct
  source 1..27: mol_type = other RNA; note = Synthetic
gtcagtccaccatttgggtagtattct

SEQ ID NO: 573 - RNA, 27 nt, synthetic construct
  source 1..27: mol_type = other RNA; note = Synthetic
ttcagttggtgcttcttcagaaggaag

SEQ ID NO: 574 - RNA, 27 nt, synthetic construct
  source 1..27: mol_type = other RNA; note = Synthetic
tttcatttggtgcttcttcagaaggaa

SEQ ID NO: 575 - RNA, 27 nt, synthetic construct
  source 1..27: mol_type = other RNA; note = Synthetic
ttttctgttggtgcttcttcagaagga

SEQ ID NO: 576 - RNA, 27 nt, synthetic construct
  source 1..27: mol_type = other RNA; note = Synthetic
gtttttagttggtgcttcttcagaagg

SEQ ID NO: 577 - RNA, 27 nt, synthetic construct
  source 1..27: mol_type = other RNA; note = Synthetic
tgttttcagttggtgcttcttcagaag

SEQ ID NO: 578 - RNA, 27 nt, synthetic construct
  source 1..27: mol_type = other RNA; note = Synthetic
ctgttttcagttggtgcttcttcagaa

SEQ ID NO: 579 - RNA, 27 nt, synthetic construct
  source 1..27: mol_type = other RNA; note = Synthetic
gctgttttcagttggtgcttcttcaga

SEQ ID NO: 580 - RNA, 27 nt, synthetic construct
  source 1..27: mol_type = other RNA; note = Synthetic
tgctgttttcagttggtgcttcttcag

SEQ ID NO: 581 - RNA, 27 nt, synthetic construct
  source 1..27: mol_type = other RNA; note = Synthetic
gtgcttttttcagttggtgcttcttca

SEQ ID NO: 582 - RNA, 27 nt, synthetic construct
  source 1..27: mol_type = other RNA; note = Synthetic
agtgctgttttcagttggtgcttcttc

SEQ ID NO: 583 - RNA, 27 nt, synthetic construct
  source 1..27: mol_type = other RNA; note = Synthetic
cagtgttgttttcagttggtgcttctt

SEQ ID NO: 584 - RNA, 27 nt, synthetic construct
  source 1..27: mol_type = other RNA; note = Synthetic
ccagttctgttttcagttggtgcttct

SEQ ID NO: 585 - RNA, 27 nt, synthetic construct
  source 1..27: mol_type = other RNA; note = Synthetic
gcctctataactctgtccatcacctcg

SEQ ID NO: 586 - RNA, 27 nt, synthetic construct
  source 1..27: mol_type = other RNA; note = Synthetic
tgttcttcctgtgatagtggtggagag

SEQ ID NO: 587 - RNA, 27 nt, synthetic construct
  source 1..27: mol_type = other RNA; note = Synthetic
atgtttttcctgtgatagtggtggaga

SEQ ID NO: 588 - RNA, 27 nt, synthetic construct
  source 1..27: mol_type = other RNA; note = Synthetic
catgttcttcctgtgatagtggtggag

SEQ ID NO: 589 - RNA, 27 nt, synthetic construct
  source 1..27: mol_type = other RNA; note = Synthetic
acatgttcttcctgtgatagtggtgga

SEQ ID NO: 590 - RNA, 27 nt, synthetic construct
  source 1..27: mol_type = other RNA; note = Synthetic
gacattttcttcctgtgatagtggtgg

SEQ ID NO: 591 - RNA, 27 nt, synthetic construct
  source 1..27: mol_type = other RNA; note = Synthetic
tgacatgttcttcctgtgatagtggtg

SEQ ID NO: 592 - RNA, 27 nt, synthetic construct
  source 1..27: mol_type = other RNA; note = Synthetic
ctgacttgttcttcctgtgatagtggt

SEQ ID NO: 593 - RNA, 27 nt, synthetic construct
  source 1..27: mol_type = other RNA; note = Synthetic
actgatatgttcttcctgtgatagtgg

SEQ ID NO: 594 - RNA, 27 nt, synthetic construct
  source 1..27: mol_type = other RNA; note = Synthetic
gactgtcatgttcttcctgtgatagtg

SEQ ID NO: 595 - RNA, 27 nt, synthetic construct
  source 1..27: mol_type = other RNA; note = Synthetic
agacttacatgttcttcctgtgatagt

SEQ ID NO: 596 - RNA, 27 nt, synthetic construct
  source 1..27: mol_type = other RNA; note = Synthetic
aagactgacatgttcttcctgtgatag

SEQ ID NO: 597 - RNA, 27 nt, synthetic construct
  source 1..27: mol_type = other RNA; note = Synthetic
caagattgacatgttcttcctgtgata

SEQ ID NO: 598 - RNA, 27 nt, synthetic construct
  source 1..27: mol_type = other RNA; note = Synthetic
ccaagtctgacatgttcttcctgtgat

SEQ ID NO: 599 - RNA, 27 nt, synthetic construct
  source 1..27: mol_type = other RNA; note = Synthetic
accaatactgacatgttcttcctgtga

SEQ ID NO: 600 - RNA, 27 nt, synthetic construct
  source 1..27: mol_type = other RNA; note = Synthetic
acgactaagactgacatgttcttcctg

SEQ ID NO: 601 - RNA, 27 nt, synthetic construct
  source 1..27: mol_type = other RNA; note = Synthetic
gacgatcaagactgacatgttcttcct

SEQ ID NO: 602 - RNA, 27 nt, synthetic construct
  source 1..27: mol_type = other RNA; note = Synthetic
agacgtccaagactgacatgttcttcc

SEQ ID NO: 603 - RNA, 27 nt, synthetic construct
  source 1..27: mol_type = other RNA; note = Synthetic
tagactaccaagactgacatgttcttc

SEQ ID NO: 604 - RNA, 27 nt, synthetic construct
  source 1..27: mol_type = other RNA; note = Synthetic
tagtataatgggatcctccgatgccaa

SEQ ID NO: 605 - RNA, 27 nt, synthetic construct
  source 1..27: mol_type = other RNA; note = Synthetic
ctggttaggccagcatttggatagtat

SEQ ID NO: 606 - RNA, 27 nt, synthetic construct
  source 1..27: mol_type = other RNA; note = Synthetic
gcttgttcagaaggagcctctgtgctt

SEQ ID NO: 607 - RNA, 27 nt, synthetic construct
  source 1..27: mol_type = other RNA; note = Synthetic
ctcagttggtgcttgttcagaaggagc

SEQ ID NO: 608 - RNA, 27 nt, synthetic construct
  source 1..27: mol_type = other RNA; note = Synthetic
tctcatgtggtgcttgttcagaaggag

SEQ ID NO: 609 - RNA, 27 nt, synthetic construct
  source 1..27: mol_type = other RNA; note = Synthetic
ttctctggtggtgcttgttcagaagga

SEQ ID NO: 610 - RNA, 27 nt, synthetic construct
  source 1..27: mol_type = other RNA; note = Synthetic
tttcttaggtggtgcttgttcagaagg

SEQ ID NO: 611 - RNA, 27 nt, synthetic construct
  source 1..27: mol_type = other RNA; note = Synthetic
ttttctcaggtggtgcttgttcagaag

SEQ ID NO: 612 - RNA, 27 nt, synthetic construct
  source 1..27: mol_type = other RNA; note = Synthetic
cttttttcaggtggtgcttgttcagaa

SEQ ID NO: 613 - RNA, 27 nt, synthetic construct
  source 1..27: mol_type = other RNA; note = Synthetic
gcttttctcaggtggtgcttgttcaga

SEQ ID NO: 614 - RNA, 27 nt, synthetic construct
  source 1..27: mol_type = other RNA; note = Synthetic
ggcttttctcaggtggtgcttgttcag

SEQ ID NO: 615 - RNA, 27 nt, synthetic construct
  source 1..27: mol_type = other RNA; note = Synthetic
gggcttttctcaggtggtgcttgttca

SEQ ID NO: 616 - RNA, 27 nt, synthetic construct
  source 1..27: mol_type = other RNA; note = Synthetic
cagggtttttctcaggtggtgcttgtt

SEQ ID NO: 617 - RNA, 27 nt, synthetic construct
  source 1..27: mol_type = other RNA; note = Synthetic
acaggtcttttctcaggtggtgcttgt

SEQ ID NO: 618 - RNA, 27 nt, synthetic construct
  source 1..27: mol_type = other RNA; note = Synthetic
cacagtgcttttctcaggtggtgcttg

SEQ ID NO: 619 - RNA, 27 nt, synthetic construct
  source 1..27: mol_type = other RNA; note = Synthetic
tcctgtaccacagggcttttctcaggt

SEQ ID NO: 620 - RNA, 27 nt, synthetic construct
  source 1..27: mol_type = other RNA; note = Synthetic
tggtatcaatcctggaccacagggctt

SEQ ID NO: 621 - RNA, 27 nt, synthetic construct
  source 1..27: mol_type = other RNA; note = Synthetic
ccatgttagcaatcctggaccacaggg

SEQ ID NO: 622 - RNA, 27 nt, synthetic construct
  source 1..27: mol_type = other RNA; note = Synthetic
ggtccttcctgtgacagtggtggagga

SEQ ID NO: 623 - RNA, 27 nt, synthetic construct
  source 1..27: mol_type = other RNA; note = Synthetic
aggtctttcctgtgacagtggtggagg

SEQ ID NO: 624 - RNA, 27 nt, synthetic construct
  source 1..27: mol_type = other RNA; note = Synthetic
agtgttgtatcatagatgaccaagatt

SEQ ID NO: 625 - RNA, 27 nt, synthetic construct
  source 1..27: mol_type = other RNA; note = Synthetic
cagtgtggtatcatagatgaccaagat

SEQ ID NO: 626 - RNA, 27 nt, synthetic construct
  source 1..27: mol_type = other RNA; note = Synthetic
ccagtttggtatcatagatgaccaaga

SEQ ID NO: 627 - RNA, 27 nt, synthetic construct
  source 1..27: mol_type = other RNA; note = Synthetic
gccagtgtggtatcatagatgaccaag

SEQ ID NO: 628 - RNA, 27 nt, synthetic construct
  source 1..27: mol_type = other RNA; note = Synthetic
tgccattgtggtatcatagatgaccaa

SEQ ID NO: 629 - RNA, 27 nt, synthetic construct
  source 1..27: mol_type = other RNA; note = Synthetic
atgcctgtgtggtatcatagatgacca

SEQ ID NO: 630 - RNA, 27 nt, synthetic construct
  source 1..27: mol_type = other RNA; note = Synthetic
gatgctagtgtggtatcatagatgacc

SEQ ID NO: 631 - RNA, 27 nt, synthetic construct
  source 1..27: mol_type = other RNA; note = Synthetic
tgatgtcagtgtggtatcatagatgac

SEQ ID NO: 632 - RNA, 27 nt, synthetic construct
  source 1..27: mol_type = other RNA; note = Synthetic
ctgattccagtgtggtatcatagatga

SEQ ID NO: 633 - RNA, 27 nt, synthetic construct
  source 1..27: mol_type = other RNA; note = Synthetic
tctgatgccagtgtggtatcatagatg

SEQ ID NO: 634 - RNA, 27 nt, synthetic construct
  source 1..27: mol_type = other RNA; note = Synthetic
ctctgttgccagtgtggtatcatagat

SEQ ID NO: 635 - RNA, 27 nt, synthetic construct
  source 1..27: mol_type = other RNA; note = Synthetic
gtcctttgatgccagtgtggtatcata

SEQ ID NO: 636 - RNA, 27 nt, synthetic construct
  source 1..27: mol_type = other RNA; note = Synthetic
gggtcttctgatgccagtgtggtatca

SEQ ID NO: 637 - RNA, 27 nt, synthetic construct
  source 1..27: mol_type = other RNA; note = Synthetic
tttggttagttttctggggtcctctga

SEQ ID NO: 638 - RNA, 27 nt, synthetic construct
  source 1..27: mol_type = other RNA; note = Synthetic
atttgtgtagttttctggggtcctctg

SEQ ID NO: 639 - RNA, 27 nt, synthetic construct
  source 1..27: mol_type = other RNA; note = Synthetic
gaatctggattcctgcagtagttctcg

SEQ ID NO: 640 - RNA, 27 nt, synthetic construct
  source 1..27: mol_type = other RNA; note = Synthetic
ccagattctggattcctgcagtagttc

SEQ ID NO: 641 - RNA, 27 nt, synthetic construct
  source 1..27: mol_type = other RNA; note = Synthetic
tcccataatctggattcctgcagtagt

SEQ ID NO: 642 - RNA, 27 nt, synthetic construct
  source 1..27: mol_type = other RNA; note = Synthetic
ttccctgaatctggattcctgcagtag

SEQ ID NO: 643 - RNA, 27 nt, synthetic construct
  source 1..27: mol_type = other RNA; note = Synthetic
tttcctagaatctggattcctgcagta

SEQ ID NO: 644 - RNA, 27 nt, synthetic construct
  source 1..27: mol_type = other RNA; note = Synthetic
gtttctcagaatctggattcctgcagt

SEQ ID NO: 645 - RNA, 27 nt, synthetic construct
  source 1..27: mol_type = other RNA; note = Synthetic
tgttttccagaatctggattcctgcag

SEQ ID NO: 646 - RNA, 27 nt, synthetic construct
  source 1..27: mol_type = other RNA; note = Synthetic
ggttgtttcccagaatctggattcctg

SEQ ID NO: 647 - RNA, 27 nt, synthetic construct
  source 1..27: mol_type = other RNA; note = Synthetic
ttgtgtaacaccagggttgtttcccag

SEQ ID NO: 648 - RNA, 27 nt, synthetic construct
  source 1..27: mol_type = other RNA; note = Synthetic
gttgtttaacaccagggttgtttccca

SEQ ID NO: 649 - RNA, 27 nt, synthetic construct
  source 1..27: mol_type = other RNA; note = Synthetic
gattgtagtactcccacctcacacacg

SEQ ID NO: 650 - RNA, 27 nt, synthetic construct
  source 1..27: mol_type = other RNA; note = Synthetic
agatttcagtactcccacctcacacac

SEQ ID NO: 651 - RNA, 27 nt, synthetic construct
  source 1..27: mol_type = other RNA; note = Synthetic
tcagattgcagtactcccacctcacac

SEQ ID NO: 652 - RNA, 27 nt, synthetic construct
  source 1..27: mol_type = other RNA; note = Synthetic
gtcagtttgcagtactcccacctcaca

SEQ ID NO: 653 - RNA, 27 nt, synthetic construct
  source 1..27: mol_type = other RNA; note = Synthetic
attctttttctgagcattgtgtcagat

SEQ ID NO: 654 - RNA, 27 nt, synthetic construct
  source 1..27: mol_type = other RNA; note = Synthetic
gattctgtttctgagcattgtgtcaga

SEQ ID NO: 655 - RNA, 27 nt, synthetic construct
  source 1..27: mol_type = other RNA; note = Synthetic
tgattttgtttctgagcattgtgtcag

SEQ ID NO: 656 - RNA, 27 nt, synthetic construct
  source 1..27: mol_type = other RNA; note = Synthetic
ctgattctgtttctgagcattgtgtca

SEQ ID NO: 657 - RNA, 27 nt, synthetic construct
  source 1..27: mol_type = other RNA; note = Synthetic
cctgattctgtttctgagcattgtgtc

SEQ ID NO: 658 - RNA, 27 nt, synthetic construct
  source 1..27: mol_type = other RNA; note = Synthetic
acctgtttctgtttctgagcattgtgt

SEQ ID NO: 659 - RNA, 27 nt, synthetic construct
  source 1..27: mol_type = other RNA; note = Synthetic
caccttattctgtttctgagcattgtg

SEQ ID NO: 660 - RNA, 27 nt, synthetic construct
  source 1..27: mol_type = other RNA; note = Synthetic
acacctgattctgtttctgagcattgt

SEQ ID NO: 661 - RNA, 27 nt, synthetic construct
  source 1..27: mol_type = other RNA; note = Synthetic
gacacttgattctgtttctgagcattg

SEQ ID NO: 662 - RNA, 27 nt, synthetic construct
  source 1..27: mol_type = other RNA; note = Synthetic
ggacatctgattctgtttctgagcatt

SEQ ID NO: 663 - RNA, 27 nt, synthetic construct
  source 1..27: mol_type = other RNA; note = Synthetic
taggatacctgattctgtttctgagca

SEQ ID NO: 664 - RNA, 27 nt, synthetic construct
  source 1..27: mol_type = other RNA; note = Synthetic
tctagtacacctgattctgtttctgag

SEQ ID NO: 665 - RNA, 27 nt, synthetic construct
  source 1..27: mol_type = other RNA; note = Synthetic
ctctatgacacctgattctgtttctga

SEQ ID NO: 666 - RNA, 27 nt, synthetic construct
  source 1..27: mol_type = other RNA; note = Synthetic
tctcttggacacctgattctgtttctg

SEQ ID NO: 667 - RNA, 27 nt, synthetic construct
  source 1..27: mol_type = other RNA; note = Synthetic
gtctctaggacacctgattctgtttct

SEQ ID NO: 668 - RNA, 27 nt, synthetic construct
  source 1..27: mol_type = other RNA; note = Synthetic
agtctttaggacacctgattctgtttc

SEQ ID NO: 669 - RNA, 27 nt, synthetic construct
  source 1..27: mol_type = other RNA; note = Synthetic
ggagtttctaggacacctgattctgtt

SEQ ID NO: 670 - RNA, 27 nt, synthetic construct
  source 1..27: mol_type = other RNA; note = Synthetic
tgggattctctaggacacctgattctg

SEQ ID NO: 671 - RNA, 27 nt, synthetic construct
  source 1..27: mol_type = other RNA; note = Synthetic
agtggtagtctctaggacacctgattc

SEQ ID NO: 672 - RNA, 27 nt, synthetic construct
  source 1..27: mol_type = other RNA; note = Synthetic
caacattgggagtctctaggacacctg

SEQ ID NO: 673 - RNA, 27 nt, synthetic construct
  source 1..27: mol_type = other RNA; note = Synthetic
tggaataacagtgggagtctctaggac

SEQ ID NO: 674 - RNA, 27 nt, synthetic construct
  source 1..27: mol_type = other RNA; note = Synthetic
ctggatcaacagtgggagtctctagga

SEQ ID NO: 675 - RNA, 27 nt, synthetic construct
  source 1..27: mol_type = other RNA; note = Synthetic
actggtacaacagtgggagtctctagg

SEQ ID NO: 676 - RNA, 27 nt, synthetic construct
  source 1..27: mol_type = other RNA; note = Synthetic
aactgtaacaacagtgggagtctctag

SEQ ID NO: 677 - RNA, 27 nt, synthetic construct
  source 1..27: mol_type = other RNA; note = Synthetic
gaacttgaacaacagtgggagtctcta

SEQ ID NO: 678 - RNA, 27 nt, synthetic construct
  source 1..27: mol_type = other RNA; note = Synthetic
ggaactggaacaacagtgggagtctct

SEQ ID NO: 679 - RNA, 27 nt, synthetic construct
  source 1..27: mol_type = other RNA; note = Synthetic
cagtttgtgctgcttcagaatgagcct

SEQ ID NO: 680 - RNA, 27 nt, synthetic construct
  source 1..27: mol_type = other RNA; note = Synthetic
tcagttggtgctgcttcagaatgagcc

SEQ ID NO: 681 - RNA, 27 nt, synthetic construct
  source 1..27: mol_type = other RNA; note = Synthetic
ctcagttggtgctgcttcagaatgagc

SEQ ID NO: 682 - RNA, 27 nt, synthetic construct
  source 1..27: mol_type = other RNA; note = Synthetic
gctcatttggtgctgcttcagaatgag

SEQ ID NO: 683 - RNA, 27 nt, synthetic construct
  source 1..27: mol_type = other RNA; note = Synthetic
tgctctgttggtgctgcttcagaatga

SEQ ID NO: 684 - RNA, 27 nt, synthetic construct
  source 1..27: mol_type = other RNA; note = Synthetic
ttgcttagttggtgctgcttcagaatg

SEQ ID NO: 685 - RNA, 27 nt, synthetic construct
  source 1..27: mol_type = other RNA; note = Synthetic
tttgctcagttggtgctgcttcagaat

SEQ ID NO: 686 - RNA, 27 nt, synthetic construct
  source 1..27: mol_type = other RNA; note = Synthetic
gtttgttcagttggtgctgcttcagaa

SEQ ID NO: 687 - RNA, 27 nt, synthetic construct
  source 1..27: mol_type = other RNA; note = Synthetic
ggttttctcagttggtgctgcttcaga

SEQ ID NO: 688 - RNA, 27 nt, synthetic construct
  source 1..27: mol_type = other RNA; note = Synthetic
gggtttgctcagttggtgctgcttcag

SEQ ID NO: 689 - RNA, 27 nt, synthetic construct
  source 1..27: mol_type = other RNA; note = Synthetic
ggggtttgctcagttggtgctgcttca

SEQ ID NO: 690 - RNA, 27 nt, synthetic construct
  source 1..27: mol_type = other RNA; note = Synthetic
tctggtcattaccatggtagcactgcc

SEQ ID NO: 691 - RNA, 27 nt, synthetic construct
  source 1..27: mol_type = other RNA; note = Synthetic
ctctgtccattaccatggtagcactgc

SEQ ID NO: 692 - RNA, 27 nt, synthetic construct
  source 1..27: mol_type = other RNA; note = Synthetic
ttcctttgacagtggtggagaatgtgc

SEQ ID NO: 693 - RNA, 27 nt, synthetic construct
  source 1..27: mol_type = other RNA; note = Synthetic
ttgacttgtccttcctgtgacagtggt

SEQ ID NO: 694 - RNA, 27 nt, synthetic construct
  source 1..27: mol_type = other RNA; note = Synthetic
attgatatgtccttcctgtgacagtgg

SEQ ID NO: 695 - RNA, 27 nt, synthetic construct
  source 1..27: mol_type = other RNA; note = Synthetic
gattgtcatgtccttcctgtgacagtg

SEQ ID NO: 696 - RNA, 27 nt, synthetic construct
  source 1..27: mol_type = other RNA; note = Synthetic
agatttacatgtccttcctgtgacagt

SEQ ID NO: 697 - RNA, 27 nt, synthetic construct
  source 1..27: mol_type = other RNA; note = Synthetic
aagattgacatgtccttcctgtgacag

SEQ ID NO: 698 - RNA, 27 nt, synthetic construct
  source 1..27: mol_type = other RNA; note = Synthetic
caagattgacatgtccttcctgtgaca

SEQ ID NO: 699 - RNA, 27 nt, synthetic construct
  source 1..27: mol_type = other RNA; note = Synthetic
accaatattgacatgtccttcctgtga

SEQ ID NO: 700 - RNA, 27 nt, synthetic construct
  source 1..27: mol_type = other RNA; note = Synthetic
gaccatgattgacatgtccttcctgtg

SEQ ID NO: 701 - RNA, 27 nt, synthetic construct
  source 1..27: mol_type = other RNA; note = Synthetic
tgacctagattgacatgtccttcctgt

SEQ ID NO: 702 - RNA, 27 nt, synthetic construct
  source 1..27: mol_type = other RNA; note = Synthetic
atgactaagattgacatgtccttcctg

SEQ ID NO: 703 - RNA, 27 nt, synthetic construct
  source 1..27: mol_type = other RNA; note = Synthetic
gatgatcaagattgacatgtccttcct

SEQ ID NO: 704 - RNA, 27 nt, synthetic construct
  source 1..27: mol_type = other RNA; note = Synthetic
ggatgtccaagattgacatgtccttcc

SEQ ID NO: 705 - RNA, 27 nt, synthetic construct
  source 1..27: mol_type = other RNA; note = Synthetic
tggattaccaagattgacatgtccttc

SEQ ID NO: 706 - RNA, 27 nt, synthetic construct
  source 1..27: mol_type = other RNA; note = Synthetic
atggatgaccaagattgacatgtcctt

SEQ ID NO: 707 - RNA, 27 nt, synthetic construct
  source 1..27: mol_type = other RNA; note = Synthetic
catggttgaccaagattgacatgtcct

SEQ ID NO: 708 - RNA, 27 nt, synthetic construct
  source 1..27: mol_type = other RNA; note = Synthetic
tcatgtatgaccaagattgacatgtcc

SEQ ID NO: 709 - RNA, 27 nt, synthetic construct
  source 1..27: mol_type = other RNA; note = Synthetic
gtcattgatgaccaagattgacatgtc

SEQ ID NO: 710 - RNA, 27 nt, synthetic construct
  source 1..27: mol_type = other RNA; note = Synthetic
tgtcatggatgaccaagattgacatgt

SEQ ID NO: 711 - RNA, 27 nt, synthetic construct
  source 1..27: mol_type = other RNA; note = Synthetic
gtgtcttggatgaccaagattgacatg

SEQ ID NO: 712 - RNA, 27 nt, synthetic construct
  source 1..27: mol_type = other RNA; note = Synthetic
ggtgttatggatgaccaagattgacat

SEQ ID NO: 713 - RNA, 27 nt, synthetic construct
  source 1..27: mol_type = other RNA; note = Synthetic
tggtgtcatggatgaccaagattgaca

SEQ ID NO: 714 - RNA, 27 nt, synthetic construct
  source 1..27: mol_type = other RNA; note = Synthetic
gtggtttcatggatgaccaagattgac

SEQ ID NO: 715 - RNA, 27 nt, synthetic construct
  source 1..27: mol_type = other RNA; note = Synthetic
tgtggtgtcatggatgaccaagattga

SEQ ID NO: 716 - RNA, 27 nt, synthetic construct
  source 1..27: mol_type = other RNA; note = Synthetic
gtgtgttgtcatggatgaccaagattg

SEQ ID NO: 717 - RNA, 27 nt, synthetic construct
  source 1..27: mol_type = other RNA; note = Synthetic
ggatttctgcagtagttcattgtcagg

SEQ ID NO: 718 - RNA, 27 nt, synthetic construct
  source 1..27: mol_type = other RNA; note = Synthetic
tggattcctgcagtagttcattgtcag

SEQ ID NO: 719 - RNA, 27 nt, synthetic construct
  source 1..27: mol_type = other RNA; note = Synthetic
ctggattcctgcagtagttcattgtca

SEQ ID NO: 720 - RNA, 27 nt, synthetic construct
  source 1..27: mol_type = other RNA; note = Synthetic
tctggtttcctgcagtagttcattgtc

SEQ ID NO: 721 - RNA, 27 nt, synthetic construct
  source 1..27: mol_type = other RNA; note = Synthetic
atctgtattcctgcagtagttcattgt

SEQ ID NO: 722 - RNA, 27 nt, synthetic construct
  source 1..27: mol_type = other RNA; note = Synthetic
catcttgattcctgcagtagttcattg

SEQ ID NO: 723 - RNA, 27 nt, synthetic construct
  source 1..27: mol_type = other RNA; note = Synthetic
gcatctggattcctgcagtagttcatt

SEQ ID NO: 724 - RNA, 27 nt, synthetic construct
  source 1..27: mol_type = other RNA; note = Synthetic
atcggtatctggattcctgcagtagtt

SEQ ID NO: 725 - RNA, 27 nt, synthetic construct
  source 1..27: mol_type = other RNA; note = Synthetic
tccattgtaaaacaccaagggcctgta

SEQ ID NO: 726 - RNA, 27 nt, synthetic construct
  source 1..27: mol_type = other RNA; note = Synthetic
ctgggttccatggtaaaacaccaaggg

SEQ ID NO: 727 - RNA, 27 nt, synthetic construct
  source 1..27: mol_type = other RNA; note = Synthetic
gctggtgtccatggtaaaacaccaagg

SEQ ID NO: 728 - RNA, 27 nt, synthetic construct
  source 1..27: mol_type = other RNA; note = Synthetic
tgctgtggtccatggtaaaacaccaag

SEQ ID NO: 729 - RNA, 27 nt, synthetic construct
  source 1..27: mol_type = other RNA; note = Synthetic
atgcttgggtccatggtaaaacaccaa

SEQ ID NO: 730 - RNA, 27 nt, synthetic construct
  source 1..27: mol_type = other RNA; note = Synthetic
gatgctggggtccatggtaaaacacca

SEQ ID NO: 731 - RNA, 27 nt, synthetic construct
  source 1..27: mol_type = other RNA; note = Synthetic
ctgattctggggtccatggtaaaacac

SEQ ID NO: 732 - RNA, 27 nt, synthetic construct
  source 1..27: mol_type = other RNA; note = Synthetic
gcatctcgtcaggttgcagtactccca

SEQ ID NO: 733 - RNA, 27 nt, synthetic construct
  source 1..27: mol_type = other RNA; note = Synthetic
agcattgcgtcaggttgcagtactccc

SEQ ID NO: 734 - RNA, 27 nt, synthetic construct
  source 1..27: mol_type = other RNA; note = Synthetic
tgagcttcgcgtcaggttgcagtactc

SEQ ID NO: 735 - RNA, 27 nt, synthetic construct
  source 1..27: mol_type = other RNA; note = Synthetic
ctgagtatcgcgtcaggttgcagtact

SEQ ID NO: 736 - RNA, 27 nt, synthetic construct
  source 1..27: mol_type = other RNA; note = Synthetic
tgtcttagcatcgcgtcaggttgcagt

SEQ ID NO: 737 - RNA, 27 nt, synthetic construct
  source 1..27: mol_type = other RNA; note = Synthetic
ttctgtgtctgagcatcgcgtcaggtt

SEQ ID NO: 738 - RNA, 27 nt, synthetic construct
  source 1..27: mol_type = other RNA; note = Synthetic
cttctttgtctgagcatcgcgtcaggt

SEQ ID NO: 739 - RNA, 27 nt, synthetic construct
  source 1..27: mol_type = other RNA; note = Synthetic
acagttccttctgtgtctgagcatcgc

SEQ ID NO: 740 - RNA, 27 nt, synthetic construct
  source 1..27: mol_type = other RNA; note = Synthetic
cacagtcccttctgtgtctgagcatcg

SEQ ID NO: 741 - RNA, 27 nt, synthetic construct
  source 1..27: mol_type = other RNA; note = Synthetic
agcgatcacagtcccttctgtgtctga

SEQ ID NO: 742 - RNA, 27 nt, synthetic construct
  source 1..27: mol_type = other RNA; note = Synthetic
tcccataatcaaatgaagaggatgcac

SEQ ID NO: 743 - RNA, 27 nt, synthetic construct
  source 1..27: mol_type = other RNA; note = Synthetic
ttccctcaatcaaatgaagaggatgca

SEQ ID NO: 744 - RNA, 27 nt, synthetic construct
  source 1..27: mol_type = other RNA; note = Synthetic
cttcctacaatcaaatgaagaggatgc

SEQ ID NO: 745 - RNA, 27 nt, synthetic construct
  source 1..27: mol_type = other RNA; note = Synthetic
gcttctcacaatcaaatgaagaggatg

SEQ ID NO: 746 - RNA, 27 nt, synthetic construct
  source 1..27: mol_type = other RNA; note = Synthetic
ggctttccacaatcaaatgaagaggat

SEQ ID NO: 747 - RNA, 27 nt, synthetic construct
  source 1..27: mol_type = other RNA; note = Synthetic
tgaggtttcccacaatcaaatgaagag

SEQ ID NO: 748 - RNA, 27 nt, synthetic construct
  source 1..27: mol_type = other RNA; note = Synthetic
ttgagtcttcccacaatcaaatgaaga

SEQ ID NO: 749 - RNA, 27 nt, synthetic construct
  source 1..27: mol_type = other RNA; note = Synthetic
cttgatgcttcccacaatcaaatgaag

SEQ ID NO: 750 - RNA, 27 nt, synthetic construct
  source 1..27: mol_type = other RNA; note = Synthetic
acttgtggcttcccacaatcaaatgaa

SEQ ID NO: 751 - RNA, 27 nt, synthetic construct
  source 1..27: mol_type = other RNA; note = Synthetic
cactttaggcttcccacaatcaaatga

SEQ ID NO: 752 - RNA, 27 nt, synthetic construct
  source 1..27: mol_type = other RNA; note = Synthetic
ttcggttccacttgaggcttcccacaa

SEQ ID NO: 753 - RNA, 27 nt, synthetic construct
  source 1..27: mol_type = other RNA; note = Synthetic
ctacattgcttccaggacatttcttcg

SEQ ID NO: 754 - RNA, 27 nt, synthetic construct
  source 1..27: mol_type = other RNA; note = Synthetic
cctactatgcttccaggacatttcttc

SEQ ID NO: 755 - RNA, 27 nt, synthetic construct
  source 1..27: mol_type = other RNA; note = Synthetic
ccctataatgcttccaggacatttctt

SEQ ID NO: 756 - RNA, 27 nt, synthetic construct
  source 1..27: mol_type = other RNA; note = Synthetic
ccccctacaatgcttccaggacatttc

SEQ ID NO: 757 - RNA, 27 nt, synthetic construct
  source 1..27: mol_type = other RNA; note = Synthetic
cccccttacaatgcttccaggacattt

SEQ ID NO: 758 - RNA, 27 nt, synthetic construct
  source 1..27: mol_type = other RNA; note = Synthetic
agaagtgctttccaaaccttgttctga

SEQ ID NO: 759 - RNA, 27 nt, synthetic construct
  source 1..27: mol_type = other RNA; note = Synthetic
cagaattgctttccaaaccttgttctg

SEQ ID NO: 760 - RNA, 27 nt, synthetic construct
  source 1..27: mol_type = other RNA; note = Synthetic
acagatgtgctttccaaaccttgttct

SEQ ID NO: 761 - RNA, 27 nt, synthetic construct
  source 1..27: mol_type = other RNA; note = Synthetic
cacagtagtgctttccaaaccttgttc

SEQ ID NO: 762 - RNA, 27 nt, synthetic construct
  source 1..27: mol_type = other RNA; note = Synthetic
ccacataagtgctttccaaaccttgtt

SEQ ID NO: 763 - RNA, 27 nt, synthetic construct
  source 1..27: mol_type = other RNA; note = Synthetic
tccactgaagtgctttccaaaccttgt

SEQ ID NO: 764 - RNA, 27 nt, synthetic construct
  source 1..27: mol_type = other RNA; note = Synthetic
ctccatagaagtgctttccaaaccttg

SEQ ID NO: 765 - RNA, 27 nt, synthetic construct
  source 1..27: mol_type = other RNA; note = Synthetic
ggtgcttccacagaagtgctttccaaa

SEQ ID NO: 766 - RNA, 27 nt, synthetic construct
  source 1..27: mol_type = other RNA; note = Synthetic
tctggtgatattaaggtgcctccacag

SEQ ID NO: 767 - RNA, 27 nt, synthetic construct
  source 1..27: mol_type = other RNA; note = Synthetic
cagcatccactctggggatattaaggt

SEQ ID NO: 768 - RNA, 27 nt, synthetic construct
  source 1..27: mol_type = other RNA; note = Synthetic
gtcagtacccactctggggatattaag

SEQ ID NO: 769 - RNA, 27 nt, synthetic construct
  source 1..27: mol_type = other RNA; note = Synthetic
gtgagtagcagtcagcacccactctgg

SEQ ID NO: 770 - RNA, 27 nt, synthetic construct
  source 1..27: mol_type = other RNA; note = Synthetic
ttggtttgcacccaggatgaccttgta

SEQ ID NO: 771 - RNA, 27 nt, synthetic construct
  source 1..27: mol_type = other RNA; note = Synthetic
gttcatttcttggtgtgcacccaggat

SEQ ID NO: 772 - RNA, 27 nt, synthetic construct
  source 1..27: mol_type = other RNA; note = Synthetic
tcgagtttcacttcttggtgtgcaccc

SEQ ID NO: 773 - RNA, 27 nt, synthetic construct
  source 1..27: mol_type = other RNA; note = Synthetic
tagctttagcaaggcaatatctgcttg

SEQ ID NO: 774 - RNA, 27 nt, synthetic construct
  source 1..27: mol_type = other RNA; note = Synthetic
ttagctttagcaaggcaatatctgctt

SEQ ID NO: 775 - RNA, 27 nt, synthetic construct
  source 1..27: mol_type = other RNA; note = Synthetic
cttagttttagcaaggcaatatctgct

SEQ ID NO: 776 - RNA, 27 nt, synthetic construct
  source 1..27: mol_type = other RNA; note = Synthetic
gcttatctttagcaaggcaatatctgc

SEQ ID NO: 777 - RNA, 27 nt, synthetic construct
  source 1..27: mol_type = other RNA; note = Synthetic
tgctttgctttagcaaggcaatatctg

SEQ ID NO: 778 - RNA, 27 nt, synthetic construct
  source 1..27: mol_type = other RNA; note = Synthetic
ctgcttagctttagcaaggcaatatct

SEQ ID NO: 779 - RNA, 27 nt, synthetic construct
  source 1..27: mol_type = other RNA; note = Synthetic
cctgcttagctttagcaaggcaatatc

SEQ ID NO: 780 - RNA, 27 nt, synthetic construct
  source 1..27: mol_type = other RNA; note = Synthetic
gctggtattactttgtcagtgatgacg

SEQ ID NO: 781 - RNA, 27 nt, synthetic construct
  source 1..27: mol_type = other RNA; note = Synthetic
cagcctgtgatgtaacattcagtcctg

SEQ ID NO: 782 - RNA, 27 nt, synthetic construct
  source 1..27: mol_type = other RNA; note = Synthetic
ccagctagtgatgtaacattcagtcct

SEQ ID NO: 783 - RNA, 27 nt, synthetic construct
  source 1..27: mol_type = other RNA; note = Synthetic
cccagtcagtgatgtaacattcagtcc

SEQ ID NO: 784 - RNA, 27 nt, synthetic construct
  source 1..27: mol_type = other RNA; note = Synthetic
ccccatccagtgatgtaacattcagtc

SEQ ID NO: 785 - RNA, 27 nt, synthetic construct
  source 1..27: mol_type = other RNA; note = Synthetic
tcccctgccagtgatgtaacattcagt

SEQ ID NO: 786 - RNA, 27 nt, synthetic construct
  source 1..27: mol_type = other RNA; note = Synthetic
tctcctcagccagtgatgtaacattca

SEQ ID NO: 787 - RNA, 27 nt, synthetic construct
  source 1..27: mol_type = other RNA; note = Synthetic
cagtctcaaaggtaccttgggtttctc

SEQ ID NO: 788 - RNA, 22 nt, synthetic construct
  source 1..22: mol_type = other RNA; note = Synthetic
tttcctgtgacagtggtggagg

SEQ ID NO: 789 - RNA, 22 nt, synthetic construct
  source 1..22: mol_type = other RNA; note = Synthetic
tataactctgtccattaccagg

SEQ ID NO: 790 - RNA, 22 nt, synthetic construct
  source 1..22: mol_type = other RNA; note = Synthetic
tatcgtgtcaggttgcagtagg

SEQ ID NO: 791 - RNA, 22 nt, synthetic construct
  source 1..22: mol_type = other RNA; note = Synthetic
taccaagcttggcaagttctgg

SEQ ID NO: 792 - RNA, 22 nt, synthetic construct
  source 1..22: mol_type = other RNA; note = Synthetic
ttgaccaagcttggcaagttgg

SEQ ID NO: 793 - RNA, 22 nt, synthetic construct
  source 1..22: mol_type = other RNA; note = Synthetic
tagatgaccaagcttggcaagg

SEQ ID NO: 794 - RNA, 22 nt, synthetic construct
  source 1..22: mol_type = other RNA; note = Synthetic
tcctcgataactctgtccatgg

SEQ ID NO: 795 - RNA, 22 nt, synthetic construct
  source 1..22: mol_type = other RNA; note = Synthetic
tgcctcgataactctgtccagg

SEQ ID NO: 796 - RNA, 22 nt, synthetic construct
  source 1..22: mol_type = other RNA; note = Synthetic
tgtggtatcatagatgaccagg

SEQ ID NO: 797 - RNA, 22 nt, synthetic construct
  source 1..22: mol_type = other RNA; note = Synthetic
taatctggattcctgcagtagg

SEQ ID NO: 798 - RNA, 22 nt, synthetic construct
  source 1..22: mol_type = other RNA; note = Synthetic
tatgcttccaggacatttctgg

SEQ ID NO: 799 - RNA, 22 nt, synthetic construct
  source 1..22: mol_type = other RNA; note = Synthetic
ttggataatattctgttgtcgg

SEQ ID NO: 800 - RNA, 22 nt, synthetic construct
  source 1..22: mol_type = other RNA; note = Synthetic
tccttgataactctgtccatgg

SEQ ID NO: 801 - RNA, 22 nt, synthetic construct
  source 1..22: mol_type = other RNA; note = Synthetic
ttggataatattctgttgtcgg

SEQ ID NO: 802 - RNA, 22 nt, synthetic construct
  source 1..22: mol_type = other RNA; note = Synthetic
tccttgataactctgtccatgg

SEQ ID NO: 803 - RNA, 22 nt, synthetic construct
  source 1..22: mol_type = other RNA; note = Synthetic
ttggataatattctgttgtcgg

SEQ ID NO: 804 - RNA, 19 nt, synthetic construct
  source 1..19: mol_type = other RNA; note = Synthetic
ctgagcaaagccatgtggt

SEQ ID NO: 805 - RNA, 19 nt, synthetic construct
  source 1..19: mol_type = other RNA; note = Synthetic
agcaaagccatgtggtcca

SEQ ID NO: 806 - RNA, 19 nt, synthetic construct
  source 1..19: mol_type = other RNA; note = Synthetic
aagccatgtggtccaggat

SEQ ID NO: 807 - RNA, 19 nt, synthetic construct
  source 1..19: mol_type = other RNA; note = Synthetic
agccatgtggtccaggatt

SEQ ID NO: 808 - RNA, 19 nt, synthetic construct
  source 1..19: mol_type = other RNA; note = Synthetic
gccatgtggtccaggattg

SEQ ID NO: 809 - RNA, 19 nt, synthetic construct
  source 1..19: mol_type = other RNA; note = Synthetic
ccatgtggtccaggattgc

SEQ ID NO: 810 - RNA, 19 nt, synthetic construct
  source 1..19: mol_type = other RNA; note = Synthetic
catgtggtccaggattgct

SEQ ID NO: 811 - RNA, 19 nt, synthetic construct
  source 1..19: mol_type = other RNA; note = Synthetic
atgtggtccaggattgcta

SEQ ID NO: 812 - RNA, 19 nt, synthetic construct
  source 1..19: mol_type = other RNA; note = Synthetic
tgtggtccaggattgctac

SEQ ID NO: 813 - RNA, 19 nt, synthetic construct
  source 1..19: mol_type = other RNA; note = Synthetic
ggtgatggacagagttatc

SEQ ID NO: 814 - RNA, 19 nt, synthetic construct
  source 1..19: mol_type = other RNA; note = Synthetic
tccaccactgtcacaggaa

SEQ ID NO: 815 - RNA, 19 nt, synthetic construct
  source 1..19: mol_type = other RNA; note = Synthetic
ccaccactgtcacaggaag

SEQ ID NO: 816 - RNA, 19 nt, synthetic construct
  source 1..19: mol_type = other RNA; note = Synthetic
ctgtcacaggaaggacctg

SEQ ID NO: 817 - RNA, 19 nt, synthetic construct
  source 1..19: mol_type = other RNA; note = Synthetic
ggaaggacctgccaagctt

SEQ ID NO: 818 - RNA, 19 nt, synthetic construct
  source 1..19: mol_type = other RNA; note = Synthetic
gaaggacctgccaagcttg

SEQ ID NO: 819 - RNA, 19 nt, synthetic construct
  source 1..19: mol_type = other RNA; note = Synthetic
aggacctgccaagcttggt

SEQ ID NO: 820 - RNA, 19 nt, synthetic construct
  source 1..19: mol_type = other RNA; note = Synthetic
ggacctgccaagcttggtc

SEQ ID NO: 821 - RNA, 19 nt, synthetic construct
  source 1..19: mol_type = other RNA; note = Synthetic
gacctgccaagcttggtca

SEQ ID NO: 822 - RNA, 19 nt, synthetic construct
  source 1..19: mol_type = other RNA; note = Synthetic
acctgccaagcttggtcat

SEQ ID NO: 823 - RNA, 19 nt, synthetic construct
  source 1..19: mol_type = other RNA; note = Synthetic
cctgccaagcttggtcatc

SEQ ID NO: 824 - RNA, 19 nt, synthetic construct
  source 1..19: mol_type = other RNA; note = Synthetic
agcttggtcatctatgaca

SEQ ID NO: 825 - RNA, 19 nt, synthetic construct
  source 1..19: mol_type = other RNA; note = Synthetic
gtcatctatgacaccacat

SEQ ID NO: 826 - RNA, 19 nt, synthetic construct
  source 1..19: mol_type = other RNA; note = Synthetic
cacagaaaactacccaaat

SEQ ID NO: 827 - RNA, 19 nt, synthetic construct
  source 1..19: mol_type = other RNA; note = Synthetic
agaaaactacccaaatgct

SEQ ID NO: 828 - RNA, 19 nt, synthetic construct
  source 1..19: mol_type = other RNA; note = Synthetic
aaaactacccaaatgctgg

SEQ ID NO: 829 - RNA, 19 nt, synthetic construct
  source 1..19: mol_type = other RNA; note = Synthetic
acccaaatgctggcttgat

SEQ ID NO: 830 - RNA, 19 nt, synthetic construct
  source 1..19: mol_type = other RNA; note = Synthetic
cccaaatgctggcttgatc

SEQ ID NO: 831 - RNA, 19 nt, synthetic construct
  source 1..19: mol_type = other RNA; note = Synthetic
gaactactgcaggaatcca

SEQ ID NO: 832 - RNA, 19 nt, synthetic construct
  source 1..19: mol_type = other RNA; note = Synthetic
tactgcaggaatccagatg

SEQ ID NO: 833 - RNA, 19 nt, synthetic construct
  source 1..19: mol_type = other RNA; note = Synthetic
actgcaggaatccagatgc

SEQ ID NO: 834 - RNA, 19 nt, synthetic construct
  source 1..19: mol_type = other RNA; note = Synthetic
tgcaggaatccagatgctg

SEQ ID NO: 835 - RNA, 19 nt, synthetic construct
  source 1..19: mol_type = other RNA; note = Synthetic
aggtgggagtactgcaacc

SEQ ID NO: 836 - RNA, 19 nt, synthetic construct
  source 1..19: mol_type = other RNA; note = Synthetic
aatgctcagacgcagaagg

SEQ ID NO: 837 - RNA, 19 nt, synthetic construct
  source 1..19: mol_type = other RNA; note = Synthetic
gactgttaccccggttcca

SEQ ID NO: 838 - RNA, 19 nt, synthetic construct
  source 1..19: mol_type = other RNA; note = Synthetic
actgttaccccggttccaa

SEQ ID NO: 839 - RNA, 19 nt, synthetic construct
  source 1..19: mol_type = other RNA; note = Synthetic
ctgttaccccggttccaag

SEQ ID NO: 840 - RNA, 19 nt, synthetic construct
  source 1..19: mol_type = other RNA; note = Synthetic
tgttaccccggttccaagc

SEQ ID NO: 841 - RNA, 19 nt, synthetic construct
  source 1..19: mol_type = other RNA; note = Synthetic
gttaccccggttccaagcc

SEQ ID NO: 842 - RNA, 19 nt, synthetic construct
  source 1..19: mol_type = other RNA; note = Synthetic
ttaccccggttccaagcct

SEQ ID NO: 843 - RNA, 19 nt, synthetic construct
  source 1..19: mol_type = other RNA; note = Synthetic
taccccggttccaagccta

SEQ ID NO: 844 - RNA, 19 nt, synthetic construct
  source 1..19: mol_type = other RNA; note = Synthetic
gtgctaccatggtaatgga

SEQ ID NO: 845 - RNA, 19 nt, synthetic construct
  source 1..19: mol_type = other RNA; note = Synthetic
tgctaccatggtaatggac

SEQ ID NO: 846 - RNA, 19 nt, synthetic construct
  source 1..19: mol_type = other RNA; note = Synthetic
gctaccatggtaatggaca

SEQ ID NO: 847 - RNA, 19 nt, synthetic construct
  source 1..19: mol_type = other RNA; note = Synthetic
ctaccatggtaatggacag

SEQ ID NO: 848 - RNA, 19 nt, synthetic construct
  source 1..19: mol_type = other RNA; note = Synthetic
taccatggtaatggacaga

SEQ ID NO: 849 - RNA, 19 nt, synthetic construct
  source 1..19: mol_type = other RNA; note = Synthetic
accatggtaatggacagag

SEQ ID NO: 850 - RNA, 19 nt, synthetic construct
  source 1..19: mol_type = other RNA; note = Synthetic
ccatggtaatggacagagt

SEQ ID NO: 851 - RNA, 19 nt, synthetic construct
  source 1..19: mol_type = other RNA; note = Synthetic
catggtaatggacagagtt

SEQ ID NO: 852 - RNA, 19 nt, synthetic construct
  source 1..19: mol_type = other RNA; note = Synthetic
atggtaatggacagagtta

SEQ ID NO: 853 - RNA, 19 nt, synthetic construct
  source 1..19: mol_type = other RNA; note = Synthetic
tggtaatggacagagttat

SEQ ID NO: 854 - RNA, 19 nt, synthetic construct
  source 1..19: mol_type = other RNA; note = Synthetic
ggtaatggacagagttatc

SEQ ID NO: 855 - RNA, 19 nt, synthetic construct
  source 1..19: mol_type = other RNA; note = Synthetic
gtaatggacagagttatcg

SEQ ID NO: 856 - RNA, 19 nt, synthetic construct
  source 1..19: mol_type = other RNA; note = Synthetic
ggcacatactccaccactg

SEQ ID NO: 857 - RNA, 19 nt, synthetic construct
  source 1..19: mol_type = other RNA; note = Synthetic
cttggtcatctatgacacc

SEQ ID NO: 858 - RNA, 19 nt, synthetic construct
  source 1..19: mol_type = other RNA; note = Synthetic
gtcatctatgacaccacac

SEQ ID NO: 859 - RNA, 19 nt, synthetic construct
  source 1..19: mol_type = other RNA; note = Synthetic
tcatctatgacaccacact

SEQ ID NO: 860 - RNA, 19 nt, synthetic construct
  source 1..19: mol_type = other RNA; note = Synthetic
catctatgacaccacactc

SEQ ID NO: 861 - RNA, 19 nt, synthetic construct
  source 1..19: mol_type = other RNA; note = Synthetic
gcacatactccaccactgt

SEQ ID NO: 862 - RNA, 19 nt, synthetic construct
  source 1..19: mol_type = other RNA; note = Synthetic
agccccttattgttatacg

SEQ ID NO: 863 - RNA, 19 nt, synthetic construct
  source 1..19: mol_type = other RNA; note = Synthetic
gccccttattgttatacga

SEQ ID NO: 864 - RNA, 19 nt, synthetic construct
  source 1..19: mol_type = other RNA; note = Synthetic
ccaagcctagaggctcctt

SEQ ID NO: 865 - RNA, 19 nt, synthetic construct
  source 1..19: mol_type = other RNA; note = Synthetic
aggctccttctgaacaagc

SEQ ID NO: 866 - RNA, 19 nt, synthetic construct
  source 1..19: mol_type = other RNA; note = Synthetic
atggacagagttatcaagg

SEQ ID NO: 867 - RNA, 19 nt, synthetic construct
  source 1..19: mol_type = other RNA; note = Synthetic
tggacagagttatcaaggc

SEQ ID NO: 868 - RNA, 19 nt, synthetic construct
  source 1..19: mol_type = other RNA; note = Synthetic
ggacagagttatcaaggca

SEQ ID NO: 869 - RNA, 19 nt, synthetic construct
  source 1..19: mol_type = other RNA; note = Synthetic
gacagagttatcaaggcac

SEQ ID NO: 870 - RNA, 19 nt, synthetic construct
  source 1..19: mol_type = other RNA; note = Synthetic
acagagttatcaaggcaca

SEQ ID NO: 871 - RNA, 19 nt, synthetic construct
  source 1..19: mol_type = other RNA; note = Synthetic
cagagttatcaaggcacat

SEQ ID NO: 872 - RNA, 19 nt, synthetic construct
  source 1..19: mol_type = other RNA; note = Synthetic
tacccaaatgctggcttga

SEQ ID NO: 873 - RNA, 19 nt, synthetic construct
  source 1..19: mol_type = other RNA; note = Synthetic
ccaaatgctggcttgatca

SEQ ID NO: 874 - RNA, 19 nt, synthetic construct
  source 1..19: mol_type = other RNA; note = Synthetic
tcaagaactactgccgaaa

SEQ ID NO: 875 - RNA, 19 nt, synthetic construct
  source 1..19: mol_type = other RNA; note = Synthetic
caagaactactgccgaaat

SEQ ID NO: 876 - RNA, 19 nt, synthetic construct
  source 1..19: mol_type = other RNA; note = Synthetic
aagaactactgccgaaatc

SEQ ID NO: 877 - RNA, 19 nt, synthetic construct
  source 1..19: mol_type = other RNA; note = Synthetic
gaactactgccgaaatcca

SEQ ID NO: 878 - RNA, 19 nt, synthetic construct
  source 1..19: mol_type = other RNA; note = Synthetic
ctactgccgaaatccagat

SEQ ID NO: 879 - RNA, 19 nt, synthetic construct
  source 1..19: mol_type = other RNA; note = Synthetic
tgtggcagccccttggtgt

SEQ ID NO: 880 - RNA, 19 nt, synthetic construct
  source 1..19: mol_type = other RNA; note = Synthetic
gtggcagccccttggtgtt

SEQ ID NO: 881 - RNA, 19 nt, synthetic construct
  source 1..19: mol_type = other RNA; note = Synthetic
tggcagccccttggtgtta

SEQ ID NO: 882 - RNA, 19 nt, synthetic construct
  source 1..19: mol_type = other RNA; note = Synthetic
ggcagccccttggtgttat

SEQ ID NO: 883 - RNA, 19 nt, synthetic construct
  source 1..19: mol_type = other RNA; note = Synthetic
gcagccccttggtgttata

SEQ ID NO: 884 - RNA, 19 nt, synthetic construct
  source 1..19: mol_type = other RNA; note = Synthetic
cagccccttggtgttatac

SEQ ID NO: 885 - RNA, 19 nt, synthetic construct
  source 1..19: mol_type = other RNA; note = Synthetic
agccccttggtgttataca

SEQ ID NO: 886 - RNA, 19 nt, synthetic construct
  source 1..19: mol_type = other RNA; note = Synthetic
gccccttggtgttatacaa

SEQ ID NO: 887 - RNA, 19 nt, synthetic construct
  source 1..19: mol_type = other RNA; note = Synthetic
ccccttggtgttatacaac

SEQ ID NO: 888 - RNA, 19 nt, synthetic construct
  source 1..19: mol_type = other RNA; note = Synthetic
ggtgggagtactgcaacct

SEQ ID NO: 889 - RNA, 19 nt, synthetic construct
  source 1..19: mol_type = other RNA; note = Synthetic
gtgggagtactgcaacctg

SEQ ID NO: 890 - RNA, 19 nt, synthetic construct
  source 1..19: mol_type = other RNA; note = Synthetic
ggagtactgcaacctgaca

SEQ ID NO: 891 - RNA, 19 nt, synthetic construct
  source 1..19: mol_type = other RNA; note = Synthetic
gagtactgcaacctgacac

SEQ ID NO: 892 - RNA, 19 nt, synthetic construct
  source 1..19: mol_type = other RNA; note = Synthetic
gtactgcaacctgacacga

SEQ ID NO: 893 - RNA, 19 nt, synthetic construct
  source 1..19: mol_type = other RNA; note = Synthetic
tactgcaacctgacacgat

SEQ ID NO: 894 - RNA, 19 nt, synthetic construct
  source 1..19: mol_type = other RNA; note = Synthetic
actgcaacctgacacgatg

SEQ ID NO: 895 - RNA, 19 nt, synthetic construct
  source 1..19: mol_type = other RNA; note = Synthetic
ctgcaacctgacacgatgc

SEQ ID NO: 896 - RNA, 19 nt, synthetic construct
  source 1..19: mol_type = other RNA; note = Synthetic
tgcaacctgacacgatgct

SEQ ID NO: 897 - RNA, 19 nt, synthetic construct
  source 1..19: mol_type = other RNA; note = Synthetic
caacctgacacgatgctca

SEQ ID NO: 898 - RNA, 19 nt, synthetic construct
  source 1..19: mol_type = other RNA; note = Synthetic
acctgacacgatgctcaga

SEQ ID NO: 899 - RNA, 19 nt, synthetic construct
  source 1..19: mol_type = other RNA; note = Synthetic
cctgacacgatgctcagat

SEQ ID NO: 900 - RNA, 19 nt, synthetic construct
  source 1..19: mol_type = other RNA; note = Synthetic
ctgacacgatgctcagatg

SEQ ID NO: 901 - RNA, 19 nt, synthetic construct
  source 1..19: mol_type = other RNA; note = Synthetic
tgacacgatgctcagatgc

SEQ ID NO: 902 - RNA, 19 nt, synthetic construct
  source 1..19: mol_type = other RNA; note = Synthetic
gacacgatgctcagatgca

SEQ ID NO: 903 - RNA, 19 nt, synthetic construct
  source 1..19: mol_type = other RNA; note = Synthetic
acacgatgctcagatgcag

SEQ ID NO: 904 - RNA, 19 nt, synthetic construct
  source 1..19: mol_type = other RNA; note = Synthetic
cacgatgctcagatgcaga

SEQ ID NO: 905 - RNA, 19 nt, synthetic construct
  source 1..19: mol_type = other RNA; note = Synthetic
tgctactaccattatggac

SEQ ID NO: 906 - RNA, 19 nt, synthetic construct
  source 1..19: mol_type = other RNA; note = Synthetic
gctactaccattatggaca

SEQ ID NO: 907 - RNA, 19 nt, synthetic construct
  source 1..19: mol_type = other RNA; note = Synthetic
ctactaccattatggacag

SEQ ID NO: 908 - RNA, 19 nt, synthetic construct
  source 1..19: mol_type = other RNA; note = Synthetic
tactaccattatggacaga

SEQ ID NO: 909 - RNA, 19 nt, synthetic construct
  source 1..19: mol_type = other RNA; note = Synthetic
actaccattatggacagag

SEQ ID NO: 910 - RNA, 19 nt, synthetic construct
  source 1..19: mol_type = other RNA; note = Synthetic
ctaccattatggacagagt

SEQ ID NO: 911 - RNA, 19 nt, synthetic construct
  source 1..19: mol_type = other RNA; note = Synthetic
taccattatggacagagtt

SEQ ID NO: 912 - RNA, 19 nt, synthetic construct
  source 1..19: mol_type = other RNA; note = Synthetic
accattatggacagagtta

SEQ ID NO: 913 - RNA, 19 nt, synthetic construct
  source 1..19: mol_type = other RNA; note = Synthetic
gaggcacatactccaccac

SEQ ID NO: 914 - RNA, 19 nt, synthetic construct
  source 1..19: mol_type = other RNA; note = Synthetic
ctccaccactgtcacagga

SEQ ID NO: 915 - RNA, 19 nt, synthetic construct
  source 1..19: mol_type = other RNA; note = Synthetic
acaggaagaacttgccaag

SEQ ID NO: 916 - RNA, 19 nt, synthetic construct
  source 1..19: mol_type = other RNA; note = Synthetic
caggaagaacttgccaagc

SEQ ID NO: 917 - RNA, 19 nt, synthetic construct
  source 1..19: mol_type = other RNA; note = Synthetic
aggaagaacttgccaagct

SEQ ID NO: 918 - RNA, 19 nt, synthetic construct
  source 1..19: mol_type = other RNA; note = Synthetic
ggaagaacttgccaagctt

SEQ ID NO: 919 - RNA, 19 nt, synthetic construct
  source 1..19: mol_type = other RNA; note = Synthetic
gaagaacttgccaagcttg

SEQ ID NO: 920 - RNA, 19 nt, synthetic construct
  source 1..19: mol_type = other RNA; note = Synthetic
aagaacttgccaagcttgg

SEQ ID NO: 921 - RNA, 19 nt, synthetic construct
  source 1..19: mol_type = other RNA; note = Synthetic
agaacttgccaagcttggt

SEQ ID NO: 922 - RNA, 19 nt, synthetic construct
  source 1..19: mol_type = other RNA; note = Synthetic
gaacttgccaagcttggtc

SEQ ID NO: 923 - RNA, 19 nt, synthetic construct
  source 1..19: mol_type = other RNA; note = Synthetic
aacttgccaagcttggtca

SEQ ID NO: 924 - RNA, 19 nt, synthetic construct
  source 1..19: mol_type = other RNA; note = Synthetic
acttgccaagcttggtcat

SEQ ID NO: 925 - RNA, 19 nt, synthetic construct
  source 1..19: mol_type = other RNA; note = Synthetic
cttgccaagcttggtcatc

SEQ ID NO: 926 - RNA, 19 nt, synthetic construct
  source 1..19: mol_type = other RNA; note = Synthetic
ttgccaagcttggtcatct

SEQ ID NO: 927 - RNA, 19 nt, synthetic construct
  source 1..19: mol_type = other RNA; note = Synthetic
tgccaagcttggtcatcta

SEQ ID NO: 928 - RNA, 19 nt, synthetic construct
  source 1..19: mol_type = other RNA; note = Synthetic
gcttggtcatctatgacac

SEQ ID NO: 929 - RNA, 19 nt, synthetic construct
  source 1..19: mol_type = other RNA; note = Synthetic
ttggtcatctatgacacca

SEQ ID NO: 930 - RNA, 19 nt, synthetic construct
  source 1..19: mol_type = other RNA; note = Synthetic
tggtcatctatgacaccac

SEQ ID NO: 931 - RNA, 19 nt, synthetic construct
  source 1..19: mol_type = other RNA; note = Synthetic
gtcatctatgacaccacac

SEQ ID NO: 932 - RNA, 19 nt, synthetic construct
  source 1..19: mol_type = other RNA; note = Synthetic
catctatgacaccacacca

SEQ ID NO: 933 - RNA, 19 nt, synthetic construct
  source 1..19: mol_type = other RNA; note = Synthetic
atctatgacaccacaccag

SEQ ID NO: 934 - RNA, 19 nt, synthetic construct
  source 1..19: mol_type = other RNA; note = Synthetic
ctatgacaccacaccagca

SEQ ID NO: 935 - RNA, 19 nt, synthetic construct
  source 1..19: mol_type = other RNA; note = Synthetic
gtcggaccccagaaaacta

SEQ ID NO: 936 - RNA, 19 nt, synthetic construct
  source 1..19: mol_type = other RNA; note = Synthetic
tcggaccccagaaaactac

SEQ ID NO: 937 - RNA, 19 nt, synthetic construct
  source 1..19: mol_type = other RNA; note = Synthetic
gaaaactacccaaatgctg

SEQ ID NO: 938 - RNA, 19 nt, synthetic construct
  source 1..19: mol_type = other RNA; note = Synthetic
gctgagattcgcccttggt

SEQ ID NO: 939 - RNA, 19 nt, synthetic construct
  source 1..19: mol_type = other RNA; note = Synthetic
ctgagattcgcccttggtg

SEQ ID NO: 940 - RNA, 19 nt, synthetic construct
  source 1..19: mol_type = other RNA; note = Synthetic
gagattcgcccttggtgtt

SEQ ID NO: 941 - RNA, 19 nt, synthetic construct
  source 1..19: mol_type = other RNA; note = Synthetic
agattcgcccttggtgtta

SEQ ID NO: 942 - RNA, 19 nt, synthetic construct
  source 1..19: mol_type = other RNA; note = Synthetic
ttcgcccttggtgttacac

SEQ ID NO: 943 - RNA, 19 nt, synthetic construct
  source 1..19: mol_type = other RNA; note = Synthetic
cttggtgttacaccatgga

SEQ ID NO: 944 - RNA, 19 nt, synthetic construct
  source 1..19: mol_type = other RNA; note = Synthetic
ttggtgttacaccatggat

SEQ ID NO: 945 - RNA, 19 nt, synthetic construct
  source 1..19: mol_type = other RNA; note = Synthetic
tggtgttacaccatggatc

SEQ ID NO: 946 - RNA, 19 nt, synthetic construct
  source 1..19: mol_type = other RNA; note = Synthetic
ggtgttacaccatggatcc

SEQ ID NO: 947 - RNA, 19 nt, synthetic construct
  source 1..19: mol_type = other RNA; note = Synthetic
tgttacaccatggatccca

SEQ ID NO: 948 - RNA, 19 nt, synthetic construct
  source 1..19: mol_type = other RNA; note = Synthetic
gaatcaagtgtccttgcaa

SEQ ID NO: 949 - RNA, 19 nt, synthetic construct
  source 1..19: mol_type = other RNA; note = Synthetic
aatcaagtgtccttgcaac

SEQ ID NO: 950 - RNA, 19 nt, synthetic construct
  source 1..19: mol_type = other RNA; note = Synthetic
atcaagtgtccttgcaact

SEQ ID NO: 951 - RNA, 19 nt, synthetic construct
  source 1..19: mol_type = other RNA; note = Synthetic
atggacagagttatcgagg

SEQ ID NO: 952 - RNA, 19 nt, synthetic construct
  source 1..19: mol_type = other RNA; note = Synthetic
tggacagagttatcgaggc

SEQ ID NO: 953 - RNA, 19 nt, synthetic construct
  source 1..19: mol_type = other RNA; note = Synthetic
acaccacactggcatcaga

SEQ ID NO: 954 - RNA, 19 nt, synthetic construct
  source 1..19: mol_type = other RNA; note = Synthetic
ttggtgttataccatggat

SEQ ID NO: 955 - RNA, 19 nt, synthetic construct
  source 1..19: mol_type = other RNA; note = Synthetic
tggtgttataccatggatc

SEQ ID NO: 956 - RNA, 19 nt, synthetic construct
  source 1..19: mol_type = other RNA; note = Synthetic
ggtgttataccatggatcc

SEQ ID NO: 957 - RNA, 19 nt, synthetic construct
  source 1..19: mol_type = other RNA; note = Synthetic
gtgttataccatggatccc

SEQ ID NO: 958 - RNA, 19 nt, synthetic construct
  source 1..19: mol_type = other RNA; note = Synthetic
tcagatgggagtactgcaa

SEQ ID NO: 959 - RNA, 19 nt, synthetic construct
  source 1..19: mol_type = other RNA; note = Synthetic
gatgggagtactgcaacct

SEQ ID NO: 960 - RNA, 19 nt, synthetic construct
  source 1..19: mol_type = other RNA; note = Synthetic
atgggagtactgcaacctg

SEQ ID NO: 961 - RNA, 19 nt, synthetic construct
  source 1..19: mol_type = other RNA; note = Synthetic
tgggagtactgcaacctga

SEQ ID NO: 962 - RNA, 19 nt, synthetic construct
  source 1..19: mol_type = other RNA; note = Synthetic
gggagtactgcaacctgac

SEQ ID NO: 963 - RNA, 19 nt, synthetic construct
  source 1..19: mol_type = other RNA; note = Synthetic
ggctgtttctgaacaagca

SEQ ID NO: 964 - RNA, 19 nt, synthetic construct
  source 1..19: mol_type = other RNA; note = Synthetic
gtttctgaacaagcaccaa

SEQ ID NO: 965 - RNA, 19 nt, synthetic construct
  source 1..19: mol_type = other RNA; note = Synthetic
ctccaccactgttacagga

SEQ ID NO: 966 - RNA, 19 nt, synthetic construct
  source 1..19: mol_type = other RNA; note = Synthetic
tccaccactgttacaggaa

SEQ ID NO: 967 - RNA, 19 nt, synthetic construct
  source 1..19: mol_type = other RNA; note = Synthetic
ccaccactgttacaggaag

SEQ ID NO: 968 - RNA, 19 nt, synthetic construct
  source 1..19: mol_type = other RNA; note = Synthetic
gacaccacactggcatcag

SEQ ID NO: 969 - RNA, 19 nt, synthetic construct
  source 1..19: mol_type = other RNA; note = Synthetic
acaccacactggcatcaga

SEQ ID NO: 970 - RNA, 19 nt, synthetic construct
  source 1..19: mol_type = other RNA; note = Synthetic
agaatactacccaaatggt

SEQ ID NO: 971 - RNA, 19 nt, synthetic construct
  source 1..19: mol_type = other RNA; note = Synthetic
gaatactacccaaatggtg

SEQ ID NO: 972 - RNA, 19 nt, synthetic construct
  source 1..19: mol_type = other RNA; note = Synthetic
aatactacccaaatggtgg

SEQ ID NO: 973 - RNA, 19 nt, synthetic construct
  source 1..19: mol_type = other RNA; note = Synthetic
tccttctgaagaagcacca

SEQ ID NO: 974 - RNA, 19 nt, synthetic construct
  source 1..19: mol_type = other RNA; note = Synthetic
ccttctgaagaagcaccaa

SEQ ID NO: 975 - RNA, 19 nt, synthetic construct
  source 1..19: mol_type = other RNA; note = Synthetic
cttctgaagaagcaccaac

SEQ ID NO: 976 - RNA, 19 nt, synthetic construct
  source 1..19: mol_type = other RNA; note = Synthetic
ttctgaagaagcaccaact

SEQ ID NO: 977 - RNA, 19 nt, synthetic construct
  source 1..19: mol_type = other RNA; note = Synthetic
tctgaagaagcaccaactg

SEQ ID NO: 978 - RNA, 19 nt, synthetic construct
  source 1..19: mol_type = other RNA; note = Synthetic
ctgaagaagcaccaactga

SEQ ID NO: 979 - RNA, 19 nt, synthetic construct
  source 1..19: mol_type = other RNA; note = Synthetic
tgaagaagcaccaactgaa

SEQ ID NO: 980 - RNA, 19 nt, synthetic construct
  source 1..19: mol_type = other RNA; note = Synthetic
gaagaagcaccaactgaaa

SEQ ID NO: 981 - RNA, 19 nt, synthetic construct
  source 1..19: mol_type = other RNA; note = Synthetic
aagaagcaccaactgaaaa

SEQ ID NO: 982 - RNA, 19 nt, synthetic construct
  source 1..19: mol_type = other RNA; note = Synthetic
agaagcaccaactgaaaac

SEQ ID NO: 983 - RNA, 19 nt, synthetic construct
  source 1..19: mol_type = other RNA; note = Synthetic
gaagcaccaactgaaaaca

SEQ ID NO: 984 - RNA, 19 nt, synthetic construct
  source 1..19: mol_type = other RNA; note = Synthetic
aagcaccaactgaaaacag

SEQ ID NO: 985 - RNA, 19 nt, synthetic construct
  source 1..19: mol_type = other RNA; note = Synthetic
aggtgatggacagagttat

SEQ ID NO: 986 - RNA, 19 nt, synthetic construct
  source 1..19: mol_type = other RNA; note = Synthetic
ctccaccactatcacagga

SEQ ID NO: 987 - RNA, 19 nt, synthetic construct
  source 1..19: mol_type = other RNA; note = Synthetic
tccaccactatcacaggaa

SEQ ID NO: 988 - RNA, 19 nt, synthetic construct
  source 1..19: mol_type = other RNA; note = Synthetic
ccaccactatcacaggaag

SEQ ID NO: 989 - RNA, 19 nt, synthetic construct
  source 1..19: mol_type = other RNA; note = Synthetic
caccactatcacaggaaga

SEQ ID NO: 990 - RNA, 19 nt, synthetic construct
  source 1..19: mol_type = other RNA; note = Synthetic
accactatcacaggaagaa

SEQ ID NO: 991 - RNA, 19 nt, synthetic construct
  source 1..19: mol_type = other RNA; note = Synthetic
ccactatcacaggaagaac

SEQ ID NO: 992 - RNA, 19 nt, synthetic construct
  source 1..19: mol_type = other RNA; note = Synthetic
cactatcacaggaagaaca

SEQ ID NO: 993 - RNA, 19 nt, synthetic construct
  source 1..19: mol_type = other RNA; note = Synthetic
actatcacaggaagaacat

SEQ ID NO: 994 - RNA, 19 nt, synthetic construct
  source 1..19: mol_type = other RNA; note = Synthetic
ctatcacaggaagaacatg

SEQ ID NO: 995 - RNA, 19 nt, synthetic construct
  source 1..19: mol_type = other RNA; note = Synthetic
tatcacaggaagaacatgt

SEQ ID NO: 996 - RNA, 19 nt, synthetic construct
  source 1..19: mol_type = other RNA; note = Synthetic
atcacaggaagaacatgtc

SEQ ID NO: 997 - RNA, 19 nt, synthetic construct
  source 1..19: mol_type = other RNA; note = Synthetic
tcacaggaagaacatgtca

SEQ ID NO: 998 - RNA, 19 nt, synthetic construct
  source 1..19: mol_type = other RNA; note = Synthetic
cacaggaagaacatgtcag

SEQ ID NO: 999 - RNA, 19 nt, synthetic construct
  source 1..19: mol_type = other RNA; note = Synthetic
acaggaagaacatgtcagt

SEQ ID NO: 1000 - RNA, 19 nt, synthetic construct
  source 1..19: mol_type = other RNA; note = Synthetic
ggaagaacatgtcagtctt

SEQ ID NO: 1001 - RNA, 19 nt, synthetic construct
  source 1..19: mol_type = other RNA; note = Synthetic
gaagaacatgtcagtcttg

SEQ ID NO: 1002 - RNA, 19 nt, synthetic construct
  source 1..19: mol_type = other RNA; note = Synthetic
aagaacatgtcagtcttgg

SEQ ID NO: 1003 - RNA, 19 nt, synthetic construct
  source 1..19: mol_type = other RNA; note = Synthetic
agaacatgtcagtcttggt

SEQ ID NO: 1004 - RNA, 19 nt, synthetic construct
  source 1..19: mol_type = other RNA; note = Synthetic
ggcatcggaggatcccatt

SEQ ID NO: 1005 - RNA, 19 nt, synthetic construct
  source 1..19: mol_type = other RNA; note = Synthetic
actatccaaatgctggcct

SEQ ID NO: 1006 - RNA, 19 nt, synthetic construct
  source 1..19: mol_type = other RNA; note = Synthetic
gcacagaggctccttctga

SEQ ID NO: 1007 - RNA, 19 nt, synthetic construct
  source 1..19: mol_type = other RNA; note = Synthetic
tccttctgaacaagcacca

SEQ ID NO: 1008 - RNA, 19 nt, synthetic construct
  source 1..19: mol_type = other RNA; note = Synthetic
ccttctgaacaagcaccac

SEQ ID NO: 1009 - RNA, 19 nt, synthetic construct
  source 1..19: mol_type = other RNA; note = Synthetic
cttctgaacaagcaccacc

SEQ ID NO: 1010 - RNA, 19 nt, synthetic construct
  source 1..19: mol_type = other RNA; note = Synthetic
ttctgaacaagcaccacct

SEQ ID NO: 1011 - RNA, 19 nt, synthetic construct
  source 1..19: mol_type = other RNA; note = Synthetic
tctgaacaagcaccacctg

SEQ ID NO: 1012 - RNA, 19 nt, synthetic construct
  source 1..19: mol_type = other RNA; note = Synthetic
ctgaacaagcaccacctga

SEQ ID NO: 1013 - RNA, 19 nt, synthetic construct
  source 1..19: mol_type = other RNA; note = Synthetic
tgaacaagcaccacctgag

SEQ ID NO: 1014 - RNA, 19 nt, synthetic construct
  source 1..19: mol_type = other RNA; note = Synthetic
gaacaagcaccacctgaga

SEQ ID NO: 1015 - RNA, 19 nt, synthetic construct
  source 1..19: mol_type = other RNA; note = Synthetic
aacaagcaccacctgagaa

SEQ ID NO: 1016 - RNA, 19 nt, synthetic construct
  source 1..19: mol_type = other RNA; note = Synthetic
caagcaccacctgagaaaa

SEQ ID NO: 1017 - RNA, 19 nt, synthetic construct
  source 1..19: mol_type = other RNA; note = Synthetic
aagcaccacctgagaaaag

SEQ ID NO: 1018 - RNA, 19 nt, synthetic construct
  source 1..19: mol_type = other RNA; note = Synthetic
agcaccacctgagaaaagc

SEQ ID NO: 1019 - RNA, 19 nt, synthetic construct
  source 1..19: mol_type = other RNA; note = Synthetic
ctgagaaaagccctgtggt

SEQ ID NO: 1020 - RNA, 19 nt, synthetic construct
  source 1..19: mol_type = other RNA; note = Synthetic
gccctgtggtccaggattg

SEQ ID NO: 1021 - RNA, 19 nt, synthetic construct
  source 1..19: mol_type = other RNA; note = Synthetic
ctgtggtccaggattgcta

SEQ ID NO: 1022 - RNA, 19 nt, synthetic construct
  source 1..19: mol_type = other RNA; note = Synthetic
ctccaccactgtcacagga

SEQ ID NO: 1023 - RNA, 19 nt, synthetic construct
  source 1..19: mol_type = other RNA; note = Synthetic
tccaccactgtcacaggaa

SEQ ID NO: 1024 - RNA, 19 nt, synthetic construct
  source 1..19: mol_type = other RNA; note = Synthetic
tcttggtcatctatgatac

SEQ ID NO: 1025 - RNA, 19 nt, synthetic construct
  source 1..19: mol_type = other RNA; note = Synthetic
cttggtcatctatgatacc

SEQ ID NO: 1026 - RNA, 19 nt, synthetic construct
  source 1..19: mol_type = other RNA; note = Synthetic
ttggtcatctatgatacca

SEQ ID NO: 1027 - RNA, 19 nt, synthetic construct
  source 1..19: mol_type = other RNA; note = Synthetic
tggtcatctatgataccac

SEQ ID NO: 1028 - RNA, 19 nt, synthetic construct
  source 1..19: mol_type = other RNA; note = Synthetic
ggtcatctatgataccaca

SEQ ID NO: 1029 - RNA, 19 nt, synthetic construct
  source 1..19: mol_type = other RNA; note = Synthetic
gtcatctatgataccacac

SEQ ID NO: 1030 - RNA, 19 nt, synthetic construct
  source 1..19: mol_type = other RNA; note = Synthetic
tcatctatgataccacact

SEQ ID NO: 1031 - RNA, 19 nt, synthetic construct
  source 1..19: mol_type = other RNA; note = Synthetic
catctatgataccacactg

SEQ ID NO: 1032 - RNA, 19 nt, synthetic construct
  source 1..19: mol_type = other RNA; note = Synthetic
atctatgataccacactgg

SEQ ID NO: 1033 - RNA, 19 nt, synthetic construct
  source 1..19: mol_type = other RNA; note = Synthetic
tctatgataccacactggc

SEQ ID NO: 1034 - RNA, 19 nt, synthetic construct
  source 1..19: mol_type = other RNA; note = Synthetic
ctatgataccacactggca

SEQ ID NO: 1035 - RNA, 19 nt, synthetic construct
  source 1..19: mol_type = other RNA; note = Synthetic
tgataccacactggcatca

SEQ ID NO: 1036 - RNA, 19 nt, synthetic construct
  source 1..19: mol_type = other RNA; note = Synthetic
ataccacactggcatcaga

SEQ ID NO: 1037 - RNA, 19 nt, synthetic construct
  source 1..19: mol_type = other RNA; note = Synthetic
agaggaccccagaaaacta

SEQ ID NO: 1038 - RNA, 19 nt, synthetic construct
  source 1..19: mol_type = other RNA; note = Synthetic
gaggaccccagaaaactac

SEQ ID NO: 1039 - RNA, 19 nt, synthetic construct
  source 1..19: mol_type = other RNA; note = Synthetic
agaactactgcaggaatcc

SEQ ID NO: 1040 - RNA, 19 nt, synthetic construct
  source 1..19: mol_type = other RNA; note = Synthetic
actactgcaggaatccaga

SEQ ID NO: 1041 - RNA, 19 nt, synthetic construct
  source 1..19: mol_type = other RNA; note = Synthetic
tactgcaggaatccagatt

SEQ ID NO: 1042 - RNA, 19 nt, synthetic construct
  source 1..19: mol_type = other RNA; note = Synthetic
actgcaggaatccagattc

SEQ ID NO: 1043 - RNA, 19 nt, synthetic construct
  source 1..19: mol_type = other RNA; note = Synthetic
ctgcaggaatccagattct

SEQ ID NO: 1044 - RNA, 19 nt, synthetic construct
  source 1..19: mol_type = other RNA; note = Synthetic
tgcaggaatccagattctg

SEQ ID NO: 1045 - RNA, 19 nt, synthetic construct
  source 1..19: mol_type = other RNA; note = Synthetic
gcaggaatccagattctgg

SEQ ID NO: 1046 - RNA, 19 nt, synthetic construct
  source 1..19: mol_type = other RNA; note = Synthetic
ggaatccagattctgggaa

SEQ ID NO: 1047 - RNA, 19 nt, synthetic construct
  source 1..19: mol_type = other RNA; note = Synthetic
gggaaacaaccctggtgtt

SEQ ID NO: 1048 - RNA, 19 nt, synthetic construct
  source 1..19: mol_type = other RNA; note = Synthetic
ggaaacaaccctggtgtta

SEQ ID NO: 1049 - RNA, 19 nt, synthetic construct
  source 1..19: mol_type = other RNA; note = Synthetic
tgtgtgaggtgggagtact

SEQ ID NO: 1050 - RNA, 19 nt, synthetic construct
  source 1..19: mol_type = other RNA; note = Synthetic
gtgtgaggtgggagtactg

SEQ ID NO: 1051 - RNA, 19 nt, synthetic construct
  source 1..19: mol_type = other RNA; note = Synthetic
gtgaggtgggagtactgca

SEQ ID NO: 1052 - RNA, 19 nt, synthetic construct
  source 1..19: mol_type = other RNA; note = Synthetic
tgaggtgggagtactgcaa

SEQ ID NO: 1053 - RNA, 19 nt, synthetic construct
  source 1..19: mol_type = other RNA; note = Synthetic
ctgacacaatgctcagaaa

SEQ ID NO: 1054 - RNA, 19 nt, synthetic construct
  source 1..19: mol_type = other RNA; note = Synthetic
tgacacaatgctcagaaac

SEQ ID NO: 1055 - RNA, 19 nt, synthetic construct
  source 1..19: mol_type = other RNA; note = Synthetic
gacacaatgctcagaaaca

SEQ ID NO: 1056 - RNA, 19 nt, synthetic construct
  source 1..19: mol_type = other RNA; note = Synthetic
acacaatgctcagaaacag

SEQ ID NO: 1057 - RNA, 19 nt, synthetic construct
  source 1..19: mol_type = other RNA; note = Synthetic
cacaatgctcagaaacaga

SEQ ID NO: 1058 - RNA, 19 nt, synthetic construct
  source 1..19: mol_type = other RNA; note = Synthetic
acaatgctcagaaacagaa

SEQ ID NO: 1059 - RNA, 19 nt, synthetic construct
  source 1..19: mol_type = other RNA; note = Synthetic
caatgctcagaaacagaat

SEQ ID NO: 1060 - RNA, 19 nt, synthetic construct
  source 1..19: mol_type = other RNA; note = Synthetic
aatgctcagaaacagaatc

SEQ ID NO: 1061 - RNA, 19 nt, synthetic construct
  source 1..19: mol_type = other RNA; note = Synthetic
atgctcagaaacagaatca

SEQ ID NO: 1062 - RNA, 19 nt, synthetic construct
  source 1..19: mol_type = other RNA; note = Synthetic
tgctcagaaacagaatcag

SEQ ID NO: 1063 - RNA, 19 nt, synthetic construct
  source 1..19: mol_type = other RNA; note = Synthetic
ctcagaaacagaatcaggt

SEQ ID NO: 1064 - RNA, 19 nt, synthetic construct
  source 1..19: mol_type = other RNA; note = Synthetic
cagaaacagaatcaggtgt

SEQ ID NO: 1065 - RNA, 19 nt, synthetic construct
  source 1..19: mol_type = other RNA; note = Synthetic
agaaacagaatcaggtgtc

SEQ ID NO: 1066 - RNA, 19 nt, synthetic construct
  source 1..19: mol_type = other RNA; note = Synthetic
gaaacagaatcaggtgtcc

SEQ ID NO: 1067 - RNA, 19 nt, synthetic construct
  source 1..19: mol_type = other RNA; note = Synthetic
aaacagaatcaggtgtcct

SEQ ID NO: 1068 - RNA, 19 nt, synthetic construct
  source 1..19: mol_type = other RNA; note = Synthetic
aacagaatcaggtgtccta

SEQ ID NO: 1069 - RNA, 19 nt, synthetic construct
  source 1..19: mol_type = other RNA; note = Synthetic
cagaatcaggtgtcctaga

SEQ ID NO: 1070 - RNA, 19 nt, synthetic construct
  source 1..19: mol_type = other RNA; note = Synthetic
gaatcaggtgtcctagaga

SEQ ID NO: 1071 - RNA, 19 nt, synthetic construct
  source 1..19: mol_type = other RNA; note = Synthetic
atcaggtgtcctagagact

SEQ ID NO: 1072 - RNA, 19 nt, synthetic construct
  source 1..19: mol_type = other RNA; note = Synthetic
ggtgtcctagagactccca

SEQ ID NO: 1073 - RNA, 19 nt, synthetic construct
  source 1..19: mol_type = other RNA; note = Synthetic
cctagagactcccactgtt

SEQ ID NO: 1074 - RNA, 19 nt, synthetic construct
  source 1..19: mol_type = other RNA; note = Synthetic
ctagagactcccactgttg

SEQ ID NO: 1075 - RNA, 19 nt, synthetic construct
  source 1..19: mol_type = other RNA; note = Synthetic
tagagactcccactgttgt

SEQ ID NO: 1076 - RNA, 19 nt, synthetic construct
  source 1..19: mol_type = other RNA; note = Synthetic
agagactcccactgttgtt

SEQ ID NO: 1077 - RNA, 19 nt, synthetic construct
  source 1..19: mol_type = other RNA; note = Synthetic
gagactcccactgttgttc

SEQ ID NO: 1078 - RNA, 19 nt, synthetic construct
  source 1..19: mol_type = other RNA; note = Synthetic
agactcccactgttgttcc

SEQ ID NO: 1079 - RNA, 19 nt, synthetic construct
  source 1..19: mol_type = other RNA; note = Synthetic
gctcattctgaagcagcac

SEQ ID NO: 1080 - RNA, 19 nt, synthetic construct
  source 1..19: mol_type = other RNA; note = Synthetic
ctcattctgaagcagcacc

SEQ ID NO: 1081 - RNA, 19 nt, synthetic construct
  source 1..19: mol_type = other RNA; note = Synthetic
tcattctgaagcagcacca

SEQ ID NO: 1082 - RNA, 19 nt, synthetic construct
  source 1..19: mol_type = other RNA; note = Synthetic
cattctgaagcagcaccaa

SEQ ID NO: 1083 - RNA, 19 nt, synthetic construct
  source 1..19: mol_type = other RNA; note = Synthetic
attctgaagcagcaccaac

SEQ ID NO: 1084 - RNA, 19 nt, synthetic construct
  source 1..19: mol_type = other RNA; note = Synthetic
ttctgaagcagcaccaact

SEQ ID NO: 1085 - RNA, 19 nt, synthetic construct
  source 1..19: mol_type = other RNA; note = Synthetic
tctgaagcagcaccaactg

SEQ ID NO: 1086 - RNA, 19 nt, synthetic construct
  source 1..19: mol_type = other RNA; note = Synthetic
ctgaagcagcaccaactga

SEQ ID NO: 1087 - RNA, 19 nt, synthetic construct
  source 1..19: mol_type = other RNA; note = Synthetic
tgaagcagcaccaactgag

SEQ ID NO: 1088 - RNA, 19 nt, synthetic construct
  source 1..19: mol_type = other RNA; note = Synthetic
gaagcagcaccaactgagc

SEQ ID NO: 1089 - RNA, 19 nt, synthetic construct
  source 1..19: mol_type = other RNA; note = Synthetic
aagcagcaccaactgagca

SEQ ID NO: 1090 - RNA, 19 nt, synthetic construct
  source 1..19: mol_type = other RNA; note = Synthetic
cagtgctaccatggtaatg

SEQ ID NO: 1091 - RNA, 19 nt, synthetic construct
  source 1..19: mol_type = other RNA; note = Synthetic
agtgctaccatggtaatgg

SEQ ID NO: 1092 - RNA, 19 nt, synthetic construct
  source 1..19: mol_type = other RNA; note = Synthetic
acattctccaccactgtca

SEQ ID NO: 1093 - RNA, 19 nt, synthetic construct
  source 1..19: mol_type = other RNA; note = Synthetic
cactgtcacaggaaggaca

SEQ ID NO: 1094 - RNA, 19 nt, synthetic construct
  source 1..19: mol_type = other RNA; note = Synthetic
actgtcacaggaaggacat

SEQ ID NO: 1095 - RNA, 19 nt, synthetic construct
  source 1..19: mol_type = other RNA; note = Synthetic
ctgtcacaggaaggacatg

SEQ ID NO: 1096 - RNA, 19 nt, synthetic construct
  source 1..19: mol_type = other RNA; note = Synthetic
tgtcacaggaaggacatgt

SEQ ID NO: 1097 - RNA, 19 nt, synthetic construct
  source 1..19: mol_type = other RNA; note = Synthetic
gtcacaggaaggacatgtc

SEQ ID NO: 1098 - RNA, 19 nt, synthetic construct
  source 1..19: mol_type = other RNA; note = Synthetic
tcacaggaaggacatgtca

SEQ ID NO: 1099 - RNA, 19 nt, synthetic construct
  source 1..19: mol_type = other RNA; note = Synthetic
acaggaaggacatgtcaat

SEQ ID NO: 1100 - RNA, 19 nt, synthetic construct
  source 1..19: mol_type = other RNA; note = Synthetic
caggaaggacatgtcaatc

SEQ ID NO: 1101 - RNA, 19 nt, synthetic construct
  source 1..19: mol_type = other RNA; note = Synthetic
aggaaggacatgtcaatct

SEQ ID NO: 1102 - RNA, 19 nt, synthetic construct
  source 1..19: mol_type = other RNA; note = Synthetic
ggaaggacatgtcaatctt

SEQ ID NO: 1103 - RNA, 19 nt, synthetic construct
  source 1..19: mol_type = other RNA; note = Synthetic
gaaggacatgtcaatcttg

SEQ ID NO: 1104 - RNA, 19 nt, synthetic construct
  source 1..19: mol_type = other RNA; note = Synthetic
aaggacatgtcaatcttgg

SEQ ID NO: 1105 - RNA, 19 nt, synthetic construct
  source 1..19: mol_type = other RNA; note = Synthetic
aggacatgtcaatcttggt

SEQ ID NO: 1106 - RNA, 19 nt, synthetic construct
  source 1..19: mol_type = other RNA; note = Synthetic
ggacatgtcaatcttggtc

SEQ ID NO: 1107 - RNA, 19 nt, synthetic construct
  source 1..19: mol_type = other RNA; note = Synthetic
gacatgtcaatcttggtca

SEQ ID NO: 1108 - RNA, 19 nt, synthetic construct
  source 1..19: mol_type = other RNA; note = Synthetic
acatgtcaatcttggtcat

SEQ ID NO: 1109 - RNA, 19 nt, synthetic construct
  source 1..19: mol_type = other RNA; note = Synthetic
catgtcaatcttggtcatc

SEQ ID NO: 1110 - RNA, 19 nt, synthetic construct
  source 1..19: mol_type = other RNA; note = Synthetic
atgtcaatcttggtcatcc

SEQ ID NO: 1111 - RNA, 19 nt, synthetic construct
  source 1..19: mol_type = other RNA; note = Synthetic
tgtcaatcttggtcatcca

SEQ ID NO: 1112 - RNA, 19 nt, synthetic construct
  source 1..19: mol_type = other RNA; note = Synthetic
gtcaatcttggtcatccat

SEQ ID NO: 1113 - RNA, 19 nt, synthetic construct
  source 1..19: mol_type = other RNA; note = Synthetic
tcaatcttggtcatccatg

SEQ ID NO: 1114 - RNA, 19 nt, synthetic construct
  source 1..19: mol_type = other RNA; note = Synthetic
caatcttggtcatccatga

SEQ ID NO: 1115 - RNA, 19 nt, synthetic construct
  source 1..19: mol_type = other RNA; note = Synthetic
aatcttggtcatccatgac

SEQ ID NO: 1116 - RNA, 19 nt, synthetic construct
  source 1..19: mol_type = other RNA; note = Synthetic
atcttggtcatccatgaca

SEQ ID NO: 1117 - RNA, 19 nt, synthetic construct
  source 1..19: mol_type = other RNA; note = Synthetic
tgacaatgaactactgcag

SEQ ID NO: 1118 - RNA, 19 nt, synthetic construct
  source 1..19: mol_type = other RNA; note = Synthetic
gacaatgaactactgcagg

SEQ ID NO: 1119 - RNA, 19 nt, synthetic construct
  source 1..19: mol_type = other RNA; note = Synthetic
acaatgaactactgcagga

SEQ ID NO: 1120 - RNA, 19 nt, synthetic construct
  source 1..19: mol_type = other RNA; note = Synthetic
caatgaactactgcaggaa

SEQ ID NO: 1121 - RNA, 19 nt, synthetic construct
  source 1..19: mol_type = other RNA; note = Synthetic
aatgaactactgcaggaat

SEQ ID NO: 1122 - RNA, 19 nt, synthetic construct
  source 1..19: mol_type = other RNA; note = Synthetic
atgaactactgcaggaatc

SEQ ID NO: 1123 - RNA, 19 nt, synthetic construct
  source 1..19: mol_type = other RNA; note = Synthetic
tgaactactgcaggaatcc

SEQ ID NO: 1124 - RNA, 19 nt, synthetic construct
  source 1..19: mol_type = other RNA; note = Synthetic
ctactgcaggaatccagat

SEQ ID NO: 1125 - RNA, 19 nt, synthetic construct
  source 1..19: mol_type = other RNA; note = Synthetic
caggcccttggtgttttac

SEQ ID NO: 1126 - RNA, 19 nt, synthetic construct
  source 1..19: mol_type = other RNA; note = Synthetic
cttggtgttttaccatgga

SEQ ID NO: 1127 - RNA, 19 nt, synthetic construct
  source 1..19: mol_type = other RNA; note = Synthetic
ttggtgttttaccatggac

SEQ ID NO: 1128 - RNA, 19 nt, synthetic construct
  source 1..19: mol_type = other RNA; note = Synthetic
tggtgttttaccatggacc

SEQ ID NO: 1129 - RNA, 19 nt, synthetic construct
  source 1..19: mol_type = other RNA; note = Synthetic
ggtgttttaccatggaccc

SEQ ID NO: 1130 - RNA, 19 nt, synthetic construct
  source 1..19: mol_type = other RNA; note = Synthetic
gtgttttaccatggacccc

SEQ ID NO: 1131 - RNA, 19 nt, synthetic construct
  source 1..19: mol_type = other RNA; note = Synthetic
gttttaccatggaccccag

SEQ ID NO: 1132 - RNA, 19 nt, synthetic construct
  source 1..19: mol_type = other RNA; note = Synthetic
ggagtactgcaacctgacg

SEQ ID NO: 1133 - RNA, 19 nt, synthetic construct
  source 1..19: mol_type = other RNA; note = Synthetic
gagtactgcaacctgacgc

SEQ ID NO: 1134 - RNA, 19 nt, synthetic construct
  source 1..19: mol_type = other RNA; note = Synthetic
gtactgcaacctgacgcga

SEQ ID NO: 1135 - RNA, 19 nt, synthetic construct
  source 1..19: mol_type = other RNA; note = Synthetic
tactgcaacctgacgcgat

SEQ ID NO: 1136 - RNA, 19 nt, synthetic construct
  source 1..19: mol_type = other RNA; note = Synthetic
tgcaacctgacgcgatgct

SEQ ID NO: 1137 - RNA, 19 nt, synthetic construct
  source 1..19: mol_type = other RNA; note = Synthetic
cctgacgcgatgctcagac

SEQ ID NO: 1138 - RNA, 19 nt, synthetic construct
  source 1..19: mol_type = other RNA; note = Synthetic
ctgacgcgatgctcagaca

SEQ ID NO: 1139 - RNA, 19 nt, synthetic construct
  source 1..19: mol_type = other RNA; note = Synthetic
gatgctcagacacagaagg

SEQ ID NO: 1140 - RNA, 19 nt, synthetic construct
  source 1..19: mol_type = other RNA; note = Synthetic
atgctcagacacagaaggg

SEQ ID NO: 1141 - RNA, 19 nt, synthetic construct
  source 1..19: mol_type = other RNA; note = Synthetic
agacacagaagggactgtg

SEQ ID NO: 1142 - RNA, 19 nt, synthetic construct
  source 1..19: mol_type = other RNA; note = Synthetic
gcatcctcttcatttgatt

SEQ ID NO: 1143 - RNA, 19 nt, synthetic construct
  source 1..19: mol_type = other RNA; note = Synthetic
catcctcttcatttgattg

SEQ ID NO: 1144 - RNA, 19 nt, synthetic construct
  source 1..19: mol_type = other RNA; note = Synthetic
atcctcttcatttgattgt

SEQ ID NO: 1145 - RNA, 19 nt, synthetic construct
  source 1..19: mol_type = other RNA; note = Synthetic
tcctcttcatttgattgtg

SEQ ID NO: 1146 - RNA, 19 nt, synthetic construct
  source 1..19: mol_type = other RNA; note = Synthetic
cctcttcatttgattgtgg

SEQ ID NO: 1147 - RNA, 19 nt, synthetic construct
  source 1..19: mol_type = other RNA; note = Synthetic
cttcatttgattgtgggaa

SEQ ID NO: 1148 - RNA, 19 nt, synthetic construct
  source 1..19: mol_type = other RNA; note = Synthetic
ttcatttgattgtgggaag

SEQ ID NO: 1149 - RNA, 19 nt, synthetic construct
  source 1..19: mol_type = other RNA; note = Synthetic
tcatttgattgtgggaagc

SEQ ID NO: 1150 - RNA, 19 nt, synthetic construct
  source 1..19: mol_type = other RNA; note = Synthetic
catttgattgtgggaagcc

SEQ ID NO: 1151 - RNA, 19 nt, synthetic construct
  source 1..19: mol_type = other RNA; note = Synthetic
atttgattgtgggaagcct

SEQ ID NO: 1152 - RNA, 19 nt, synthetic construct
  source 1..19: mol_type = other RNA; note = Synthetic
gtgggaagcctcaagtgga

SEQ ID NO: 1153 - RNA, 19 nt, synthetic construct
  source 1..19: mol_type = other RNA; note = Synthetic
aagaaatgtcctggaagca

SEQ ID NO: 1154 - RNA, 19 nt, synthetic construct
  source 1..19: mol_type = other RNA; note = Synthetic
agaaatgtcctggaagcat

SEQ ID NO: 1155 - RNA, 19 nt, synthetic construct
  source 1..19: mol_type = other RNA; note = Synthetic
gaaatgtcctggaagcatt

SEQ ID NO: 1156 - RNA, 19 nt, synthetic construct
  source 1..19: mol_type = other RNA; note = Synthetic
aatgtcctggaagcattgt

SEQ ID NO: 1157 - RNA, 19 nt, synthetic construct
  source 1..19: mol_type = other RNA; note = Synthetic
atgtcctggaagcattgta

SEQ ID NO: 1158 - RNA, 19 nt, synthetic construct
  source 1..19: mol_type = other RNA; note = Synthetic
agaacaaggtttggaaagc

SEQ ID NO: 1159 - RNA, 19 nt, synthetic construct
  source 1..19: mol_type = other RNA; note = Synthetic
gaacaaggtttggaaagca

SEQ ID NO: 1160 - RNA, 19 nt, synthetic construct
  source 1..19: mol_type = other RNA; note = Synthetic
aacaaggtttggaaagcac

SEQ ID NO: 1161 - RNA, 19 nt, synthetic construct
  source 1..19: mol_type = other RNA; note = Synthetic
acaaggtttggaaagcact

SEQ ID NO: 1162 - RNA, 19 nt, synthetic construct
  source 1..19: mol_type = other RNA; note = Synthetic
caaggtttggaaagcactt

SEQ ID NO: 1163 - RNA, 19 nt, synthetic construct
  source 1..19: mol_type = other RNA; note = Synthetic
aaggtttggaaagcacttc

SEQ ID NO: 1164 - RNA, 19 nt, synthetic construct
  source 1..19: mol_type = other RNA; note = Synthetic
aggtttggaaagcacttct

SEQ ID NO: 1165 - RNA, 19 nt, synthetic construct
  source 1..19: mol_type = other RNA; note = Synthetic
tggaaagcacttctgtgga

SEQ ID NO: 1166 - RNA, 19 nt, synthetic construct
  source 1..19: mol_type = other RNA; note = Synthetic
gtggaggcaccttaatatc

SEQ ID NO: 1167 - RNA, 19 nt, synthetic construct
  source 1..19: mol_type = other RNA; note = Synthetic
cttaatatccccagagtgg

SEQ ID NO: 1168 - RNA, 19 nt, synthetic construct
  source 1..19: mol_type = other RNA; note = Synthetic
taatatccccagagtgggt

SEQ ID NO: 1169 - RNA, 19 nt, synthetic construct
  source 1..19: mol_type = other RNA; note = Synthetic
agagtgggtgctgactgct

SEQ ID NO: 1170 - RNA, 19 nt, synthetic construct
  source 1..19: mol_type = other RNA; note = Synthetic
caaggtcatcctgggtgca

SEQ ID NO: 1171 - RNA, 19 nt, synthetic construct
  source 1..19: mol_type = other RNA; note = Synthetic
cctgggtgcacaccaagaa

SEQ ID NO: 1172 - RNA, 19 nt, synthetic construct
  source 1..19: mol_type = other RNA; note = Synthetic
gtgcacaccaagaagtgaa

SEQ ID NO: 1173 - RNA, 19 nt, synthetic construct
  source 1..19: mol_type = other RNA; note = Synthetic
agcagatattgccttgcta

SEQ ID NO: 1174 - RNA, 19 nt, synthetic construct
  source 1..19: mol_type = other RNA; note = Synthetic
gcagatattgccttgctaa

SEQ ID NO: 1175 - RNA, 19 nt, synthetic construct
  source 1..19: mol_type = other RNA; note = Synthetic
cagatattgccttgctaaa

SEQ ID NO: 1176 - RNA, 19 nt, synthetic construct
  source 1..19: mol_type = other RNA; note = Synthetic
agatattgccttgctaaag

SEQ ID NO: 1177 - RNA, 19 nt, synthetic construct
  source 1..19: mol_type = other RNA; note = Synthetic
gatattgccttgctaaagc

SEQ ID NO: 1178 - RNA, 19 nt, synthetic construct
  source 1..19: mol_type = other RNA; note = Synthetic
atattgccttgctaaagct

SEQ ID NO: 1179 - RNA, 19 nt, synthetic construct
  source 1..19: mol_type = other RNA; note = Synthetic
tattgccttgctaaagcta

SEQ ID NO: 1180 - RNA, 19 nt, synthetic construct
  source 1..19: mol_type = other RNA; note = Synthetic
tcatcactgacaaagtaat

SEQ ID NO: 1181 - RNA, 19 nt, synthetic construct
  source 1..19: mol_type = other RNA; note = Synthetic
ggactgaatgttacatcac

SEQ ID NO: 1182 - RNA, 19 nt, synthetic construct
  source 1..19: mol_type = other RNA; note = Synthetic
gactgaatgttacatcact

SEQ ID NO: 1183 - RNA, 19 nt, synthetic construct
  source 1..19: mol_type = other RNA; note = Synthetic
actgaatgttacatcactg

SEQ ID NO: 1184 - RNA, 19 nt, synthetic construct
  source 1..19: mol_type = other RNA; note = Synthetic
ctgaatgttacatcactgg

SEQ ID NO: 1185 - RNA, 19 nt, synthetic construct
  source 1..19: mol_type = other RNA; note = Synthetic
tgaatgttacatcactggc

SEQ ID NO: 1186 - RNA, 19 nt, synthetic construct
  source 1..19: mol_type = other RNA; note = Synthetic
aatgttacatcactggctg

SEQ ID NO: 1187 - RNA, 19 nt, synthetic construct
  source 1..19: mol_type = other RNA; note = Synthetic
gaaacccaaggtacctttg

SEQ ID NO: 1188 - RNA, 36 nt, synthetic construct; identical to SEQ ID NO: 399
  source 1..36: mol_type = other RNA; note = Synthetic

SEQ ID NO: 1189 - RNA, 22 nt, synthetic construct
  source 1..22: mol_type = other RNA; note = Synthetic
ttggataatattctgttgtcgg

SEQ ID NO: 1190 - RNA, 19 nt, synthetic construct
  source 1..19: mol_type = other RNA; note = Synthetic
gacaacagaatattatcca

SEQ ID NO: 1191 - RNA, 24 nt, synthetic construct
  source 1..24: mol_type = other RNA; note = Synthetic
cgttaatcgcgtataatacgcgta

SEQ ID NO: 1192 - RNA, 27 nt, synthetic construct
  source 1..27: mol_type = other RNA; note = Synthetic
atacgcgtattatacgcgattaacgac

SEQ ID NO: 1193 - RNA, 25 nt, synthetic construct
  source 1..25: mol_type = other RNA; note = Synthetic
catattgcgcgtatagtcgcgttag

SEQ ID NO: 1194 - RNA, 27 nt, synthetic construct
  source 1..27: mol_type = other RNA; note = Synthetic
ctaacgcgactatacgcgcaatatggt

SEQ ID NO: 1195 - RNA, 24 nt, synthetic construct
  source 1..24: mol_type = other RNA; note = Synthetic
ggcgcgtatagtcgcgcgtatagc

SEQ ID NO: 1196 - RNA, 27 nt, synthetic construct
  source 1..27: mol_type = other RNA; note = Synthetic
gactatacgcgcgactatacgcgcctc

SEQ ID NO: 1197 - RNA, 16 nt, synthetic construct
  source 1..16: mol_type = other RNA; note = Synthetic
gcagccgaaaggctgc

(19) United States
(12) Patent Application Publication
(10) Pub. No.: US 2024/0401053 A1
(43) Pub. Date: Dec. 5, 2024

(54) COMPOSITIONS AND METHODS FOR INHIBITING LPA EXPRESSION

(71) Applicant: Dicema Pharmaceuticals, Inc., Lexington, MA (US)

(72) Inventors: Bob Dale Brown, Littleton, MA (US); Henryk T. Dudek, Belmont, MA (US); Marc Abrams, Natick, MA (US); Wen Han, Boston, MA (US); Anton Turanov, Revere, MA (US)

(21) Appl. No.: 18/807,591

(22) Filed: Aug. 16, 2024

Related U.S. Application Data
(63) Continuation of application No. 18/040,302, filed on Oct. 2, 2023.
(60) Provisional application No. 63/074,779, filed on Sep. 4, 2020.
(60) Provisional application No. 63/061,676, filed on Aug. 5, 2020.

(51) Int. Cl. C12N 15/113

(52) U.S. Cl. CPC C12N 15/113; C12N 2310/14; C12N 2310/315; C12N 2310/321; C12N 2310/322; C12N 2310/351; C12N 2310/531

(57) ABSTRACT

Oligonucleotides are provided herein that inhibit apolipoprotein (a) (LPA) expression. Also provided are compositions including the same and uses thereof, particularly uses relating to treating diseases, disorders and/or conditions associated with LPA expression.

### CROSS-REFERENCE TO RELATED APPLICATIONS

This application is a continuation application of U.S. application Ser. No. 18/040,302, filed Feb. 2, 2023, which is a National Stage of International Application No. PCT/US2021/071109, filed Aug. 5, 2021, which claims the benefit under 35 U.S.C. § 119 (e) of U.S. Provisional Patent Application No. 63/061,676, filed Aug. 5, 2020, and claims the benefit under 35 U.S.C. § 119 (e) of U.S. Provisional Patent Application No. 63/074,779, filed Sep. 4, 2020, the contents of each of which are incorporated herein by reference in their entirety.

### TECHNICAL FIELD

The disclosure relates to oligonucleotides that inhibit apolipoprotein (a) (“LPA”) expression and uses thereof, particularly uses relating to treating diseases, disorders and/or conditions associated with LPA expression.

### REFERENCE TO SEQUENCE LISTING

The instant application contains a Sequence Listing which has been submitted electronically in XML file format and is hereby incorporated by reference in its entirety. Said XML copy, created on Aug. 15, 2024, is named “400930_026USC1_211985_SL.xml” and is 1,325,553 bytes in size.

### BACKGROUND

Lipoprotein (a) (Lp(a)) is a heterogeneous low density lipoprotein (LDL)-like particle containing a lipid core and apolipoprotein B (apoB-100) with a unique constituent, apolipoprotein (a) (apo(a)), that is attached to apoB-100 through a disulfide bond. The apo(a) gene (LPA) is expressed predominantly in the liver and expression is restricted to human and non-human primates. Lp(a) levels in humans are genetically defined and do not change significantly with diet, exercise, or other lifestyle changes. LPA varies in length depending upon the number of Kringle KIV2 domains present and its expression is inversely correlated with the number of domains present. Normal Lp(a) levels range from 0.1-25 mg/dl, with about 25% of the population in the United States of America having Lp(a) levels of 30 mg/dl or higher. Analysis of Lp(a) levels in multiple studies have implicated high Lp(a) levels as an independent risk factor for cardiovascular disease, stroke, and other related disorders including atherosclerotic stenosis. In addition, genome-wide association analyses have also implicated LPA as a genetic risk factor for diseases such as atherosclerotic stenosis. When therapeutic lipoprotein apheresis is used to lower both Lp(a) and LDL levels in hyperlipidemic patients, significant reductions of cardiovascular events have been observed.

Therefore, there exists a need for therapeutics and treatments related to these and other LPA-related diseases.

### BRIEF SUMMARY OF THE INVENTION

Embodiments of the disclosure relate to compositions and methods for treating a disease, disorder and/or condition related to LPA expression. The disclosure is based, in part, on the discovery and development of oligonucleotides that selectively inhibit and/or reduce LPA expression in the liver. Accordingly, target sequences within LPA mRNA were identified and RNAi oligonucleotides that bind to these target sequences and inhibit LPA mRNA expression were generated. As demonstrated herein, the RNAi oligonucleotides inhibited monkey and human LPA expression in the liver. Without being bound by theory, the RNAi oligonucleotides described herein are useful for treating a disease, disorder or condition associated with LPA expression (e.g., cardiometabolic diseases, atherosclerosis, dyslipidemia, NAFLD and NASH).

Accordingly, in some embodiments, the present disclosure provides an RNAi oligonucleotide for reducing LPA expression, the oligonucleotide comprising a sense strand and an antisense strand, wherein the sense strand and the antisense strand form a duplex region, wherein the antisense strand comprises a region of complementarity to a LPA mRNA target sequence of any one of SEQ ID NOs: 4-387, and wherein the region of complementarity is at least 15 contiguous nucleotides in length.

In any of the foregoing or related embodiments, the sense strand is 15 to 50 nucleotides in length. In some embodiments, the sense strand is 18 to 36 nucleotides in length.

In any of the foregoing or related aspects, the antisense strand is 15 to 30 nucleotides in length.

In any of the foregoing or related aspects, the antisense strand is 22 nucleotides in length and wherein antisense strand and the sense strand form a duplex region of at least 19 nucleotides in length, optionally at least 20 nucleotides in length.

In any of the foregoing or related aspects, the region of complementarity is at least 19 contiguous nucleotides in length, optionally at least 20 nucleotides in length.

In any of the foregoing or related aspects, the 3′ end of the sense strand comprises a stem-loop set forth as S1-L-S2, wherein S1 is complementary to S2, and wherein L forms a loop between S1 and S2 of 3-5 nucleotides in length.

In some aspects, the disclosure provides an RNAi oligonucleotide for reducing LPA expression, the oligonucleotide comprising a sense strand of 15 to 50 nucleotides in length and an antisense strand, wherein the sense strand and the antisense strand form a duplex region, wherein the wherein the antisense strand comprises a region of complementarity to a LPA mRNA target sequence of any one of SEQ ID NOs: 4-387, and wherein the region of complementarity is at least 15 contiguous nucleotides in length.

In other aspects, the disclosure provides an RNAi oligonucleotide for reducing LPA expression, the oligonucleotide comprising a sense strand of 15 to 50 nucleotides in length and an antisense strand of 15 to 30 nucleotides in length, wherein the sense strand and the antisense strand form a duplex region, wherein the antisense strand comprises a region of complementarity to a LPA mRNA target sequence of any one of SEQ ID NOs: 4-387, and wherein the region of complementarity is at least 15 contiguous nucleotides in length.

In yet other aspects, the disclosure provides an RNAi oligonucleotide for reducing LPA expression, the oligonucleotide comprising a sense strand of 15 to 50 nucleotides in length and an antisense strand, wherein the sense strand and the antisense strand form a duplex region, wherein the antisense strand comprises a region of complementarity to a LPA mRNA target sequence of any one of SEQ ID NOs: 4-387, and wherein the region of complementarity is 19 contiguous nucleotides in length, optionally 20 nucleotides in length.

In further aspects, the disclosure provides an RNAi oligonucleotide for reducing LPA expression, the oligonucleotide comprising a sense strand of 18 to 36 nucleotides in length and an antisense strand, wherein the sense strand and the antisense strand form a duplex region, wherein the antisense strand comprises a region of complementarity to a LPA mRNA target sequence of any one of SEQ ID NOs: 4-387, and wherein the region of complementarity is 19 contiguous nucleotides in length, optionally 20 nucleotides in length.

In other aspects, the disclosure provides an RNAi oligonucleotide for reducing LPA expression, the oligonucleotide comprising a sense strand of 18 to 36 nucleotides in length and an antisense strand of 22 nucleotides in length, wherein the sense strand and the antisense strand form a duplex region, wherein the antisense strand comprises a region of complementarity to a LPA mRNA target sequence of any one of SEQ ID NOs: 4-387, and wherein the region of complementarity is 19 contiguous nucleotides in length, optionally 20 nucleotides in length.

In some aspects, the disclosure provides an RNAi oligonucleotide for reducing LPA expression, the oligonucleotide comprising a sense strand of 18 to 36 nucleotides in length and an antisense strand of 22 nucleotides in length, wherein the sense strand and the antisense strand form a duplex region, wherein the 3′ end of the sense strand comprises a stem-loop set forth as S1-L-S2, wherein S1 is complementary to S2, and wherein L forms a loop between S1 and S2 of 3-5 nucleotides in length, wherein the antisense strand comprises a region of complementarity to a LPA mRNA target sequence of any one of SEQ ID NOs: 4-387, and wherein the region of complementarity is 19 contiguous nucleotides in length, optionally 20 nucleotides in length.

In other aspects, the disclosure provides an RNAi oligonucleotide for reducing LPA expression, the oligonucleotide comprising a sense strand of 36 nucleotides in length and an antisense strand of 22 nucleotides in length, wherein the sense strand and the antisense strand form a duplex region, wherein the 3′ end of the sense strand comprises a stem-loop set forth as S1-L-S2, wherein S1 is complementary to S2, and wherein L forms a loop between S1 and S2 of 3-5 nucleotides in length, wherein the antisense strand comprises a region of complementarity to a LPA mRNA target sequence of any one of SEQ ID NOs: 4-387, and wherein the region of complementarity is 19 contiguous nucleotides in length, optionally 20 nucleotides in length.

In yet other aspects, the disclosure provides an RNAi oligonucleotide for reducing LPA expression, the oligonucleotide comprising a sense strand of 36 nucleotides in length and an antisense strand of 22 nucleotides in length, wherein the sense strand and the antisense strand form a duplex region of at least 19 nucleotides in length, optionally 20 nucleotides in length, wherein the 3′ end of the sense strand comprises a stem-loop set forth as S1-L-S2, wherein S1 is complementary to S2, and wherein L forms a loop between S1 and S2 of 3-5 nucleotides in length, wherein the antisense strand comprises a region of complementarity to a LPA mRNA target sequence of any one of SEQ ID NOs: 4-387, and wherein the region of complementarity is 19 contiguous nucleotides in length, optionally 20 nucleotides in length.

In any of the foregoing or related aspects, L is a triloop or a tetraloop. In some embodiments, L is a tetraloop. In some embodiments, the tetraloop comprises the sequence 5′-GAAA-3′.

In any of the foregoing or related embodiments, the S1 and S2 are 1-10 nucleotides in length and have the same length. In some embodiments, S1 and S2 are 1 nucleotide, 2 nucleotides, 3 nucleotides, 4 nucleotides, 5 nucleotides, 6 nucleotides, 7 nucleotides, 8 nucleotides, 9 nucleotides, or 10 nucleotides in length. In some embodiments, S1 and S2 are 6 nucleotides in length. In some embodiments, the stem-loop comprises the sequence 5′-GCAGCCGAAAGGCUGC-3′ (SEQ ID NO: 1197).

In any of the foregoing or related embodiments, the antisense strand comprises a 3′ overhang sequence of one or more nucleotides in length. In some embodiments, the 3′ overhang sequence is 2 nucleotides in length, optionally wherein the 3′ overhang sequence is GG.

In any of the foregoing or related embodiments, the oligonucleotide comprises at least one modified nucleotide. In some embodiments, the modified nucleotide comprises a 2′-modification. In some embodiments, the 2′-modification is a modification selected from 2′-aminoethyl, 2′-fluoro, 2′-O-methyl, 2′-O-methoxyethyl, and 2′-deoxy-2′-fluoro-β-d-arabinonucleic acid. In some embodiments, all nucleotides comprising the oligonucleotide are modified, optionally wherein the modification is a 2′-modification selected from 2′-fluoro and 2′-O-methyl.

In any of the foregoing or related embodiments, the oligonucleotide comprises at least one modified internucleotide linkage. In some embodiments, the at least one modified internucleotide linkage is a phosphorothioate linkage.

In any of the foregoing or related embodiments, the 4′-carbon of the sugar of the 5′-nucleotide of the antisense strand comprises a phosphate analog. In some embodiments, the phosphate analog is oxymethylphosphonate, vinylphosphonate or malonyl phosphonate, optionally wherein the phosphate analog is a 4′-phosphate analog comprising 5′-methoxyphosphonate-4′-oxy.

In any of the foregoing or related embodiments, at least one nucleotide of the oligonucleotide is conjugated to one or more targeting ligands. In some embodiments, each targeting ligand comprises a carbohydrate, amino sugar, cholesterol, polypeptide, or lipid. In some embodiments, each targeting ligand comprises a N-acetylgalactosamine (GalNAc) moiety. In some embodiments, the GalNAc moiety is a monovalent GalNAc moiety, a bivalent GalNAc moiety, a trivalent GalNAc moiety or a tetravalent GalNAc moiety. In some embodiments, up to 4 nucleotides of L of the stem-loop are each conjugated to a monovalent GalNAc moiety.

In any of the foregoing or related embodiments, the sense strand comprises a nucleotide sequence of any one of SEQ ID NOs: 388, 389, 390, 391, 392, 393, 394, 395, 396, 397, 398, 399, 400, 401, 402, and 403.

In any of the foregoing or related embodiments, the antisense strand comprises a nucleotide sequence of any one of SEQ ID NOs: 788, 789, 790, 791, 792, 793, 794, 795, 796, 797, 798, 799, 800, 801, 802, and 803.

In any of the foregoing or related embodiments, the sense strand and antisense strands comprise nucleotide sequences selected from the group consisting of:
(a) SEQ ID NOs: 393 and 793, respectively;
(b) SEQ ID NOs: 388 and 788, respectively;
(c) SEQ ID NOs: 389 and 789, respectively;
(d) SEQ ID NOs: 390 and 790, respectively;
(e) SEQ ID NOs: 391 and 791, respectively;
(f) SEQ ID NOs: 392 and 792, respectively;
(g) SEQ ID NOs: 394 and 794, respectively;
(h) SEQ ID NOs: 395 and 795, respectively;
(i) SEQ ID NOs: 396 and 796, respectively;
(j) SEQ ID NOs: 397 and 797, respectively;
(k) SEQ ID NOs: 398 and 798, respectively;
(l) SEQ ID NOs: 399 and 799, respectively;
(m) SEQ ID NOs: 400 and 800, respectively;
(n) SEQ ID NOs: 401 and 801, respectively;
(o) SEQ ID NOs: 402 and 802, respectively; and
(p) SEQ ID NOs: 403 and 803, respectively.

In some embodiments, the sense strand comprises a nucleotide sequence as set forth in SEQ ID NO: 393, wherein the antisense strand comprises a nucleotide sequence as set forth in SEQ ID NO: 793. In some embodiments, the sense strand comprises a nucleotide sequence as set forth in SEQ ID NO: 388, wherein the antisense strand comprises a nucleotide sequence as set forth in SEQ ID NO: 788. In some embodiments, the sense strand comprises a nucleotide sequence as set forth in SEQ ID NO: 389, wherein the antisense strand comprises a nucleotide sequence as set forth in SEQ ID NO: 789. In some embodiments, the sense strand comprises a nucleotide sequence as set forth in SEQ ID NO: 390, wherein the antisense strand comprises a nucleotide sequence as set forth in SEQ ID NO: 790. In some embodiments, the sense strand comprises a nucleotide sequence as set forth in SEQ ID NO: 391, wherein the antisense strand comprises a nucleotide sequence as set forth in SEQ ID NO: 791. In some embodiments, the sense strand comprises a nucleotide sequence as set forth in SEQ ID NO: 392, wherein the antisense strand comprises a nucleotide sequence as set forth in SEQ ID NO: 792. In some embodiments, the sense strand comprises a nucleotide sequence as set forth in SEQ ID NO: 394, wherein the antisense strand comprises a nucleotide sequence as set forth in SEQ ID NO: 794. In some embodiments, the sense strand comprises a nucleotide sequence as set forth in SEQ ID NO: 395, wherein the antisense strand comprises a nucleotide sequence as set forth in SEQ ID NO: 795. In some embodiments, the sense strand comprises a nucleotide sequence as set forth in SEQ ID NO: 396, wherein the antisense strand comprises a nucleotide sequence as set forth in SEQ ID NO: 796. In some embodiments, the sense strand comprises a nucleotide sequence as set forth in SEQ ID NO: 397, wherein the antisense strand comprises a nucleotide sequence as set forth in SEQ ID NO: 797. In some embodiments, the sense strand comprises a nucleotide sequence as set forth in SEQ ID NO: 398, wherein the antisense strand comprises a nucleotide sequence as set forth in SEQ ID NO: 798. In some embodiments, the sense strand comprises a nucleotide sequence as set forth in SEQ ID NO: 399, wherein the antisense strand comprises a nucleotide sequence as set forth in SEQ ID NO: 799. In some embodiments, the sense strand comprises a nucleotide sequence as set forth in SEQ ID NO: 400, wherein the antisense strand comprises a nucleotide sequence as set forth in SEQ ID NO: 800. In some embodiments, the sense strand comprises a nucleotide sequence as set forth in SEQ ID NO: 401, wherein the antisense strand comprises a nucleotide sequence as set forth in SEQ ID NO: 801. In some embodiments, the sense strand comprises a nucleotide sequence as set forth in SEQ ID NO: 402, wherein the antisense strand comprises a nucleotide sequence as set forth in SEQ ID NO: 802. In some embodiments, the sense strand comprises a nucleotide sequence as set forth in SEQ ID NO: 403, wherein the antisense strand comprises a nucleotide sequence as set forth in SEQ ID NO: 803.

In some embodiments, the disclosure provides an RNAi oligonucleotide for reducing LPA expression, the oligonucleotide comprising a sense strand and an antisense strand, wherein the sense strand and the antisense strand form a duplex region, wherein all nucleotides comprising the sense strand and antisense strand are modified, wherein the antisense strand comprises a region of complementarity to a LPA mRNA target sequence of any one of SEQ ID NOs: 4-387, and wherein the region of complementarity is at least 15 contiguous nucleotides in length.

In further embodiments, the disclosure provides an RNAi oligonucleotide for reducing LPA expression, the oligonucleotide comprising a sense strand and an antisense strand, wherein the sense strand and the antisense strand form a duplex region, wherein all nucleotides comprising the sense strand and antisense strand are modified, wherein the 4′-carbon of the sugar of the 5′-nucleotide of the antisense strand comprises a phosphate analog, wherein the antisense strand comprises a region of complementarity to a LPA mRNA target sequence of any one of SEQ ID NOs: 4-387, and wherein the region of complementarity is at least 15 contiguous nucleotides in length.

In other embodiments, the disclosure provides an RNAi oligonucleotide for reducing LPA expression, the oligonucleotide comprising a sense strand and an antisense strand, wherein the sense strand and the antisense strand form a duplex region, wherein all nucleotides comprising the sense strand and antisense strand are modified, wherein the 4′-carbon of the sugar of the 5′-nucleotide of the antisense strand comprises a phosphate analog, wherein the antisense strand comprises a region of complementarity to a LPA mRNA target sequence of any one of SEQ ID NOs: 4-387, and wherein the region of complementarity is at least 15 contiguous nucleotides in length.

In some embodiments, the disclosure provides an RNAi oligonucleotide for reducing LPA expression, the oligonucleotide comprising a sense strand and an antisense strand, wherein the sense strand and the antisense strand form a duplex region, wherein all nucleotides comprising the sense strand and the antisense strand are modified, wherein the antisense strand and the sense strand comprise one or more 2′-fluoro and 2′-O-methyl modified nucleotides and at least one phosphorothioate linkage, wherein the 4′-carbon of the sugar of the 5′-nucleotide of the antisense strand comprises a phosphate analog, wherein the antisense strand comprises a region of complementarity to a LPA mRNA target sequence of any one of SEQ ID NOs: 4-387, and wherein the region of complementarity is at least 15 contiguous nucleotides in length.

In some embodiments, the disclosure provides a method for treating a subject having a disease, disorder or condition associated with LPA expression, the method comprising administering to the subject a therapeutically effective amount of the RNAi oligonucleotide of any one of the preceding claims, or pharmaceutical composition thereof, thereby treating the subject.

In other embodiments, the disclosure provides a pharmaceutical composition comprising a RNAi oligonucleotide described herein, and a pharmaceutically acceptable carrier, delivery agent or excipient.

In other embodiments, the disclosure provides a method of delivering an oligonucleotide to a subject, the method comprising administering a pharmaceutical composition described herein to the subject.

In another embodiments, the disclosure provides a method for reducing LPA expression in a cell, a population of cells or a subject, the method comprising the step of:
i. contacting the cell or the population of cells with a RNAi oligonucleotide or pharmaceutical composition described herein; or
ii. administering to the subject a RNAi oligonucleotide or pharmaceutical composition described herein. In some embodiments, reducing LPA expression comprises reducing an amount or level of LPA mRNA, an amount or level of LPA protein, or both. In some embodiments, the subject has a disease, disorder or condition associated with LPA expression. In some embodiments, the disease, disorder, or condition associated with LPA expression is a cardiometabolic disease, optionally atherosclerosis, dyslipidemia, NAFLD and NASH. In some embodiments, the RNAi oligonucleotide, or pharmaceutical composition, is administered in combination with a second composition or therapeutic agent.

In another aspect, the disclosure provides a method for treating a subject having a disease, disorder or condition associated with LPA expression, the method comprising administering to the subject a therapeutically effective amount of an RNAi oligonucleotide comprising a sense strand and an antisense strand, wherein the sense strand and the antisense strand form a duplex region, wherein the antisense strand comprises a region of complementarity to a LPA mRNA target sequence of any one of SEQ ID NOs: 4-387, and wherein the region of complementarity is at least 15 contiguous nucleotides in length.

In another aspect, the disclosure provides a method for treating a subject having a disease, disorder or condition associated with LPA expression, the method comprising administering to the subject a therapeutically effective amount of an RNAi oligonucleotide comprising a sense strand and an antisense strand selected from a row set forth in Table 5, or pharmaceutical composition thereof, thereby treating the subject.

In other embodiments, the disclosure provides a method for treating a subject having a disease, disorder or condition associated with LPA expression, the method comprising administering to the subject a therapeutically effective amount of an RNAi oligonucleotide comprising a sense strand and an antisense strand, wherein the sense strand and antisense strands comprise nucleotide sequences selected from the group consisting of:
(a) SEQ ID NOs: 393 and 793, respectively;
(b) SEQ ID NOs: 388 and 788, respectively;
(c) SEQ ID NOs: 389 and 789, respectively;
(d) SEQ ID NOs: 390 and 790, respectively;
(c) SEQ ID NOs: 391 and 791, respectively;
(f) SEQ ID NOs: 392 and 792, respectively;
(g) SEQ ID NOs: 394 and 794, respectively;
(h) SEQ ID NOs: 395 and 795, respectively;
(i) SEQ ID NOs: 396 and 796, respectively;
(j) SEQ ID NOs: 397 and 797, respectively;
(k) SEQ ID NOs: 398 and 798, respectively;
(l) SEQ ID NOs: 399 and 799, respectively;
(m) SEQ ID NOs: 400 and 800, respectively;
(n) SEQ ID NOs: 401 and 801, respectively;
(o) SEQ ID NOs: 402 and 802, respectively; and
(p) SEQ ID NOs: 403 and 803, respectively.

In some embodiments, the sense strand comprises a nucleotide sequence as set forth in SEQ ID NO: 393, wherein the antisense strand comprises a nucleotide sequence as set forth in SEQ ID NO: 793. In some embodiments, the sense strand comprises a nucleotide sequence as set forth in SEQ ID NO: 388, wherein the antisense strand comprises a nucleotide sequence as set forth in SEQ ID NO: 788. In some embodiments, the sense strand comprises a nucleotide sequence as set forth in SEQ ID NO: 389, wherein the antisense strand comprises a nucleotide sequence as set forth in SEQ ID NO: 789. In some embodiments, the sense strand comprises a nucleotide sequence as set forth in SEQ ID NO: 390, wherein the antisense strand comprises a nucleotide sequence as set forth in SEQ ID NO: 790. In some embodiments, the sense strand comprises a nucleotide sequence as set forth in SEQ ID NO: 391, wherein the antisense strand comprises a nucleotide sequence as set forth in SEQ ID NO: 791. In some embodiments, the sense strand comprises a nucleotide sequence as set forth in SEQ ID NO: 392, wherein the antisense strand comprises a nucleotide sequence as set forth in SEQ ID NO: 792. In some embodiments, the sense strand comprises a nucleotide sequence as set forth in SEQ ID NO: 394, wherein the antisense strand comprises a nucleotide sequence as set forth in SEQ ID NO: 794. In some embodiments, the sense strand comprises a nucleotide sequence as set forth in SEQ ID NO: 395, wherein the antisense strand comprises a nucleotide sequence as set forth in SEQ ID NO: 795. In some embodiments, the sense strand comprises a nucleotide sequence as set forth in SEQ ID NO: 396, wherein the antisense strand comprises a nucleotide sequence as set forth in SEQ ID NO: 796. In some embodiments, the sense strand comprises a nucleotide sequence as set forth in SEQ ID NO: 397, wherein the antisense strand comprises a nucleotide sequence as set forth in SEQ ID NO: 797. In some embodiments, the sense strand comprises a nucleotide sequence as set forth in SEQ ID NO: 398, wherein the antisense strand comprises a nucleotide sequence as set forth in SEQ ID NO: 798. In some embodiments, the sense strand comprises a nucleotide sequence as set forth in SEQ ID NO: 399, wherein the antisense strand comprises a nucleotide sequence as set forth in SEQ ID NO: 799. In some embodiments, the sense strand comprises a nucleotide sequence as set forth in SEQ ID NO: 400, wherein the antisense strand comprises a nucleotide sequence as set forth in SEQ ID NO: 800. In some embodiments, the sense strand comprises a nucleotide sequence as set forth in SEQ ID NO: 401, wherein the antisense strand comprises a nucleotide sequence as set forth in SEQ ID NO: 801. In some embodiments, the sense strand comprises a nucleotide sequence as set forth in SEQ ID NO: 402, wherein the antisense strand comprises a nucleotide sequence as set forth in SEQ ID NO: 802. In some embodiments, the sense strand comprises a nucleotide sequence as set forth in SEQ ID NO: 403, wherein the antisense strand comprises a nucleotide sequence as set forth in SEQ ID NO: 803.

In some embodiments, the disease, disorder, or condition associated with LPA expression is a cardiometabolic disease, optionally atherosclerosis, dyslipidemia, NAFLD and NASH.

In some embodiments, the disclosure provides use of an RNAi oligonucleotide or pharmaceutical composition described herein, in the manufacture of a medicament for the treatment of a disease, disorder or condition associated with LPA expression, optionally for the treatment of a cardiometabolic disease, optionally atherosclerosis, dyslipidemia, NAFLD and NASH.

In some embodiments, the disclosure provides use of an RNAi oligonucleotide or pharmaceutical composition described herein, for use, or adaptable for use, in the treatment of a disease, disorder or condition associated with LPA expression, optionally for the treatment of a cardiometabolic disease, optionally atherosclerosis, dyslipidemia, NAFLD and NASH.

In other embodiments, the disclosure provides a kit comprising an RNAi oligonucleotide described herein, an optional pharmaceutically acceptable carrier, and a package insert comprising instructions for administration to a subject having a disease, disorder or condition associated with LPA expression.

In any of the foregoing or related embodiments, the disease, disorder, or condition associated with LPA expression is a cardiometabolic disease, optionally atherosclerosis, dyslipidemia, NAFLD and NASH.

### BRIEF DESCRIPTION OF THE DRAWINGS

FIGS. 1-4 provide graphs depicting the percent (%) of LPA mRNA in HEK293-LPA cells transfected with the indicated DsiRNAs relative to the % of LPA mRNA control mock-treated cells.

FIG. 5 provides a graph depicting the percent (%) of LPA mRNA in HepG2-LPA cells transfected with the indicated DsiRNAs relative to the % of LPA mRNA control mock-treated cells.

FIGS. 6-7 provide graphs depicting the percent (%) of LPA mRNA in HEK293-LPA cells transfected with the indicated DsiRNAs relative to the % of LPA mRNA control mock-treated cells.

FIGS. 8-9 provide graphs depicting the percent (%) of LPA mRNA in liver samples from mice treated with the indicated GalNAc-conjugated LPA oligonucleotides relative to mice treated with phosphate buffered saline (PBS).

FIG. 10 provides a schematic depicting the structure and chemical modification patterns of generic N-Acetylgalactosamine (GalNAc)-conjugated LPA oligonucleotides.

FIGS. 11A-11C provide graphs depicting the percent (%) of LPA mRNA in liver samples from non-human primates (NHPs) treated with the indicated GalNAc-conjugated LPA oligonucleotides relative to NHPs treated with PBS on day 28 (FIG. 11A), day 56 (FIG. 11B) and day 84 (FIG. 11C) following treatment.

FIG. 11D provides a graph depicting the percent (%) of PLG mRNA in liver samples from NHPs treated with the indicated GalNAc-conjugated LPA oligonucleotides relative to NHPs treated with PBS on day 28.

FIG. 12 provides a graph depicting the mean percent (%) of apo(a) protein in serum from NHPs treated with the indicated GalNAc-conjugated LPA oligonucleotides relative to NHPs treated with PBS over time.

### DETAILED DESCRIPTION

### I. Definitions

As used herein, “about,” as applied to one or more values of interest, refers to a value that is similar to a stated reference value. In certain embodiments, “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1% or less in either direction (greater than or less than) of the stated reference value unless otherwise stated or otherwise evident from the context (except where such number would exceed 100% of a possible value).

As used herein, “administer,” “administering,” “administration” and the like refers to providing a substance (e.g., an oligonucleotide) to a subject in a manner that is pharmacologically useful (e.g., to treat a condition in the subject).

As used herein, the term “apolipoprotein (a)” and abbreviated as “apo(a)”, refers to the apolipoprotein (a) polypeptide, which is a member of the apolipoprotein class of polypeptides that bind lipids to form lipoproteins. Apo(a) is a polymorphic glycoprotein encoded by the LPA gene in humans. LPA mRNA and apo(a) polypeptide are expressed predominantly in the liver. Lipoprotein (a) (abbreviated as Lp(a)) is a class of lipoprotein formed in the liver and comprises a single copy of apolipoprotein (apo) B-100 (Apo-B100) covalently tethered to apo(a). In humans, apo(a) includes at least 10 subtypes of KIV repeats, composed of 1 copy each of KIV₁, multiple copies of KIV₂, and 1 copy each of KIV₃-KIV₁₀, KV, and an inactive protease-like domain. The presence of apo(a) distinguishes Lp(a) from all other lipoprotein classes (Marcovina et al., (1995) Clin Chem. 41 (2): 246-55). For the purposes of the disclosure, “apolipoprotein (a)” or “apo(a)” refers to the apo(a) polypeptide from any vertebrate or mammal including, but not limited to, human, mouse, primate, monkey, bovine, chicken, rodent, rat, porcine, ovine and guinea pig. “Apo(a)” also refers to fragments and variants of native apo(a) that maintain at least one in vivo or in vitro activity of a native apo(a). Apo (a) encompasses full-length, unprocessed precursor forms of Apo (a), as well as mature forms resulting from post-translational processing. An exemplary sequence of a human LPA mRNA transcript is publicly available (GenBank Accession No. NM_005577.3) and disclosed herein (SEQ ID NO: 1). An exemplary sequence of cynomolgus monkey LPA mRNA is publicly available (GenBank Accession No. XM_015448517.1) and disclosed herein (SEQ ID NO: 2).

As used herein, “asialoglycoprotein receptor” or “ASGPR” refers to a bipartite C-type lectin formed by a major 48 kDa subunit (ASGPR-1) and minor 40 kDa subunit (ASGPR-2). ASGPR is primarily expressed on the sinusoidal surface of hepatocyte cells and has a major role in binding, internalizing and subsequent clearing of circulating glycoproteins that contain terminal galactose or GalNAc residues (asialoglycoproteins).

As used herein, “attenuate,” “attenuating,” “attenuation” and the like refers to reducing or effectively halting. As a non-limiting example, one or more of the treatments herein may reduce or effectively halt the onset or progression of cardiometabolic diseases including atherosclerosis, dyslipidemia, NAFLD and NASH in a subject. This attenuation may be exemplified by, for example, a decrease in one or more aspects (e.g., symptoms, tissue characteristics, and cellular, inflammatory or immunological activity, etc.) of cardiometabolic diseases including atherosclerosis, dyslipidemia, NAFLD and NASH, no detectable progression (worsening) of one or more aspects of cardiometabolic diseases including atherosclerosis, dyslipidemia, NAFLD and NASH, or no detectable aspects of cardiometabolic diseases including atherosclerosis, dyslipidemia, NAFLD and NASH in a subject when they might otherwise be expected.

As used herein, “complementary” refers to a structural relationship between two nucleotides (e.g., on two opposing nucleic acids or on opposing regions of a single nucleic acid strand) that permits the two nucleotides to form base pairs with one another. For example, a purine nucleotide of one nucleic acid that is complementary to a pyrimidine nucleotide of an opposing nucleic acid may base pair together by forming hydrogen bonds with one another. In some embodiments, complementary nucleotides can base pair in the Watson-Crick manner or in any other manner that allows for the formation of stable duplexes. In some embodiments, two nucleic acids may have regions of multiple nucleotides that are complementary with each other to form regions of complementarity, as described herein.

As used herein, “deoxyribonucleotide” refers to a nucleotide having a hydrogen in place of a hydroxyl at the 2′ position of its pentose sugar when compared with a ribonucleotide. A modified deoxyribonucleotide is a deoxyribonucleotide having one or more modifications or substitutions of atoms other than at the 2′ position, including modifications or substitutions in or of the sugar, phosphate group or base.

As used herein, “double-stranded oligonucleotide” or “ds oligonucleotide” refers to an oligonucleotide that is substantially in a duplex form. In some embodiments, the complementary base-pairing of duplex region(s) of a ds oligonucleotide is formed between antiparallel sequences of nucleotides of covalently separate nucleic acid strands. In some embodiments, complementary base-pairing of duplex region(s) of a ds oligonucleotide is formed between antiparallel sequences of nucleotides of nucleic acid strands that are covalently linked. In some embodiments, complementary base-pairing of duplex region(s) of a ds oligonucleotide is formed from single nucleic acid strand that is folded (e.g., via a hairpin) to provide complementary antiparallel sequences of nucleotides that base pair together. In some embodiments, a ds oligonucleotide comprises two covalently separate nucleic acid strands that are fully duplexed with one another. However, in some embodiments, a ds oligonucleotide comprises two covalently separate nucleic acid strands that are partially duplexed (e.g., having overhangs at one or both ends). In some embodiments, a ds oligonucleotide comprises antiparallel sequence of nucleotides that are partially complementary, and thus, may have one or more mismatches, which may include internal mismatches or end mismatches.

As used herein, “duplex,” in reference to nucleic acids (e.g., oligonucleotides), refers to a structure formed through complementary base pairing of two antiparallel sequences of nucleotides.

As used herein, “excipient” refers to a non-therapeutic agent that may be included in a composition, for example, to provide or contribute to a desired consistency or stabilizing effect.

As used herein, “hepatocyte” or “hepatocytes” refers to cells of the parenchymal tissues of the liver. These cells make up about 70%-85% of the liver's mass and manufacture serum albumin, FBN and the prothrombin group of clotting factors (except for Factors 3 and 4). Markers for hepatocyte lineage cells include, but are not limited to, transthyretin (Ttr), glutamine synthetase (Glu1), hepatocyte nuclear factor 1a (Hnf1a) and hepatocyte nuclear factor 4a (Hnf4a). Markers for mature hepatocytes may include, but are not limited to, cytochrome P450 (Cyp3al1), fumarylacetoacetate hydrolase (Fah), glucose 6-phosphate (G6p), albumin (Alb) and OC2-2F8. See, e.g., Huch et al. (2013) Nature 494:247-250.

As used herein, a “hepatotoxic agent” refers to a chemical compound, virus or other substance that is itself toxic to the liver or can be processed to form a metabolite that is toxic to the liver. Hepatotoxic agents may include, but are not limited to, carbon tetrachloride (CCl₄), acetaminophen (paracetamol), vinyl chloride, arsenic, chloroform, nonsteroidal anti-inflammatory drugs (such as aspirin and phenylbutazone).

As used herein, “labile linker” refers to a linker that can be cleaved (e.g., by acidic pH). A “fairly stable linker” refers to a linker that cannot be cleaved.

As used herein, “liver inflammation” or “hepatitis” refers to a physical condition in which the liver becomes swollen, dysfunctional and/or painful, especially as a result of injury or infection, as may be caused by exposure to a hepatotoxic agent. Symptoms may include jaundice (yellowing of the skin or eyes), fatigue, weakness, nausea, vomiting, appetite reduction and weight loss. Liver inflammation, if left untreated, may progress to fibrosis, cirrhosis, liver failure or liver cancer.

As used herein, “liver fibrosis” or “fibrosis of the liver” refers to an excessive accumulation in the liver of extracellular matrix proteins, which could include collagens (I, III, and IV), FBN, undulin, elastin, laminin, hyaluronan and proteoglycans resulting from inflammation and liver cell death. Liver fibrosis, if left untreated, may progress to cirrhosis, liver failure or liver cancer.

As used herein, “loop” refers to a unpaired region of a nucleic acid (e.g., oligonucleotide) that is flanked by two antiparallel regions of the nucleic acid that are sufficiently complementary to one another, such that under appropriate hybridization conditions (e.g., in a phosphate buffer, in a cells), the two antiparallel regions, which flank the unpaired region, hybridize to form a duplex (referred to as a “stem”).

As used herein, “modified internucleotide linkage” refers to an internucleotide linkage having one or more chemical modifications when compared with a reference internucleotide linkage comprising a phosphodiester bond. In some embodiments, a modified nucleotide is a non-naturally occurring linkage. Typically, a modified internucleotide linkage confers one or more desirable properties to a nucleic acid in which the modified internucleotide linkage is present. For example, a modified nucleotide may improve thermal stability, resistance to degradation, nuclease resistance, solubility, bioavailability, bioactivity, reduced immunogenicity, etc.

As used herein, “modified nucleotide” refers to a nucleotide having one or more chemical modifications when compared with a corresponding reference nucleotide selected from: adenine ribonucleotide, guanine ribonucleotide, cytosine ribonucleotide, uracil ribonucleotide, adenine deoxyribonucleotide, guanine deoxyribonucleotide, cytosine deoxyribonucleotide and thymidine deoxyribonucleotide. In some embodiments, a modified nucleotide is a non-naturally occurring nucleotide. In some embodiments, a modified nucleotide has one or more chemical modification in its sugar, nucleobase and/or phosphate group. In some embodiments, a modified nucleotide has one or more chemical moieties conjugated to a corresponding reference nucleotide. Typically, a modified nucleotide confers one or more desirable properties to a nucleic acid in which the modified nucleotide is present. For example, a modified nucleotide may improve thermal stability, resistance to degradation, nuclease resistance, solubility, bioavailability, bioactivity, reduced immunogenicity, etc.

As used herein, “nicked tetraloop structure” refers to a structure of a RNAi oligonucleotide that is characterized by separate sense (passenger) and antisense (guide) strands, in which the sense strand has a region of complementarity with the antisense strand, and in which at least one of the strands, generally the sense strand, has a tetraloop configured to stabilize an adjacent stem region formed within the at least one strand.

As used herein, “oligonucleotide” refers to a short nucleic acid (e.g., less than about 100 nucleotides in length). An oligonucleotide may be single-stranded (ss) or ds. An oligonucleotide may or may not have duplex regions. As a set of non-limiting examples, an oligonucleotide may be, but is not limited to, a small interfering RNA (siRNA), microRNA (miRNA), short hairpin RNA (shRNA), dicer substrate interfering RNA (dsiRNA), antisense oligonucleotide, short siRNA or ss siRNA. In some embodiments, a ds oligonucleotide is an RNAi oligonucleotide.

As used herein, “overhang” refers to terminal non-base pairing nucleotide(s) resulting from one strand or region extending beyond the terminus of a complementary strand with which the one strand or region forms a duplex. In some embodiments, an overhang comprises one or more unpaired nucleotides extending from a duplex region at the 5′ terminus or 3′ terminus of a ds oligonucleotide. In certain embodiments, the overhang is a 3′ or 5′ overhang on the antisense strand or sense strand of a ds oligonucleotides.

As used herein, “phosphate analog” refers to a chemical moiety that mimics the electrostatic and/or steric properties of a phosphate group. In some embodiments, a phosphate analog is positioned at the 5′ terminal nucleotide of an oligonucleotide in place of a 5′-phosphate, which is often susceptible to enzymatic removal. In some embodiments, a 5′ phosphate analog contains a phosphatase-resistant linkage. Examples of phosphate analogs include, but are not limited to, 5′ phosphonates, such as 5′ methylenephosphonate (5′-MP) and 5′-(E)-vinylphosphonate (5′-VP). In some embodiments, an oligonucleotide has a phosphate analog at a 4′-carbon position of the sugar (referred to as a “4′-phosphate analog”) at a 5′-terminal nucleotide. An example of a 4′-phosphate analog is oxymethylphosphonate, in which the oxygen atom of the oxymethyl group is bound to the sugar moiety (e.g., at its 4′-carbon) or analog thereof. See, e.g., U.S. Provisional Patent Application Nos. 62/383,207 (filed on 2 Sep. 2016) and 62/393,401 (filed on 12 Sep. 2016). Other modifications have been developed for the 5′ end of oligonucleotides (see, e.g., Intl. Patent Application No. WO 2011/133871; U.S. Pat. No. 8,927,513; and Prakash et al. (2015) Nucleic Acids Res. 43:2993-3011).

As used herein, “reduced expression” of a gene (e.g., LPA) refers to a decrease in the amount or level of RNA transcript (e.g., LPA mRNA) or protein encoded by the gene and/or a decrease in the amount or level of activity of the gene in a cell, a population of cells, a sample or a subject, when compared to an appropriate reference (e.g., a reference cell, population of cells, sample or subject). For example, the act of contacting a cell with an oligonucleotide herein (e.g., an oligonucleotide comprising an antisense strand having a nucleotide sequence that is complementary to a nucleotide sequence comprising LPA mRNA) may result in a decrease in the amount or level of LPA mRNA, apo(a) protein and/or apo(a) activity (e.g., via inactivation and/or degradation of LPA mRNA by the RNAi pathway) when compared to a cell that is not treated with the ds oligonucleotide. Similarly, and as used herein, “reducing expression” refers to an act that results in reduced expression of a gene (e.g., LPA). As used herein, “reduction of LPA expression” refers to a decrease in the amount or level of LPA mRNA, apo(a) protein and/or apo(a) activity in a cell, a population of cells, a sample or a subject when compared to an appropriate reference (e.g., a reference cell, population of cells, sample, or subject).

As used herein, “region of complementarity” refers to a sequence of nucleotides of a nucleic acid (e.g., a ds oligonucleotide) that is sufficiently complementary to an antiparallel sequence of nucleotides to permit hybridization between the two sequences of nucleotides under appropriate hybridization conditions (e.g., in a phosphate buffer, in a cell, etc.). In some embodiments, an oligonucleotide herein comprises a targeting sequence having a region of complementary to a mRNA target sequence.

As used herein, “ribonucleotide” refers to a nucleotide having a ribose as its pentose sugar, which contains a hydroxyl group at its 2′ position. A modified ribonucleotide is a ribonucleotide having one or more modifications or substitutions of atoms other than at the 2′ position, including modifications or substitutions in or of the ribose, phosphate group or base.

As used herein, “RNAi oligonucleotide” refers to either (a) a ds oligonucleotide having a sense strand (passenger) and antisense strand (guide), in which the antisense strand or part of the antisense strand is used by the Argonaute 2 (Ago2) endonuclease in the cleavage of a target mRNA (e.g., LPA mRNA) or (b) a ss oligonucleotide having a single antisense strand, where that antisense strand (or part of that antisense strand) is used by the Ago2 endonuclease in the cleavage of a target mRNA (e.g., LPA mRNA).

As used herein, “strand” refers to a single, contiguous sequence of nucleotides linked together through internucleotide linkages (e.g., phosphodiester linkages or phosphorothioate linkages). In some embodiments, a strand has two free ends (e.g., a 5′ end and a 3′ end).

As used herein, “subject” means any mammal, including mice, rabbits and humans. In one embodiment, the subject is a human or NHP. Moreover, “individual” or “patient” may be used interchangeably with “subject.”

As used herein, “synthetic” refers to a nucleic acid or other molecule that is artificially synthesized (e.g., using a machine (e.g., a solid-state nucleic acid synthesizer)) or that is otherwise not derived from a natural source (e.g., a cell or organism) that normally produces the molecule.

As used herein, “targeting ligand” refers to a molecule (e.g., a carbohydrate, amino sugar, cholesterol, polypeptide or lipid) that selectively binds to a cognate molecule (e.g., a receptor) of a tissue or cell of interest and that is conjugatable to another substance for purposes of targeting the other substance to the tissue or cell of interest. For example, in some embodiments, a targeting ligand may be conjugated to an oligonucleotide for purposes of targeting the oligonucleotide to a specific tissue or cell of interest. In some embodiments, a targeting ligand selectively binds to a cell surface receptor. Accordingly, in some embodiments, a targeting ligand when conjugated to an oligonucleotide facilitates delivery of the oligonucleotide into a particular cell through selective binding to a receptor expressed on the surface of the cell and endosomal internalization by the cell of the complex comprising the oligonucleotide, targeting ligand and receptor. In some embodiments, a targeting ligand is conjugated to an oligonucleotide via a linker that is cleaved following or during cellular internalization such that the oligonucleotide is released from the targeting ligand in the cell.

As used herein, “tetraloop” refers to a loop that increases stability of an adjacent duplex formed by hybridization of flanking sequences of nucleotides. The increase in stability is detectable as an increase in melting temperature (Tₘ) of an adjacent stem duplex that is higher than the Tₘ of the adjacent stem duplex expected, on average, from a set of loops of comparable length consisting of randomly selected sequences of nucleotides. For example, a tetraloop can confer a Tₘ of at least about 50° C., at least about 55° C., at least about 56° C., at least about 58° C., at least about 60° C., at least about 65° C. or at least about 75° C. in 10 mM NaHPO₄ to a hairpin comprising a duplex of at least 2 base pairs (bp) in length. In some embodiments, a tetraloop may stabilize a bp in an adjacent stem duplex by stacking interactions. In addition, interactions among the nucleotides in a tetraloop include, but are not limited to, non-Watson-Crick base pairing, stacking interactions, hydrogen bonding and contact interactions (Cheong et al. (1990) NATURE 346:680-82; Heus & Pardi (1991) SCIENCE 253:191-94). In some embodiments, a tetraloop comprises or consists of 3 to 6 nucleotides and is typically 4 to 5 nucleotides. In certain embodiments, a tetraloop comprises or consists of 3, 4, 5 or 6 nucleotides, which may or may not be modified (e.g., which may or may not be conjugated to a targeting moiety). In one embodiment, a tetraloop consists of 4 nucleotides. Any nucleotide may be used in the tetraloop and standard IUPAC-IUB symbols for such nucleotides may be used as described in Cornish-Bowden (1985) Nucleic Acids Res. 13:3021-3030. For example, the letter “N” may be used to mean that any base may be in that position, the letter “R” may be used to show that A (adenine) or G (guanine) may be in that position, and “B” may be used to show that C (cytosine), G (guanine), T (thymine) or U (uracil) may be in that position. Examples of tetraloops include the UNCG family of tetraloops (e.g., UUCG), the GNRA family of tetraloops (e.g., GAAA), and the CUUG tetraloop (Woese et al. (1990) Proc. Natl. Acad. Sci. USA 87:8467-8471; Antao et al. (1991) Nucleic Acids Res. 19:5901-5905). Examples of DNA tetraloops include the d (GNNA) family of tetraloops (e.g., d (GTTA), the d (GNRA)) family of tetraloops, the d (GNAB) family of tetraloops, the d (CNNG) family of tetraloops, and the d (TNCG) family of tetraloops (e.g., d (TTCG)). See, e.g., Nakano et al. (2002) Biochem. 41:4281-14292; Shinji et al. (2000) Nippon Kagakkai Koen Yokoshu 78:731. In some embodiments, the tetraloop is contained within a nicked tetraloop structure.

As used herein, “treat” or “treating” refers to the act of providing care to a subject in need thereof, for example, by administering a therapeutic agent (e.g., an oligonucleotide herein) to the subject, for purposes of improving the health and/or well-being of the subject with respect to an existing condition (e.g., a disease, disorder) or to prevent or decrease the likelihood of the occurrence of a condition. In some embodiments, treatment involves reducing the frequency or severity of at least one sign, symptom or contributing factor of a condition (e.g., disease, disorder) experienced by a subject.

### II. Oligonucleotide Inhibitors of LPA Expression

The disclosure provides, inter alia, oligonucleotides that inhibit LPA expression. In some embodiments, an oligonucleotide that inhibits LPA expression herein is targeted to an LPA mRNA.

i. LPA Target Sequences

In some embodiments, the oligonucleotide is targeted to a target sequence comprising an LPA mRNA. In some embodiments, the oligonucleotide, or a portion, fragment or strand thereof (e.g., an antisense strand or a guide strand of a ds oligonucleotide) binds or anneals to a target sequence comprising an LPA mRNA, thereby inhibiting LPA expression. In some embodiments, the oligonucleotide is targeted to an LPA target sequence for the purpose of inhibiting LPA expression in vivo. In some embodiments, the amount or extent of inhibition of LPA expression by an oligonucleotide targeted to an LPA target sequence correlates with the potency of the oligonucleotide. In some embodiments, the amount or extent of inhibition of LPA expression by an oligonucleotide targeted to an LPA target sequence correlates with the amount or extent of therapeutic benefit in a subject or patient having a disease, disorder or condition associated with the expression of LPA treated with the oligonucleotide.

Through examination and analysis of the nucleotide sequence of LPA mRNAs encoding apo(a), including mRNAs of multiple different species (e.g., human, cynomolgus monkey, and rhesus monkey; see, e.g., Example 1) and as a result of in vitro and in vivo testing (see, e.g., Example 2 and Example 3), it has been discovered that certain nucleotide sequences of LPA mRNA are more amenable than others to oligonucleotide-based inhibition of LPA expression and are thus useful as target sequences for the oligonucleotides herein. In some embodiments, a sense strand of an oligonucleotide (e.g., a ds oligonucleotide) described herein (e.g., in Table 5) comprises an LPA target sequence. In some embodiments, a portion or region of the sense strand of a ds oligonucleotide described herein (e.g., in Table 5) comprises an LPA target sequence. In some embodiments, an LPA target sequence comprises, or consists of, a sequence of any one of SEQ ID Nos: 4-387.

ii. LPA Targeting Sequences

In some embodiments, the oligonucleotides herein have regions of complementarity to LPA mRNA (e.g., within a target sequence of LPA mRNA) for purposes of targeting the LPA mRNA in cells and inhibiting LPA expression. In some embodiments, the oligonucleotides herein comprise an LPA targeting sequence (e.g., an antisense strand or a guide strand of a ds oligonucleotide) having a region of complementarity that binds or anneals to an LPA target sequence by complementary (Watson-Crick) base pairing. The targeting sequence or region of complementarity is generally of a suitable length and base content to enable binding or annealing of the oligonucleotide (or a strand thereof) to an LPA mRNA for purposes of inhibiting its expression. In some embodiments, the targeting sequence or region of complementarity is at least about 12, at least about 13, at least about 14, at least about 15, at least about 16, at least about 17, at least about 18, at least about 19, at least about 20, at least about 21, at least about 22, at least about 23, at least about 24, at least about 25, at least about 26, at least about 27, at least about 28, at least about 29 or at least about 30 nucleotides in length. In some embodiments, the targeting sequence or region of complementarity is about 12 to about 30 (e.g., 12 to 30, 12 to 22, 15 to 25, 17 to 21, 18 to 27, 19 to 27, or 15 to 30) nucleotides in length. In some embodiments, the targeting sequence or region of complementarity is about 12, 13, 14, 15, 16, 17, 18, 19, 20, 21, 22, 23, 24, 25, 26, 27, 28, 29 or 30 nucleotides in length. In some embodiments, the targeting sequence or region of complementarity is 18 nucleotides in length. In some embodiments, the targeting sequence or region of complementarity is 19 nucleotides in length. In some embodiments, the targeting sequence or region of complementarity is 20 nucleotides in length. In some embodiments, the targeting sequence or region of complementarity is 21 nucleotides in length. In some embodiments, the targeting sequence or region of complementarity is 22 nucleotides in length. In some embodiments, the targeting sequence or region of complementarity is 23 nucleotides in length. In some embodiments, the targeting sequence or region of complementarity is 24 nucleotides in length.

In some embodiments, an oligonucleotide herein comprises a targeting sequence or a region of complementarity (e.g., an antisense strand or a guide strand of a double-stranded oligonucleotide) that is fully complementary to an LPA target sequence. In some embodiments, the targeting sequence or region of complementarity is partially complementary to an LPA target sequence. In some embodiments, the oligonucleotide comprises a targeting sequence or region of complementarity that is fully complementary to a sequence of any one of SEQ ID NOs: 4-387. In some embodiments, the oligonucleotide comprises a targeting sequence or region of complementarity that is partially complementary to a sequence of any one of SEQ ID NOs: 4-387.

In some embodiments, the oligonucleotide herein comprises a targeting sequence or region of complementarity that is complementary to a contiguous sequence of nucleotides comprising an LPA mRNA, wherein the contiguous sequence of nucleotides is about 12 to about 30 nucleotides in length (e.g., 12 to 30, 12 to 28, 12 to 26, 12 to 24, 12 to 20, 12 to 18, 12 to 16, 14 to 22, 16 to 20, 18 to 20 or 18 to 19 nucleotides in length). In some embodiments, the oligonucleotide comprises a targeting sequence or region of complementarity that is complementary to a contiguous sequence of nucleotides comprising an LPA mRNA, wherein the contiguous sequence of nucleotides is 10, 11, 12, 13, 14, 15, 16, 17, 18, 19 or 20 nucleotides in length. In some embodiments, the oligonucleotide comprises a targeting sequence or region of complementarity that is complementary to a contiguous sequence of nucleotides comprising an LPA mRNA, wherein the contiguous sequence of nucleotides is 19 nucleotides in length. In some embodiments, the oligonucleotide comprises a targeting sequence or region of complementarity that is complementary to a contiguous sequence of nucleotides comprising an LPA mRNA, wherein the contiguous sequence of nucleotides is 20 nucleotides in length. In some embodiments, the oligonucleotide comprises a targeting sequence or a region of complementarity that is complementary to a contiguous sequence of nucleotides of any one of SEQ ID NOs: 4-387, optionally wherein the contiguous sequence of nucleotides is 19 nucleotides in length.

In some embodiments, a targeting sequence or region of complementarity of an oligonucleotide that is complementary to contiguous nucleotides of a sequence as set forth in any one of SEQ ID NOs: 4-387 and spans the entire length of an antisense strand. In some embodiments, a region of complementarity of an oligonucleotide that is complementary to contiguous nucleotides of a sequence as set forth in any one of SEQ ID NOs: 4-387 and spans a portion of the entire length of an antisense strand. In some embodiments, an oligonucleotide herein comprises a region of complementarity (e.g., on an antisense strand of a ds oligonucleotide) that is at least partially (e.g., fully) complementary to a contiguous stretch of nucleotides spanning nucleotides 1-20 of a sequence as set forth in any one of SEQ ID NOs: 4-387.

In some embodiments, an oligonucleotide herein comprises a targeting sequence or region of complementarity having one or more base pair (bp) mismatches with the corresponding LPA target sequence. In some embodiments, the targeting sequence or region of complementarity may have up to about 1, up to about 2, up to about 3, up to about 4, up to about 5, etc. mismatches with the corresponding LPA target sequence provided that the ability of the targeting sequence or region of complementarity to bind or anneal to the LPA mRNA under appropriate hybridization conditions and/or the ability of the oligonucleotide to reduce or inhibit LPA expression is maintained. Alternatively, in some embodiments, the targeting sequence or region of complementarity comprises no more than 1, no more than 2, no more than 3, no more than 4, or no more than 5 mismatches with the corresponding LPA target sequence provided that the ability of the targeting sequence or region of complementarity to bind or anneal to the LPA mRNA under appropriate hybridization conditions and/or the ability of the oligonucleotide to reduce or inhibit LPA expression is maintained. In some embodiments, the oligonucleotide comprises a targeting sequence or region of complementarity having 1 mismatch with the corresponding target sequence. In some embodiments, the oligonucleotide comprises a targeting sequence or region of complementarity having 2 mismatches with the corresponding target sequence. In some embodiments, the oligonucleotide comprises a targeting sequence or region of complementarity having 3 mismatches with the corresponding target sequence. In some embodiments, the oligonucleotide comprises a targeting sequence or region of complementarity having 4 mismatches with the corresponding target sequence. In some embodiments, the oligonucleotide comprises a targeting sequence or region of complementarity having 5 mismatches with the corresponding target sequence. In some embodiments, the oligonucleotide comprises a targeting sequence or region of complementarity more than one mismatch (e.g., 2, 3, 4, 5 or more mismatches) with the corresponding target sequence, wherein at least 2 (e.g., all) of the mismatches are positioned consecutively (e.g., 2, 3, 4, 5 or more mismatches in a row), or wherein the mismatches are interspersed in any position throughout the targeting sequence or region of complementarity. In some embodiments, the oligonucleotide comprises a targeting sequence or region of complementarity more than one mismatch (e.g., 2, 3, 4, 5 or more mismatches) with the corresponding target sequence, wherein at least 2 (e.g., all) of the mismatches are positioned consecutively (e.g., 2, 3, 4, 5 or more mismatches in a row), or wherein at least one or more non-mismatched base pair is located between the mismatches, or a combination thereof.

iii. Types of Oligonucleotides

A variety of oligonucleotide types and/or structures are useful for targeting LPA mRNA in the methods herein including, but not limited to, RNAi oligonucleotides, antisense oligonucleotides, miRNAs, etc. Any of the oligonucleotide types described herein or elsewhere are contemplated for use as a framework to incorporate an LPA mRNA targeting sequence herein for the purposes of inhibiting LPA expression.

In some embodiments, the oligonucleotides herein inhibit LPA expression by engaging with RNA interference (RNAi) pathways upstream or downstream of Dicer involvement. For example, RNAi oligonucleotides have been developed with each strand having sizes of about 19-25 nucleotides with at least one 3′ overhang of 1 to 5 nucleotides (see, e.g., U.S. Pat. No. 8,372,968). Longer oligonucleotides also have been developed that are processed by Dicer to generate active RNAi products (see, e.g., U.S. Pat. No. 8,883,996). Further work produced extended ds oligonucleotides where at least one end of at least one strand is extended beyond a duplex targeting region, including structures where one of the strands includes a thermodynamically-stabilizing tetraloop structure (see, e.g., U.S. Pat. Nos. 8,513,207 and 8,927,705, as well as Intl. Patent Application Publication No. WO 2010/033225). Such structures may include ss extensions (on one or both sides of the molecule) as well as ds extensions.

In some embodiments, the oligonucleotides herein engage with the RNAi pathway downstream of the involvement of Dicer (e.g., Dicer cleavage). In some embodiments, the oligonucleotide has an overhang (e.g., of 1, 2, or 3 nucleotides in length) in the 3′ end of the sense strand. In some embodiments, the oligonucleotide (e.g., siRNA) comprises a 21-nucleotide guide strand that is antisense to a target mRNA (e.g., LPA mRNA) and a complementary passenger strand, in which both strands anneal to form a 19-bp duplex and 2 nucleotide overhangs at either or both 3′ ends. Longer oligonucleotide designs also are contemplated including oligonucleotides having a guide strand of 23 nucleotides and a passenger strand of 21 nucleotides, where there is a blunt end on the right side of the molecule (3′ end of passenger strand/5′ end of guide strand) and a two nucleotide 3′-guide strand overhang on the left side of the molecule (5′ end of the passenger strand/3′ end of the guide strand). In such molecules, there is a 21 bp duplex region. See, e.g., U.S. Pat. Nos. 9,012,138; 9,012,621 and 9,193,753.

In some embodiments, the oligonucleotides herein comprise sense and antisense strands that are both in the range of about 17 to 26 (e.g., 17 to 26, 20 to 25 or 21-23) nucleotides in length. In some embodiments, an oligonucleotide herein comprises a sense and antisense strand that are both in the range of about 19-22 nucleotides in length. In some embodiments, the sense and antisense strands are of equal length. In some embodiments, an oligonucleotide comprises sense and antisense strands, such that there is a 3′-overhang on either the sense strand or the antisense strand, or both the sense and antisense strand. In some embodiments, for oligonucleotides that have sense and antisense strands that are both in the range of about 21-23 nucleotides in length, a 3′ overhang on the sense, antisense, or both sense and antisense strands is 1 or 2 nucleotides in length. In some embodiments, the oligonucleotide has a guide strand of 22 nucleotides and a passenger strand of 20 nucleotides, where there is a blunt end on the right side of the molecule (3′ end of passenger strand/5′ end of guide strand) and a 2 nucleotide 3′-guide strand overhang on the left side of the molecule (5′ end of the passenger strand/3′ end of the guide strand). In such molecules, there is a 20 bp duplex region.

Other oligonucleotide designs for use with the compositions and methods herein include: 16-mer siRNAs (see, e.g., NUCLEIC ACIDS IN CHEMISTRY AND BIOLOGY, Blackburn (ed.), Royal Society of Chemistry, 2006), shRNAs (e.g., having 19 bp or shorter stems; see, e.g., Moore et al. (2010) METHODS MOL. BIOL. 629:141-58), blunt siRNAs (e.g., of 19 bps in length; see, e.g., Kraynack & Baker (2006) RNA 12:163-76), asymmetrical siRNAs (aiRNA; see, e.g., Sun et al. (2008) NAT. BIOTECHNOL. 26:1379-82), asymmetric shorter-duplex siRNA (see, e.g., Chang et al. (2009) MOL. THER. 17:725-32), fork siRNAs (see, e.g., Hohjoh (2004) FEBS LETT. 557:193-198), ss siRNAs (Elsner (2012) NAT. BIOTECHNOL. 30:1063), dumbbell-shaped circular siRNAs (see, e.g., Abe et al. (2007) J. AM. CHEM. SOC. 129:15108-09), and small internally segmented interfering RNA (siRNA; see, e.g., Bramsen et al. (2007) NUCLEIC ACIDS RES. 35:5886-97). Further non-limiting examples of an oligonucleotide structures that may be used in some embodiments to reduce or inhibit the expression of LPA are microRNA (miRNA), short hairpin RNA (shRNA) and short siRNA (see, e.g., Hamilton et al. (2002) EMBO J. 21:4671-79; see also, US Patent Application Publication No. 2009/0099115).

Still, in some embodiments, an oligonucleotide for reducing or inhibiting LPA expression herein is single-stranded (ss). Such structures may include but are not limited to ss RNAi molecules. Recent efforts have demonstrated the activity of ss RNAi molecules (see, e.g., Matsui et al. (2016) Mol. Ther. 24:946-955). However, in some embodiments, oligonucleotides herein are antisense oligonucleotides (ASOs). An antisense oligonucleotide is a ss oligonucleotide that has a nucleobase sequence which, when written or depicted in the 5′ to 3′ direction, comprises the reverse complement of a targeted segment of a particular nucleic acid and is suitably modified (e.g., as a gapmer) so as to induce RNaseH-mediated cleavage of its target RNA in cells or (e.g., as a mixmer) so as to inhibit translation of the target mRNA in cells. ASOs for use herein may be modified in any suitable manner known in the art including, for example, as shown in U.S. Pat. No. 9,567,587 (including, e.g., length, sugar moieties of the nucleobase (pyrimidine, purine), and alterations of the heterocyclic portion of the nucleobase). Further, ASOs have been used for decades to reduce expression of specific target genes (see, e.g., Bennett et al. (2017) Annu. Rev. Pharmacol. 57:81-105).

iv. Double-Stranded Oligonucleotides

The disclosure provides double-stranded (ds) oligonucleotides for targeting LPA mRNA and inhibiting LPA expression (e.g., via the RNAi pathway) comprising a sense strand (also referred to herein as a passenger strand) and an antisense strand (also referred to herein as a guide strand). In some embodiments, the sense strand and antisense strand are separate strands and are not covalently linked. In some embodiments, the sense strand and antisense strand are covalently linked.

In some embodiments, the sense strand has a first region (R1) and a second region (R2), wherein R2 comprises a first subregion (S1), a tetraloop (L) or triloop (triL), and a second subregion (S2), wherein L or triL is located between S1 and S2, and wherein S1 and S2 form a second duplex (D2). D2 may have various lengths. In some embodiments, D2 is about 1-6 bp in length. In some embodiments, D2 is 2-6, 3-6, 4-6, 5-6, 1-5, 2-5, 3-5 or 4-5 bp in length. In some embodiments, D2 is 1, 2, 3, 4, 5 or 6 bp in length. In some embodiments, D2 is 6 bp in length.

In some embodiments, R1 of the sense strand and the antisense strand form a first duplex (D1). In some embodiments, D1 is at least about 15 (e.g., at least 15, at least 16, at least 17, at least 18, at least 19, at least 20 or at least 21) nucleotides in length. In some embodiments, D1 is in the range of about 12 to 30 nucleotides in length (e.g., 12 to 30, 12 to 27, 15 to 22, 18 to 22, 18 to 25, 18 to 27, 18 to 30 or 21 to 30 nucleotides in length). In some embodiments, D1 is at least 12 nucleotides in length (e.g., at least 12, at least 15, at least 20, at least 25, or at least 30 nucleotides in length). In some embodiments, D1 is 12, 13, 14, 15, 16, 17, 18, 19, 20, 21, 22, 23, 24, 25, 26, 27, 28, 29 or 30 nucleotides in length. In some embodiments, D1 is 20 nucleotides in length. In some embodiments, D1 comprising sense strand and antisense strand does not span the entire length of the sense strand and/or antisense strand. In some embodiments, D1 comprising the sense strand and antisense strand spans the entire length of either the sense strand or antisense strand or both. In certain embodiments, D1 comprising the sense strand and antisense strand spans the entire length of both the sense strand and the antisense strand.

In some embodiments, a ds oligonucleotide herein comprises a sense strand having a sequence of any one of SEQ ID NOs: 388-403 and an antisense strand comprising a complementary sequence selected from SEQ ID NOs: 788-803, as is arranged Table 3. In some embodiments, the sense strand comprises the sequence of SEQ ID NO: 393 and the antisense strand comprises the sequence of SEQ ID NO: 793.

It should be appreciated that, in some embodiments, sequences presented in the Sequence Listing may be referred to in describing the structure of an oligonucleotide (e.g., a ds oligonucleotide) or other nucleic acid. In such embodiments, the actual oligonucleotide or other nucleic acid may have one or more alternative nucleotides (e.g., an RNA counterpart of a DNA nucleotide or a DNA counterpart of an RNA nucleotide) and/or one or more modified nucleotides and/or one or more modified internucleotide linkages and/or one or more other modification when compared with the specified sequence while retaining essentially same or similar complementary properties as the specified sequence.

In some embodiments, a ds oligonucleotide herein comprises a 25-nucleotide sense strand and a 27-nucleotide antisense strand that when acted upon by a Dicer enzyme results in an antisense strand that is incorporated into the mature RISC. In some embodiments, the sense strand of the ds oligonucleotide is longer than 27 nucleotides (e.g., 28, 29, 30, 31, 32, 33, 34, 35, 36, 37, 38, 39 or 40 nucleotides). In some embodiments, the sense strand of the ds oligonucleotide is longer than 25 nucleotides (e.g., 26, 27, 28, 29 or 30 nucleotides).

In some embodiments, the ds oligonucleotides herein have one 5′ end that is thermodynamically less stable when compared to the other 5′ end. In some embodiments, an asymmetric ds oligonucleotide is provided that comprises a blunt end at the 3′ end of a sense strand and a 3′-overhang at the 3′ end of an antisense strand. In some embodiments, the 3′-overhang on the antisense strand is about 1-8 nucleotides in length (e.g., 1, 2, 3, 4, 5, 6, 7 or 8 nucleotides in length). Typically, a ds oligonucleotide for RNAi has a two-nucleotide overhang on the 3′ end of the antisense (guide) strand. However, other overhangs are possible. In some embodiments, an overhang is a 3′-overhang comprising a length of between 1 and 6 nucleotides, optionally 1 to 5, 1 to 4, 1 to 3, 1 to 2, 2 to 6, 2 to 5, 2 to 4, 2 to 3, 3 to 6, 3 to 5, 3 to 4, 4 to 6, 4 to 5, 5 to 6 nucleotides, or 1, 2, 3, 4, 5 or 6 nucleotides. However, in some embodiments, the overhang is a 5′-overhang comprising a length of between 1 and 6 nucleotides, optionally 1 to 5, 1 to 4, 1 to 3, 1 to 2, 2 to 6, 2 to 5, 2 to 4, 2 to 3, 3 to 6, 3 to 5, 3 to 4, 4 to 6, 4 to 5, 5 to 6 nucleotides, or 1, 2, 3, 4, 5 or 6 nucleotides.

In some embodiments, two terminal nucleotides on the 3′ end of an antisense strand are modified. In some embodiments, the two terminal nucleotides on the 3′ end of the antisense strand are complementary with the target mRNA (e.g., LPA mRNA). In some embodiments, the two terminal nucleotides on the 3′ end of the antisense strand are not complementary with the target mRNA. In some embodiments, two terminal nucleotides on each 3′ end of an oligonucleotide in the nicked tetraloop structure are GG. Typically, one or both of the two terminal GG nucleotides on each 3′ end of a ds oligonucleotide is not complementary with the target mRNA.

In some embodiments, there is one or more (e.g., 1, 2, 3, 4 or 5) mismatch(s) between a sense and antisense strand. If there is more than one mismatch between a sense and antisense strand, they may be positioned consecutively (e.g., 2, 3 or more in a row), or interspersed throughout the region of complementarity. In some embodiments, the 3′ end of the sense strand contains one or more mismatches. In one embodiment, two mismatches are incorporated at the 3′ end of the sense strand. In some embodiments, base mismatches, or destabilization of segments at the 3′ end of the sense strand of the oligonucleotide improves or increases the potency of the ds oligonucleotide.

a. Antisense Strands

In some embodiments, an oligonucleotide (e.g., a ds oligonucleotide) disclosed herein for targeting LPA mRNA and inhibiting LPA expression comprises an antisense strand comprising or consisting of a sequence as set forth in any one of SEQ ID NOs: 404-803. In some embodiments, an oligonucleotide herein comprises an antisense strand comprising or consisting of at least about 12 (e.g., at least 12, at least 13, at least 14, at least 15, at least 16, at least 17, at least 18, at least 19, at least 20, at least 21, at least 22 or at least 23) contiguous nucleotides of a sequence as set forth in any one of SEQ ID NOs: 404-803.

In some embodiments, an oligonucleotide (e.g., a ds oligonucleotide) herein comprises an antisense strand of up to about 40 nucleotides in length (e.g., up to 40, up to 35, up to 30, up to 27, up to 25, up to 21, up to 19, up to 17 or up to 12 nucleotides in length). In some embodiments, an oligonucleotide may have an antisense strand of at least about 12 nucleotides in length (e.g., at least 12, at least 15, at least 19, at least 21, at least 22, at least 25, at least 27, at least 30, at least 35 or at least 38 nucleotides in length). In some embodiments, an oligonucleotide may have an antisense strand in a range of about 12 to about 40 (e.g., 12 to 40, 12 to 36, 12 to 32, 12 to 28, 15 to 40, 15 to 36, 15 to 32, 15 to 28, 17 to 22, 17 to 25, 19 to 27, 19 to 30, 20 to 40, 22 to 40, 25 to 40 or 32 to 40) nucleotides in length. In some embodiments, an oligonucleotide may have an antisense strand of 12, 13, 14, 15, 16, 17, 18, 19, 20, 21, 22, 23, 24, 25, 26, 27, 28, 29, 30, 31, 32, 33, 34, 35, 36, 37, 38, 39 or 40 nucleotides in length.

In some embodiments, an antisense strand of an oligonucleotide is referred to as a “guide strand.” For example, an antisense strand that engages with RNA-induced silencing complex (RISC) and binds to an Argonaute protein such as Ago2, or engages with or binds to one or more similar factors, and directs silencing of a target gene, the antisense strand is referred to as a guide strand. In some embodiments, a sense strand complementary to a guide strand is referred to as a “passenger strand.”

b. Sense Strands

In some embodiments, an oligonucleotide (e.g., a ds oligonucleotide) herein for targeting LPA mRNA and inhibiting LPA expression comprises or consists of a sense strand sequence as set forth in in any one of SEQ ID NOs: 4-403. In some embodiments, an oligonucleotide has a sense strand that comprises or consists of at least about 12 (e.g., at least 13, at least 14, at least 15, at least 16, at least 17, at least 18, at least 19, at least 20, at least 21, at least 22 or at least 23) contiguous nucleotides of a sequence as set forth in in any one of SEQ ID NOs: 4-403.

In some embodiments, an oligonucleotide (e.g., a ds oligonucleotide) herein comprises a sense strand (or passenger strand) of up to about 40 nucleotides in length (e.g., up to 40, up to 36, up to 30, up to 27, up to 25, up to 21, up to 19, up to 17 or up to 12 nucleotides in length). In some embodiments, an oligonucleotide may have a sense strand of at least about 12 nucleotides in length (e.g., at least 12, at least 15, at least 19, at least 21, at least 25, at least 27, at least 30, at least 36 or at least 38 nucleotides in length). In some embodiments, an oligonucleotide may have a sense strand in a range of about 12 to about 40 (e.g., 12 to 40, 12 to 36, 12 to 32, 12 to 28, 15 to 40, 15 to 36, 15 to 32, 15 to 28, 17 to 21, 17 to 25, 19 to 27, 19 to 30, 20 to 40, 22 to 40, 25 to 40 or 32 to 40) nucleotides in length. In some embodiments, an oligonucleotide may have a sense strand of 12, 13, 14, 15, 16, 17, 18, 19, 20, 21, 22, 23, 24, 25, 26, 27, 28, 29, 30, 31, 32, 33, 34, 35, 36, 37, 38, 39 or 40 nucleotides in length.

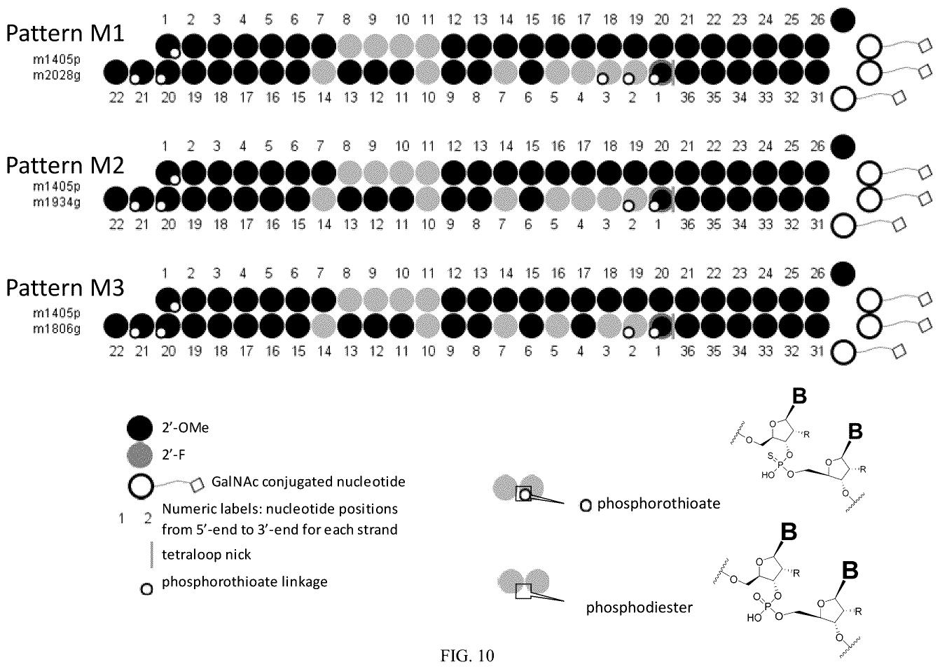

In some embodiments, a sense strand comprises a stem-loop structure at its 3′ end. In some embodiments, a sense strand comprises a stem-loop structure at its 5′ end. In some embodiments, a stem is a duplex of 2, 3, 4, 5, 6, 7, 8, 9, 10, 11, 12, 13 or 14 bp in length. In some embodiments, a stem-loop provides the oligonucleotide protection against degradation (e.g., enzymatic degradation) and facilitates or improves targeting and/or delivery to a target cell, tissue, or organ (e.g, the liver), or both. For example, in some embodiments, the loop of a stem-loop provides nucleotides comprising one or more modifications that facilitate, improve, or increase targeting to a target mRNA (e.g., an LPA mRNA), inhibition of target gene expression (e.g., LPA expression), and/or delivery to a target cell, tissue, or organ (e.g., the liver), or both. In some embodiments, the stem-loop itself or modification(s) to the stem-loop do not substantially affect the inherent gene expression inhibition activity of the oligonucleotide, but facilitates, improves, or increases stability (e.g., provides protection against degradation) and/or delivery of the oligonucleotide to a target cell, tissue, or organ (e.g., the liver). In certain embodiments, an oligonucleotide comprises a sense strand comprising (e.g., at its 3′ end) a stem-loop set forth as: S1-L-S2, in which S1 is complementary to S2, and in which L forms a single-stranded loop between S1 and S2 of up to about 10 nucleotides in length (e.g., 3, 4, 5, 6, 7, 8, 9 or 10 nucleotides in length). In some embodiments, the loop (L) is 4 nucleotides in length. FIG. 10 depicts a non-limiting example of such an oligonucleotide. In some embodiments, a loop (L) of a stem-loop having the structure S1-L-S2 as described above is a tetraloop (e.g., within a nicked tetraloop structure). In some embodiments, the tetraloop comprises ribonucleotides, deoxyribonucleotides, modified nucleotides, delivery ligands, and combinations thereof.

### V. Oligonucleotide Modifications

a. Sugar Modifications

In some embodiments, a modified sugar (also referred herein to a sugar analog) includes a modified deoxyribose or ribose moiety in which, for example, one or more modifications occur at the 2′, 3′, 4′ and/or 5′ carbon position of the sugar. In some embodiments, a modified sugar may also include non-natural alternative carbon structures such as those present in locked nucleic acids (“LNA”; see, e.g., Koshkin et al. (1998) TETRAHEDON 54:3607-30), unlocked nucleic acids (“UNA”; see, e.g., Snead et al. (2013) MOL. THER-NUCL. ACIDS 2: e103) and bridged nucleic acids (“BNA”; see, e.g., Imanishi & Obika (2002) CHEM COMMUN. (CAMB) 21:1653-59).

In some embodiments, a nucleotide modification in a sugar comprises a 2′-modification. In some embodiments, a 2′-modification may be 2′-O-propargyl, 2′-O-propylamin, 2′-amino, 2′-ethyl, 2′-fluoro (2′-F), 2′-aminoethyl (EA), 2′-O-methyl (2′-OMe), 2′-O-methoxyethyl (2′-MOE), 2′-O-[2-(methylamino)-2-oxoethyl] (2′-O-NMA) or 2′-deoxy-2′-fluoro-β-d-arabinonucleic acid (2′-FANA). In some embodiments, the modification is 2′-F, 2′-OMe or 2′-MOE. In some embodiments, a modification in a sugar comprises a modification of the sugar ring, which may comprise modification of one or more carbons of the sugar ring. For example, a modification of a sugar of a nucleotide may comprise a 2′-oxygen of a sugar is linked to a 1′-carbon or 4′-carbon of the sugar, or a 2′-oxygen is linked to the 1′-carbon or 4′-carbon via an ethylene or methylene bridge. In some embodiments, a modified nucleotide has an acyclic sugar that lacks a 2′-carbon to 3′-carbon bond. In some embodiments, a modified nucleotide has a thiol group, e.g., in the 4′ position of the sugar.

In some embodiments, the oligonucleotide described herein comprises at least about 1 modified nucleotide (e.g., at least 1, at least 5, at least 10, at least 15, at least 20, at least 25, at least 30, at least 35, at least 40, at least 45, at least 50, at least 55, at least 60, or more). In some embodiments, the sense strand of the oligonucleotide comprises at least about 1 modified nucleotide (e.g., at least 1, at least 5, at least 10, at least 15, at least 20, at least 25, at least 30, at least 35, or more). In some embodiments, the antisense strand of the oligonucleotide comprises at least about 1 modified nucleotide (e.g., at least 1, at least 5, at least 10, at least 15, at least 20, or more).

In some embodiments, all the nucleotides of the sense strand of the oligonucleotide are modified. In some embodiments, all the nucleotides of the antisense strand of the oligonucleotide are modified. In some embodiments, all the nucleotides of the oligonucleotide (i.e., both the sense strand and the antisense strand) are modified. In some embodiments, the modified nucleotide comprises a 2′-modification (e.g., a 2′-F or 2′-OMe, 2′-MOE, and 2′-deoxy-2′-fluoro-β-d-arabinonucleic acid). In some embodiments, the modified nucleotide comprises a 2′-modification (e.g., a 2′-F or 2′-OMe)

The disclosure provides oligonucleotides having different modification patterns. In some embodiments, the modified oligonucleotides comprise a sense strand sequence having a modification pattern as set forth in any one of Tables 3 and 4 (as well as FIG. 10) and an antisense strand having a modification pattern as set forth in any one of Tables 3 and 4 (as well as FIG. 10). In some embodiments, for these oligonucleotides, one or more of positions 8, 9, 10 or 11 of the sense strand is modified with a 2′-F group. In other embodiments, for these oligonucleotides, the sugar moiety at each of nucleotides at positions 1-7 and 12-20 in the sense strand is modified with a 2′-OMe.

In some embodiments, the antisense strand has 3 nucleotides that are modified at the 2′-position of the sugar moiety with a 2′-F. In some embodiments, the sugar moiety at positions 2, 5 and 14 and optionally up to 3 of the nucleotides at positions 1, 3, 7 and 10 of the antisense strand are modified with a 2′-F. In other embodiments, the sugar moiety at each of the positions at positions 2, 5 and 14 of the antisense strand is modified with the 2′-F. In other embodiments, the sugar moiety at each of the positions at positions 1, 2, 5 and 14 of the antisense strand is modified with the 2′-F. In still other embodiments, the sugar moiety at each of the positions at positions 1, 2, 3, 5, 7 and 14 of the antisense strand is modified with the 2′-F. In yet another embodiment, the sugar moiety at each of the positions at positions 1, 2, 3, 5, 10 and 14 of the antisense strand is modified with the 2′-F. In another embodiment, the sugar moiety at each of the positions at positions 2, 3, 5, 7, 10 and 14 of the antisense strand is modified with the 2′-F.

### b. 5′ Terminal Phosphates

In some embodiments, 5′-terminal phosphate groups of an RNAi oligonucleotide enhance the interaction with Ago2. However, oligonucleotides comprising a 5′-phosphate group may be susceptible to degradation via phosphatases or other enzymes, which can limit their bioavailability in vivo. In some embodiments, an oligonucleotide (e.g., a ds oligonucleotide) herein includes analogs of 5′ phosphates that are resistant to such degradation. In some embodiments, the phosphate analog is oxymethylphosphonate, vinylphosphonate or malonyl phosphonate, or a combination thereof. In certain embodiments, the 3′ end of an oligonucleotide strand is attached to chemical moiety that mimics the electrostatic and steric properties of a natural 5′-phosphate group (“phosphate mimic”).

In some embodiments, an oligonucleotide has a phosphate analog at a 4′-carbon position of the sugar (referred to as a “4′-phosphate analog”). See, e.g., Intl. Patent Application Publication No. WO 2018/045317. In some embodiments, an oligonucleotide herein comprises a 4′-phosphate analog at a 5′-terminal nucleotide. In some embodiments, a phosphate analog is an oxymethylphosphonate, in which the oxygen atom of the oxymethyl group is bound to the sugar moiety (e.g., at its 4′-carbon) or analog thereof. In other embodiments, a 4′-phosphate analog is a thiomethylphosphonate or an aminomethylphosphonate, in which the sulfur atom of the thiomethyl group or the nitrogen atom of the amino methyl group is bound to the 4′-carbon of the sugar moiety or analog thereof. In certain embodiments, a 4′-phosphate analog is an oxymethylphosphonate. In some embodiments, an oxymethylphosphonate is represented by the formula —O—CH₂—PO(OH)₂ or —O—CH₂—PO(OR)₂, in which R is independently selected from H, CH₃, an alkyl group, CH₂CH₂CN, CH₂OCOC(CH₃)₃, CH₂OCH₂CH₂Si(CH₃)₃ or a protecting group. In certain embodiments, the alkyl group is CH₂CH₃. More typically, R is independently selected from H, CH₃ or CH₂CH₃.

c. Modified Internucleoside Linkages

In some embodiments, an oligonucleotide comprises a modified internucleoside linkage. In some embodiments, phosphate modifications or substitutions result in an oligonucleotide that comprises at least about 1 (e.g., at least 1, at least 2, at least 3 or at least 5) modified internucleotide linkage. In some embodiments, any one of the oligonucleotides disclosed herein comprises about 1 to about 10 (e.g., 1 to 10, 2 to 8, 4 to 6, 3 to 10, 5 to 10, 1 to 5, 1 to 3 or 1 to 2) modified internucleotide linkages. In some embodiments, any one of the oligonucleotides disclosed herein comprises 1, 2, 3, 4, 5, 6, 7, 8, 9 or 10 modified internucleotide linkages.

A modified internucleotide linkage may be a phosphorodithioate linkage, a phosphorothioate linkage, a phosphotriester linkage, a thionoalkylphosphonate linkage, a thionalkylphosphotriester linkage, a phosphoramidite linkage, a phosphonate linkage or a boranophosphate linkage. In some embodiments, at least one modified internucleotide linkage of any one of the oligonucleotides as disclosed herein is a phosphorothioate linkage.

In some embodiments, the oligonucleotide described herein has a phosphorothioate linkage between one or more of positions 1 and 2 of the sense strand, positions 1 and 2 of the antisense strand, positions 2 and 3 of the antisense strand, positions 3 and 4 of the antisense strand, positions 20 and 21 of the antisense strand, and positions 21 and 22 of the antisense strand. In some embodiments, the oligonucleotide described herein has a phosphorothioate linkage between each of positions 1 and 2 of the sense strand, positions 1 and 2 of the antisense strand, positions 2 and 3 of the antisense strand, positions 20 and 21 of the antisense strand, and positions 21 and 22 of the antisense strand.

d. Base Modifications

In some embodiments, oligonucleotides herein have one or more modified nucleobases. In some embodiments, modified nucleobases (also referred to herein as base analogs) are linked at the 1′ position of a nucleotide sugar moiety. In certain embodiments, a modified nucleobase is a nitrogenous base. In certain embodiments, a modified nucleobase does not contain nitrogen atom. See, e.g., US Patent Application Publication No. 2008/0274462. In some embodiments, a modified nucleotide comprises a universal base. However, in certain embodiments, a modified nucleotide does not contain a nucleobase (abasic).

In some embodiments, a universal base is a heterocyclic moiety located at the 1′ position of a nucleotide sugar moiety in a modified nucleotide, or the equivalent position in a nucleotide sugar moiety substitution, that, when present in a duplex, can be positioned opposite more than one type of base without substantially altering structure of the duplex. In some embodiments, compared to a reference single-stranded nucleic acid (e.g., oligonucleotide) that is fully complementary to a target nucleic acid, a single-stranded nucleic acid containing a universal base forms a duplex with the target nucleic acid that has a lower Tₘ than a duplex formed with the complementary nucleic acid. However, in some embodiments, when compared to a reference single-stranded nucleic acid in which the universal base has been replaced with a base to generate a single mismatch, the single-stranded nucleic acid containing the universal base forms a duplex with the target nucleic acid that has a higher Tₘ than a duplex formed with the nucleic acid comprising the mismatched base.

Non-limiting examples of universal-binding nucleotides include, but are not limited to, inosine, 1-β-D-ribofuranosyl-5-nitroindole and/or 1-β-D-ribofuranosyl-3-nitropyrrole (see, US Patent Application Publication No. 2007/0254362; Van Aerschot et al. (1995) NUCLEIC ACIDS RES. 23:4363-70; Loakes et al. (1995) NUCLEIC ACIDS RES. 23:2361-66; and Loakes & Brown (1994) NUCLEIC ACIDS RES. 22:4039-43).

c. Reversible Modifications

While certain modifications to protect an oligonucleotide from the in vivo environment before reaching target cells can be made, they can reduce the potency or activity of the oligonucleotide once it reaches the cytosol of the target cell. Reversible modifications can be made such that the molecule retains desirable properties outside of the cell, which are then removed upon entering the cytosolic environment of the cell. Reversible modification can be removed, for example, by the action of an intracellular enzyme or by the chemical conditions inside of a cell (e.g., through reduction by intracellular glutathione).

In some embodiments, a reversibly modified nucleotide comprises a glutathione-sensitive moiety. Typically, nucleic acid molecules have been chemically modified with cyclic disulfide moieties to mask the negative charge created by the internucleotide diphosphate linkages and improve cellular uptake and nuclease resistance. See US Patent Application Publication No. 2011/0294869, Intl. Patent Application Publication Nos. WO 2014/088920 and WO 2015/188197, and Meade et al. (2014) NAT. BIOTECHNOL. 32:1256-63. This reversible modification of the internucleotide diphosphate linkages is designed to be cleaved intracellularly by the reducing environment of the cytosol (e.g. glutathione). Earlier examples include neutralizing phosphotriester modifications that were reported to be cleavable inside cells (see, Dellinger et al. (2003) J. AM. CHEM. SOC. 125:940-50).

In some embodiments, such a reversible modification allows protection during in vivo administration (e.g., transit through the blood and/or lysosomal/endosomal compartments of a cell) where the oligonucleotide will be exposed to nucleases and other harsh environmental conditions (e.g., pH). When released into the cytosol of a cell where the levels of glutathione are higher compared to extracellular space, the modification is reversed, and the result is a cleaved oligonucleotide. Using reversible, glutathione-sensitive moieties, it is possible to introduce sterically larger chemical groups into the oligonucleotide of interest when compared to the options available using irreversible chemical modifications. This is because these larger chemical groups will be removed in the cytosol and, therefore, should not interfere with the biological activity of the oligonucleotides inside the cytosol of a cell. As a result, these larger chemical groups can be engineered to confer various advantages to the nucleotide or oligonucleotide, such as nuclease resistance, lipophilicity, charge, thermal stability, specificity and reduced immunogenicity. In some embodiments, the structure of the glutathione-sensitive moiety can be engineered to modify the kinetics of its release.

In some embodiments, a glutathione-sensitive moiety is attached to the sugar of the nucleotide. In some embodiments, a glutathione-sensitive moiety is attached to the 2′-carbon of the sugar of a modified nucleotide. In some embodiments, the glutathione-sensitive moiety is located at the 5′-carbon of a sugar, particularly when the modified nucleotide is the 5′-terminal nucleotide of the oligonucleotide. In some embodiments, the glutathione-sensitive moiety is located at the 3′-carbon of sugar, particularly when the modified nucleotide is the 3′-terminal nucleotide of the oligonucleotide. In some embodiments, the glutathione-sensitive moiety comprises a sulfonyl group. See, e.g., U.S. Provisional Patent Application No. 62/378,635, entitled Compositions Comprising Reversibly Modified Oligonucleotides and Uses Thereof, which was filed on Aug. 23, 2016.

vi. Targeting Ligands

In some embodiments, it is desirable to target the oligonucleotides of the disclosure to one or more cells or one or more organs. Such a strategy can help to avoid undesirable effects in other organs or avoid undue loss of the oligonucleotide to cells, tissue or organs that would not benefit from the oligonucleotide. Accordingly, in some embodiments, oligonucleotides disclosed herein are modified to facilitate targeting and/or delivery to a tissue, cell or organ (e.g., to facilitate delivery of the oligonucleotide to the liver). In certain embodiments, oligonucleotides disclosed herein are modified to facilitate delivery of the oligonucleotide to the hepatocytes of the liver. In some embodiments, an oligonucleotide comprises at least one nucleotide (e.g., 1, 2, 3, 4, 5, 6 or more nucleotides) conjugated to one or more targeting ligand(s).

In some embodiments, the targeting ligand comprises a carbohydrate, amino sugar, cholesterol, peptide, polypeptide, protein or part of a protein (e.g., an antibody or antibody fragment), or lipid. In some embodiments, the targeting ligand is an aptamer. For example, a targeting ligand may be an RGD peptide that is used to target tumor vasculature or glioma cells, CREKA peptide to target tumor vasculature or stoma, transferring, lactoferrin, or an aptamer to target transferrin receptors expressed on CNS vasculature, or an anti-EGFR antibody to target EGFR on glioma cells. In certain embodiments, the targeting ligand is one or more GalNAc moieties.

In some embodiments, 1 or more (e.g., 1, 2, 3, 4, 5 or 6) nucleotides of an oligonucleotide are each conjugated to a separate targeting ligand. In some embodiments, 2 to 4 nucleotides of an oligonucleotide are each conjugated to a separate targeting ligand. In some embodiments, targeting ligands are conjugated to 2 to 4 nucleotides at either ends of the sense or antisense strand (e.g., targeting ligands are conjugated to a 2 to 4 nucleotide overhang or extension on the 5′ or 3′ end of the sense or antisense strand) such that the targeting ligands resemble bristles of a toothbrush and the oligonucleotide resembles a toothbrush. For example, an oligonucleotide may comprise a stem-loop at either the 5′ or 3′ end of the sense strand and 1, 2, 3 or 4 nucleotides of the loop of the stem may be individually conjugated to a targeting ligand. In some embodiments, an oligonucleotide (e.g., a ds oligonucleotide) provided by the disclosure comprises a stem-loop at the 3′ end of the sense strand, wherein the loop of the stem-loop comprises a triloop or a tetraloop, and wherein the 3 or 4 nucleotides comprising the triloop or tetraloop, respectfully, are individually conjugated to a targeting ligand.

GalNAc is a high affinity ligand for the ASGPR, which is primarily expressed on the sinusoidal surface of hepatocyte cells and has a major role in binding, internalizing and subsequent clearing circulating glycoproteins that contain terminal galactose or GalNAc residues (asialoglycoproteins). Conjugation (either indirect or direct) of GalNAc moieties to oligonucleotides of the instant disclosure can be used to target these oligonucleotides to the ASGPR expressed on cells. In some embodiments, an oligonucleotide of the instant disclosure is conjugated to at least one or more GalNAc moieties, wherein the GalNAc moieties target the oligonucleotide to an ASGPR expressed on human liver cells (e.g. human hepatocytes). In some embodiments, the GalNAc moiety target the oligonucleotide to the liver.

In some embodiments, an oligonucleotide of the instant disclosure is conjugated directly or indirectly to a monovalent GalNAc. In some embodiments, the oligonucleotide is conjugated directly or indirectly to more than one monovalent GalNAc (i.e., is conjugated to 2, 3 or 4 monovalent GalNAc moieties, and is typically conjugated to 3 or 4 monovalent GalNAc moieties). In some embodiments, an oligonucleotide is conjugated to one or more bivalent GalNAc, trivalent GalNAc or tetravalent GalNAc moieties.

In some embodiments, 1 or more (e.g., 1, 2, 3, 4, 5 or 6) nucleotides of an oligonucleotide are each conjugated to a GalNAc moiety. In some embodiments, 2 to 4 nucleotides of a tetraloop are each conjugated to a separate GalNAc. In some embodiments, 1 to 3 nucleotides of a triloop are each conjugated to a separate GalNAc. In some embodiments, targeting ligands are conjugated to 2 to 4 nucleotides at either ends of the sense or antisense strand (e.g., ligands are conjugated to a 2 to 4 nucleotide overhang or extension on the 5′ or 3′ end of the sense or antisense strand) such that the GalNAc moieties resemble bristles of a toothbrush and the oligonucleotide resembles a toothbrush. In some embodiments, GalNAc moieties are conjugated to a nucleotide of the sense strand. For example, 4 GalNAc moieties can be conjugated to nucleotides in the tetraloop of the sense strand where each GalNAc moiety is conjugated to 1 nucleotide.

In some embodiments, an oligonucleotide herein comprises a monovalent GalNAc attached to a guanine nucleotide referred to as [ademG-GalNAc] or 2′-aminodiethoxymethanol-Guanine-GalNAc, as depicted below:

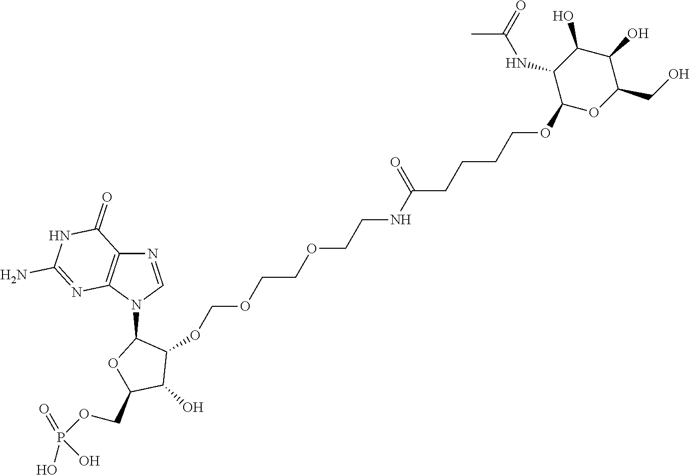 [SMILES: CC(=O)N[C@H]1[C@H](OCCCCC(=O)NCCOCCOCO[C@@H]2[C@H](O)[C@@H](COP(=O)(O)O)O[C@H]2n2cnc3c(=O)[nH]c(N)nc32)O[C@H](CO)[C@H](O)[C@@H]1O]

In some embodiments, an oligonucleotide herein comprises a monovalent GalNAc attached to an adenine nucleotide, referred to as [ademA-GalNAc] or 2′-aminodiethoxymethanol-Adenine-GalNAc, as depicted below:

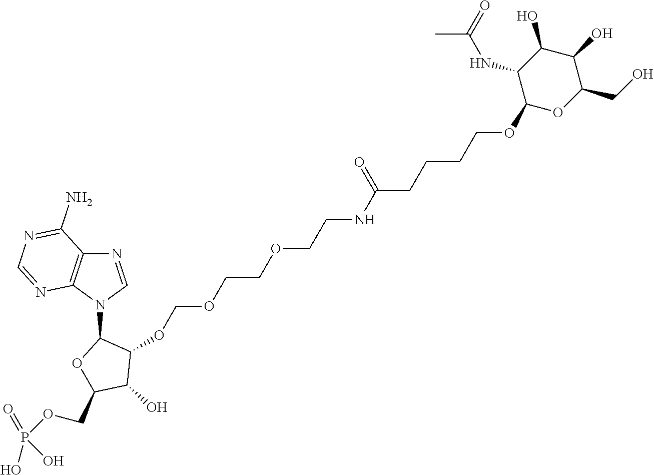

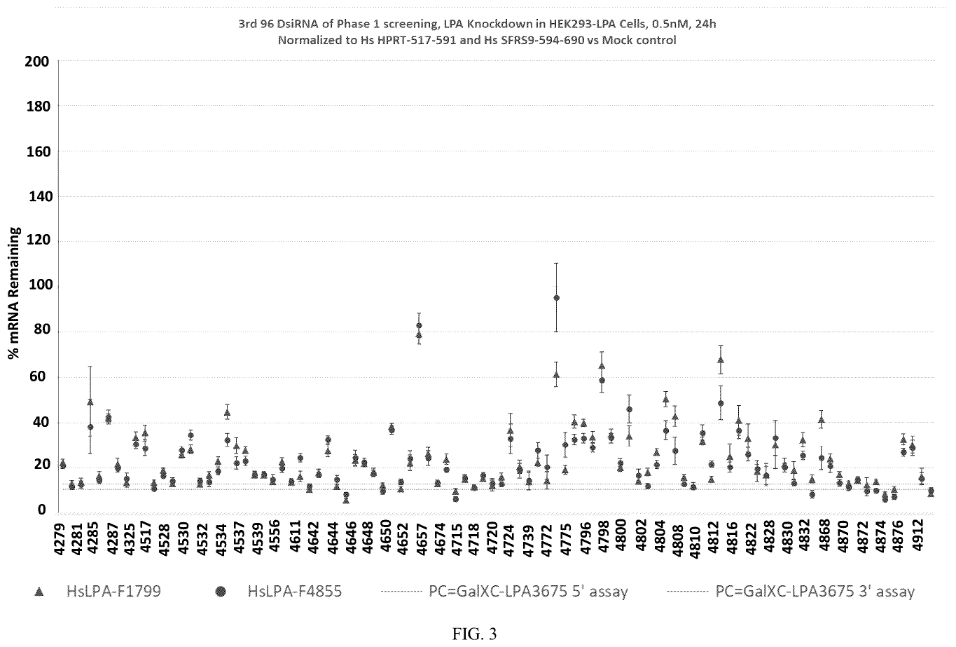

An example of such conjugation is shown below for a loop comprising from 5′ to 3′ the nucleotide sequence GAAA (L=linker, X=heteroatom) stem attachment points are shown. Such a loop may be present, for example, at positions 27-30 of the sense strand listed in Table 5 and as shown in FIG. 3. In the chemical formula,

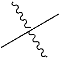

is used to describe an attachment point to the oligonucleotide strand.

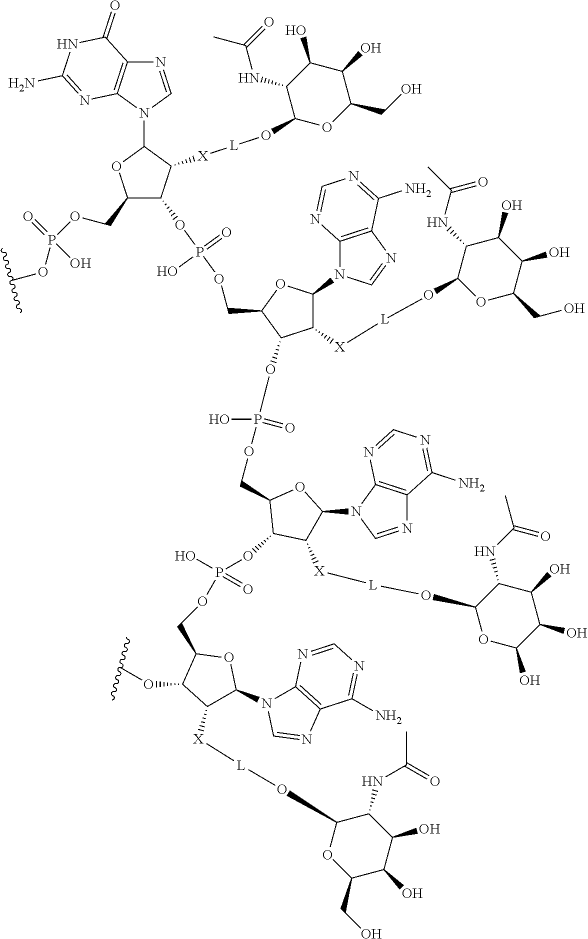

Appropriate methods or chemistry (e.g., click chemistry) can be used to link a targeting ligand to a nucleotide. In some embodiments, a targeting ligand is conjugated to a nucleotide using a click linker. In some embodiments, an acetal-based linker is used to conjugate a targeting ligand to a nucleotide of any one of the oligonucleotides described herein. Acetal-based linkers are disclosed, for example, in Intl. Patent Application Publication No. WO 2016/100401. In some embodiments, the linker is a labile linker. However, in other embodiments, the linker is stable. An example is shown below for a loop comprising from 5′ to 3′ the nucleotides GAAA, in which GalNAc moieties are attached to nucleotides of the loop using an acetal linker. Such a loop may be present, for example, at positions 27-30 of the any one of the sense strand listed in Tables 3 or 4 and as shown in FIG. 10. In the chemical formula,

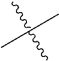

is an attachment point to the oligonucleotide strand.

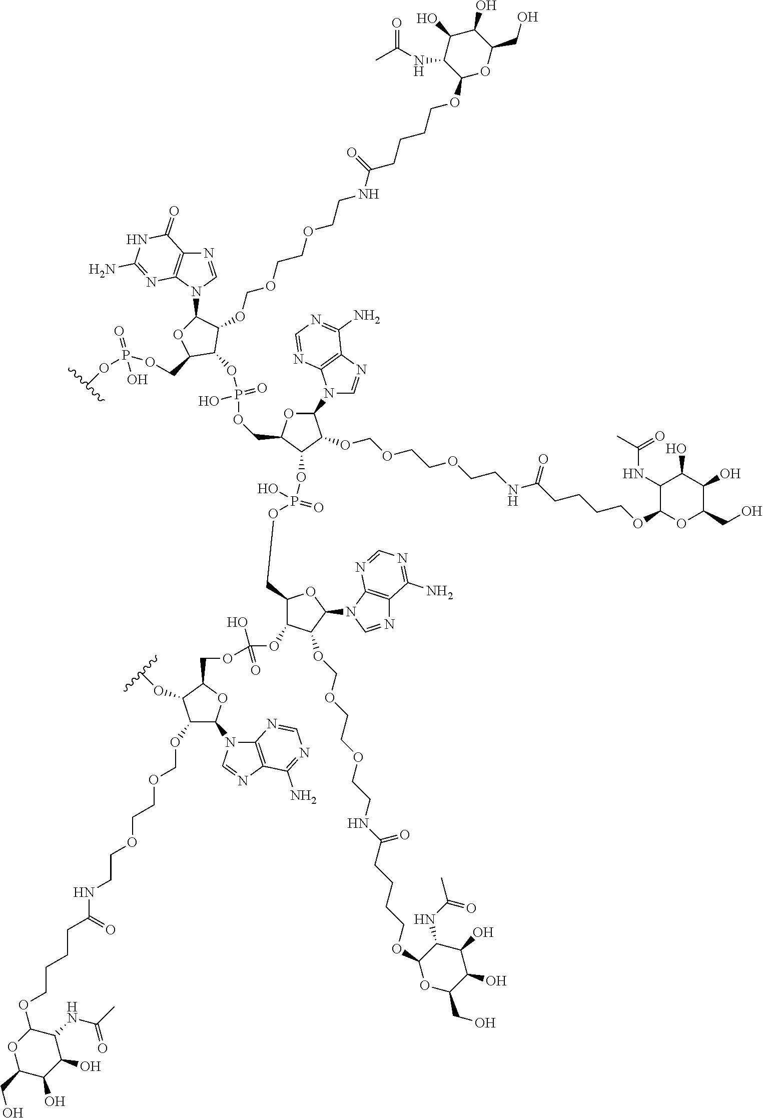

As mentioned, various appropriate methods or chemistry synthetic techniques (e.g., click chemistry) can be used to link a targeting ligand to a nucleotide. In some embodiments, a targeting ligand is conjugated to a nucleotide using a click linker. In some embodiments, an acetal-based linker is used to conjugate a targeting ligand to a nucleotide of any one of the oligonucleotides described herein. Acetal-based linkers are disclosed, for example, in Intl. Patent Application Publication No. WO 2016/100401. In some embodiments, the linker is a labile linker. However, in other embodiments, the linker is a stable linker.

In some embodiments, a duplex extension (e.g., of up to 3, 4, 5 or 6 bp in length) is provided between a targeting ligand (e.g., a GalNAc moiety) and a ds oligonucleotide. In some embodiments, the oligonucleotides herein do not have a GalNAc conjugated thereto.

### III. Formulations

Various formulations have been developed to facilitate oligonucleotide use. For example, oligonucleotides can be delivered to a subject or a cellular environment using a formulation that minimizes degradation, facilitates delivery and/or uptake, or provides another beneficial property to the oligonucleotides in the formulation. In some embodiments, an oligonucleotide is formulated in buffer solutions such as phosphate buffered saline solutions, liposomes, micellar structures and capsids.

Formulations of oligonucleotides with cationic lipids can be used to facilitate transfection of the oligonucleotides into cells. For example, cationic lipids, such as lipofectin, cationic glycerol derivatives, and polycationic molecules (e.g., polylysine, can be used. Suitable lipids include Oligofectamine, Lipofectamine (Life Technologies), NC388 (Ribozyme Pharmaceuticals, Inc., Boulder, Colo.), or FuGene 6 (Roche) all of which can be used according to the manufacturer's instructions.

Accordingly, in some embodiments, a formulation comprises a lipid nanoparticle. In some embodiments, an excipient comprises a liposome, a lipid, a lipid complex, a microsphere, a microparticle, a nanosphere or a nanoparticle, or may be otherwise formulated for administration to the cells, tissues, organs, or body of a subject in need thereof (see, e.g., Remington: THE SCIENCE AND PRACTICE OF PHARMACY, 22nd edition, Pharmaceutical Press, 2013).

In some embodiments, the formulations herein comprise an excipient. In some embodiments, an excipient confers to a composition improved stability, improved absorption, improved solubility and/or therapeutic enhancement of the active ingredient. In some embodiments, an excipient is a buffering agent (e.g., sodium citrate, sodium phosphate, a tris base, or sodium hydroxide) or a vehicle (e.g., a buffered solution, petrolatum, dimethyl sulfoxide or mineral oil). In some embodiments, an oligonucleotide is lyophilized for extending its shelf-life and then made into a solution before use (e.g., administration to a subject). Accordingly, an excipient in a composition comprising any one of the oligonucleotides described herein may be a lyoprotectant (e.g., mannitol, lactose, polyethylene glycol or polyvinylpyrrolidone) or a collapse temperature modifier (e.g., dextran, Ficoll™ or gelatin).

In some embodiments, a pharmaceutical composition is formulated to be compatible with its intended route of administration. Examples of routes of administration include parenteral (e.g., intravenous, intramuscular, intraperitoneal, intradermal, subcutaneous), oral (e.g., inhalation), transdermal (e.g., topical), transmucosal and rectal administration.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). The carrier can be a solvent or dispersion medium containing, for example, water, ethanol, polyol (e.g., glycerol, propylene glycol, and liquid polyethylene glycol, and the like), and suitable mixtures thereof. In many cases, it will be preferable to include isotonic agents, for example, sugars, polyalcohols such as mannitol, sorbitol, sodium chloride in the composition. Sterile injectable solutions can be prepared by incorporating the oligonucleotides in a required amount in a selected solvent with one or a combination of ingredients enumerated above, as required, followed by filtered sterilization.

In some embodiments, a composition may contain at least about 0.1% of the therapeutic agent or more, although the percentage of the active ingredient(s) may be between about 1% to about 80% or more of the weight or volume of the total composition. Factors such as solubility, bioavailability, biological half-life, route of administration, product shelf life, as well as other pharmacological considerations will be contemplated by one skilled in the art of preparing such pharmaceutical formulations, and as such, a variety of dosages and treatment regimens may be desirable.

Even though several embodiments are directed to liver-targeted delivery of any of the oligonucleotides herein, targeting of other tissues is also contemplated.

### IV. Methods of Use

i. Reducing LPA Expression in Cells

The disclosure provides methods for contacting or delivering to a cell or population of cells an effective amount any of the oligonucleotides (e.g., a ds oligonucleotide) herein for purposes of reducing LPA expression. In some embodiments, a reduction of LPA expression is determined by measuring a reduction in the amount or level of LPA mRNA, apo(a) protein, or apo(a) activity in a cell. The methods can include the steps described herein, and these may be be, but not necessarily, carried out in the sequence as described. Other sequences, however, also are conceivable. Moreover, individual or multiple steps bay be carried out either in parallel and/or overlapping in time and/or individually or in multiply repeated steps. Furthermore, the methods may include additional, unspecified steps.

Methods herein are useful in any appropriate cell type. In some embodiments, a cell is any cell that expresses mRNA (e.g., hepatocytes, macrophages, monocyte-derived cells, prostate cancer cells, cells of the brain, endocrine tissue, bone marrow, lymph nodes, lung, gall bladder, liver, duodenum, small intestine, pancreas, kidney, gastrointestinal tract, bladder, adipose and soft tissue and skin). In some embodiments, the cell is a primary cell obtained from a subject. In some embodiments, the primary cell has undergone a limited number of passages such that the cell substantially maintains is natural phenotypic properties. In some embodiments, a cell to which the oligonucleotide is delivered is ex vivo or in vitro (i.e., can be delivered to a cell in culture or to an organism in which the cell resides).

In some embodiments, the oligonucleotides herein are delivered to a cell or population of cells using a nucleic acid delivery method known in the art including, but not limited to, injection of a solution containing the oligonucleotide, bombardment by particles covered by the oligonucleotide, exposing the cell or population of cells to a solution containing the oligonucleotide, or electroporation of cell membranes in the presence of the oligonucleotide. Other methods known in the art for delivering oligonucleotides to cells may be used, such as lipid-mediated carrier transport, chemical-mediated transport, and cationic liposome transfection such as calcium phosphate, and others.

In some embodiments, reduction of LPA expression is determined by an assay or technique that evaluates one or more molecules, properties or characteristics of a cell or population of cells associated with LPA expression (e.g., using an LPA expression biomarker) or by an assay or technique that evaluates molecules that are directly indicative of LPA expression in a cell or population of cells (e.g., LPA mRNA or apo(a) protein). In some embodiments, the extent to which an oligonucleotide herein reduces LPA expression is evaluated by comparing LPA expression in a cell or population of cells contacted with the oligonucleotide to a control cell or population of cells (e.g., a cell or population of cells not contacted with the oligonucleotide or contacted with a control oligonucleotide). In some embodiments, a control amount or level of LPA expression in a control cell or population of cells is predetermined, such that the control amount or level need not be measured in every instance the assay or technique is performed. The predetermined level or value can take a variety of forms. In some embodiments, a predetermined level or value can be single cut-off value, such as a median or mean.

In some embodiments, contacting or delivering an oligonucleotide (e.g., a ds oligonucleotide) herein to a cell or a population of cells results in a reduction in LPA expression. In some embodiments, the reduction in LPA expression is relative to a control amount or level of LPA expression in cell or population of cells not contacted with the oligonucleotide or contacted with a control oligonucleotide. In some embodiments, the reduction in LPA expression is about 1% or lower, about 5% or lower, about 10% or lower, about 15% or lower, about 20% or lower, about 25% or lower, about 30% or lower, about 35% or lower, about 40% or lower, about 45% or lower, about 50% or lower, about 55% or lower, about 60% or lower, about 70% or lower, about 80% or lower, or about 90% or lower relative to a control amount or level of LPA expression. In some embodiments, the control amount or level of LPA expression is an amount or level of LPA mRNA and/or apo(a) protein in a cell or population of cells that has not been contacted with an oligonucleotide herein. In some embodiments, the effect of delivery of an oligonucleotide to a cell or population of cells according to a method herein is assessed after any finite period or amount of time (e.g., minutes, hours, days, weeks, months). For example, in some embodiments, LPA expression is determined in a cell or population of cells at least about 4 hours, about 8 hours, about 12 hours, about 18 hours, about 24 hours; or at least about 1 day, about 2 days, about 3 days, about 4 days, about 5 days, about 6 days, about 7 days, about 8 days, about 9 days, about 10 days, about 11 days, about 12 days, about 13 days, about 14 days, about 21 days, about 28 days, about 35 days, about 42 days, about 49 days, about 56 days, about 63 days, about 70 days, about 77 days, or about 84 days or more after contacting or delivering the oligonucleotide to the cell or population of cells. In some embodiments, LPA expression is determined in a cell or population of cells at least about 1 month, about 2 months, about 3 months, about 4 months, about 5 months, or about 6 months or more after contacting or delivering the oligonucleotide to the cell or population of cells.

In some embodiments, an oligonucleotide is delivered in the form of a transgene that is engineered to express in a cell the oligonucleotide or strands comprising the oligonucleotide (e.g., its sense and antisense strands). In some embodiments, an oligonucleotide is delivered using a transgene engineered to express any oligonucleotide disclosed herein. Transgenes may be delivered using viral vectors (e.g., adenovirus, retrovirus, vaccinia virus, poxvirus, adeno-associated virus or herpes simplex virus) or non-viral vectors (e.g., plasmids or synthetic mRNAs). In some embodiments, transgenes can be injected directly to a subject.

ii. Medical Use

The disclosure also provides oligonucleotides for use, or adaptable for use, to treat a subject (e.g., a human having a disease, disorder or condition associated with LPA expression) that would benefit from reducing LPA expression. In some embodiments, the disclosure provides oligonucleotides for use, or adapted for use, to treat a subject having a disease, disorder or condition associated with expression of LPA. The disclosure also provides oligonucleotides for use, or adaptable for use, in the manufacture of a medicament or pharmaceutical composition for treating a disease, disorder or condition associated with LPA expression. In some embodiments, the oligonucleotides for use, or adaptable for use, target LPA mRNA and reduce LPA expression (e.g., via the RNAi pathway). In some embodiments, the oligonucleotides for use, or adaptable for use, target LPA mRNA and reduce the amount or level of LPA mRNA, apo(a) protein and/or apo(a) activity.

In addition, in some embodiments of the methods herein, a subject having a disease, disorder or condition associated with LPA expression or is predisposed to the same is selected for treatment with an oligonucleotide (e.g., a ds oligonucleotide) herein. In some embodiments, the method comprises selecting an individual having a marker (e.g., a biomarker) for a disease, disorder or condition associated with LPA expression, or predisposed to the same, such as, but not limited to, LPA mRNA, apo(a) protein, lipoprotein (a), or a combination thereof. Likewise, and as detailed below, some embodiments of the methods provided by the disclosure include steps such as measuring or obtaining a baseline value for a marker of LPA expression (e.g., lipoprotein (a)), and then comparing such obtained value to one or more other baseline values or values obtained after the subject is administered the oligonucleotide to assess the effectiveness of treatment.

iii. Methods of Treatment

The disclosure also provides methods of treating a subject having, suspected of having, or at risk of developing a disease, disorder or condition associated with LPA expression with an oligonucleotide herein. In some embodiments, the disclosure provides methods of treating or attenuating the onset or progression of a disease, disorder or condition associated with LPA expression using the oligonucleotides herein. In other embodiments, the disclosure provides methods to achieve one or more therapeutic benefits in a subject having a disease, disorder or condition associated with LPA expression using the oligonucleotides herein. In some embodiments of the methods herein, the subject is treated by administering a therapeutically effective amount of any one or more of the oligonucleotides herein. In some embodiments, treatment comprises reducing LPA expression. In some embodiments, the subject is treated therapeutically. In some embodiments, the subject is treated prophylactically.

In some embodiments of the methods herein, an oligonucleotide herein, or a pharmaceutical composition comprising the oligonucleotide, is administered to a subject having a disease, disorder or condition associated with LPA expression such that LPA expression is reduced in the subject, thereby treating the subject. In some embodiments, an amount or level of LPA mRNA is reduced in the subject. In some embodiments, an amount or level of apo(a) protein is reduced in the subject. In some embodiments, an amount or level of lipoprotein (a) is reduced in the subject. In some embodiments, an amount or level of apo(a) activity is reduced in the subject. In some embodiments, an amount or level of triglyceride (TG) (e.g., one or more TG(s) or total TGs) is reduced in the subject. In some embodiments, an amount or level of cholesterol (e.g., total cholesterol, LDL cholesterol, and/or HDL cholesterol) is reduced in the subject. In some embodiments, an amount or level of low-density lipoprotein (LDL) cholesterol is reduced in the subject. In some embodiments, an amount or activity of OxPL is reduced or altered in the subject. In some embodiments, an amount or activity of LDL-C is reduced or altered in the subject. In some embodiments, an amount or activity of apoB-100 is reduced or altered in the subject. In some embodiments, any combination of the following is reduced or altered in the subject: LPA expression, an amount or level of LPA mRNA, an amount or level of apo(a) protein, an amount or level of apo(a) activity, an amount or level of TG, an amount or level of cholesterol, an amount or activity of OxPL, an amount or activity of LDL-C, and/or an amount or activity of apoB-100.

In some embodiments of the methods herein, an oligonucleotide herein, or a pharmaceutical composition comprising the oligonucleotide, is administered to a subject having a disease, disorder or condition associated with LPA expression such that LPA expression is reduced in the subject by at least about 30%, about 35%, about 40%, about 45%, about 50%, about 55%, about 60%, about 65%, about 70%, about 75%, about 80%, about 85%, about 90%, about 95%, about 99% or greater than 99% when compared to LPA expression prior to administration of the oligonucleotide or pharmaceutical composition. In some embodiments, LPA expression is reduced in the subject by at least about 30%, about 35%, about 40%, about 45%, about 50%, about 55%, about 60%, about 65%, about 70%, about 75%, about 80%, about 85%, about 90%, about 95%, about 99% or greater than 99% when compared to LPA expression in a subject (e.g., a reference or control subject) not receiving the oligonucleotide or pharmaceutical composition or receiving a control oligonucleotide, pharmaceutical composition or treatment.

In some embodiments of the methods herein, an oligonucleotide herein, or a pharmaceutical composition comprising the oligonucleotide, is administered to a subject having a disease, disorder or condition associated with LPA expression such that an amount or level of LPA mRNA is reduced in the subject by at least about 30%, about 35%, about 40%, about 45%, about 50%, about 55%, about 60%, about 65%, about 70%, about 75%, about 80%, about 85%, about 90%, about 95%, about 99% or greater than 99% when compared to the amount or level of LPA mRNA prior to administration of the oligonucleotide or pharmaceutical composition. In some embodiments, an amount or level of LPA mRNA is reduced in the subject by at least about 30%, about 35%, about 40%, about 45%, about 50%, about 55%, about 60%, about 65%, about 70%, about 75%, about 80%, about 85%, about 90%, about 95%, about 99% or greater than 99% when compared to an amount or level of LPA mRNA in a subject (e.g., a reference or control subject) not receiving the oligonucleotide or pharmaceutical composition or receiving a control oligonucleotide, pharmaceutical composition or treatment.

In some embodiments of the methods herein, an oligonucleotide herein, or a pharmaceutical composition comprising the oligonucleotide, is administered to a subject having a disease, disorder or condition associated with LPA expression such that an amount or level of apo(a) protein is reduced in the subject by at least about 30%, about 35%, about 40%, about 45%, about 50%, about 55%, about 60%, about 65%, about 70%, about 75%, about 80%, about 85%, about 90%, about 95%, about 99% or greater than 99% when compared to the amount or level of apo(a) protein prior to administration of the oligonucleotide or pharmaceutical composition. In some embodiments, an amount or level of apo(a) protein is reduced in the subject by at least about 30%, about 35%, about 40%, about 45%, about 50%, about 55%, about 60%, about 65%, about 70%, about 75%, about 80%, about 85%, about 90%, about 95%, about 99% or greater than 99% when compared to an amount or level of apo(a) protein in a subject (e.g., a reference or control subject) not receiving the oligonucleotide or pharmaceutical composition or receiving a control oligonucleotide, pharmaceutical composition or treatment.

In some embodiments of the methods herein, an oligonucleotide herein, or a pharmaceutical composition comprising the oligonucleotide, is administered to a subject having a disease, disorder or condition associated with LPA expression such that an amount or level of apo(a) activity is reduced in the subject by at least about 30%, about 35%, about 40%, about 45%, about 50%, about 55%, about 60%, about 65%, about 70%, about 75%, about 80%, about 85%, about 90%, about 95%, about 99% or greater than 99% when compared to the amount or level of apo(a) activity prior to administration of the oligonucleotide or pharmaceutical composition. In some embodiments, an amount or level of apo(a) activity is reduced in the subject by at least about 30%, about 35%, about 40%, about 45%, about 50%, about 55%, about 60%, about 65%, about 70%, about 75%, about 80%, about 85%, about 90%, about 95%, about 99% or greater than 99% when compared to an amount or level of apo(a) activity in a subject (e.g., a reference or control subject) not receiving the oligonucleotide or pharmaceutical composition or receiving a control oligonucleotide, pharmaceutical composition or treatment.

In some embodiments of the methods herein, an oligonucleotide herein, or a pharmaceutical composition comprising the oligonucleotide, is administered to a subject having a disease, disorder or condition associated with LPA expression such that an amount or level of lipoprotein (a) is reduced in the subject by at least about 30%, about 35%, about 40%, about 45%, about 50%, about 55%, about 60%, about 65%, about 70%, about 75%, about 80%, about 85%, about 90%, about 95%, about 99% or greater than 99% when compared to the amount or level of lipoprotein (a) prior to administration of the oligonucleotide or pharmaceutical composition. In some embodiments, an amount or level of lipoprotein (a) is reduced in the subject by at least about 30%, about 35%, about 40%, about 45%, about 50%, about 55%, about 60%, about 65%, about 70%, about 75%, about 80%, about 85%, about 90%, about 95%, about 99% or greater than 99% when compared to an amount or level of lipoprotein (a) in a subject (e.g., a reference or control subject) not receiving the oligonucleotide or pharmaceutical composition or receiving a control oligonucleotide, pharmaceutical composition or treatment.

Lipoprotein (a) levels range widely in human adults with plasma levels ranging from <0.1 mg/dL to >200 mg/dL, thus exhibiting up to three orders of magnitude difference among individuals (Schmidt et al., (2016) J Lipid Res. 57 (8): 1339-1359). Lipoprotein (a) levels<30 mg/dl are considered optimal in the United States and Canada (Anderson et al., (2016) CAN J CARDIOL 32:1263-82). The European Atherosclerosis Society (EAS) has proposed <50 mg/dL as optimal, and lipoprotein (a) levels>60 mg/dl are used as a cutoff for the reimbursement of apheresis in Germany and the United Kingdom (Tsimikas (2017) J AM COLL CARDIOL. 69 (6): 692-711). In some embodiments, a subject selected for treatment or treated with an oligonucleotide herein is identified or determined to have an amount or level of lipoprotein (a) of about 30 mg/dL or greater. In some embodiments, a subject selected for treatment or treated with an oligonucleotide herein is identified or determined to have an amount or level of lipoprotein (a) of >30 mg/dL. In some embodiments, a subject selected for treatment or treated with an oligonucleotide herein is identified or determined to have an amount or level of lipoprotein (a) of about 50 mg/dL or greater. In some embodiments, a subject selected for treatment or treated with an oligonucleotide herein is identified or determined to have an amount or level of lipoprotein (a) of about 60 mg/dL or greater. In some embodiments, a subject selected for treatment or treated with an oligonucleotide herein is identified or determined to have an amount or level of lipoprotein (a) in the range of 30 mg/dL to 300 mg/dL.

Generally, a normal or desirable TG range for a human patient is ≤150 mg/dl of blood, with <100 being considered ideal. In some embodiments, the patient selected for treatment or treated is identified or determined to have an amount or level of TG of ≥150 mg/dL. In some embodiments, the patient selected for treatment or treated is identified or determined to have an amount or level of TG in the range of 150 to 199 mg/dL, which is considered borderline high TG levels. In some embodiments, the patient selected for treatment or treated is identified or determined to have an amount or level of TG in the range of 200 to 499 mg/dl, which is considered high TG levels. In some embodiments, the patient selected for treatment or treated is identified or determined to have an amount or level of TG in the range of 500 mg/dL or higher (i.e., ≥500 mg/dL), which is considered very high TG levels. In some embodiments, the patient selected for treatment or treated is identified or determined to have an amount or level of TG which is ≥150 mg/dL, ≥200 mg/dL or ≥500 mg/dL. In some embodiments, the patient selected for treatment or treated is identified or determined to have an amount of level of TG of 200 to 499 mg/dL, or 500 mg/dL or higher. In some embodiments, the patient selected for treatment or treated is identified or determined to have an amount or level of TG which is ≥200 mg/dL.

In some embodiments of the methods herein, an oligonucleotide herein, or a pharmaceutical composition comprising the oligonucleotide, is administered to a subject having a disease, disorder or condition associated with LPA expression such that an amount or level of cholesterol (e.g., total cholesterol, LDL cholesterol, and/or HDL cholesterol) is reduced in the subject by at least about 30%, about 35%, about 40%, about 45%, about 50%, about 55%, about 60%, about 65%, about 70%, about 75%, about 80%, about 85%, about 90%, about 95%, about 99% or greater than 99% when compared to the amount or level of cholesterol prior to administration of the oligonucleotide or pharmaceutical composition. In some embodiments, an amount or level of cholesterol is reduced in the subject by at least about 30%, about 35%, about 40%, about 45%, about 50%, about 55%, about 60%, about 65%, about 70%, about 75%, about 80%, about 85%, about 90%, about 95%, about 99% or greater than 99% when compared to an amount or level of cholesterol in a subject (e.g., a reference or control subject) not receiving the oligonucleotide or pharmaceutical composition or receiving a control oligonucleotide, pharmaceutical composition or treatment.

Generally, a normal or desirable cholesterol range (total cholesterol) for an adult human patient is <200 mg/dL of blood. In some embodiments, the patient selected for treatment or treated is identified or determined to have an amount or level of cholesterol of ≥200 mg/dL. In some embodiments, the patient selected for treatment or treated is identified or determined to have an amount or level of cholesterol in the range of 200 to 239 mg/dL, which is considered borderline high cholesterol levels. In some embodiments, the patient selected for treatment or treated is identified or determined to have an amount or level of cholesterol in the range of 240 mg/dL and higher (i.e., ≥240 mg/dL), which is considered high cholesterol levels. In some embodiments, the patient selected from treatment or treated is identified or determined to have an amount or level of cholesterol of 200 to 239 mg/dL, or 240 mg/dL or higher. In some embodiments, the patient selected for treatment or treated is identified or determined to have an amount or level of cholesterol which is ≥200 mg/dL or ≥240 mg/dL or higher.

In some embodiments of the methods herein, an oligonucleotide herein, or a pharmaceutical composition comprising the oligonucleotide, is administered to a subject having a disease, disorder, or condition associated with LPA expression such that an amount or level of LDL cholesterol is reduced in the subject by at least about 30%, about 35%, about 40%, about 45%, about 50%, about 55%, about 60%, about 65%, about 70%, about 75%, about 80%, about 85%, about 90%, about 95%, about 99% or greater than 99% when compared to the amount or level of LDL cholesterol prior to administration of the oligonucleotide or pharmaceutical composition. In some embodiments, an amount or level of LDL cholesterol is reduced in the subject by at least about 30%, about 35%, about 40%, about 45%, about 50%, about 55%, about 60%, about 65%, about 70%, about 75%, about 80%, about 85%, about 90%, about 95%, about 99% or greater than 99% when compared to an amount or level of LDL cholesterol in a subject (e.g., a reference or control subject) not receiving the oligonucleotide or pharmaceutical composition or receiving a control oligonucleotide, pharmaceutical composition or treatment.

Generally, a normal or desirable LDL cholesterol range for an adult human patient is <100 mg/dL of blood. In some embodiments, the patient selected for treatment or treated is identified or determined to have an amount or level of cholesterol of ≥100 mg/dL. In some embodiments, the patient selected for treatment or treated is identified or determined to have an amount or level of LDL cholesterol in the range of 100 to 129 mg/dL, which is considered above optimal. In some embodiments, the patient selected for treatment or treated is identified or determined to have an amount or level of LDL cholesterol in the range of 130 to 159 mg/dL, which is considered borderline high levels. In some embodiments, the patient selected for treatment or treated is identified or determined to have an amount or level of LDL cholesterol in the range of 160 to 189 mg/dL, which is considered high LDL cholesterol levels. In some embodiments, the patient selected for treatment or treated is identified or determined to have an amount or level of LDL cholesterol in the range of 190 mg/dL and higher (i.e., ≥190 mg/dL), which is considered very high LDL cholesterol levels. In some embodiments, the patient selected for treatment or treated is identified or determined to have an amount or level of LDL cholesterol which is ≥100 mg/dL, ≥130 mg/dL, ≥160 mg/dL, or ≥190 mg/dL or higher, preferably ≥160 mg/dL, or ≥190 mg/dL or higher. In some embodiments, the patient selected for treatment or treated is identified or determined to have an amount or level of LDL cholesterol of 100 to 129 mg/dL, 130 to 159 mg/dL, 160 to 189 mg/dL, or 190 mg/dL and higher.

Suitable methods for determining LPA expression, an amount or level of LPA mRNA, an amount or level of apo(a) protein, an amount or level of apo(a) activity, an amount or level of lipoprotein (a), and/or an amount or level of OxPL, LDL-C, apoB-100, TG and/or LDL cholesterol in the subject, or in a sample from the subject, are known in the art. Further, the Examples set forth herein illustrate exemplary methods for determining LPA expression.

In some embodiments, LPA expression, the amount or level of LPA mRNA, apo(a) protein, apo(a) activity, OxPL, LDL-C, apoB-100, TG, LDL cholesterol, or any combination thereof, is reduced in a cell (e.g., a hepatocyte), a population or a group of cells (e.g., an organoid), an organ (e.g., liver), blood or a fraction thereof (e.g., plasma), a tissue (e.g., liver tissue), a sample (e.g., a liver biopsy sample), or any other biological material obtained or isolated from the subject. In some embodiments, LPA expression, the amount or level of LPA mRNA, apo(a) protein, apo(a) activity, OxPL, LDL-C, apoB-100, TG, LDL cholesterol, or any combination thereof, is reduced in more than one type of cell (e.g., a hepatocyte and one or more other type(s) of cell), more than one groups of cells, more than one organ (e.g., liver and one or more other organ(s)), more than one fraction of blood (e.g., plasma and one or more other blood fraction(s)), more than one type of tissue (e.g., liver tissue and one or more other type(s) of tissue), more than one type of sample (e.g., a liver biopsy sample and one or more other type(s) of biopsy sample) obtained or isolated from the subject.

Examples of a disease, disorder or condition associated with LPA expression include, but are not limited to, Berger's disease, peripheral artery disease, coronary artery disease, metabolic syndrome, acute coronary syndrome, aortic valve stenosis, aortic valve regurgitation, aortic dissection, retinal artery occlusion, cerebrovascular disease, mesenteric ischemia, superior mesenteric artery occlusion, renal artery stenosis, stable/unstable angina, acute coronary syndrome, heterozygous or homozygous familial hypercholesterolemia, hyperapobetalipoproteinemia, cerebrovascular atherosclerosis, cerebrovascular disease, and venous thrombosis, or a combination thereof.

Because of their high specificity, the oligonucleotides herein specifically target mRNAs of target genes of cells, tissues, or organs (e.g., liver). In preventing disease, the target gene may be one which is required for initiation or maintenance of the disease or which has been identified as being associated with a higher risk of contracting the disease. In treating disease, the oligonucleotide can be brought into contact with the cells or tissue exhibiting or responsible for mediating the disease. For example, an oligonucleotide substantially identical to all or part of a wild-type (i.e., native) or mutated gene associated with a disorder or condition associated with LPA expression may be brought into contact with or introduced into a cell or tissue type of interest such as a hepatocyte or other liver cell.

In some embodiments, the target gene may be a target gene from any mammal, such as a human. Any gene may be silenced according to the method described herein.

Methods described herein are typically involve administering to a subject a therapeutically effective amount of an oligonucleotide herein, that is, an amount capable of producing a desirable therapeutic result. A therapeutically acceptable amount may be an amount that can therapeutically treat a disease or disorder. The appropriate dosage for any one subject will depend on certain factors, including the subject's size, body surface area, age, the particular composition to be administered, the active ingredient(s) in the composition, time and route of administration, general health, and other drugs being administered concurrently.

In some embodiments, a subject is administered any one of the compositions herein either enterally (e.g., orally, by gastric feeding tube, by duodenal feeding tube, via gastrostomy or rectally), parenterally (e.g., subcutaneous injection, intravenous injection or infusion, intra-arterial injection or infusion, intraosseous infusion, intramuscular injection, intracerebral injection, intracerebroventricular injection, intrathecal), topically (e.g., epicutancous, inhalational, via eye drops, or through a mucous membrane), or by direct injection into a target organ (e.g., the liver of a subject). Typically, oligonucleotides herein are administered intravenously or subcutaneously.

As a non-limiting set of examples, the oligonucleotides herein would typically be administered quarterly (once every three months), bi-monthly (once every two months), monthly or weekly. For example, the oligonucleotides may be administered every week or at intervals of two, or three weeks. Alternatively, the oligonucleotides may be administered daily. In some embodiments, a subject is administered one or more loading doses of the oligonucleotide followed by one or more maintenance doses of the oligonucleotide.

In some embodiments, the subject to be treated is a human or non-human primate or other mammalian subject. Other exemplary subjects include domesticated animals such as dogs and cats; livestock such as horses, cattle, pigs, sheep, goats, and chickens; and animals such as mice, rats, guinea pigs, and hamsters.

### V. Kits

In some embodiments, the disclosure provides a kit comprising an oligonucleotide herein, and instructions for use. In some embodiments, the kit comprises an oligonucleotide herein, and a package insert containing instructions for use of the kit and/or any component thereof. In some embodiments, the kit comprises, in a suitable container, an oligonucleotide herein, one or more controls, and various buffers, reagents, enzymes and other standard ingredients well known in the art. In some embodiments, the container comprises at least one vial, well, test tube, flask, bottle, syringe or other container means, into which the oligonucleotide is placed, and in some instances, suitably aliquoted. In some embodiments where an additional component is provided, the kit contains additional containers into which this component is placed. The kits can also include a means for containing the oligonucleotide and any other reagent in close confinement for commercial sale. Such containers may include injection or blow-molded plastic containers into which the desired vials are retained. Containers and/or kits can include labeling with instructions for use and/or warnings.

In some embodiments, a kit comprises an oligonucleotide herein, and a pharmaceutically acceptable carrier, or a pharmaceutical composition comprising the oligonucleotide and instructions for treating or delaying progression of a disease, disorder or condition associated with LPA expression in a subject in need thereof.

### EXAMPLES

While the disclosure has been described with reference to the specific embodiments set forth in the following Examples, it should be understood by those skilled in the art that various changes may be made, and equivalents may be substituted without departing from the true spirit and scope of the disclosure. Further, the following Examples are offered by way of illustration and are not intended to limit the scope of the disclosure in any manner. In addition, modifications may be made to adapt to a situation, material, composition of matter, process, process step or steps, to the objective, spirit and scope of the disclosure. All such modifications are intended to be within the scope of the disclosure. Standard techniques well known in the art or the techniques specifically described below are utilized.

### Example 1: Preparation of Double-Stranded RNAi Oligonucleotides

### Oligonucleotide Synthesis and Purification

The ds RNAi oligonucleotides described in the foregoing Examples are chemically synthesized using methods described herein. Generally, ds RNAi oligonucleotides are synthesized using solid phase oligonucleotide synthesis methods as described for 19-23mer siRNAs (see, e.g., Scaringe et al. (1990) NUCLEIC ACIDS RES. 18:5433-41 and Usman et al. (1987) J. AM. CHEM. SOC. 109:7845-45; see also, U.S. Pat. Nos. 5,804,683; 5,831,071; 5,998,203; 6,008,400; 6,111,086; 6,117,657; 6,353,098; 6,362,323; 6,437,117 and 6,469,158).

Individual RNA strands are synthesized and HPLC purified according to standard methods (Integrated DNA Technologies; Coralville, IA). For example, RNA oligonucleotides are synthesized using solid phase phosphoramidite chemistry, deprotected and desalted on NAP-5 columns (Amersham Pharmacia Biotech; Piscataway, NJ) using standard techniques (Damha & Olgivie (1993) METHODS MOL. BIOL. 20:81-114; Wincott et al. (1995) NUCLEIC ACIDS RES. 23:2677-84). The oligomers are purified using ion-exchange high performance liquid chromatography (IE-HPLC) on an Amersham Source 15Q column (1.0 cm×25 cm; Amersham Pharmacia Biotech) using a 15 min step-linear gradient. The gradient varies from 90:10 Buffers A:B to 52:48 Buffers A:B, where Buffer A is 100 mM Tris pH 8.5 and Buffer B is 100 mM Tris pH 8.5, 1 M NaCl. Samples are monitored at 260 nm and peaks corresponding to the full-length oligonucleotide species are collected, pooled, desalted on NAP-5 columns, and lyophilized.

The purity of each oligomer is determined by capillary electrophoresis (CE) on a Beckman PACE 5000 (Beckman Coulter, Inc.; Fullerton, CA). The CE capillaries have a 100 μm inner diameter and contain ssDNA 100R Gel (Beckman-Coulter). Typically, about 0.6 nmole of oligonucleotide is injected into a capillary, is run in an electric field of 444 V/cm and is detected by UV absorbance at 260 nm. Denaturing Tris-Borate-7 M-urea running buffer is purchased from Beckman-Coulter. Oligoribonucleotides are obtained that are at least 90% pure as assessed by CE for use in experiments described below. Compound identity is verified by matrix-assisted laser desorption ionization time-of-flight (MALDI-TOF) mass spectroscopy on a Voyager DE™ Biospectometry Work Station (Applied Biosystems; Foster City, CA) following the manufacturer's recommended protocol. Relative molecular masses of all oligomers are obtained, often within 0.2% of expected molecular mass.

### Preparation of Duplexes

ssRNA oligomers are resuspended (e.g., at 100 μM concentration) in duplex buffer consisting of 100 mM potassium acetate, 30 mM HEPES, pH 7.5. Complementary sense and antisense strands are mixed in equal molar amounts to yield a final solution of, for example, 50 μM duplex. Samples are heated to 100° ° C. for 5′ in RNA buffer (IDT) and are allowed to cool to room temperature before use. The ds RNA oligonucleotides are stored at −20° C. ss RNA oligomers are stored lyophilized or in nuclease-free water at −80° C.

### Example 2: RNAi Oligonucleotide Inhibition of LPA Expression In Vitro

LPA mRNA Target Sequence Identification

To identify RNAi oligonucleotide inhibitors of LPA expression, a computer-based algorithm was used to computationally identify LPA mRNA target sequences suitable for assaying inhibition of LPA expression by the RNAi pathway. The algorithm provides RNAi oligonucleotide guide (antisense) strand sequences each having a region of complementarity to a suitable LPA target sequence of human LPA mRNA (e.g., SEQ ID NO: 1; Table 1). Some of the guide strand sequences identified by the algorithm are also complementary to the corresponding LPA target sequence of monkey LPA mRNA (SEQ ID NO: 2; Table 1). RNAi oligonucleotides (formatted as DsiRNA oligonucleotides) were generated (Table 2), each with a unique guide strand having a region of complementarity to an LPA target sequence identified by the algorithm. The passenger (sense) strands of the DsiRNAs provided in Table 2 comprise a unique human LPA mRNA target sequence identified by the algorithm.

TABLE 1
Sequences of Human and NHP (Monkey) mRNA

| Species | GenBank Ref Seq # | SEQ ID NO: |
|---|---|---|
| Human (Hs) | NM_005577.3 | 1 |
| Cynomolgus | XM_015448517.1 | 2 |
| monkey (Mf) | | |
| Rhesus monkey | XM_028847001.1 | 3 |

TABLE 2
DsiRNAs Targeting Human LPA mRNA and
Controls Evaluated in Cells

| | | SEQ | | SEQ | | SEQ |
|---|---|---|---|---|---|---|
| | | ID | Guide | ID | Target | ID |
| | Passenger (Sense) | NO: | (Antisense) | NO: | Sequence | NO: |
| Dsi | | | | | | |
| RNA | | | | | | |
| LPA- | CUGAGCAAAGCCAUGUGGU | 4 | UCCUGUACCACAUG | 404 | CUGAGCAAAG | 804 |
| 125 | ACAGGA | | GCUUUGCUCAGGU | | CCAUGUGGU | |
| LPA- | AGCAAAGCCAUGUGGUCCA | 5 | CAAUCUUGGACCAC | 405 | AGCAAAGCCA | 805 |
| 128 | AGAUTG | | AUGGCUUUGCUCA | | UGUGGUCCA | |
| LPA- | AAGCCAUGUGGUCCAGGAU | 6 | GUAGCUAUCCUGGA | 406 | AAGCCAUGUG | 806 |
| 132 | AGCUAC | | CCACAUGGCUUUG | | GUCCAGGAU | |
| LPA- | AGCCAUGUGGUCCAGGAUU | 7 | GGUAGUAAUCCUGG | 407 | AGCCAUGUGG | 807 |
| 133 | ACUACC | | ACCACAUGGCUUU | | UCCAGGAUU | |
| LPA- | GCCAUGUGGUCCAGGAUUG | 8 | UGGUAUCAAUCCUG | 408 | GCCAUGUGGU | 808 |
| 134 | AUACCA | | GACCACAUGGCUU | | CCAGGAUUG | |
| LPA- | CCAUGUGGUCCAGGAUUGC | 9 | AUGGUUGCAAUCCU | 409 | CCAUGUGGUC | 809 |
| 135 | AACCAT | | GGACCACAUGGCU | | CAGGAUUGC | |
| LPA- | CAUGUGGUCCAGGAUUGCU | 10 | CAUGGUAGCAAUCC | 410 | CAUGUGGUCC | 810 |
| 136 | ACCATG | | UGGACCACAUGGC | | AGGAUUGCU | |
| LPA- | AUGUGGUCCAGGAUUGCUA | 11 | CCAUGUUAGCAAUC | 411 | AUGUGGUCCA | 811 |
| 137 | ACAUGG | | CUGGACCACAUGG | | GGAUUGCUA | |
| LPA- | UGUGGUCCAGGAUUGCUAC | 12 | ACCAUUGUAGCAAU | 412 | UGUGGUCCAG | 812 |
| 138 | AAUGGT | | CCUGGACCACAUG | | GAUUGCUAC | |
| LPA- | GGUGAUGGACAGAGUUAUC | 13 | UGCCUUGAUAACUC | 413 | GGUGAUGGAC | 813 |
| 160 | AAGGCA | | UGUCCAUCACCAU | | AGAGUUAUC | |
| LPA- | UCCACCACUGUCACAGGAA | 14 | AGGUCUUUCCUGUG | 414 | UCCACCACUG | 814 |
| 190 | AGACCT | | ACAGUGGUGGAGU | | UCACAGGAA | |
| LPA- | CCACCACUGUCACAGGAAG | 15 | CAGGUUCUUCCUGU | 415 | CCACCACUGU | 815 |
| 191 | AACCTG | | GACAGUGGUGGAG | | CACAGGAAG | |
| LPA- | CUGUCACAGGAAGGACCUG | 16 | GCUUGUCAGGUCCU | 416 | CUGUCACAGG | 816 |
| 197 | ACAAGC | | UCCUGUGACAGUG | | AAGGACCUG | |
| LPA- | GGAAGGACCUGCCAAGCUU | 17 | AUGACUAAGCUUGG | 417 | GGAAGGACCU | 817 |
| 205 | AGUCAT | | CAGGUCCUUCCUG | | GCCAAGCUU | |
| LPA- | GAAGGACCUGCCAAGCUUG | 18 | GAUGAUCAAGCUUG | 418 | GAAGGACCUG | 818 |
| 206 | AUCATC | | GCAGGUCCUUCCU | | CCAAGCUUG | |
| LPA- | AGGACCUGCCAAGCUUGGU | 19 | UAGAUUACCAAGCU | 419 | AGGACCUGCC | 819 |
| 208 | AAUCTA | | UGGCAGGUCCUUC | | AAGCUUGGU | |
| LPA- | GGACCUGCCAAGCUUGGUC | 20 | AUAGAUGACCAAGC | 420 | GGACCUGCCA | 820 |
| 209 | AUCUAT | | UUGGCAGGUCCUU | | AGCUUGGUC | |
| LPA- | GACCUGCCAAGCUUGGUCA | 21 | CAUAGUUGACCAAG | 421 | GACCUGCCAA | 821 |
| 210 | ACUATG | | CUUGGCAGGUCCU | | GCUUGGUCA | |
| LPA- | ACCUGCCAAGCUUGGUCAU | 22 | UCAUAUAUGACCAA | 422 | ACCUGCCAAG | 822 |
| 211 | AUAUGA | | GCUUGGCAGGUCC | | CUUGGUCAU | |
| LPA- | CCUGCCAAGCUUGGUCAUC | 23 | GUCAUUGAUGACCA | 423 | CCUGCCAAGC | 823 |
| 212 | AAUGAC | | AGCUUGGCAGGUC | | UUGGUCAUC | |
| LPA- | AGCUUGGUCAUCUAUGACA | 24 | AUGUGUUGUCAUAG | 424 | AGCUUGGUCA | 824 |
| 219 | ACACAT | | AUGACCAAGCUUG | | UCUAUGACA | |
| LPA- | GUCAUCUAUGACACCACAU | 25 | AUGUUUAUGUGGUG | 425 | GUCAUCUAUG | 825 |
| 225 | AAACAT | | UCAUAGAUGACCA | | ACACCACAU | |
| LPA- | CACAGAAAACUACCCAAAU | 26 | GCCAGUAUUUGGGU | 426 | CACAGAAAAC | 826 |
| 258 | ACUGGC | | AGUUUUCUGUGGU | | UACCCAAAU | |
| LPA- | AGAAAACUACCCAAAUGCU | 27 | CAAGCUAGCAUUUG | 427 | AGAAAACUAC | 827 |
| 261 | AGCUTG | | GGUAGUUUUCUGU | | CCAAAUGCU | |
| LPA- | AAAACUACCCAAAUGCUGG | 28 | AUCAAUCCAGCAUU | 428 | AAAACUACCC | 828 |
| 263 | AUUGAT | | UGGGUAGUUUUCU | | AAAUGCUGG | |
| LPA- | ACCCAAAUGCUGGCUUGAU | 29 | UUCAUUAUCAAGCC | 429 | ACCCAAAUGC | 829 |
| 269 | AAUGAA | | AGCAUUUGGGUAG | | UGGCUUGAU | |
| LPA- | CCCAAAUGCUGGCUUGAUC | 30 | GUUCAUGAUCAAGC | 430 | CCCAAAUGCU | 830 |
| 270 | AUGAAC | | CAGCAUUUGGGUA | | GGCUUGAUC | |
| LPA- | GAACUACUGCAGGAAUCCA | 31 | AGCAUUUGGAUUCC | 431 | GAACUACUGC | 831 |
| 291 | AAUGCT | | UGCAGUAGUUCAU | | AGGAAUCCA | |
| LPA- | UACUGCAGGAAUCCAGAUG | 32 | CCACAUCAUCUGGA | 432 | UACUGCAGGA | 832 |
| 295 | AUGUGG | | UUCCUGCAGUAGU | | AUCCAGAUG | |
| LPA- | ACUGCAGGAAUCCAGAUGC | 33 | GCCACUGCAUCUGG | 433 | ACUGCAGGAA | 833 |
| 296 | AGUGGC | | AUUCCUGCAGUAG | | UCCAGAUGC | |
| LPA- | UGCAGGAAUCCAGAUGCUG | 34 | CUGCCUCAGCAUCU | 434 | UGCAGGAAUC | 834 |
| 298 | AGGCAG | | GGAUUCCUGCAGU | | CAGAUGCUG | |
| LPA- | AGGUGGGAGUACUGCAACC | 35 | GCGUCUGGUUGCAG | 435 | AGGUGGGAGU | 835 |
| 355 | AGACGC | | UACUCCCACCUGA | | ACUGCAACC | |
| LPA- | AAUGCUCAGACGCAGAAGG | 36 | GCAGUUCCUUCUGC | 436 | AAUGCUCAGA | 836 |
| 380 | AACUGC | | GUCUGAGCAUUGC | | CGCAGAAGG | |
| LPA- | GACUGUUACCCCGGUUCCA | 37 | UAGGCUUGGAACCG | 437 | GACUGUUACC | 837 |
| 417 | AGCCTA | | GGGUAACAGUCGG | | CCGGUUCCA | |
| LPA- | ACUGUUACCCCGGUUCCAA | 38 | CUAGGUUUGGAACC | 438 | ACUGUUACCC | 838 |
| 418 | ACCUAG | | GGGGUAACAGUCG | | CGGUUCCAA | |
| LPA- | CUGUUACCCCGGUUCCAAG | 39 | UCUAGUCUUGGAAC | 439 | CUGUUACCCC | 839 |
| 419 | ACUAGA | | CGGGGUAACAGUC | | GGUUCCAAG | |
| LPA- | UGUUACCCCGGUUCCAAGC | 40 | CUCUAUGCUUGGAA | 440 | UGUUACCCCG | 840 |
| 420 | AUAGAG | | CCGGGGUAACAGU | | GUUCCAAGC | |
| LPA- | GUUACCCCGGUUCCAAGCC | 41 | CCUCUUGGCUUGGA | 441 | GUUACCCCGG | 841 |
| 421 | AAGAGG | | ACCGGGGUAACAG | | UUCCAAGCC | |
| LPA- | UUACCCCGGUUCCAAGCCU | 42 | GCCUCUAGGCUUGG | 442 | UUACCCCGGU | 842 |
| 422 | AGAGGC | | AACCGGGGUAACA | | UCCAAGCCU | |
| LPA- | UACCCCGGUUCCAAGCCUA | 43 | AGCCUUUAGGCUUG | 443 | UACCCCGGUU | 843 |
| 423 | AAGGCT | | GAACCGGGGUAAC | | CCAAGCCUA | |
| LPA- | GUGCUACCAUGGUAAUGGA | 44 | ACUCUUUCCAUUAC | 444 | GUGCUACCAU | 844 |
| 492 | AAGAGT | | CAUGGUAGCACUC | | GGUAAUGGA | |
| LPA- | UGCUACCAUGGUAAUGGAC | 45 | AACUCUGUCCAUUA | 445 | UGCUACCAUG | 845 |
| 493 | AGAGTT | | CCAUGGUAGCACU | | GUAAUGGAC | |
| LPA- | GCUACCAUGGUAAUGGACA | 46 | UAACUUUGUCCAUU | 446 | GCUACCAUGG | 846 |
| 494 | AAGUTA | | ACCAUGGUAGCAC | | UAAUGGACA | |
| LPA- | CUACCAUGGUAAUGGACAG | 47 | AUAACUCUGUCCAU | 447 | CUACCAUGGU | 847 |
| 495 | AGUUAT | | UACCAUGGUAGCA | | AAUGGACAG | |
| LPA- | UACCAUGGUAAUGGACAGA | 48 | GAUAAUUCUGUCCA | 448 | UACCAUGGUA | 848 |
| 496 | AUUATC | | UUACCAUGGUAGC | | AUGGACAGA | |
| LPA- | ACCAUGGUAAUGGACAGAG | 49 | CGAUAUCUCUGUCC | 449 | ACCAUGGUAA | 849 |
| 497 | AUAUCG | | AUUACCAUGGUAG | | UGGACAGAG | |
| LPA- | CCAUGGUAAUGGACAGAGU | 50 | UCGAUUACUCUGUC | 450 | CCAUGGUAAU | 850 |
| 498 | AAUCGA | | CAUUACCAUGGUA | | GGACAGAGU | |
| LPA- | CAUGGUAAUGGACAGAGUU | 51 | CUCGAUAACUCUGU | 451 | CAUGGUAAUG | 851 |
| 499 | AUCGAG | | CCAUUACCAUGGU | | GACAGAGUU | |
| LPA- | AUGGUAAUGGACAGAGUUA | 52 | CCUCGUUAACUCUG | 452 | AUGGUAAUGG | 852 |
| 500 | ACGAGG | | UCCAUUACCAUGG | | ACAGAGUUA | |
| LPA- | UGGUAAUGGACAGAGUUAU | 53 | GCCUCUAUAACUCU | 453 | UGGUAAUGGA | 853 |
| 501 | AGAGGC | | GUCCAUUACCAUG | | CAGAGUUAU | |
| LPA- | GGUAAUGGACAGAGUUAUC | 54 | UGCCUUGAUAACUC | 454 | GGUAAUGGAC | 854 |
| 502 | AAGGCA | | UGUCCAUUACCAU | | AGAGUUAUC | |
| LPA- | GUAAUGGACAGAGUUAUCG | 55 | GUGCCUCGAUAACU | 455 | GUAAUGGACA | 855 |
| 503 | AGGCAC | | CUGUCCAUUACCA | | GAGUUAUCG | |
| LPA- | GGCACAUACUCCACCACUG | 56 | CUGUGUCAGUGGUG | 456 | GGCACAUACU | 856 |
| 523 | ACACAG | | GAGUAUGUGCCUC | | CCACCACUG | |
| LPA- | CUUGGUCAUCUAUGACACC | 57 | GAGUGUGGUGUCAU | 457 | CUUGGUCAUC | 857 |
| 563 | ACACTC | | AGAUGACCAAGCU | | UAUGACACC | |
| LPA- | GUCAUCUAUGACACCACAC | 58 | AUGCGUGUGUGGUG | 458 | GUCAUCUAUG | 858 |
| 567 | ACGCAT | | UCAUAGAUGACCA | | ACACCACAC | |
| LPA- | UCAUCUAUGACACCACACU | 59 | UAUGCUAGUGUGGU | 459 | UCAUCUAUGA | 859 |
| 568 | AGCATA | | GUCAUAGAUGACC | | CACCACACU | |
| LPA- | CAUCUAUGACACCACACUC | 60 | CUAUGUGAGUGUGG | 460 | CAUCUAUGAC | 860 |
| 569 | ACAUAG | | UGUCAUAGAUGAC | | ACCACACUC | |
| LPA- | GCACAUACUCCACCACUGU | 61 | CCAGUUACAGUGGU | 461 | GCACAUACUC | 861 |
| 1208 | AACUGG | | GGAGUAUGUGCCU | | CACCACUGU | |
| LPA- | AGCCCCUUAUUGUUAUACG | 62 | AUCCCUCGUAUAAC | 462 | AGCCCCUUAU | 862 |
| 2715 | AGGGAT | | AAUAAGGGGCUGC | | UGUUAUACG | |
| LPA- | GCCCCUUAUUGUUAUACGA | 63 | GAUCCUUCGUAUAA | 463 | GCCCCUUAUU | 863 |
| 2716 | AGGATC | | CAAUAAGGGGCUG | | GUUAUACGA | |
| LPA- | CCAAGCCUAGAGGCUCCUU | 64 | GUUCAUAAGGAGCC | 464 | CCAAGCCUAG | 864 |
| 2827 | AUGAAC | | UCUAGGCUUGGAA | | AGGCUCCUU | |
| LPA- | AGGCUCCUUCUGAACAAGC | 65 | GUUGGUGCUUGUUC | 465 | AGGCUCCUUC | 865 |
| 2837 | ACCAAC | | AGAAGGAGCCUCU | | UGAACAAGC | |
| LPA- | AUGGACAGAGUUAUCAAGG | 66 | UAUGUUCCUUGAUA | 466 | AUGGACAGAG | 866 |
| 2900 | AACATA | | ACUCUGUCCAUUU | | UUAUCAAGG | |
| LPA- | UGGACAGAGUUAUCAAGGC | 67 | GUAUGUGCCUUGAU | 467 | UGGACAGAGU | 867 |
| 2901 | ACAUAC | | AACUCUGUCCAUU | | UAUCAAGGC | |
| LPA- | GGACAGAGUUAUCAAGGCA | 68 | AGUAUUUGCCUUGA | 468 | GGACAGAGUU | 868 |
| 2902 | AAUACT | | UAACUCUGUCCAU | | AUCAAGGCA | |
| LPA- | GACAGAGUUAUCAAGGCAC | 69 | AAGUAUGUGCCUUG | 469 | GACAGAGUUA | 869 |
| 2903 | AUACTT | | AUAACUCUGUCCA | | UCAAGGCAC | |
| LPA- | ACAGAGUUAUCAAGGCACA | 70 | GAAGUUUGUGCCUU | 470 | ACAGAGUUAU | 870 |
| 2904 | AACUTC | | GAUAACUCUGUCC | | CAAGGCACA | |
| LPA- | CAGAGUUAUCAAGGCACAU | 71 | UGAAGUAUGUGCCU | 471 | CAGAGUUAUC | 871 |
| 2905 | ACUUCA | | UGAUAACUCUGUC | | AAGGCACAU | |
| LPA- | UACCCAAAUGCUGGCUUGA | 72 | UCUUGUUCAAGCCA | 472 | UACCCAAAUG | 872 |
| 3004 | ACAAGA | | GCAUUUGGGUAGU | | CUGGCUUGA | |
| LPA- | CCAAAUGCUGGCUUGAUCA | 73 | AGUUCUUGAUCAAG | 473 | CCAAAUGCUG | 873 |
| 3007 | AGAACT | | CCAGCAUUUGGGU | | GCUUGAUCA | |
| LPA- | UCAAGAACUACUGCCGAAA | 74 | UCUGGUUUUCGGCA | 474 | UCAAGAACUA | 874 |
| 3023 | ACCAGA | | GUAGUUCUUGAUC | | CUGCCGAAA | |
| LPA- | CAAGAACUACUGCCGAAAU | 75 | AUCUGUAUUUCGGC | 475 | CAAGAACUAC | 875 |
| 3024 | ACAGAT | | AGUAGUUCUUGAU | | UGCCGAAAU | |
| LPA- | AAGAACUACUGCCGAAAUC | 76 | GAUCUUGAUUUCGG | 476 | AAGAACUACU | 876 |
| 3025 | AAGATC | | CAGUAGUUCUUGA | | GCCGAAAUC | |
| LPA- | GAACUACUGCCGAAAUCCA | 77 | AGGAUUUGGAUUUC | 477 | GAACUACUGC | 877 |
| 3027 | AAUCCT | | GGCAGUAGUUCUU | | CGAAAUCCA | |
| LPA- | CUACUGCCGAAAUCCAGAU | 78 | CACAGUAUCUGGAU | 478 | CUACUGCCGA | 878 |
| 3030 | ACUGTG | | UUCGGCAGUAGUU | | AAUCCAGAU | |
| LPA- | UGUGGCAGCCCCUUGGUGU | 79 | UGUAUUACACCAAG | 479 | UGUGGCAGCC | 879 |
| 3051 | AAUACA | | GGGCUGCCACAGG | | CCUUGGUGU | |
| LPA- | GUGGCAGCCCCUUGGUGUU | 80 | UUGUAUAACACCAA | 480 | GUGGCAGCCC | 880 |
| 3052 | AUACAA | | GGGGCUGCCACAG | | CUUGGUGUU | |
| LPA- | UGGCAGCCCCUUGGUGUUA | 81 | GUUGUUUAACACCA | 481 | UGGCAGCCCC | 881 |
| 3053 | AACAAC | | AGGGGCUGCCACA | | UUGGUGUUA | |
| LPA- | GGCAGCCCCUUGGUGUUAU | 82 | UGUUGUAUAACACC | 482 | GGCAGCCCCU | 882 |
| 3054 | ACAACA | | AAGGGGCUGCCAC | | UGGUGUUAU | |
| LPA- | GCAGCCCCUUGGUGUUAUA | 83 | CUGUUUUAUAACAC | 483 | GCAGCCCCUU | 883 |
| 3055 | AAACAG | | CAAGGGGCUGCCA | | GGUGUUAUA | |
| LPA- | CAGCCCCUUGGUGUUAUAC | 84 | UCUGUUGUAUAACA | 484 | CAGCCCCUUG | 884 |
| 3056 | AACAGA | | CCAAGGGGCUGCC | | GUGUUAUAC | |
| LPA- | AGCCCCUUGGUGUUAUACA | 85 | AUCUGUUGUAUAAC | 485 | AGCCCCUUGG | 885 |
| 3057 | ACAGAT | | ACCAAGGGGCUGC | | UGUUAUACA | |
| LPA- | GCCCCUUGGUGUUAUACAA | 86 | GAUCUUUUGUAUAA | 486 | GCCCCUUGGU | 886 |
| 3058 | AAGATC | | CACCAAGGGGCUG | | GUUAUACAA | |
| LPA- | CCCCUUGGUGUUAUACAAC | 87 | GGAUCUGUUGUAUA | 487 | CCCCUUGGUG | 887 |
| 3059 | AGAUCC | | ACACCAAGGGGCU | | UUAUACAAC | |
| LPA- | GGUGGGAGUACUGCAACCU | 88 | CGUGUUAGGUUGCA | 488 | GGUGGGAGUA | 888 |
| 3092 | AACACG | | GUACUCCCACCUG | | CUGCAACCU | |
| LPA- | GUGGGAGUACUGCAACCUG | 89 | UCGUGUCAGGUUGC | 489 | GUGGGAGUAC | 889 |
| 3093 | ACACGA | | AGUACUCCCACCU | | UGCAACCUG | |
| LPA- | GGAGUACUGCAACCUGACA | 90 | GCAUCUUGUCAGGU | 490 | GGAGUACUGC | 890 |
| 3096 | AGAUGC | | UGCAGUACUCCCA | | AACCUGACA | |
| LPA- | GAGUACUGCAACCUGACAC | 91 | AGCAUUGUGUCAGG | 491 | GAGUACUGCA | 891 |
| 3097 | AAUGCT | | UUGCAGUACUCCC | | ACCUGACAC | |
| LPA- | GUACUGCAACCUGACACGA | 92 | UGAGCUUCGUGUCA | 492 | GUACUGCAAC | 892 |
| 3099 | AGCUCA | | GGUUGCAGUACUC | | CUGACACGA | |
| LPA- | UACUGCAACCUGACACGAU | 93 | CUGAGUAUCGUGUC | 493 | UACUGCAACC | 893 |
| 3100 | ACUCAG | | AGGUUGCAGUACU | | UGACACGAU | |
| LPA- | ACUGCAACCUGACACGAUG | 94 | UCUGAUCAUCGUGU | 494 | ACUGCAACCU | 894 |
| 3101 | AUCAGA | | CAGGUUGCAGUAC | | GACACGAUG | |
| LPA- | CUGCAACCUGACACGAUGC | 95 | AUCUGUGCAUCGUG | 495 | CUGCAACCUG | 895 |
| 3102 | ACAGAT | | UCAGGUUGCAGUA | | ACACGAUGC | |
| LPA- | UGCAACCUGACACGAUGCU | 96 | CAUCUUAGCAUCGU | 496 | UGCAACCUGA | 896 |
| 3103 | AAGATG | | GUCAGGUUGCAGU | | CACGAUGCU | |
| LPA- | CAACCUGACACGAUGCUCA | 97 | UGCAUUUGAGCAUC | 497 | CAACCUGACA | 897 |
| 3105 | AAUGCA | | GUGUCAGGUUGCA | | CGAUGCUCA | |
| LPA- | ACCUGACACGAUGCUCAGA | 98 | UCUGCUUCUGAGCA | 498 | ACCUGACACG | 898 |
| 3107 | AGCAGA | | UCGUGUCAGGUUG | | AUGCUCAGA | |
| LPA- | CCUGACACGAUGCUCAGAU | 99 | UUCUGUAUCUGAGC | 499 | CCUGACACGA | 899 |
| 3108 | ACAGAA | | AUCGUGUCAGGUU | | UGCUCAGAU | |
| LPA- | CUGACACGAUGCUCAGAUG | 100 | AUUCUUCAUCUGAG | 500 | CUGACACGAU | 900 |
| 3109 | AAGAAT | | CAUCGUGUCAGGU | | GCUCAGAUG | |
| LPA- | UGACACGAUGCUCAGAUGC | 101 | CAUUCUGCAUCUGA | 501 | UGACACGAUG | 901 |
| 3110 | AGAATG | | GCAUCGUGUCAGG | | CUCAGAUGC | |
| LPA- | GACACGAUGCUCAGAUGCA | 102 | CCAUUUUGCAUCUG | 502 | GACACGAUGC | 902 |
| 3111 | AAAUGG | | AGCAUCGUGUCAG | | UCAGAUGCA | |
| LPA- | ACACGAUGCUCAGAUGCAG | 103 | UCCAUUCUGCAUCU | 503 | ACACGAUGCU | 903 |
| 3112 | AAUGGA | | GAGCAUCGUGUCA | | CAGAUGCAG | |
| LPA- | CACGAUGCUCAGAUGCAGA | 104 | GUCCAUUCUGCAUC | 504 | CACGAUGCUC | 904 |
| 3113 | AUGGAC | | UGAGCAUCGUGUC | | AGAUGCAGA | |
| LPA- | UGCUACUACCAUUAUGGAC | 105 | AACUCUGUCCAUAA | 505 | UGCUACUACC | 905 |
| 3229 | AGAGTT | | UGGUAGUAGCAGU | | AUUAUGGAC | |
| LPA- | GCUACUACCAUUAUGGACA | 106 | UAACUUUGUCCAUA | 506 | GCUACUACCA | 906 |
| 3230 | AAGUTA | | AUGGUAGUAGCAG | | UUAUGGACA | |
| LPA- | CUACUACCAUUAUGGACAG | 107 | GUAACUCUGUCCAU | 507 | CUACUACCAU | 907 |
| 3231 | AGUUAC | | AAUGGUAGUAGCA | | UAUGGACAG | |
| LPA- | UACUACCAUUAUGGACAGA | 108 | GGUAAUUCUGUCCA | 508 | UACUACCAUU | 908 |
| 3232 | AUUACC | | UAAUGGUAGUAGC | | AUGGACAGA | |
| LPA- | ACUACCAUUAUGGACAGAG | 109 | CGGUAUCUCUGUCC | 509 | ACUACCAUUA | 909 |
| 3233 | AUACCG | | AUAAUGGUAGUAG | | UGGACAGAG | |
| LPA- | CUACCAUUAUGGACAGAGU | 110 | UCGGUUACUCUGUC | 510 | CUACCAUUAU | 910 |
| 3234 | AACCGA | | CAUAAUGGUAGUA | | GGACAGAGU | |
| LPA- | UACCAUUAUGGACAGAGUU | 111 | CUCGGUAACUCUGU | 511 | UACCAUUAUG | 911 |
| 3235 | ACCGAG | | CCAUAAUGGUAGU | | GACAGAGUU | |
| LPA- | ACCAUUAUGGACAGAGUUA | 112 | CCUCGUUAACUCUG | 512 | ACCAUUAUGG | 912 |
| 3236 | ACGAGG | | UCCAUAAUGGUAG | | ACAGAGUUA | |
| LPA- | GAGGCACAUACUCCACCAC | 113 | GUGACUGUGGUGGA | 513 | GAGGCACAUA | 913 |
| 3257 | AGUCAC | | GUAUGUGCCUCGG | | CUCCACCAC | |
| LPA- | CUCCACCACUGUCACAGGA | 114 | AGUUCUUCCUGUGA | 514 | CUCCACCACU | 914 |
| 3267 | AGAACT | | CAGUGGUGGAGUA | | GUCACAGGA | |
| LPA- | ACAGGAAGAACUUGCCAAG | 115 | ACCAAUCUUGGCAA | 515 | ACAGGAAGAA | 915 |
| 3280 | AUUGGT | | GUUCUUCCUGUGA | | CUUGCCAAG | |
| LPA- | CAGGAAGAACUUGCCAAGC | 116 | GACCAUGCUUGGCA | 516 | CAGGAAGAAC | 916 |
| 3281 | AUGGTC | | AGUUCUUCCUGUG | | UUGCCAAGC | |
| LPA- | AGGAAGAACUUGCCAAGCU | 117 | UGACCUAGCUUGGC | 517 | AGGAAGAACU | 917 |
| 3282 | AGGUCA | | AAGUUCUUCCUGU | | UGCCAAGCU | |
| LPA- | GGAAGAACUUGCCAAGCUU | 118 | AUGACUAAGCUUGG | 518 | GGAAGAACUU | 918 |
| 3283 | AGUCAT | | CAAGUUCUUCCUG | | GCCAAGCUU | |
| LPA- | GAAGAACUUGCCAAGCUUG | 119 | GAUGAUCAAGCUUG | 519 | GAAGAACUUG | 919 |
| 3284 | AUCATC | | GCAAGUUCUUCCU | | CCAAGCUUG | |
| LPA- | AAGAACUUGCCAAGCUUGG | 120 | AGAUGUCCAAGCUU | 520 | AAGAACUUGC | 920 |
| 3285 | ACAUCT | | GGCAAGUUCUUCC | | CAAGCUUGG | |
| LPA- | AGAACUUGCCAAGCUUGGU | 121 | UAGAUUACCAAGCU | 521 | AGAACUUGCC | 921 |
| 3286 | AAUCTA | | UGGCAAGUUCUUC | | AAGCUUGGU | |
| LPA- | GAACUUGCCAAGCUUGGUC | 122 | AUAGAUGACCAAGC | 522 | GAACUUGCCA | 922 |
| 3287 | AUCUAT | | UUGGCAAGUUCUU | | AGCUUGGUC | |
| LPA- | AACUUGCCAAGCUUGGUCA | 123 | CAUAGUUGACCAAG | 523 | AACUUGCCAA | 923 |
| 3288 | ACUATG | | CUUGGCAAGUUCU | | GCUUGGUCA | |
| LPA- | ACUUGCCAAGCUUGGUCAU | 124 | UCAUAUAUGACCAA | 524 | ACUUGCCAAG | 924 |
| 3289 | AUAUGA | | GCUUGGCAAGUUC | | CUUGGUCAU | |
| LPA- | CUUGCCAAGCUUGGUCAUC | 125 | GUCAUUGAUGACCA | 525 | CUUGCCAAGC | 925 |
| 3290 | AAUGAC | | AGCUUGGCAAGUU | | UUGGUCAUC | |
| LPA- | UUGCCAAGCUUGGUCAUCU | 126 | UGUCAUAGAUGACC | 526 | UUGCCAAGCU | 926 |
| 3291 | AUGACA | | AAGCUUGGCAAGU | | UGGUCAUCU | |
| LPA- | UGCCAAGCUUGGUCAUCUA | 127 | GUGUCUUAGAUGAC | 527 | UGCCAAGCUU | 927 |
| 3292 | AGACAC | | CAAGCUUGGCAAG | | GGUCAUCUA | |
| LPA- | GCUUGGUCAUCUAUGACAC | 128 | GGUGUUGUGUCAUA | 528 | GCUUGGUCAU | 928 |
| 3298 | AACACC | | GAUGACCAAGCUU | | CUAUGACAC | |
| LPA- | UUGGUCAUCUAUGACACCA | 129 | CUGGUUUGGUGUCA | 529 | UUGGUCAUCU | 929 |
| 3300 | AACCAG | | UAGAUGACCAAGC | | AUGACACCA | |
| LPA- | UGGUCAUCUAUGACACCAC | 130 | GCUGGUGUGGUGUC | 530 | UGGUCAUCUA | 930 |
| 3301 | ACCAGC | | AUAGAUGACCAAG | | UGACACCAC | |
| LPA- | GUCAUCUAUGACACCACAC | 131 | AUGCUUGUGUGGUG | 531 | GUCAUCUAUG | 931 |
| 3303 | AAGCAT | | UCAUAGAUGACCA | | ACACCACAC | |
| LPA- | CAUCUAUGACACCACACCA | 132 | CUAUGUUGGUGUGG | 532 | CAUCUAUGAC | 932 |
| 3305 | ACAUAG | | UGUCAUAGAUGAC | | ACCACACCA | |
| LPA- | AUCUAUGACACCACACCAG | 133 | ACUAUUCUGGUGUG | 533 | AUCUAUGACA | 933 |
| 3306 | AAUAGT | | GUGUCAUAGAUGA | | CCACACCAG | |
| LPA- | CUAUGACACCACACCAGCA | 134 | CGACUUUGCUGGUG | 534 | CUAUGACACC | 934 |
| 3308 | AAGUCG | | UGGUGUCAUAGAU | | ACACCAGCA | |
| LPA- | GUCGGACCCCAGAAAACUA | 135 | UUUGGUUAGUUUUC | 535 | GUCGGACCCC | 935 |
| 3329 | ACCAAA | | UGGGGUCCGACUA | | AGAAAACUA | |
| LPA- | UCGGACCCCAGAAAACUAC | 136 | AUUUGUGUAGUUUU | 536 | UCGGACCCCA | 936 |
| 3330 | ACAAAT | | CUGGGGUCCGACU | | GAAAACUAC | |
| LPA- | GAAAACUACCCAAAUGCUG | 137 | UCAGGUCAGCAUUU | 537 | GAAAACUACC | 937 |
| 3340 | ACCUGA | | GGGUAGUUUUCUG | | CAAAUGCUG | |
| LPA- | GCUGAGAUUCGCCCUUGGU | 138 | UGUAAUACCAAGGG | 538 | GCUGAGAUUC | 938 |
| 3391 | AUUACA | | CGAAUCUCAGCAU | | GCCCUUGGU | |
| LPA- | CUGAGAUUCGCCCUUGGUG | 139 | GUGUAUCACCAAGG | 539 | CUGAGAUUCG | 939 |
| 3392 | AUACAC | | GCGAAUCUCAGCA | | CCCUUGGUG | |
| LPA- | GAGAUUCGCCCUUGGUGUU | 140 | UGGUGUAACACCAA | 540 | GAGAUUCGCC | 940 |
| 3394 | ACACCA | | GGGCGAAUCUCAG | | CUUGGUGUU | |
| LPA- | AGAUUCGCCCUUGGUGUUA | 141 | AUGGUUUAACACCA | 541 | AGAUUCGCCC | 941 |
| 3395 | AACCAT | | AGGGCGAAUCUCA | | UUGGUGUUA | |
| LPA- | UUCGCCCUUGGUGUUACAC | 142 | UCCAUUGUGUAACA | 542 | UUCGCCCUUG | 942 |
| 3398 | AAUGGA | | CCAAGGGCGAAUC | | GUGUUACAC | |
| LPA- | CUUGGUGUUACACCAUGGA | 143 | CUGGGUUCCAUGGU | 543 | CUUGGUGUUA | 943 |
| 3404 | ACCCAG | | GUAACACCAAGGG | | CACCAUGGA | |
| LPA- | UUGGUGUUACACCAUGGAU | 144 | ACUGGUAUCCAUGG | 544 | UUGGUGUUAC | 944 |
| 3405 | ACCAGT | | UGUAACACCAAGG | | ACCAUGGAU | |
| LPA- | UGGUGUUACACCAUGGAUC | 145 | CACUGUGAUCCAUG | 545 | UGGUGUUACA | 945 |
| 3406 | ACAGTG | | GUGUAACACCAAG | | CCAUGGAUC | |
| LPA- | GGUGUUACACCAUGGAUCC | 146 | ACACUUGGAUCCAU | 546 | GGUGUUACAC | 946 |
| 3407 | AAGUGT | | GGUGUAACACCAA | | CAUGGAUCC | |
| LPA- | UGUUACACCAUGGAUCCCA | 147 | UGACAUUGGGAUCC | 547 | UGUUACACCA | 947 |
| 3409 | AUGUCA | | AUGGUGUAACACC | | UGGAUCCCA | |
| LPA- | GAAUCAAGUGUCCUUGCAA | 148 | UGAGAUUUGCAAGG | 548 | GAAUCAAGUG | 948 |
| 3472 | AUCUCA | | ACACUUGAUUCUG | | UCCUUGCAA | |
| LPA- | AAUCAAGUGUCCUUGCAAC | 149 | GUGAGUGUUGCAAG | 549 | AAUCAAGUGU | 949 |
| 3473 | ACUCAC | | GACACUUGAUUCU | | CCUUGCAAC | |
| LPA- | AUCAAGUGUCCUUGCAACU | 150 | CGUGAUAGUUGCAA | 550 | AUCAAGUGUC | 950 |
| 3474 | AUCACG | | GGACACUUGAUUC | | CUUGCAACU | |
| LPA- | AUGGACAGAGUUAUCGAGG | 151 | AAUGAUCCUCGAUA | 551 | AUGGACAGAG | 951 |
| 3584 | AUCATT | | ACUCUGUCCAUCA | | UUAUCGAGG | |
| LPA- | UGGACAGAGUUAUCGAGGC | 152 | GAAUGUGCCUCGAU | 552 | UGGACAGAGU | 952 |
| 3585 | ACAUTC | | AACUCUGUCCAUC | | UAUCGAGGC | |
| LPA- | ACACCACACUGGCAUCAGA | 153 | UUGUCUUCUGAUGC | 553 | ACACCACACU | 953 |
| 3655 | AGACAA | | CAGUGUGGUGUCA | | GGCAUCAGA | |
| LPA- | UUGGUGUUAUACCAUGGAU | 154 | AUUGGUAUCCAUGG | 554 | UUGGUGUUAU | 954 |
| 3747 | ACCAAT | | UAUAACACCAAGG | | ACCAUGGAU | |
| LPA- | UGGUGUUAUACCAUGGAUC | 155 | CAUUGUGAUCCAUG | 555 | UGGUGUUAUA | 955 |
| 3748 | ACAATG | | GUAUAACACCAAG | | CCAUGGAUC | |
| LPA- | GGUGUUAUACCAUGGAUCC | 156 | ACAUUUGGAUCCAU | 556 | GGUGUUAUAC | 956 |
| 3749 | AAAUGT | | GGUAUAACACCAA | | CAUGGAUCC | |
| LPA- | GUGUUAUACCAUGGAUCCC | 157 | GACAUUGGGAUCCA | 557 | GUGUUAUACC | 957 |
| 3750 | AAUGTC | | UGGUAUAACACCA | | AUGGAUCCC | |
| LPA- | UCAGAUGGGAGUACUGCAA | 158 | GUCAGUUUGCAGUA | 558 | UCAGAUGGGA | 958 |
| 3773 | ACUGAC | | CUCCCAUCUGACA | | GUACUGCAA | |
| LPA- | GAUGGGAGUACUGCAACCU | 159 | UGUGUUAGGUUGCA | 559 | GAUGGGAGUA | 959 |
| 3776 | AACACA | | GUACUCCCAUCUG | | CUGCAACCU | |
| LPA- | AUGGGAGUACUGCAACCUG | 160 | UUGUGUCAGGUUGC | 560 | AUGGGAGUAC | 960 |
| 3777 | ACACAA | | AGUACUCCCAUCU | | UGCAACCUG | |
| LPA- | UGGGAGUACUGCAACCUGA | 161 | AUUGUUUCAGGUUG | 561 | UGGGAGUACU | 961 |
| 3778 | AACAAT | | CAGUACUCCCAUC | | GCAACCUGA | |
| LPA- | GGGAGUACUGCAACCUGAC | 162 | CAUUGUGUCAGGUU | 562 | GGGAGUACUG | 962 |
| 3779 | ACAATG | | GCAGUACUCCCAU | | CAACCUGAC | |
| LPA- | GGCUGUUUCUGAACAAGCA | 163 | CGUUGUUGCUUGUU | 563 | GGCUGUUUCU | 963 |
| 3840 | ACAACG | | CAGAAACAGCCGU | | GAACAAGCA | |
| LPA- | GUUUCUGAACAAGCACCAA | 164 | GCUCCUUUGGUGCU | 564 | GUUUCUGAAC | 964 |
| 3844 | AGGAGC | | UGUUCAGAAACAG | | AAGCACCAA | |
| LPA- | CUCCACCACUGUUACAGGA | 165 | UGUCCUUCCUGUAA | 565 | CUCCACCACU | 965 |
| 3927 | AGGACA | | CAGUGGUGGAGAA | | GUUACAGGA | |
| LPA- | UCCACCACUGUUACAGGAA | 166 | AUGUCUUUCCUGUA | 566 | UCCACCACUG | 966 |
| 3928 | AGACAT | | ACAGUGGUGGAGA | | UUACAGGAA | |
| LPA- | CCACCACUGUUACAGGAAG | 167 | CAUGUUCUUCCUGU | 567 | CCACCACUGU | 967 |
| 3929 | AACATG | | AACAGUGGUGGAG | | UACAGGAAG | |
| LPA- | GACACCACACUGGCAUCAG | 168 | GGUUCUCUGAUGCC | 568 | GACACCACAC | 968 |
| 3972 | AGAACC | | AGUGUGGUGUCAU | | UGGCAUCAG | |
| LPA- | ACACCACACUGGCAUCAGA | 169 | UGGUUUUCUGAUGC | 569 | ACACCACACU | 969 |
| 3973 | AAACCA | | CAGUGUGGUGUCA | | GGCAUCAGA | |
| LPA- | AGAAUACUACCCAAAUGGU | 170 | CAGGCUACCAUUUG | 570 | AGAAUACUAC | 970 |
| 3999 | AGCCTG | | GGUAGUAUUCUGU | | CCAAAUGGU | |
| LPA- | GAAUACUACCCAAAUGGUG | 171 | UCAGGUCACCAUUU | 571 | GAAUACUACC | 971 |
| 4000 | ACCUGA | | GGGUAGUAUUCUG | | CAAAUGGUG | |
| LPA- | AAUACUACCCAAAUGGUGG | 172 | GUCAGUCCACCAUU | 572 | AAUACUACCC | 972 |
| 4001 | ACUGAC | | UGGGUAGUAUUCU | | AAAUGGUGG | |
| LPA- | UCCUUCUGAAGAAGCACCA | 173 | UUCAGUUGGUGCUU | 573 | UCCUUCUGAA | 973 |
| 4185 | ACUGAA | | CUUCAGAAGGAAG | | GAAGCACCA | |
| LPA- | CCUUCUGAAGAAGCACCAA | 174 | UUUCAUUUGGUGCU | 574 | CCUUCUGAAG | 974 |
| 4186 | AUGAAA | | UCUUCAGAAGGAA | | AAGCACCAA | |
| LPA- | CUUCUGAAGAAGCACCAAC | 175 | UUUUCUGUUGGUGC | 575 | CUUCUGAAGA | 975 |
| 4187 | AGAAAA | | UUCUUCAGAAGGA | | AGCACCAAC | |
| LPA- | UUCUGAAGAAGCACCAACU | 176 | GUUUUUAGUUGGUG | 576 | UUCUGAAGAA | 976 |
| 4188 | AAAAAC | | CUUCUUCAGAAGG | | GCACCAACU | |
| LPA- | UCUGAAGAAGCACCAACUG | 177 | UGUUUUCAGUUGGU | 577 | UCUGAAGAAG | 977 |
| 4189 | AAAACA | | GCUUCUUCAGAAG | | CACCAACUG | |
| LPA- | CUGAAGAAGCACCAACUGA | 178 | CUGUUUUCAGUUGG | 578 | CUGAAGAAGC | 978 |
| 4190 | AAACAG | | UGCUUCUUCAGAA | | ACCAACUGA | |
| LPA- | UGAAGAAGCACCAACUGAA | 179 | GCUGUUUUCAGUUG | 579 | UGAAGAAGCA | 979 |
| 4191 | AACAGC | | GUGCUUCUUCAGA | | CCAACUGAA | |
| LPA- | GAAGAAGCACCAACUGAAA | 180 | UGCUGUUUUCAGUU | 580 | GAAGAAGCAC | 980 |
| 4192 | ACAGCA | | GGUGCUUCUUCAG | | CAACUGAAA | |
| LPA- | AAGAAGCACCAACUGAAAA | 181 | GUGCUUUUUUCAGU | 581 | AAGAAGCACC | 981 |
| 4193 | AAGCAC | | UGGUGCUUCUUCA | | AACUGAAAA | |
| LPA- | AGAAGCACCAACUGAAAAC | 182 | AGUGCUGUUUUCAG | 582 | AGAAGCACCA | 982 |
| 4194 | AGCACT | | UUGGUGCUUCUUC | | ACUGAAAAC | |
| LPA- | GAAGCACCAACUGAAAACA | 183 | CAGUGUUGUUUUCA | 583 | GAAGCACCAA | 983 |
| 4195 | ACACTG | | GUUGGUGCUUCUU | | CUGAAAACA | |
| LPA- | AAGCACCAACUGAAAACAG | 184 | CCAGUUCUGUUUUC | 584 | AAGCACCAAC | 984 |
| 4196 | AACUGG | | AGUUGGUGCUUCU | | UGAAAACAG | |
| LPA- | AGGUGAUGGACAGAGUUAU | 185 | GCCUCUAUAACUCU | 585 | AGGUGAUGGA | 985 |
| 4239 | AGAGGC | | GUCCAUCACCUCG | | CAGAGUUAU | |
| LPA- | CUCCACCACUAUCACAGGA | 186 | UGUUCUUCCUGUGA | 586 | CUCCACCACU | 986 |
| 4269 | AGAACA | | UAGUGGUGGAGAG | | AUCACAGGA | |
| LPA- | UCCACCACUAUCACAGGAA | 187 | AUGUUUUUCCUGUG | 587 | UCCACCACUA | 987 |
| 4270 | AAACAT | | AUAGUGGUGGAGA | | UCACAGGAA | |
| LPA- | CCACCACUAUCACAGGAAG | 188 | CAUGUUCUUCCUGU | 588 | CCACCACUAU | 988 |
| 4271 | AACATG | | GAUAGUGGUGGAG | | CACAGGAAG | |
| LPA- | CACCACUAUCACAGGAAGA | 189 | ACAUGUUCUUCCUG | 589 | CACCACUAUC | 989 |
| 4272 | ACAUGT | | UGAUAGUGGUGGA | | ACAGGAAGA | |
| LPA- | ACCACUAUCACAGGAAGAA | 190 | GACAUUUUCUUCCU | 590 | ACCACUAUCA | 990 |
| 4273 | AAUGTC | | GUGAUAGUGGUGG | | CAGGAAGAA | |
| LPA- | CCACUAUCACAGGAAGAAC | 191 | UGACAUGUUCUUCC | 591 | CCACUAUCAC | 991 |
| 4274 | AUGUCA | | UGUGAUAGUGGUG | | AGGAAGAAC | |
| LPA- | CACUAUCACAGGAAGAACA | 192 | CUGACUUGUUCUUC | 592 | CACUAUCACA | 992 |
| 4275 | AGUCAG | | CUGUGAUAGUGGU | | GGAAGAACA | |
| LPA- | ACUAUCACAGGAAGAACAU | 193 | ACUGAUAUGUUCUU | 593 | ACUAUCACAG | 993 |
| 4276 | AUCAGT | | CCUGUGAUAGUGG | | GAAGAACAU | |
| LPA- | CUAUCACAGGAAGAACAUG | 194 | GACUGUCAUGUUCU | 594 | CUAUCACAGG | 994 |
| 4277 | ACAGTC | | UCCUGUGAUAGUG | | AAGAACAUG | |
| LPA- | UAUCACAGGAAGAACAUGU | 195 | AGACUUACAUGUUC | 595 | UAUCACAGGA | 995 |
| 4278 | AAGUCT | | UUCCUGUGAUAGU | | AGAACAUGU | |
| LPA- | AUCACAGGAAGAACAUGUC | 196 | AAGACUGACAUGUU | 596 | AUCACAGGAA | 996 |
| 4279 | AGUCTT | | CUUCCUGUGAUAG | | GAACAUGUC | |
| LPA- | UCACAGGAAGAACAUGUCA | 197 | CAAGAUUGACAUGU | 597 | UCACAGGAAG | 997 |
| 4280 | AUCUTG | | UCUUCCUGUGAUA | | AACAUGUCA | |
| LPA- | CACAGGAAGAACAUGUCAG | 198 | CCAAGUCUGACAUG | 598 | CACAGGAAGA | 998 |
| 4281 | ACUUGG | | UUCUUCCUGUGAU | | ACAUGUCAG | |
| LPA- | ACAGGAAGAACAUGUCAGU | 199 | ACCAAUACUGACAU | 599 | ACAGGAAGAA | 999 |
| 4282 | AUUGGT | | GUUCUUCCUGUGA | | CAUGUCAGU | |
| LPA- | GGAAGAACAUGUCAGUCUU | 200 | ACGACUAAGACUGA | 600 | GGAAGAACAU | 1000 |
| 4285 | AGUCGT | | CAUGUUCUUCCUG | | GUCAGUCUU | |
| LPA- | GAAGAACAUGUCAGUCUUG | 201 | GACGAUCAAGACUG | 601 | GAAGAACAUG | 1001 |
| 4286 | AUCGTC | | ACAUGUUCUUCCU | | UCAGUCUUG | |
| LPA- | AAGAACAUGUCAGUCUUGG | 202 | AGACGUCCAAGACU | 602 | AAGAACAUGU | 1002 |
| 4287 | ACGUCT | | GACAUGUUCUUCC | | CAGUCUUGG | |
| LPA- | AGAACAUGUCAGUCUUGGU | 203 | UAGACUACCAAGAC | 603 | AGAACAUGUC | 1003 |
| 4288 | AGUCTA | | UGACAUGUUCUUC | | AGUCUUGGU | |
| LPA- | GGCAUCGGAGGAUCCCAUU | 204 | UAGUAUAAUGGGAU | 604 | GGCAUCGGAG | 1004 |
| 4325 | AUACTA | | CCUCCGAUGCCAA | | GAUCCCAUU | |
| LPA- | ACUAUCCAAAUGCUGGCCU | 205 | CUGGUUAGGCCAGC | 605 | ACUAUCCAAA | 1005 |
| 4346 | AACCAG | | AUUUGGAUAGUAU | | UGCUGGCCU | |
| LPA- | GCACAGAGGCUCCUUCUGA | 206 | GCUUGUUCAGAAGG | 606 | GCACAGAGGC | 1006 |
| 4517 | ACAAGC | | AGCCUCUGUGCUU | | UCCUUCUGA | |
| LPA- | UCCUUCUGAACAAGCACCA | 207 | CUCAGUUGGUGCUU | 607 | UCCUUCUGAA | 1007 |
| 4527 | ACUGAG | | GUUCAGAAGGAGC | | CAAGCACCA | |
| LPA- | CCUUCUGAACAAGCACCAC | 208 | UCUCAUGUGGUGCU | 608 | CCUUCUGAAC | 1008 |
| 4528 | AUGAGA | | UGUUCAGAAGGAG | | AAGCACCAC | |
| LPA- | CUUCUGAACAAGCACCACC | 209 | UUCUCUGGUGGUGC | 609 | CUUCUGAACA | 1009 |
| 4529 | AGAGAA | | UUGUUCAGAAGGA | | AGCACCACC | |
| LPA- | UUCUGAACAAGCACCACCU | 210 | UUUCUUAGGUGGUG | 610 | UUCUGAACAA | 1010 |
| 4530 | AAGAAA | | CUUGUUCAGAAGG | | GCACCACCU | |
| LPA- | UCUGAACAAGCACCACCUG | 211 | UUUUCUCAGGUGGU | 611 | UCUGAACAAG | 1011 |
| 4531 | AGAAAA | | GCUUGUUCAGAAG | | CACCACCUG | |
| LPA- | CUGAACAAGCACCACCUGA | 212 | CUUUUUUCAGGUGG | 612 | CUGAACAAGC | 1012 |
| 4532 | AAAAAG | | UGCUUGUUCAGAA | | ACCACCUGA | |
| LPA- | UGAACAAGCACCACCUGAG | 213 | GCUUUUCUCAGGUG | 613 | UGAACAAGCA | 1013 |
| 4533 | AAAAGC | | GUGCUUGUUCAGA | | CCACCUGAG | |
| LPA- | GAACAAGCACCACCUGAGA | 214 | GGCUUUUCUCAGGU | 614 | GAACAAGCAC | 1014 |
| 4531 | AAAGCC | | GGUGCUUGUUCAG | | CACCUGAGA | |
| LPA- | AACAAGCACCACCUGAGAA | 215 | GGGCUUUUCUCAGG | 615 | AACAAGCACC | 1015 |
| 4535 | AAGCCC | | UGGUGCUUGUUCA | | ACCUGAGAA | |
| LPA- | CAAGCACCACCUGAGAAAA | 216 | CAGGGUUUUUCUCA | 616 | CAAGCACCAC | 1016 |
| 4537 | ACCCTG | | GGUGGUGCUUGUU | | CUGAGAAAA | |
| LPA- | AAGCACCACCUGAGAAAAG | 217 | ACAGGUCUUUUCUC | 617 | AAGCACCACC | 1017 |
| 4538 | ACCUGT | | AGGUGGUGCUUGU | | UGAGAAAAG | |
| LPA- | AGCACCACCUGAGAAAAGC | 218 | CACAGUGCUUUUCU | 618 | AGCACCACCU | 1018 |
| 4539 | ACUGTG | | CAGGUGGUGCUUG | | GAGAAAAGC | |
| LPA- | CUGAGAAAAGCCCUGUGGU | 219 | UCCUGUACCACAGG | 619 | CUGAGAAAAG | 1019 |
| 4547 | ACAGGA | | GCUUUUCUCAGGU | | CCCUGUGGU | |
| LPA- | GCCCUGUGGUCCAGGAUUG | 220 | UGGUAUCAAUCCUG | 620 | GCCCUGUGGU | 1020 |
| 4556 | AUACCA | | GACCACAGGGCUU | | CCAGGAUUG | |
| LPA- | CUGUGGUCCAGGAUUGCUA | 221 | CCAUGUUAGCAAUC | 621 | CUGUGGUCCA | 1021 |
| 4559 | ACAUGG | | CUGGACCACAGGG | | GGAUUGCUA | |
| LPA- | CUCCACCACUGUCACAGGA | 222 | GGUCCUUCCUGUGA | 622 | CUCCACCACU | 1022 |
| 4611 | AGGACC | | CAGUGGUGGAGGA | | GUCACAGGA | |
| LPA- | UCCACCACUGUCACAGGAA | 223 | AGGUCUUUCCUGUG | 623 | UCCACCACUG | 1023 |
| 4612 | AGACCT | | ACAGUGGUGGAGG | | UCACAGGAA | |
| LPA- | UCUUGGUCAUCUAUGAUAC | 224 | AGUGUUGUAUCAUA | 624 | UCUUGGUCAU | 1024 |
| 4642 | AACACT | | GAUGACCAAGAUU | | CUAUGAUAC | |
| LPA- | CUUGGUCAUCUAUGAUACC | 225 | CAGUGUGGUAUCAU | 625 | CUUGGUCAUC | 1025 |
| 4643 | ACACTG | | AGAUGACCAAGAU | | UAUGAUACC | |
| LPA- | UUGGUCAUCUAUGAUACCA | 226 | CCAGUUUGGUAUCA | 626 | UUGGUCAUCU | 1026 |
| 4644 | AACUGG | | UAGAUGACCAAGA | | AUGAUACCA | |
| LPA- | UGGUCAUCUAUGAUACCAC | 227 | GCCAGUGUGGUAUC | 627 | UGGUCAUCUA | 1027 |
| 4645 | ACUGGC | | AUAGAUGACCAAG | | UGAUACCAC | |
| LPA- | GGUCAUCUAUGAUACCACA | 228 | UGCCAUUGUGGUAU | 628 | GGUCAUCUAU | 1028 |
| 4646 | AUGGCA | | CAUAGAUGACCAA | | GAUACCACA | |
| LPA- | GUCAUCUAUGAUACCACAC | 229 | AUGCCUGUGUGGUA | 629 | GUCAUCUAUG | 1029 |
| 4647 | AGGCAT | | UCAUAGAUGACCA | | AUACCACAC | |
| LPA- | UCAUCUAUGAUACCACACU | 230 | GAUGCUAGUGUGGU | 630 | UCAUCUAUGA | 1030 |
| 4648 | AGCATC | | AUCAUAGAUGACC | | UACCACACU | |
| LPA- | CAUCUAUGAUACCACACUG | 231 | UGAUGUCAGUGUGG | 631 | CAUCUAUGAU | 1031 |
| 4649 | ACAUCA | | UAUCAUAGAUGAC | | ACCACACUG | |
| LPA- | AUCUAUGAUACCACACUGG | 232 | CUGAUUCCAGUGUG | 632 | AUCUAUGAUA | 1032 |
| 4650 | AAUCAG | | GUAUCAUAGAUGA | | CCACACUGG | |
| LPA- | UCUAUGAUACCACACUGGC | 233 | UCUGAUGCCAGUGU | 633 | UCUAUGAUAC | 1033 |
| 4651 | AUCAGA | | GGUAUCAUAGAUG | | CACACUGGC | |
| LPA- | CUAUGAUACCACACUGGCA | 234 | CUCUGUUGCCAGUG | 634 | CUAUGAUACC | 1034 |
| 4652 | ACAGAG | | UGGUAUCAUAGAU | | ACACUGGCA | |
| LPA- | UGAUACCACACUGGCAUCA | 235 | GUCCUUUGAUGCCA | 635 | UGAUACCACA | 1035 |
| 4655 | AAGGAC | | GUGUGGUAUCAUA | | CUGGCAUCA | |
| LPA- | AUACCACACUGGCAUCAGA | 236 | GGGUCUUCUGAUGC | 636 | AUACCACACU | 1036 |
| 4657 | AGACCC | | CAGUGUGGUAUCA | | GGCAUCAGA | |
| LPA- | AGAGGACCCCAGAAAACUA | 237 | UUUGGUUAGUUUUC | 637 | AGAGGACCCC | 1037 |
| 4673 | ACCAAA | | UGGGGUCCUCUGA | | AGAAAACUA | |
| LPA- | GAGGACCCCAGAAAACUAC | 238 | AUUUGUGUAGUUUU | 638 | GAGGACCCCA | 1038 |
| 4674 | ACAAAT | | CUGGGGUCCUCUG | | GAAAACUAC | |
| LPA- | AGAACUACUGCAGGAAUCC | 239 | GAAUCUGGAUUCCU | 639 | AGAACUACUG | 1039 |
| 4712 | AGAUTC | | GCAGUAGUUCUCG | | CAGGAAUCC | |
| LPA- | ACUACUGCAGGAAUCCAGA | 240 | CCAGAUUCUGGAUU | 640 | ACUACUGCAG | 1040 |
| 4715 | AUCUGG | | CCUGCAGUAGUUC | | GAAUCCAGA | |
| LPA- | UACUGCAGGAAUCCAGAUU | 241 | UCCCAUAAUCUGGA | 641 | UACUGCAGGA | 1041 |
| 4717 | AUGGGA | | UUCCUGCAGUAGU | | AUCCAGAUU | |
| LPA- | ACUGCAGGAAUCCAGAUUC | 242 | UUCCCUGAAUCUGG | 642 | ACUGCAGGAA | 1042 |
| 4718 | AGGGAA | | AUUCCUGCAGUAG | | UCCAGAUUC | |
| LPA- | CUGCAGGAAUCCAGAUUCU | 243 | UUUCCUAGAAUCUG | 643 | CUGCAGGAAU | 1043 |
| 4719 | AGGAAA | | GAUUCCUGCAGUA | | CCAGAUUCU | |
| LPA- | UGCAGGAAUCCAGAUUCUG | 244 | GUUUCUCAGAAUCU | 644 | UGCAGGAAUC | 1044 |
| 4720 | AGAAAC | | GGAUUCCUGCAGU | | CAGAUUCUG | |
| LPA- | GCAGGAAUCCAGAUUCUGG | 245 | UGUUUUCCAGAAUC | 645 | GCAGGAAUCC | 1045 |
| 4721 | AAAACA | | UGGAUUCCUGCAG | | AGAUUCUGG | |
| LPA- | GGAAUCCAGAUUCUGGGAA | 246 | GGUUGUUUCCCAGA | 646 | GGAAUCCAGA | 1046 |
| 4724 | ACAACC | | AUCUGGAUUCCUG | | UUCUGGGAA | |
| LPA- | GGGAAACAACCCUGGUGUU | 247 | UUGUGUAACACCAG | 647 | GGGAAACAAC | 1047 |
| 4738 | ACACAA | | GGUUGUUUCCCAG | | CCUGGUGUU | |
| LPA- | GGAAACAACCCUGGUGUUA | 248 | GUUGUUUAACACCA | 648 | GGAAACAACC | 1048 |
| 4739 | AACAAC | | GGGUUGUUUCCCA | | CUGGUGUUA | |
| LPA- | UGUGUGAGGUGGGAGUACU | 249 | GAUUGUAGUACUCC | 649 | UGUGUGAGGU | 1049 |
| 4771 | ACAATC | | CACCUCACACACG | | GGGAGUACU | |
| LPA- | GUGUGAGGUGGGAGUACUG | 250 | AGAUUUCAGUACUC | 650 | GUGUGAGGUG | 1050 |
| 4772 | AAAUCT | | CCACCUCACACAC | | GGAGUACUG | |
| LPA- | GUGAGGUGGGAGUACUGCA | 251 | UCAGAUUGCAGUAC | 651 | GUGAGGUGGG | 1051 |
| 4774 | AUCUGA | | UCCCACCUCACAC | | AGUACUGCA | |
| LPA- | UGAGGUGGGAGUACUGCAA | 252 | GUCAGUUUGCAGUA | 652 | UGAGGUGGGA | 1052 |
| 4775 | ACUGAC | | CUCCCACCUCACA | | GUACUGCAA | |
| LPA- | CUGACACAAUGCUCAGAAA | 253 | AUUCUUUUUCUGAG | 653 | CUGACACAAU | 1053 |
| 4795 | AAGAAT | | CAUUGUGUCAGAU | | GCUCAGAAA | |
| LPA- | UGACACAAUGCUCAGAAAC | 254 | GAUUCUGUUUCUGA | 654 | UGACACAAUG | 1054 |
| 4796 | AGAATC | | GCAUUGUGUCAGA | | CUCAGAAAC | |
| LPA- | GACACAAUGCUCAGAAACA | 255 | UGAUUUUGUUUCUG | 655 | GACACAAUGC | 1055 |
| 4797 | AAAUCA | | AGCAUUGUGUCAG | | UCAGAAACA | |
| LPA- | ACACAAUGCUCAGAAACAG | 256 | CUGAUUCUGUUUCU | 656 | ACACAAUGCU | 1056 |
| 4798 | AAUCAG | | GAGCAUUGUGUCA | | CAGAAACAG | |
| LPA- | CACAAUGCUCAGAAACAGA | 257 | CCUGAUUCUGUUUC | 657 | CACAAUGCUC | 1057 |
| 4799 | AUCAGG | | UGAGCAUUGUGUC | | AGAAACAGA | |
| LPA- | ACAAUGCUCAGAAACAGAA | 258 | ACCUGUUUCUGUUU | 658 | ACAAUGCUCA | 1058 |
| 4800 | ACAGGT | | CUGAGCAUUGUGU | | GAAACAGAA | |
| LPA- | CAAUGCUCAGAAACAGAAU | 259 | CACCUUAUUCUGUU | 659 | CAAUGCUCAG | 1059 |
| 4801 | AAGGTG | | UCUGAGCAUUGUG | | AAACAGAAU | |
| LPA- | AAUGCUCAGAAACAGAAUC | 260 | ACACCUGAUUCUGU | 660 | AAUGCUCAGA | 1060 |
| 4802 | AGGUGT | | UUCUGAGCAUUGU | | AACAGAAUC | |
| LPA- | AUGCUCAGAAACAGAAUCA | 261 | GACACUUGAUUCUG | 661 | AUGCUCAGAA | 1061 |
| 4803 | AGUGTC | | UUUCUGAGCAUUG | | ACAGAAUCA | |
| LPA- | UGCUCAGAAACAGAAUCAG | 262 | GGACAUCUGAUUCU | 662 | UGCUCAGAAA | 1062 |
| 4804 | AUGUCC | | GUUUCUGAGCAUU | | CAGAAUCAG | |
| LPA- | CUCAGAAACAGAAUCAGGU | 263 | UAGGAUACCUGAUU | 663 | CUCAGAAACA | 1063 |
| 4806 | AUCCTA | | CUGUUUCUGAGCA | | GAAUCAGGU | |
| LPA- | CAGAAACAGAAUCAGGUGU | 264 | UCUAGUACACCUGA | 664 | CAGAAACAGA | 1064 |
| 4808 | ACUAGA | | UUCUGUUUCUGAG | | AUCAGGUGU | |
| LPA- | AGAAACAGAAUCAGGUGUC | 265 | CUCUAUGACACCUG | 665 | AGAAACAGAA | 1065 |
| 4809 | AUAGAG | | AUUCUGUUUCUGA | | UCAGGUGUC | |
| LPA- | GAAACAGAAUCAGGUGUCC | 266 | UCUCUUGGACACCU | 666 | GAAACAGAAU | 1066 |
| 4810 | AAGAGA | | GAUUCUGUUUCUG | | CAGGUGUCC | |
| LPA- | AAACAGAAUCAGGUGUCCU | 267 | GUCUCUAGGACACC | 667 | AAACAGAAUC | 1067 |
| 4811 | AGAGAC | | UGAUUCUGUUUCU | | AGGUGUCCU | |
| LPA- | AACAGAAUCAGGUGUCCUA | 268 | AGUCUUUAGGACAC | 668 | AACAGAAUCA | 1068 |
| 4812 | AAGACT | | CUGAUUCUGUUUC | | GGUGUCCUA | |
| LPA- | CAGAAUCAGGUGUCCUAGA | 269 | GGAGUUUCUAGGAC | 669 | CAGAAUCAGG | 1069 |
| 4814 | AACUCC | | ACCUGAUUCUGUU | | UGUCCUAGA | |
| LPA- | GAAUCAGGUGUCCUAGAGA | 270 | UGGGAUUCUCUAGG | 670 | GAAUCAGGUG | 1070 |
| 4816 | AUCCCA | | ACACCUGAUUCUG | | UCCUAGAGA | |
| LPA- | AUCAGGUGUCCUAGAGACU | 271 | AGUGGUAGUCUCUA | 671 | AUCAGGUGUC | 1071 |
| 4818 | ACCACT | | GGACACCUGAUUC | | CUAGAGACU | |
| LPA- | GGUGUCCUAGAGACUCCCA | 272 | CAACAUUGGGAGUC | 672 | GGUGUCCUAG | 1072 |
| 4822 | AUGUTG | | UCUAGGACACCUG | | AGACUCCCA | |
| LPA- | CCUAGAGACUCCCACUGUU | 273 | UGGAAUAACAGUGG | 673 | CCUAGAGACU | 1073 |
| 4827 | AUUCCA | | GAGUCUCUAGGAC | | CCCACUGUU | |
| LPA- | CUAGAGACUCCCACUGUUG | 274 | CUGGAUCAACAGUG | 674 | CUAGAGACUC | 1074 |
| 4828 | AUCCAG | | GGAGUCUCUAGGA | | CCACUGUUG | |
| LPA- | UAGAGACUCCCACUGUUGU | 275 | ACUGGUACAACAGU | 675 | UAGAGACUCC | 1075 |
| 4829 | ACCAGT | | GGGAGUCUCUAGG | | CACUGUUGU | |
| LPA- | AGAGACUCCCACUGUUGUU | 276 | AACUGUAACAACAG | 676 | AGAGACUCCC | 1076 |
| 4830 | ACAGTT | | UGGGAGUCUCUAG | | ACUGUUGUU | |
| LPA- | GAGACUCCCACUGUUGUUC | 277 | GAACUUGAACAACA | 677 | GAGACUCCCA | 1077 |
| 4831 | AAGUTC | | GUGGGAGUCUCUA | | CUGUUGUUC | |
| LPA- | AGACUCCCACUGUUGUUCC | 278 | GGAACUGGAACAAC | 678 | AGACUCCCAC | 1078 |
| 4832 | AGUUCC | | AGUGGGAGUCUCU | | UGUUGUUCC | |
| LPA- | GCUCAUUCUGAAGCAGCAC | 279 | CAGUUUGUGCUGCU | 679 | GCUCAUUCUG | 1079 |
| 4867 | AAACTG | | UCAGAAUGAGCCU | | AAGCAGCAC | |
| LPA- | CUCAUUCUGAAGCAGCACC | 280 | UCAGUUGGUGCUGC | 680 | CUCAUUCUGA | 1080 |
| 4868 | AACUGA | | UUCAGAAUGAGCC | | AGCAGCACC | |
| LPA- | UCAUUCUGAAGCAGCACCA | 281 | CUCAGUUGGUGCUG | 681 | UCAUUCUGAA | 1081 |
| 4869 | ACUGAG | | CUUCAGAAUGAGC | | GCAGCACCA | |
| LPA- | CAUUCUGAAGCAGCACCAA | 282 | GCUCAUUUGGUGCU | 682 | CAUUCUGAAG | 1082 |
| 4870 | AUGAGC | | GCUUCAGAAUGAG | | CAGCACCAA | |
| LPA- | AUUCUGAAGCAGCACCAAC | 283 | UGCUCUGUUGGUGC | 683 | AUUCUGAAGC | 1083 |
| 4871 | AGAGCA | | UGCUUCAGAAUGA | | AGCACCAAC | |
| LPA- | UUCUGAAGCAGCACCAACU | 284 | UUGCUUAGUUGGUG | 684 | UUCUGAAGCA | 1084 |
| 4872 | AAGCAA | | CUGCUUCAGAAUG | | GCACCAACU | |
| LPA- | UCUGAAGCAGCACCAACUG | 285 | UUUGCUCAGUUGGU | 685 | UCUGAAGCAG | 1085 |
| 4873 | AGCAAA | | GCUGCUUCAGAAU | | CACCAACUG | |
| LPA- | CUGAAGCAGCACCAACUGA | 286 | GUUUGUUCAGUUGG | 686 | CUGAAGCAGC | 1086 |
| 4874 | ACAAAC | | UGCUGCUUCAGAA | | ACCAACUGA | |
| LPA- | UGAAGCAGCACCAACUGAG | 287 | GGUUUUCUCAGUUG | 687 | UGAAGCAGCA | 1087 |
| 4875 | AAAACC | | GUGCUGCUUCAGA | | CCAACUGAG | |
| LPA- | GAAGCAGCACCAACUGAGC | 288 | GGGUUUGCUCAGUU | 688 | GAAGCAGCAC | 1088 |
| 4876 | AAACCC | | GGUGCUGCUUCAG | | CAACUGAGC | |
| LPA- | AAGCAGCACCAACUGAGCA | 289 | GGGGUUUGCUCAGU | 689 | AAGCAGCACC | 1089 |
| 4877 | AACCCC | | UGGUGCUGCUUCA | | AACUGAGCA | |
| LPA- | CAGUGCUACCAUGGUAAUG | 290 | UCUGGUCAUUACCA | 690 | CAGUGCUACC | 1090 |
| 4912 | ACCAGA | | UGGUAGCACUGCC | | AUGGUAAUG | |
| LPA- | AGUGCUACCAUGGUAAUGG | 291 | CUCUGUCCAUUACC | 691 | AGUGCUACCA | 1091 |
| 4913 | ACAGAG | | AUGGUAGCACUGC | | UGGUAAUGG | |
| LPA- | ACAUUCUCCACCACUGUCA | 292 | UUCCUUUGACAGUG | 692 | ACAUUCUCCA | 1092 |
| 4948 | AAGGAA | | GUGGAGAAUGUGC | | CCACUGUCA | |
| LPA- | CACUGUCACAGGAAGGACA | 293 | UUGACUUGUCCUUC | 693 | CACUGUCACA | 1093 |
| 4959 | AGUCAA | | CUGUGACAGUGGU | | GGAAGGACA | |
| LPA- | ACUGUCACAGGAAGGACAU | 294 | AUUGAUAUGUCCUU | 694 | ACUGUCACAG | 1094 |
| 4960 | AUCAAT | | CCUGUGACAGUGG | | GAAGGACAU | |
| LPA- | CUGUCACAGGAAGGACAUG | 295 | GAUUGUCAUGUCCU | 695 | CUGUCACAGG | 1095 |
| 4961 | ACAATC | | UCCUGUGACAGUG | | AAGGACAUG | |
| LPA- | UGUCACAGGAAGGACAUGU | 296 | AGAUUUACAUGUCC | 696 | UGUCACAGGA | 1096 |
| 4962 | AAAUCT | | UUCCUGUGACAGU | | AGGACAUGU | |
| LPA- | GUCACAGGAAGGACAUGUC | 297 | AAGAUUGACAUGUC | 697 | GUCACAGGAA | 1097 |
| 4963 | AAUCTT | | CUUCCUGUGACAG | | GGACAUGUC | |
| LPA- | UCACAGGAAGGACAUGUCA | 298 | CAAGAUUGACAUGU | 698 | UCACAGGAAG | 1098 |
| 4964 | AUCUTG | | CCUUCCUGUGACA | | GACAUGUCA | |
| LPA- | ACAGGAAGGACAUGUCAAU | 299 | ACCAAUAUUGACAU | 699 | ACAGGAAGGA | 1099 |
| 4966 | AUUGGT | | GUCCUUCCUGUGA | | CAUGUCAAU | |
| LPA- | CAGGAAGGACAUGUCAAUC | 300 | GACCAUGAUUGACA | 700 | CAGGAAGGAC | 1100 |
| 4967 | AUGGTC | | UGUCCUUCCUGUG | | AUGUCAAUC | |
| LPA- | AGGAAGGACAUGUCAAUCU | 301 | UGACCUAGAUUGAC | 701 | AGGAAGGACA | 1101 |
| 4968 | AGGUCA | | AUGUCCUUCCUGU | | UGUCAAUCU | |
| LPA- | GGAAGGACAUGUCAAUCUU | 302 | AUGACUAAGAUUGA | 702 | GGAAGGACAU | 1102 |
| 4969 | AGUCAT | | CAUGUCCUUCCUG | | GUCAAUCUU | |
| LPA- | GAAGGACAUGUCAAUCUUG | 303 | GAUGAUCAAGAUUG | 703 | GAAGGACAUG | 1103 |
| 4970 | AUCATC | | ACAUGUCCUUCCU | | UCAAUCUUG | |
| LPA- | AAGGACAUGUCAAUCUUGG | 304 | GGAUGUCCAAGAUU | 704 | AAGGACAUGU | 1104 |
| 4971 | ACAUCC | | GACAUGUCCUUCC | | CAAUCUUGG | |
| LPA- | AGGACAUGUCAAUCUUGGU | 305 | UGGAUUACCAAGAU | 705 | AGGACAUGUC | 1105 |
| 4972 | AAUCCA | | UGACAUGUCCUUC | | AAUCUUGGU | |
| LPA- | GGACAUGUCAAUCUUGGUC | 306 | AUGGAUGACCAAGA | 706 | GGACAUGUCA | 1106 |
| 4973 | AUCCAT | | UUGACAUGUCCUU | | AUCUUGGUC | |
| LPA- | GACAUGUCAAUCUUGGUCA | 307 | CAUGGUUGACCAAG | 707 | GACAUGUCAA | 1107 |
| 4974 | ACCATG | | AUUGACAUGUCCU | | UCUUGGUCA | |
| LPA- | ACAUGUCAAUCUUGGUCAU | 308 | UCAUGUAUGACCAA | 708 | ACAUGUCAAU | 1108 |
| 4975 | ACAUGA | | GAUUGACAUGUCC | | CUUGGUCAU | |
| LPA- | CAUGUCAAUCUUGGUCAUC | 309 | GUCAUUGAUGACCA | 709 | CAUGUCAAUC | 1109 |
| 4976 | AAUGAC | | AGAUUGACAUGUC | | UUGGUCAUC | |
| LPA- | AUGUCAAUCUUGGUCAUCC | 310 | UGUCAUGGAUGACC | 710 | AUGUCAAUCU | 1110 |
| 4977 | AUGACA | | AAGAUUGACAUGU | | UGGUCAUCC | |
| LPA- | UGUCAAUCUUGGUCAUCCA | 311 | GUGUCUUGGAUGAC | 711 | UGUCAAUCUU | 1111 |
| 4978 | AGACAC | | CAAGAUUGACAUG | | GGUCAUCCA | |
| LPA- | GUCAAUCUUGGUCAUCCAU | 312 | GGUGUUAUGGAUGA | 712 | GUCAAUCUUG | 1112 |
| 4979 | AACACC | | CCAAGAUUGACAU | | GUCAUCCAU | |
| LPA- | UCAAUCUUGGUCAUCCAUG | 313 | UGGUGUCAUGGAUG | 713 | UCAAUCUUGG | 1113 |
| 4980 | ACACCA | | ACCAAGAUUGACA | | UCAUCCAUG | |
| LPA- | CAAUCUUGGUCAUCCAUGA | 314 | GUGGUUUCAUGGAU | 714 | CAAUCUUGGU | 1114 |
| 4981 | AACCAC | | GACCAAGAUUGAC | | CAUCCAUGA | |
| LPA- | AAUCUUGGUCAUCCAUGAC | 315 | UGUGGUGUCAUGGA | 715 | AAUCUUGGUC | 1115 |
| 4982 | ACCACA | | UGACCAAGAUUGA | | AUCCAUGAC | |
| LPA- | AUCUUGGUCAUCCAUGACA | 316 | GUGUGUUGUCAUGG | 716 | AUCUUGGUCA | 1116 |
| 4983 | ACACAC | | AUGACCAAGAUUG | | UCCAUGACA | |
| LPA- | UGACAAUGAACUACUGCAG | 317 | GGAUUUCUGCAGUA | 717 | UGACAAUGAA | 1117 |
| 5048 | AAAUCC | | GUUCAUUGUCAGG | | CUACUGCAG | |
| LPA- | GACAAUGAACUACUGCAGG | 318 | UGGAUUCCUGCAGU | 718 | GACAAUGAAC | 1118 |
| 5049 | AAUCCA | | AGUUCAUUGUCAG | | UACUGCAGG | |
| LPA- | ACAAUGAACUACUGCAGGA | 319 | CUGGAUUCCUGCAG | 719 | ACAAUGAACU | 1119 |
| 5050 | AUCCAG | | UAGUUCAUUGUCA | | ACUGCAGGA | |
| LPA- | CAAUGAACUACUGCAGGAA | 320 | UCUGGUUUCCUGCA | 720 | CAAUGAACUA | 1120 |
| 5051 | ACCAGA | | GUAGUUCAUUGUC | | CUGCAGGAA | |
| LPA- | AAUGAACUACUGCAGGAAU | 321 | AUCUGUAUUCCUGC | 721 | AAUGAACUAC | 1121 |
| 5052 | ACAGAT | | AGUAGUUCAUUGU | | UGCAGGAAU | |
| LPA- | AUGAACUACUGCAGGAAUC | 322 | CAUCUUGAUUCCUG | 722 | AUGAACUACU | 1122 |
| 5053 | AAGATG | | CAGUAGUUCAUUG | | GCAGGAAUC | |
| LPA- | UGAACUACUGCAGGAAUCC | 323 | GCAUCUGGAUUCCU | 723 | UGAACUACUG | 1123 |
| 5054 | AGAUGC | | GCAGUAGUUCAUU | | CAGGAAUCC | |
| LPA- | CUACUGCAGGAAUCCAGAU | 324 | AUCGGUAUCUGGAU | 724 | CUACUGCAGG | 1124 |
| 5058 | ACCGAT | | UCCUGCAGUAGUU | | AAUCCAGAU | |
| LPA- | CAGGCCCUUGGUGUUUUAC | 325 | UCCAUUGUAAAACA | 725 | CAGGCCCUUG | 1125 |
| 5084 | AAUGGA | | CCAAGGGCCUGUA | | GUGUUUUAC | |
| LPA- | CUUGGUGUUUUACCAUGGA | 326 | CUGGGUUCCAUGGU | 726 | CUUGGUGUUU | 1126 |
| 5090 | ACCCAG | | AAAACACCAAGGG | | UACCAUGGA | |
| LPA- | UUGGUGUUUUACCAUGGAC | 327 | GCUGGUGUCCAUGG | 727 | UUGGUGUUUU | 1127 |
| 5091 | ACCAGC | | UAAAACACCAAGG | | ACCAUGGAC | |
| LPA- | UGGUGUUUUACCAUGGACC | 328 | UGCUGUGGUCCAUG | 728 | UGGUGUUUUA | 1128 |
| 5092 | ACAGCA | | GUAAAACACCAAG | | CCAUGGACC | |
| LPA- | GGUGUUUUACCAUGGACCC | 329 | AUGCUUGGGUCCAU | 729 | GGUGUUUUAC | 1129 |
| 5093 | AAGCAT | | GGUAAAACACCAA | | CAUGGACCC | |
| LPA- | GUGUUUUACCAUGGACCCC | 330 | GAUGCUGGGGUCCA | 730 | GUGUUUUACC | 1130 |
| 5094 | AGCATC | | UGGUAAAACACCA | | AUGGACCCC | |
| LPA- | GUUUUACCAUGGACCCCAG | 331 | CUGAUUCUGGGGUC | 731 | GUUUUACCAU | 1131 |
| 5096 | AAUCAG | | CAUGGUAAAACAC | | GGACCCCAG | |
| LPA- | GGAGUACUGCAACCUGACG | 332 | GCAUCUCGUCAGGU | 732 | GGAGUACUGC | 1132 |
| 5124 | AGAUGC | | UGCAGUACUCCCA | | AACCUGACG | |
| LPA- | GAGUACUGCAACCUGACGC | 333 | AGCAUUGCGUCAGG | 733 | GAGUACUGCA | 1133 |
| 5125 | AAUGCT | | UUGCAGUACUCCC | | ACCUGACGC | |
| LPA- | GUACUGCAACCUGACGCGA | 334 | UGAGCUUCGCGUCA | 734 | GUACUGCAAC | 1134 |
| 5127 | AGCUCA | | GGUUGCAGUACUC | | CUGACGCGA | |
| LPA- | UACUGCAACCUGACGCGAU | 335 | CUGAGUAUCGCGUC | 735 | UACUGCAACC | 1135 |
| 5128 | ACUCAG | | AGGUUGCAGUACU | | UGACGCGAU | |
| LPA- | UGCAACCUGACGCGAUGCU | 336 | UGUCUUAGCAUCGC | 736 | UGCAACCUGA | 1136 |
| 5131 | AAGACA | | GUCAGGUUGCAGU | | CGCGAUGCU | |
| LPA- | CCUGACGCGAUGCUCAGAC | 337 | UUCUGUGUCUGAGC | 737 | CCUGACGCGA | 1137 |
| 5136 | ACAGAA | | AUCGCGUCAGGUU | | UGCUCAGAC | |
| LPA- | CUGACGCGAUGCUCAGACA | 338 | CUUCUUUGUCUGAG | 738 | CUGACGCGAU | 1138 |
| 5137 | AAGAAG | | CAUCGCGUCAGGU | | GCUCAGACA | |
| LPA- | GAUGCUCAGACACAGAAGG | 339 | ACAGUUCCUUCUGU | 739 | GAUGCUCAGA | 1139 |
| 5144 | AACUGT | | GUCUGAGCAUCGC | | CACAGAAGG | |
| LPA- | AUGCUCAGACACAGAAGGG | 340 | CACAGUCCCUUCUG | 740 | AUGCUCAGAC | 1140 |
| 5145 | ACUGTG | | UGUCUGAGCAUCG | | ACAGAAGGG | |
| LPA- | AGACACAGAAGGGACUGUG | 341 | AGCGAUCACAGUCC | 741 | AGACACAGAA | 1141 |
| 5151 | AUCGCT | | CUUCUGUGUCUGA | | GGGACUGUG | |
| LPA- | GCAUCCUCUUCAUUUGAUU | 342 | UCCCAUAAUCAAAU | 742 | GCAUCCUCUU | 1142 |
| 5467 | AUGGGA | | GAAGAGGAUGCAC | | CAUUUGAUU | |
| LPA- | CAUCCUCUUCAUUUGAUUG | 343 | UUCCCUCAAUCAAA | 743 | CAUCCUCUUC | 1143 |
| 5468 | AGGGAA | | UGAAGAGGAUGCA | | AUUUGAUUG | |
| LPA- | AUCCUCUUCAUUUGAUUGU | 344 | CUUCCUACAAUCAA | 744 | AUCCUCUUCA | 1144 |
| 5469 | AGGAAG | | AUGAAGAGGAUGC | | UUUGAUUGU | |
| LPA- | UCCUCUUCAUUUGAUUGUG | 345 | GCUUCUCACAAUCA | 745 | UCCUCUUCAU | 1145 |
| 5470 | AGAAGC | | AAUGAAGAGGAUG | | UUGAUUGUG | |
| LPA- | CCUCUUCAUUUGAUUGUGG | 346 | GGCUUUCCACAAUC | 746 | CCUCUUCAUU | 1146 |
| 5471 | AAAGCC | | AAAUGAAGAGGAU | | UGAUUGUGG | |
| LPA- | CUUCAUUUGAUUGUGGGAA | 347 | UGAGGUUUCCCACA | 747 | CUUCAUUUGA | 1147 |
| 5474 | ACCUCA | | AUCAAAUGAAGAG | | UUGUGGGAA | |
| LPA- | UUCAUUUGAUUGUGGGAAG | 348 | UUGAGUCUUCCCAC | 748 | UUCAUUUGAU | 1148 |
| 5475 | ACUCAA | | AAUCAAAUGAAGA | | UGUGGGAAG | |
| LPA- | UCAUUUGAUUGUGGGAAGC | 349 | CUUGAUGCUUCCCA | 749 | UCAUUUGAUU | 1149 |
| 5476 | AUCAAG | | CAAUCAAAUGAAG | | GUGGGAAGC | |
| LPA- | CAUUUGAUUGUGGGAAGCC | 350 | ACUUGUGGCUUCCC | 750 | CAUUUGAUUG | 1150 |
| 5477 | ACAAGT | | ACAAUCAAAUGAA | | UGGGAAGCC | |
| LPA- | AUUUGAUUGUGGGAAGCCU | 351 | CACUUUAGGCUUCC | 751 | AUUUGAUUGU | 1151 |
| 5478 | AAAGTG | | CACAAUCAAAUGA | | GGGAAGCCU | |
| LPA- | GUGGGAAGCCUCAAGUGGA | 352 | UUCGGUUCCACUUG | 752 | GUGGGAAGCC | 1152 |
| 5486 | ACCGAA | | AGGCUUCCCACAA | | UCAAGUGGA | |
| LPA- | AAGAAAUGUCCUGGAAGCA | 353 | CUACAUUGCUUCCA | 753 | AAGAAAUGUC | 1153 |
| 5509 | AUGUAG | | GGACAUUUCUUCG | | CUGGAAGCA | |
| LPA- | AGAAAUGUCCUGGAAGCAU | 354 | CCUACUAUGCUUCC | 754 | AGAAAUGUCC | 1154 |
| 5510 | AGUAGG | | AGGACAUUUCUUC | | UGGAAGCAU | |
| LPA- | GAAAUGUCCUGGAAGCAUU | 355 | CCCUAUAAUGCUUC | 755 | GAAAUGUCCU | 1155 |
| 5511 | AUAGGG | | CAGGACAUUUCUU | | GGAAGCAUU | |
| LPA- | AAUGUCCUGGAAGCAUUGU | 356 | CCCCCUACAAUGCU | 756 | AAUGUCCUGG | 1156 |
| 5513 | AGGGGG | | UCCAGGACAUUUC | | AAGCAUUGU | |
| LPA- | AUGUCCUGGAAGCAUUGUA | 357 | CCCCCUUACAAUGC | 757 | AUGUCCUGGA | 1157 |
| 5514 | AGGGGG | | UUCCAGGACAUUU | | AGCAUUGUA | |
| LPA- | AGAACAAGGUUUGGAAAGC | 358 | AGAAGUGCUUUCCA | 758 | AGAACAAGGU | 1158 |
| 5581 | ACUUCT | | AACCUUGUUCUGA | | UUGGAAAGC | |
| LPA- | GAACAAGGUUUGGAAAGCA | 359 | CAGAAUUGCUUUCC | 759 | GAACAAGGUU | 1159 |
| 5582 | AUUCTG | | AAACCUUGUUCUG | | UGGAAAGCA | |
| LPA- | AACAAGGUUUGGAAAGCAC | 360 | ACAGAUGUGCUUUC | 760 | AACAAGGUUU | 1160 |
| 5583 | AUCUGT | | CAAACCUUGUUCU | | GGAAAGCAC | |
| LPA- | ACAAGGUUUGGAAAGCACU | 361 | CACAGUAGUGCUUU | 761 | ACAAGGUUUG | 1161 |
| 5584 | ACUGTG | | CCAAACCUUGUUC | | GAAAGCACU | |
| LPA- | CAAGGUUUGGAAAGCACUU | 362 | CCACAUAAGUGCUU | 762 | CAAGGUUUGG | 1162 |
| 5585 | AUGUGG | | UCCAAACCUUGUU | | AAAGCACUU | |
| LPA- | AAGGUUUGGAAAGCACUUC | 363 | UCCACUGAAGUGCU | 763 | AAGGUUUGGA | 1163 |
| 5586 | AGUGGA | | UUCCAAACCUUGU | | AAGCACUUC | |
| LPA- | AGGUUUGGAAAGCACUUCU | 364 | CUCCAUAGAAGUGC | 764 | AGGUUUGGAA | 1164 |
| 5587 | AUGGAG | | UUUCCAAACCUUG | | AGCACUUCU | |
| LPA- | UGGAAAGCACUUCUGUGGA | 365 | GGUGCUUCCACAGA | 765 | UGGAAAGCAC | 1165 |
| 5592 | AGCACC | | AGUGCUUUCCAAA | | UUCUGUGGA | |
| LPA- | GUGGAGGCACCUUAAUAUC | 366 | UCUGGUGAUAUUAA | 766 | GUGGAGGCAC | 1166 |
| 5606 | ACCAGA | | GGUGCCUCCACAG | | CUUAAUAUC | |
| LPA- | CUUAAUAUCCCCAGAGUGG | 367 | CAGCAUCCACUCUG | 767 | CUUAAUAUCC | 1167 |
| 5616 | AUGCTG | | GGGAUAUUAAGGU | | CCAGAGUGG | |
| LPA- | UAAUAUCCCCAGAGUGGGU | 36 | GUCAGUACCCACUC | 768 | UAAUAUCCCC | 1168 |
| 5618 | ACUGAC | | UGGGGAUAUUAAG | | AGAGUGGGU | |
| LPA- | AGAGUGGGUGCUGACUGCU | 369 | GUGAGUAGCAGUCA | 769 | AGAGUGGGUG | 1169 |
| 5628 | ACUCAC | | GCACCCACUCUGG | | CUGACUGCU | |
| LPA- | CAAGGUCAUCCUGGGUGCA | 370 | UUGGUUUGCACCCA | 770 | CAAGGUCAUC | 1170 |
| 5685 | AACCAA | | GGAUGACCUUGUA | | CUGGGUGCA | |
| LPA- | CCUGGGUGCACACCAAGAA | 371 | GUUCAUUUCUUGGU | 771 | CCUGGGUGCA | 1171 |
| 5694 | AUGAAC | | GUGCACCCAGGAU | | CACCAAGAA | |
| LPA- | GUGCACACCAAGAAGUGAA | 372 | UCGAGUUUCACUUC | 772 | GUGCACACCA | 1172 |
| 5699 | ACUCGA | | UUGGUGUGCACCC | | AGAAGUGAA | |
| LPA- | AGCAGAUAUUGCCUUGCUA | 373 | UAGCUUUAGCAAGG | 773 | AGCAGAUAUU | 1173 |
| 5775 | AAGCTA | | CAAUAUCUGCUUG | | GCCUUGCUA | |
| LPA- | GCAGAUAUUGCCUUGCUAA | 374 | UUAGCUUUAGCAAG | 774 | GCAGAUAUUG | 1174 |
| 5776 | AGCUAA | | GCAAUAUCUGCUU | | CCUUGCUAA | |
| LPA- | CAGAUAUUGCCUUGCUAAA | 375 | CUUAGUUUUAGCAA | 775 | CAGAUAUUGC | 1175 |
| 5777 | ACUAAG | | GGCAAUAUCUGCU | | CUUGCUAAA | |
| LPA- | AGAUAUUGCCUUGCUAAAG | 376 | GCUUAUCUUUAGCA | 776 | AGAUAUUGCC | 1176 |
| 5778 | AUAAGC | | AGGCAAUAUCUGC | | UUGCUAAAG | |
| LPA- | GAUAUUGCCUUGCUAAAGC | 377 | UGCUUUGCUUUAGC | 777 | GAUAUUGCCU | 1177 |
| 5779 | AAAGCA | | AAGGCAAUAUCUG | | UGCUAAAGC | |
| LPA- | AUAUUGCCUUGCUAAAGCU | 378 | CUGCUUAGCUUUAG | 778 | AUAUUGCCUU | 1178 |
| 5780 | AAGCAG | | CAAGGCAAUAUCU | | GCUAAAGCU | |
| LPA- | UAUUGCCUUGCUAAAGCUA | 379 | CCUGCUUAGCUUUA | 779 | UAUUGCCUUG | 1179 |
| 5781 | AGCAGG | | GCAAGGCAAUAUC | | CUAAAGCUA | |
| LPA- | UCAUCACUGACAAAGUAAU | 380 | GCUGGUAUUACUUU | 780 | UCAUCACUGA | 1180 |
| 5813 | ACCAGC | | GUCAGUGAUGACG | | CAAAGUAAU | |
| LPA- | GGACUGAAUGUUACAUCAC | 381 | CAGCCUGUGAUGUA | 781 | GGACUGAAUG | 1181 |
| 5873 | AGGCTG | | ACAUUCAGUCCUG | | UUACAUCAC | |
| LPA- | GACUGAAUGUUACAUCACU | 382 | CCAGCUAGUGAUGU | 782 | GACUGAAUGU | 1182 |
| 5874 | AGCUGG | | AACAUUCAGUCCU | | UACAUCACU | |
| LPA- | ACUGAAUGUUACAUCACUG | 383 | CCCAGUCAGUGAUG | 783 | ACUGAAUGUU | 1183 |
| 5875 | ACUGGG | | UAACAUUCAGUCC | | ACAUCACUG | |
| LPA- | CUGAAUGUUACAUCACUGG | 384 | CCCCAUCCAGUGAU | 784 | CUGAAUGUUA | 1184 |
| 5876 | AUGGGG | | GUAACAUUCAGUC | | CAUCACUGG | |
| LPA- | UGAAUGUUACAUCACUGGC | 385 | UCCCCUGCCAGUGA | 785 | UGAAUGUUAC | 1185 |
| 5877 | AGGGGA | | UGUAACAUUCAGU | | AUCACUGGC | |
| LPA- | AAUGUUACAUCACUGGCUG | 386 | UCUCCUCAGCCAGU | 786 | AAUGUUACAU | 1186 |
| 5879 | AGGAGA | | GAUGUAACAUUCA | | CACUGGCUG | |
| LPA- | GAAACCCAAGGUACCUUUG | 387 | CAGUCUCAAAGGUA | 787 | GAAACCCAAG | 1187 |
| 5902 | AGACTG | | CCUUGGGUUUCUC | | GUACCUUUG | |
| Control | | | | | | |
| Gal | GACAACAGAAUAUUAUCCA | 1188 | UUGGAUAAUAUUCU | 1189 | GACAACAGAA | 1190 |
| XC-LPA- | AGCAGCCGAAAGGCUGC | | GUUGUCGG | | UAUUAUCCA | |
| 3675 | | | | | | |
| NC1 | CGUUAAUCGCGUAUAAUAC | 1191 | AUACGCGUAUUAUA | 1192 | N/A | |
| | GCGUAT | | CGCGAUUAACGAC | | | |
| NC5 | CAUAUUGCGCGUAUAGUCG | 1193 | CUAACGCGACUAUA | 1194 | N/A | |
| | CGUUAG | | CGCGCAAUAUGGU | | | |
| NC7 | GGCGCGUAUAGUCGCGCGU | 1195 | GACUAUACGCGCGA | 1196 | N/A | |
| | AUAGTC | | CUAUACGCGCCUC | | | |

### In Vitro Cell-Based Assays

The ability of each of the DsiRNAs listed in Table 2 to inhibit LPA expression was determined using in vitro cell-based assays. Briefly, human embryonic kidney 293 (HEK293) or HepG2 cells stably expressing a human LPA gene were transfected with each of the DsiRNAs listed in Table 2 at 0.5 nM in separate wells of a multi-well cell-culture plate. Cells were maintained for 24 hours following transfection, and then the amount of remaining LPA mRNA from the transfected cells was determined using TAQMAN®-based qPCR assays. Two qPCR assays, a 3′ assay and a 5′ assay, were used to determine LPA mRNA levels as measured using PCR probes conjugated to 6-carboxyfluorescein (6-FAM).

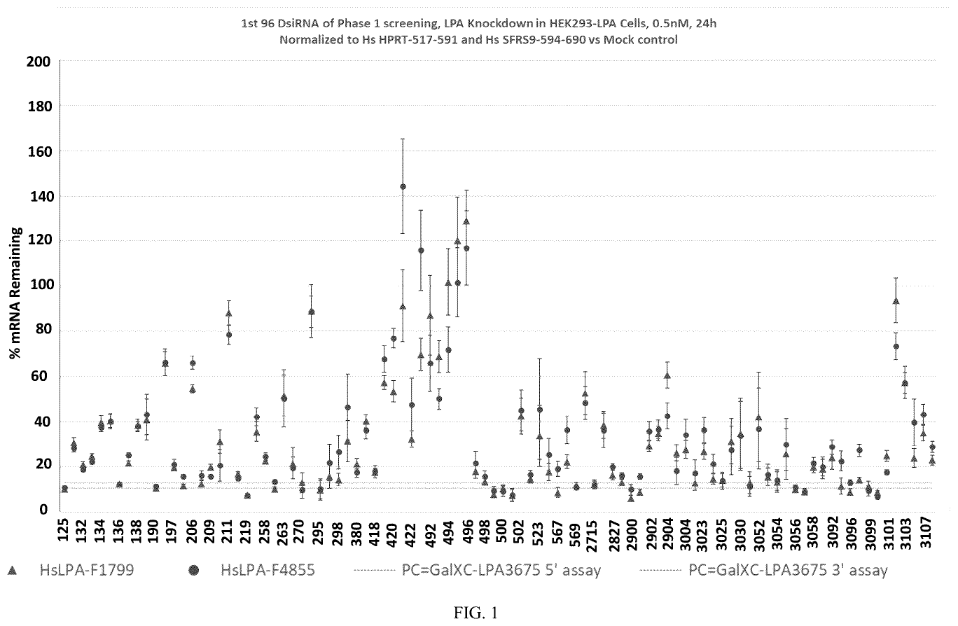

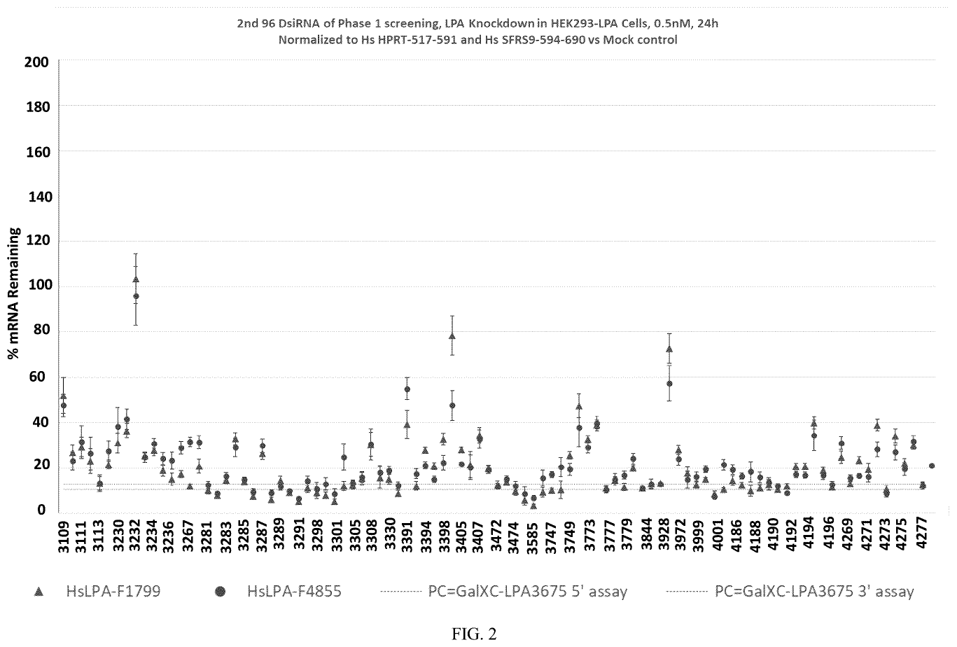

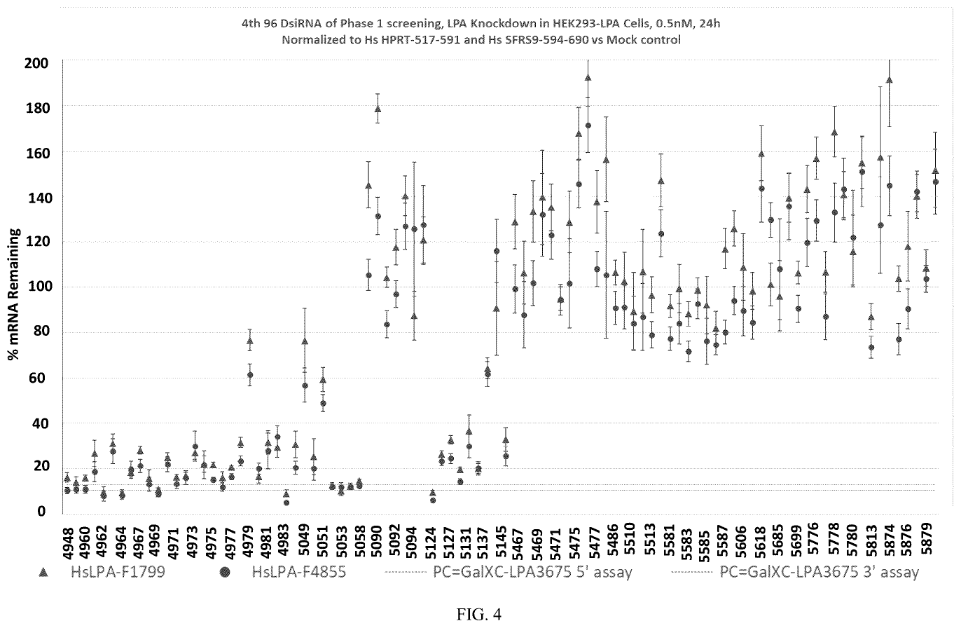

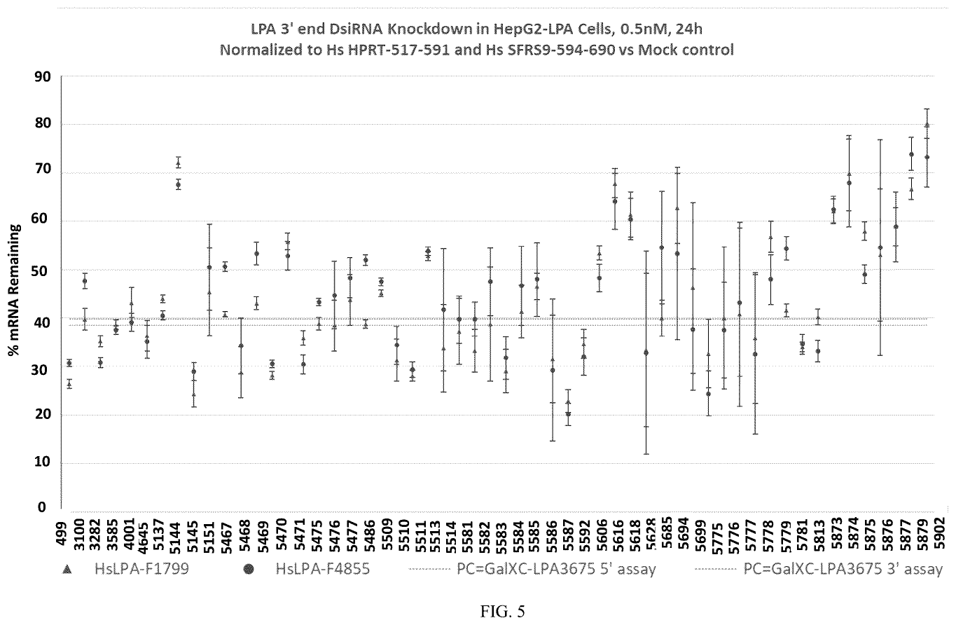

The results of the HEK293 and HepG2 cell-based assays to evaluate the ability of the DsiRNAs listed in Table 2 to inhibit LPA expression are shown in FIGS. 1-4 and FIG. 5, respectively. Cells transfected with a GalNAc-conjugated LPA oligonucleotide (GalXC-LPA-3675 SEQ ID NO: 1188 and 1189) were used as a positive control. DsiRNAs that resulted in less than or equal to about 15%-20% LPA mRNA remaining in DsiRNA-transfected cells when compared to mock-transfected cells were generally considered to comprise sequences that provide a suitable amount of knockdown or reduction of target mRNA expression for further evaluation. In FIGS. 1-5, the percent of LPA mRNA remaining in cells transfected with DsiRNAs, as indicated, relative to time-matched control cells is shown (3′ assay=circle shapes; 5′ assay=triangle shapes).

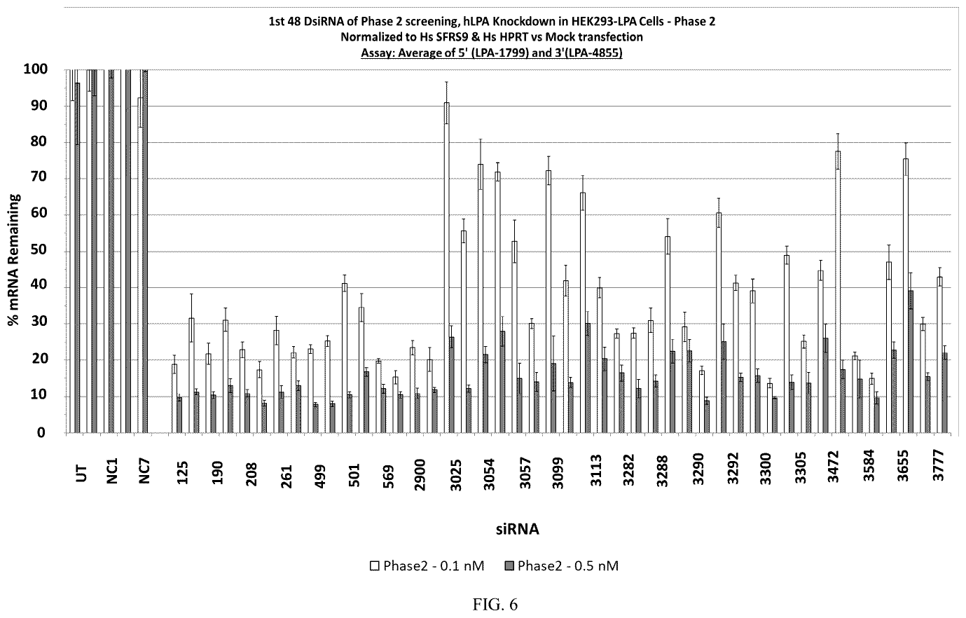

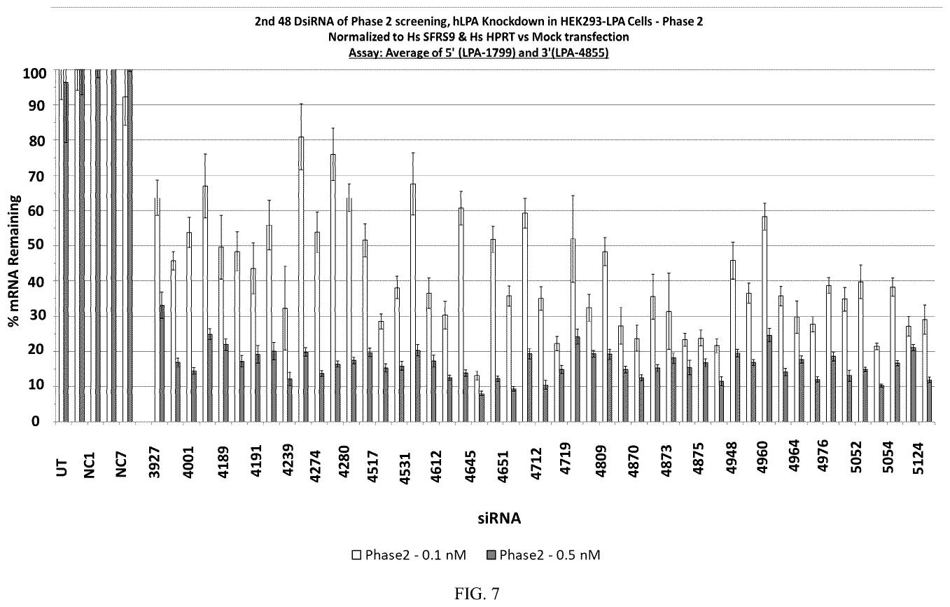

To further evaluate the DsiRNA hits, a subset of the DsiRNAs listed in Table 2 were tested to determine their ability to inhibit LPA expression using in vitro cell-based assays at two different DsiRNA concentrations (FIG. 6 and FIG. 7). Briefly, HEK293 cells stably expressing a human LPA gene were transfected with DsiRNAs at 0.1 nM and 0.5 nM in separate wells of a multi-well cell-culture plate. Cells were maintained for 24 hr. following transfection, and then the amount of remaining LPA mRNA from the transfected cells was determined using TAQMAN®-based qPCR assays. Two qPCR assays, a 3′ assay and a 5′ assay, were used to determine LPA mRNA levels as measured using PCR probes conjugated to hexachloro-fluorescein (HEX). Untransfected cells (UT), mock-transfected cells (Mock), and cells transfected with control oligonucleotides (NC1, SEQ ID NOs: 1191 and 1192; NC5, SEQ ID NO: 1193 and 1194; and NC7, SEQ ID NO: 1195 and 1196) were used as negative controls. As shown in FIGS. 6 and 7, the percent of LPA mRNA remaining in HEK293 cells transfected with the indicated DsiRNAs is an average of the LPA mRNA levels from the 3′ assay and 5′ assay and is normalized to time-matched, mock-transfected control HEK293 cells.

Taken together, these results show that DsiRNAs designed to target human LPA mRNA inhibit LPA expression in cells, as determined by a reduced amount of LPA mRNA in DsiRNA-transfected cells relative to control cells. These results demonstrate that the nucleotide sequences comprising the DsiRNA are useful for generating RNAi oligonucleotides to inhibit LPA expression. Further, these results demonstrate that multiple LPA mRNA target sequences are suitable for the RNAi-mediated inhibition of LPA expression.

### Example 3: RNAi Oligonucleotide Inhibition of LPA Expression In Vivo

Of the DsiRNAs screened in the cell-based assays described in Example 2, the nucleotide sequences of 14 DsiRNAs were selected for further evaluation in vivo. Briefly, the nucleotide sequences of the 14 selected DsiRNAs were used to generate 14 corresponding double-stranded RNAi oligonucleotides comprising a nicked tetraloop GalNAc-conjugated structure (referred to herein as “GalNAc-conjugated LPA oligonucleotides”) having a 36-mer passenger strand and a 22-mer guide strand (Table 3). Further, the nucleotide sequences comprising the passenger strand and guide strand of the GalNAc-conjugated LPA oligonucleotides have a distinct pattern of modified nucleotides and phosphorothioate linkages (e.g., see FIG. 10 for a schematic of the generic structure and chemical modification patterns (M1, M2, and M3) of the GalNAc-conjugated LPA oligonucleotides). The three adenosine nucleotides comprising the tetraloop are each conjugated to a GalNAc moiety (CAS #: 14131-60-3).

TABLE 3
GalNAc-Conjugated LPA Oligonucleotides Evaluated in Mice

| | | SEQ ID NO | SEQ ID NO |
|---|---|---|---|
| Oligonucleotide | DP # | (Sense) | (Antisense) |
| LPA-0190-M1 | DP15791P: DP15790G | 388 | 788 |
| LPA-0501-M1 | DP15634P: DP15633G | 389 | 789 |
| LPA-3100-M1 | DP15639P: DP15638G | 390 | 790 |
| LPA-3286-M1 | DP15643P: DP15642G | 391 | 791 |
| LPA-3288-M1 | DP15645P: DP15644G | 392 | 792 |
| LPA-3291-M1 | DP15647P: DP15646G | 393 | 793 |
| LPA-3584-M1 | DP15651P: DP15650G | 394 | 794 |
| LPA-3585-M1 | DP15653P: DP15652G | 395 | 795 |
| LPA-4645-M1 | DP15657P: DP15656G | 396 | 796 |
| LPA-4717-M1 | DP15801P: DP15800G | 397 | 797 |
| LPA-5510-M1 | DP15815P: DP15814G | 398 | 798 |
| LPA-3750-M1 | DP13346P: DP13385G | 399 | 799 |
| LPA-2900-M2 | DP13351P: DP14623G | 400 | 800 |
| LPA-3675-M2 | DP13346P: DP14624G | 401 | 801 |
| LPA-2900-M3 | DP13351P: DP13387G | 402 | 802 |
| LPA-3675-M3 | DP13346P: DP13385G | 403 | 803 |

### Mouse Studies

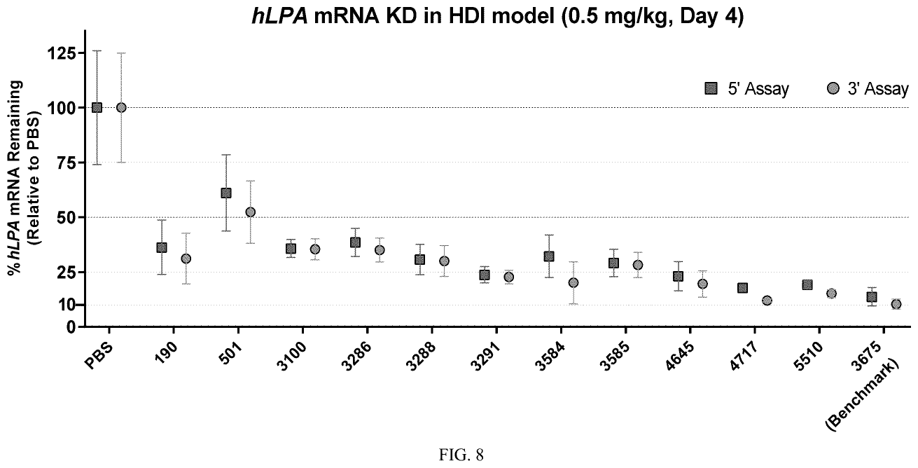

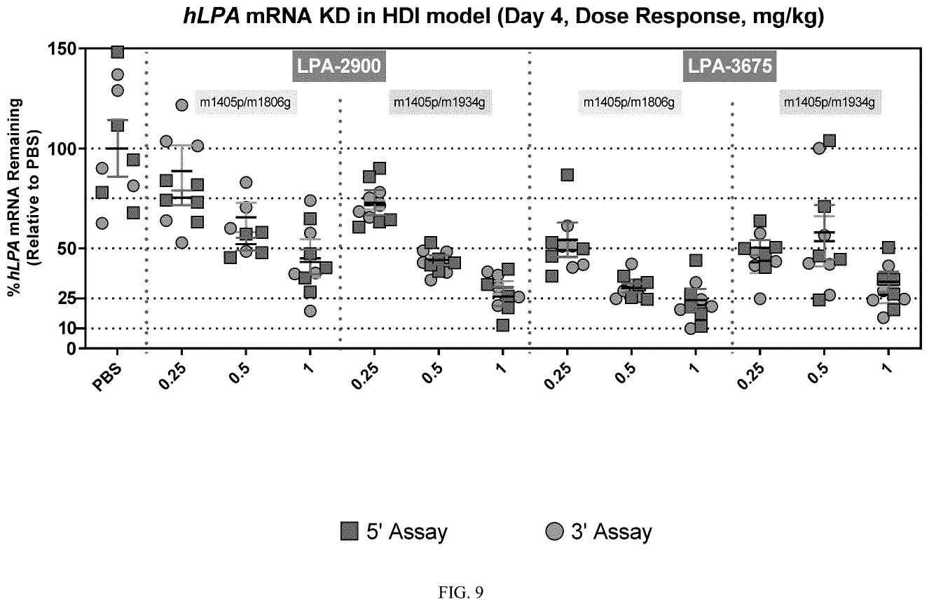

The GalNAc-conjugated LPA oligonucleotides listed in Table 3 were evaluated in an HDI mouse model, wherein HDI mice were engineered to transiently express human LPA mRNA in hepatocytes. The GalNAc-conjugated LPA oligonucleotide LPA-3675-M2 was used as a benchmark control. Briefly, 6-8-week-old female CD-1 mice (n=5) were treated subcutaneously with the indicated GalNAc-conjugated LPA oligonucleotides at a dose level of 0.5 mg/kg (FIG. 8) or at a dose level of 0.25 mg/kg, 0.5 mg/kg, and 1 mg/kg (FIG. 9). Three days later (72 h), the mice were hydrodynamically injected (HDI) with a DNA plasmid encoding the full human LPA gene under control of a ubiquitous cytomegalovirus (CMV) promoter sequence. One day after introduction of the DNA plasmid, liver samples from mice were collected. Total RNA derived from these mice were subjected to qRT-PCR analysis for LPA mRNA, relative to mice treated only with an identical volume of PBS. The values were normalized for transfection efficiency using the NeoR gene included on the plasmid.

As shown in FIG. 8, the indicated GalNAc-conjugated LPA oligonucleotides inhibited LPA expression, as determined by a reduction in the amount of LPA mRNA in liver samples from oligonucleotide-treated HDI mice relative to mice treated with PBS. To further evaluate the ability of GalNAc-conjugated LPA oligonucleotides to inhibit LPA expression, two of the GalNAc-conjugated LPA oligonucleotide sequences (LPA-2900 and LPA-3675) each having a different chemical modification pattern (M2 and M3) were tested for their ability to inhibit LPA expression in the HDI mice described above at three different concentrations (0.25 mg/kg, 0.5 mg/kg, and 1.0 mg/kg). As shown in FIG. 9, the indicated GalNAc-conjugated LPA oligonucleotides inhibited LPA expression in HDI mice in a dose-dependent manner.

Taken together, these results show that GalNAc-conjugated LPA oligonucleotides designed to target human LPA mRNA inhibit LPA expression in mice, as determined by a reduction in the amount of LPA mRNA in HDI mouse livers relative to control mice treated with PBS. Based on these results, 10 of the 14 GalNAc-conjugated LPA oligonucleotides evaluated in HDI mice were selected for evaluation of their ability to inhibit LPA expression in non-human primates (NHPs). The 10 GalNAc-conjugated LPA oligonucleotides listed in Table 4 comprise chemically modified nucleotides having pattern M1, M2, or M3 as described in FIG. 10.

TABLE 4
GalNAc-Conjugated LPA Oligonucleotides Evaluated in NHPs

| | | SEQ ID NO | SEQ ID NO |
|---|---|---|---|
| Oligonucleotide | DP # | (Sense) | (Antisense) |
| LPA-0190-M1 | DP15791P: DP15790G | 388 | 788 |
| LPA-3100-M1 | DP15639P: DP15638G | 390 | 790 |
| LPA-3288-M1 | DP15645P: DP15644G | 392 | 792 |
| LPA-3291-M1 | DP15647P: DP15646G | 393 | 793 |
| LPA-3585-M1 | DP15653P: DP15652G | 395 | 795 |
| LPA-4645-M1 | DP15657P: DP15656G | 396 | 796 |
| LPA-4717-M1 | DP15801P: DP15800G | 397 | 797 |
| LPA-5510-M1 | DP15815P: DP15814G | 398 | 798 |
| LPA-2900-M2 | DP13351P: DP14623G | 400 | 800 |
| LPA-3675-M3 | DP13346P: DP13385G | 403 | 803 |

### Non-Human Primate (NHP) Studies

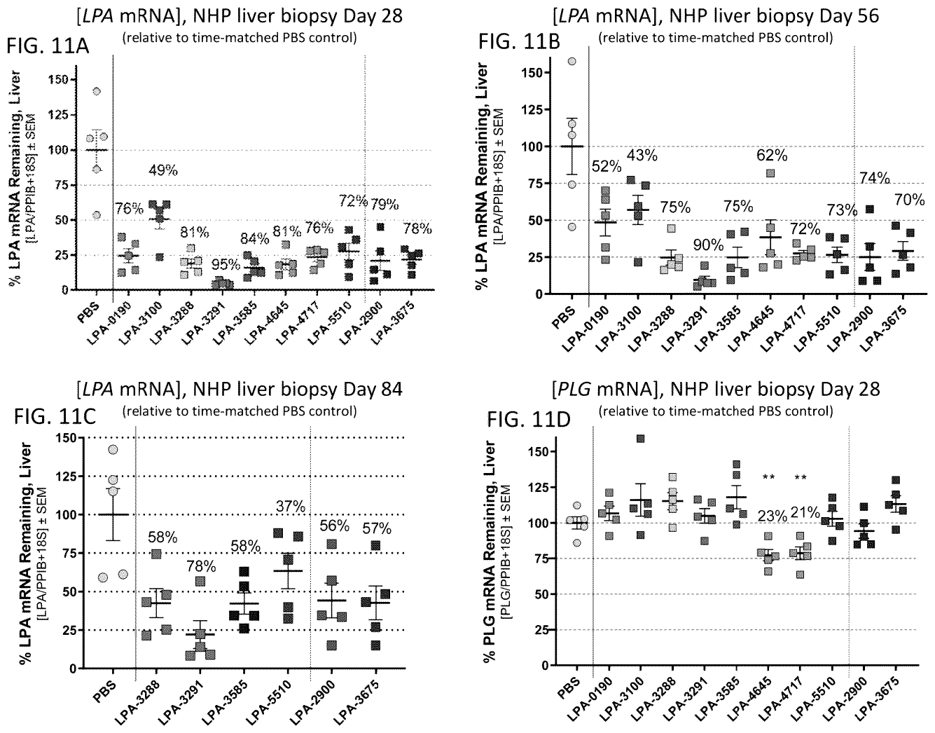

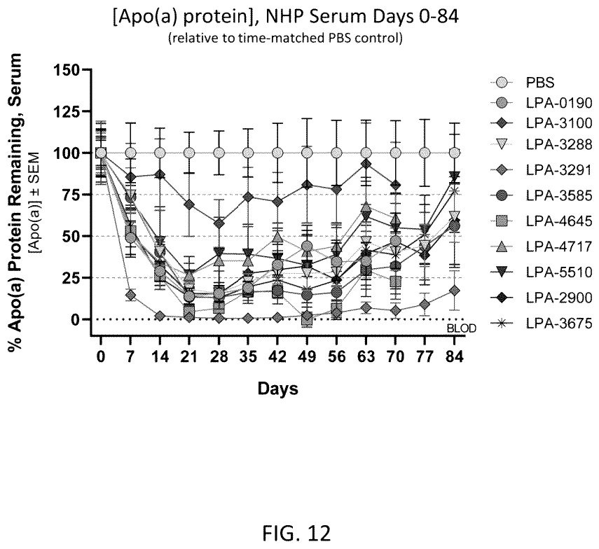

The GalNAc-conjugated LPA oligonucleotides listed in Table 4 were evaluated in cynomolgus monkeys (Macaca fascicularis). In this study, the monkeys are grouped so that their mean body weights (about 5.4 kg) are comparable between the control and experimental groups. Each cohort contains two male and three female subjects. The GalNAc-conjugated LPA oligonucleotides were administered subcutaneously on Study Day 0. Blood samples were collected on Study Days −8, −5 and 0, and weekly after dosing. Ultrasound-guided core needle liver biopsies were collected on Study Days 28, 56 and 84. At each time point, total RNA derived from the liver biopsy samples was subjected to qRT-PCR analysis to measure LPA mRNA in oligonucleotide-treated monkeys relative to monkeys treated with a comparable volume of PBS. To normalize the data, the measurements were made relative to the geometric mean of two reference genes, PPIB and 18S rRNA. As shown in FIG. 11A (Day 28), FIG. 11B (Day 56), and FIG. 11C (Day 84), treatment of NHPs with the GalNAc-conjugated LPA oligonucleotides listed in Table 4 inhibited LPA expression in the liver, as determined by a reduction in the amount of LPA mRNA in liver samples from oligonucleotide-treated NHPs relative to NHPs treated with PBS. The amount of plasminogen (PLG) mRNA in the liver samples of treated NHPs was also determined and is shown in FIG. 11D. From the same NHP study, inhibition of LPA expression was also determined by measuring apo(a) protein serum from treated NHPs by ELISA. As shown in FIG. 12, a significant reduction in serum apo(a) protein was observed in NHPs treated with GalNAc-conjugated LPA oligonucleotides compared to NHPs treated with PBS. Values from three pre-dose samples are averaged and set to 100%, and data are reported as relative values compared to the pre-dose average. Taken together, these results demonstrate that treatment of NHPs with GalNAc-conjugated LPA oligonucleotides reduced the amount of LPA mRNA in the liver and reduced the amount of apo(a) protein in the serum.

Taken together, these results show that GalNAc-conjugated LPA oligonucleotides designed to target human LPA mRNA inhibit LPA expression in vivo (as determined by the reduction of the amount of LPA mRNA and apo(a) protein in treated animals).

### SEQUENCE LISTING

The following nucleic and/or amino acid sequences are referred to in the disclosure above and are provided below for reference.

TABLE 5
LPA Oligonucleotide Sequences (Unmodified)

| | | SEQ | | SEQ |
|---|---|---|---|---|
| | Sequence | ID | Sequence | ID |
| Oligonucleotide | (Sense Strand) | NO: | (Antisense Strand) | NO: |
| LPA-125 | CUGAGCAAAGCCAUGUGGUAC | 4 | UCCUGUACCACAUGGCUUUGCUCA | 404 |
| | AGGA | | GGU | |
| LPA-128 | AGCAAAGCCAUGUGGUCCAAG | 5 | CAAUCUUGGACCACAUGGCUUUGC | 405 |
| | AUTG | | UCA | |
| LPA-132 | AAGCCAUGUGGUCCAGGAUAG | 6 | GUAGCUAUCCUGGACCACAUGGCU | 406 |
| | CUAC | | UUG | |
| LPA-133 | AGCCAUGUGGUCCAGGAUUAC | 7 | GGUAGUAAUCCUGGACCACAUGGC | 407 |
| | UACC | | UUU | |
| LPA-134 | GCCAUGUGGUCCAGGAUUGAU | 8 | UGGUAUCAAUCCUGGACCACAUGG | 408 |
| | ACCA | | CUU | |
| LPA-135 | CCAUGUGGUCCAGGAUUGCAA | 9 | AUGGUUGCAAUCCUGGACCACAUG | 409 |
| | CCAT | | GCU | |
| LPA-136 | CAUGUGGUCCAGGAUUGCUAC | 10 | CAUGGUAGCAAUCCUGGACCACAU | 410 |
| | CATG | | GGC | |
| LPA-137 | AUGUGGUCCAGGAUUGCUAAC | 11 | CCAUGUUAGCAAUCCUGGACCACA | 411 |
| | AUGG | | UGG | |
| LPA-138 | UGUGGUCCAGGAUUGCUACAA | 12 | ACCAUUGUAGCAAUCCUGGACCAC | 412 |
| | UGGT | | AUG | |
| LPA-160 | GGUGAUGGACAGAGUUAUCAA | 13 | UGCCUUGAUAACUCUGUCCAUCAC | 413 |
| | GGCA | | CAU | |
| LPA-190 | UCCACCACUGUCACAGGAAAG | 14 | AGGUCUUUCCUGUGACAGUGGUGG | 414 |
| | ACCT | | AGU | |
| LPA-191 | CCACCACUGUCACAGGAAGAA | 15 | CAGGUUCUUCCUGUGACAGUGGUG | 415 |
| | CCTG | | GAG | |
| LPA-197 | CUGUCACAGGAAGGACCUGAC | 16 | GCUUGUCAGGUCCUUCCUGUGACA | 416 |
| | AAGC | | GUG | |
| LPA-205 | GGAAGGACCUGCCAAGCUUAG | 17 | AUGACUAAGCUUGGCAGGUCCUUC | 417 |
| | UCAT | | CUG | |
| LPA-206 | GAAGGACCUGCCAAGCUUGAU | 18 | GAUGAUCAAGCUUGGCAGGUCCUU | 418 |
| | CATC | | CCU | |
| LPA-208 | AGGACCUGCCAAGCUUGGUAA | 19 | UAGAUUACCAAGCUUGGCAGGUCC | 419 |
| | UCTA | | UUC | |
| LPA-209 | GGACCUGCCAAGCUUGGUCAU | 20 | AUAGAUGACCAAGCUUGGCAGGUC | 420 |
| | CUAT | | CUU | |
| LPA-210 | GACCUGCCAAGCUUGGUCAAC | 21 | CAUAGUUGACCAAGCUUGGCAGGU | 421 |
| | UATG | | CCU | |
| LPA-211 | ACCUGCCAAGCUUGGUCAUAU | 22 | UCAUAUAUGACCAAGCUUGGCAGG | 422 |
| | AUGA | | UCC | |
| LPA-212 | CCUGCCAAGCUUGGUCAUCAA | 23 | GUCAUUGAUGACCAAGCUUGGCAG | 423 |
| | UGAC | | GUC | |
| LPA-219 | AGCUUGGUCAUCUAUGACAAC | 24 | AUGUGUUGUCAUAGAUGACCAAGC | 424 |
| | ACAT | | UUG | |
| LPA-225 | GUCAUCUAUGACACCACAUAA | 25 | AUGUUUAUGUGGUGUCAUAGAUGA | 425 |
| | ACAT | | CCA | |
| LPA-258 | CACAGAAAACUACCCAAAUAC | 26 | GCCAGUAUUUGGGUAGUUUUCUGU | 426 |
| | UGGC | | GGU | |
| LPA-261 | AGAAAACUACCCAAAUGCUAG | 27 | CAAGCUAGCAUUUGGGUAGUUUUC | 427 |
| | CUTG | | UGU | |
| LPA-263 | AAAACUACCCAAAUGCUGGAU | 28 | AUCAAUCCAGCAUUUGGGUAGUUU | 428 |
| | UGAT | | UCU | |
| LPA-269 | ACCCAAAUGCUGGCUUGAUAA | 29 | UUCAUUAUCAAGCCAGCAUUUGGG | 429 |
| | UGAA | | UAG | |
| LPA-270 | CCCAAAUGCUGGCUUGAUCAU | 30 | GUUCAUGAUCAAGCCAGCAUUUGG | 430 |
| | GAAC | | GUA | |
| LPA-291 | GAACUACUGCAGGAAUCCAAA | 31 | AGCAUUUGGAUUCCUGCAGUAGUU | 431 |
| | UGCT | | CAU | |
| LPA-295 | UACUGCAGGAAUCCAGAUGAU | 32 | CCACAUCAUCUGGAUUCCUGCAGU | 432 |
| | GUGG | | AGU | |
| LPA-296 | ACUGCAGGAAUCCAGAUGCAG | 33 | GCCACUGCAUCUGGAUUCCUGCAG | 433 |
| | UGGC | | UAG | |
| LPA-298 | UGCAGGAAUCCAGAUGCUGAG | 34 | CUGCCUCAGCAUCUGGAUUCCUGC | 434 |
| | GCAG | | AGU | |
| LPA-355 | AGGUGGGAGUACUGCAACCAG | 35 | GCGUCUGGUUGCAGUACUCCCACC | 435 |
| | ACGC | | UGA | |
| LPA-380 | AAUGCUCAGACGCAGAAGGAA | 36 | GCAGUUCCUUCUGCGUCUGAGCAU | 436 |
| | CUGC | | UGC | |
| LPA-417 | GACUGUUACCCCGGUUCCAAG | 37 | UAGGCUUGGAACCGGGGUAACAGU | 437 |
| | CCTA | | CGG | |
| LPA-418 | ACUGUUACCCCGGUUCCAAAC | 38 | CUAGGUUUGGAACCGGGGUAACAG | 438 |
| | CUAG | | UCG | |
| LPA-419 | CUGUUACCCCGGUUCCAAGAC | 39 | UCUAGUCUUGGAACCGGGGUAACA | 439 |
| | UAGA | | GUC | |
| LPA-420 | UGUUACCCCGGUUCCAAGCAU | 40 | CUCUAUGCUUGGAACCGGGGUAAC | 440 |
| | AGAG | | AGU | |
| LPA-421 | GUUACCCCGGUUCCAAGCCAA | 41 | CCUCUUGGCUUGGAACCGGGGUAA | 441 |
| | GAGG | | CAG | |
| LPA-422 | UUACCCCGGUUCCAAGCCUAG | 42 | GCCUCUAGGCUUGGAACCGGGGUA | 442 |
| | AGGC | | ACA | |
| LPA-423 | UACCCCGGUUCCAAGCCUAAA | 43 | AGCCUUUAGGCUUGGAACCGGGGU | 443 |
| | GGCT | | AAC | |
| LPA-492 | GUGCUACCAUGGUAAUGGAAA | 44 | ACUCUUUCCAUUACCAUGGUAGCA | 444 |
| | GAGT | | CUC | |
| LPA-493 | UGCUACCAUGGUAAUGGACAG | 45 | AACUCUGUCCAUUACCAUGGUAGC | 445 |
| | AGTT | | ACU | |
| LPA-494 | GCUACCAUGGUAAUGGACAAA | 46 | UAACUUUGUCCAUUACCAUGGUAG | 446 |
| | GUTA | | CAC | |
| LPA-495 | CUACCAUGGUAAUGGACAGAG | 47 | AUAACUCUGUCCAUUACCAUGGUA | 447 |
| | UUAT | | GCA | |
| LPA-496 | UACCAUGGUAAUGGACAGAAU | 48 | GAUAAUUCUGUCCAUUACCAUGGU | 448 |
| | UATC | | AGC | |
| LPA-497 | ACCAUGGUAAUGGACAGAGAU | 49 | CGAUAUCUCUGUCCAUUACCAUGG | 449 |
| | AUCG | | UAG | |
| LPA-498 | CCAUGGUAAUGGACAGAGUAA | 50 | UCGAUUACUCUGUCCAUUACCAUG | 450 |
| | UCGA | | GUA | |
| LPA-499 | CAUGGUAAUGGACAGAGUUAU | 51 | CUCGAUAACUCUGUCCAUUACCAU | 451 |
| | CGAG | | GGU | |
| LPA-500 | AUGGUAAUGGACAGAGUUAAC | 52 | CCUCGUUAACUCUGUCCAUUACCA | 452 |
| | GAGG | | UGG | |
| LPA-501 | UGGUAAUGGACAGAGUUAUAG | 53 | GCCUCUAUAACUCUGUCCAUUACC | 453 |
| | AGGC | | AUG | |
| LPA-502 | GGUAAUGGACAGAGUUAUCAA | 54 | UGCCUUGAUAACUCUGUCCAUUAC | 454 |
| | GGCA | | CAU | |
| LPA-503 | GUAAUGGACAGAGUUAUCGAG | 55 | GUGCCUCGAUAACUCUGUCCAUUA | 455 |
| | GCAC | | CCA | |
| LPA-523 | GGCACAUACUCCACCACUGAC | 56 | CUGUGUCAGUGGUGGAGUAUGUGC | 456 |
| | ACAG | | CUC | |
| LPA-563 | CUUGGUCAUCUAUGACACCAC | 57 | GAGUGUGGUGUCAUAGAUGACCAA | 457 |
| | ACTC | | GCU | |
| LPA-567 | GUCAUCUAUGACACCACACAC | 58 | AUGCGUGUGUGGUGUCAUAGAUGA | 458 |
| | GCAT | | CCA | |
| LPA-568 | UCAUCUAUGACACCACACUAG | 59 | UAUGCUAGUGUGGUGUCAUAGAUG | 459 |
| | CATA | | ACC | |
| LPA-569 | CAUCUAUGACACCACACUCAC | 60 | CUAUGUGAGUGUGGUGUCAUAGAU | 460 |
| | AUAG | | GAC | |
| LPA-1208 | GCACAUACUCCACCACUGUAA | 61 | CCAGUUACAGUGGUGGAGUAUGUG | 461 |
| | CUGG | | CCU | |
| LPA-2715 | AGCCCCUUAUUGUUAUACGAG | 62 | AUCCCUCGUAUAACAAUAAGGGGC | 462 |
| | GGAT | | UGC | |
| LPA-2716 | GCCCCUUAUUGUUAUACGAAG | 63 | GAUCCUUCGUAUAACAAUAAGGGG | 463 |
| | GATC | | CUG | |
| LPA-2827 | CCAAGCCUAGAGGCUCCUUAU | 64 | GUUCAUAAGGAGCCUCUAGGCUUG | 464 |
| | GAAC | | GAA | |
| LPA-2837 | AGGCUCCUUCUGAACAAGCAC | 65 | GUUGGUGCUUGUUCAGAAGGAGCC | 465 |
| | CAAC | | UCU | |
| LPA-2900 | AUGGACAGAGUUAUCAAGGAA | 66 | UAUGUUCCUUGAUAACUCUGUCCA | 466 |
| | CATA | | UUU | |
| LPA-2901 | UGGACAGAGUUAUCAAGGCAC | 67 | GUAUGUGCCUUGAUAACUCUGUCC | 467 |
| | AUAC | | AUU | |
| LPA-2902 | GGACAGAGUUAUCAAGGCAAA | 68 | AGUAUUUGCCUUGAUAACUCUGUC | 468 |
| | UACT | | CAU | |
| LPA-2903 | GACAGAGUUAUCAAGGCACAU | 69 | AAGUAUGUGCCUUGAUAACUCUGU | 469 |
| | ACTT | | CCA | |
| LPA-2904 | ACAGAGUUAUCAAGGCACAAA | 70 | GAAGUUUGUGCCUUGAUAACUCUG | 470 |
| | CUTC | | UCC | |
| LPA-2905 | CAGAGUUAUCAAGGCACAUAC | 71 | UGAAGUAUGUGCCUUGAUAACUCU | 471 |
| | UUCA | | GUC | |
| LPA-3004 | UACCCAAAUGCUGGCUUGAAC | 72 | UCUUGUUCAAGCCAGCAUUUGGGU | 472 |
| | AAGA | | AGU | |
| LPA-3007 | CCAAAUGCUGGCUUGAUCAAG | 73 | AGUUCUUGAUCAAGCCAGCAUUUG | 473 |
| | AACT | | GGU | |
| LPA-3023 | UCAAGAACUACUGCCGAAAAC | 74 | UCUGGUUUUCGGCAGUAGUUCUUG | 474 |
| | CAGA | | AUC | |
| LPA-3024 | CAAGAACUACUGCCGAAAUAC | 75 | AUCUGUAUUUCGGCAGUAGUUCUU | 475 |
| | AGAT | | GAU | |
| LPA-3025 | AAGAACUACUGCCGAAAUCAA | 76 | GAUCUUGAUUUCGGCAGUAGUUCU | 476 |
| | GATC | | UGA | |
| LPA-3027 | GAACUACUGCCGAAAUCCAAA | 77 | AGGAUUUGGAUUUCGGCAGUAGUU | 477 |
| | UCCT | | CUU | |
| LPA-3030 | CUACUGCCGAAAUCCAGAUAC | 78 | CACAGUAUCUGGAUUUCGGCAGUA | 478 |
| | UGTG | | GUU | |
| LPA-3051 | UGUGGCAGCCCCUUGGUGUAA | 79 | UGUAUUACACCAAGGGGCUGCCAC | 479 |
| | UACA | | AGG | |
| LPA-3052 | GUGGCAGCCCCUUGGUGUUAU | 80 | UUGUAUAACACCAAGGGGCUGCCA | 480 |
| | ACAA | | CAG | |
| LPA-3053 | UGGCAGCCCCUUGGUGUUAAA | 81 | GUUGUUUAACACCAAGGGGCUGCC | 481 |
| | CAAC | | ACA | |
| LPA-3054 | GGCAGCCCCUUGGUGUUAUAC | 82 | UGUUGUAUAACACCAAGGGGCUGC | 482 |
| | AACA | | CAC | |
| LPA-3055 | GCAGCCCCUUGGUGUUAUAAA | 83 | CUGUUUUAUAACACCAAGGGGCUG | 483 |
| | ACAG | | CCA | |
| LPA-3056 | CAGCCCCUUGGUGUUAUACAA | 84 | UCUGUUGUAUAACACCAAGGGGCU | 484 |
| | CAGA | | GCC | |
| LPA-3057 | AGCCCCUUGGUGUUAUACAAC | 85 | AUCUGUUGUAUAACACCAAGGGGC | 485 |
| | AGAT | | UGC | |
| LPA-3058 | GCCCCUUGGUGUUAUACAAAA | 86 | GAUCUUUUGUAUAACACCAAGGGG | 486 |
| | GATC | | CUG | |
| LPA-3059 | CCCCUUGGUGUUAUACAACAG | 87 | GGAUCUGUUGUAUAACACCAAGGG | 487 |
| | AUCC | | GCU | |
| LPA-3092 | GGUGGGAGUACUGCAACCUAA | 88 | CGUGUUAGGUUGCAGUACUCCCAC | 488 |
| | CACG | | CUG | |
| LPA-3093 | GUGGGAGUACUGCAACCUGAC | 89 | UCGUGUCAGGUUGCAGUACUCCCA | 489 |
| | ACGA | | CCU | |
| LPA-3096 | GGAGUACUGCAACCUGACAAG | 90 | GCAUCUUGUCAGGUUGCAGUACUC | 490 |
| | AUGC | | CCA | |
| LPA-3097 | GAGUACUGCAACCUGACACAA | 91 | AGCAUUGUGUCAGGUUGCAGUACU | 491 |
| | UGCT | | CCC | |
| LPA-3099 | GUACUGCAACCUGACACGAAG | 92 | UGAGCUUCGUGUCAGGUUGCAGUA | 492 |
| | CUCA | | CUC | |
| LPA-3100 | UACUGCAACCUGACACGAUAC | 93 | CUGAGUAUCGUGUCAGGUUGCAGU | 493 |
| | UCAG | | ACU | |
| LPA-3101 | ACUGCAACCUGACACGAUGAU | 94 | UCUGAUCAUCGUGUCAGGUUGCAG | 494 |
| | CAGA | | UAC | |
| LPA-3102 | CUGCAACCUGACACGAUGCAC | 95 | AUCUGUGCAUCGUGUCAGGUUGCA | 495 |
| | AGAT | | GUA | |
| LPA-3103 | UGCAACCUGACACGAUGCUAA | 96 | CAUCUUAGCAUCGUGUCAGGUUGC | 496 |
| | GATG | | AGU | |
| LPA-3105 | CAACCUGACACGAUGCUCAAA | 97 | UGCAUUUGAGCAUCGUGUCAGGUU | 497 |
| | UGCA | | GCA | |
| LPA-3107 | ACCUGACACGAUGCUCAGAAG | 98 | UCUGCUUCUGAGCAUCGUGUCAGG | 498 |
| | CAGA | | UUG | |
| LPA-3108 | CCUGACACGAUGCUCAGAUAC | 99 | UUCUGUAUCUGAGCAUCGUGUCAG | 499 |
| | AGAA | | GUU | |
| LPA-3109 | CUGACACGAUGCUCAGAUGAA | 100 | AUUCUUCAUCUGAGCAUCGUGUCA | 500 |
| | GAAT | | GGU | |
| LPA-3110 | UGACACGAUGCUCAGAUGCAG | 101 | CAUUCUGCAUCUGAGCAUCGUGUC | 501 |
| | AATG | | AGG | |
| LPA-3111 | GACACGAUGCUCAGAUGCAAA | 102 | CCAUUUUGCAUCUGAGCAUCGUGU | 502 |
| | AUGG | | CAG | |
| LPA-3112 | ACACGAUGCUCAGAUGCAGAA | 103 | UCCAUUCUGCAUCUGAGCAUCGUG | 503 |
| | UGGA | | UCA | |
| LPA-3113 | CACGAUGCUCAGAUGCAGAAU | 104 | GUCCAUUCUGCAUCUGAGCAUCGU | 504 |
| | GGAC | | GUC | |
| LPA-3229 | UGCUACUACCAUUAUGGACAG | 105 | AACUCUGUCCAUAAUGGUAGUAGC | 505 |
| | AGTT | | AGU | |
| LPA-3230 | GCUACUACCAUUAUGGACAAA | 106 | UAACUUUGUCCAUAAUGGUAGUAG | 506 |
| | GUTA | | CAG | |
| LPA-3231 | CUACUACCAUUAUGGACAGAG | 107 | GUAACUCUGUCCAUAAUGGUAGUA | 507 |
| | UUAC | | GCA | |
| LPA-3232 | UACUACCAUUAUGGACAGAAU | 108 | GGUAAUUCUGUCCAUAAUGGUAGU | 508 |
| | UACC | | AGC | |
| LPA-3233 | ACUACCAUUAUGGACAGAGAU | 109 | CGGUAUCUCUGUCCAUAAUGGUAG | 509 |
| | ACCG | | UAG | |
| LPA-3234 | CUACCAUUAUGGACAGAGUAA | 110 | UCGGUUACUCUGUCCAUAAUGGUA | 510 |
| | CCGA | | GUA | |
| LPA-3235 | UACCAUUAUGGACAGAGUUAC | 111 | CUCGGUAACUCUGUCCAUAAUGGU | 511 |
| | CGAG | | AGU | |
| LPA-3236 | ACCAUUAUGGACAGAGUUAAC | 112 | CCUCGUUAACUCUGUCCAUAAUGG | 512 |
| | GAGG | | UAG | |
| LPA-3257 | GAGGCACAUACUCCACCACAG | 113 | GUGACUGUGGUGGAGUAUGUGCCU | 513 |
| | UCAC | | CGG | |
| LPA-3267 | CUCCACCACUGUCACAGGAAG | 114 | AGUUCUUCCUGUGACAGUGGUGGA | 514 |
| | AACT | | GUA | |
| LPA-3280 | ACAGGAAGAACUUGCCAAGAU | 115 | ACCAAUCUUGGCAAGUUCUUCCUG | 515 |
| | UGGT | | UGA | |
| LPA-3281 | CAGGAAGAACUUGCCAAGCAU | 116 | GACCAUGCUUGGCAAGUUCUUCCU | 516 |
| | GGTC | | GUG | |
| LPA-3282 | AGGAAGAACUUGCCAAGCUAG | 117 | UGACCUAGCUUGGCAAGUUCUUCC | 517 |
| | GUCA | | UGU | |
| LPA-3283 | GGAAGAACUUGCCAAGCUUAG | 118 | AUGACUAAGCUUGGCAAGUUCUUC | 518 |
| | UCAT | | CUG | |
| LPA-3284 | GAAGAACUUGCCAAGCUUGAU | 119 | GAUGAUCAAGCUUGGCAAGUUCUU | 519 |
| | CATC | | CCU | |
| LPA-3285 | AAGAACUUGCCAAGCUUGGAC | 120 | AGAUGUCCAAGCUUGGCAAGUUCU | 520 |
| | AUCT | | UCC | |
| LPA-3286 | AGAACUUGCCAAGCUUGGUAA | 121 | UAGAUUACCAAGCUUGGCAAGUUC | 521 |
| | UCTA | | UUC | |
| LPA-3287 | GAACUUGCCAAGCUUGGUCAU | 122 | AUAGAUGACCAAGCUUGGCAAGUU | 522 |
| | CUAT | | CUU | |
| LPA-3288 | AACUUGCCAAGCUUGGUCAAC | 123 | CAUAGUUGACCAAGCUUGGCAAGU | 523 |
| | UATG | | UCU | |
| LPA-3289 | ACUUGCCAAGCUUGGUCAUAU | 124 | UCAUAUAUGACCAAGCUUGGCAAG | 524 |
| | AUGA | | UUC | |
| LPA-3290 | CUUGCCAAGCUUGGUCAUCAA | 125 | GUCAUUGAUGACCAAGCUUGGCAA | 525 |
| | UGAC | | GUU | |
| LPA-3291 | UUGCCAAGCUUGGUCAUCUAU | 126 | UGUCAUAGAUGACCAAGCUUGGCA | 526 |
| | GACA | | AGU | |
| LPA-3292 | UGCCAAGCUUGGUCAUCUAAG | 127 | GUGUCUUAGAUGACCAAGCUUGGC | 527 |
| | ACAC | | AAG | |
| LPA-3298 | GCUUGGUCAUCUAUGACACAA | 128 | GGUGUUGUGUCAUAGAUGACCAAG | 528 |
| | CACC | | CUU | |
| LPA-3300 | UUGGUCAUCUAUGACACCAAA | 129 | CUGGUUUGGUGUCAUAGAUGACCA | 529 |
| | CCAG | | AGC | |
| LPA-3301 | UGGUCAUCUAUGACACCACAC | 130 | GCUGGUGUGGUGUCAUAGAUGACC | 530 |
| | CAGC | | AAG | |
| LPA-3303 | GUCAUCUAUGACACCACACAA | 131 | AUGCUUGUGUGGUGUCAUAGAUGA | 531 |
| | GCAT | | CCA | |
| LPA-3305 | CAUCUAUGACACCACACCAAC | 132 | CUAUGUUGGUGUGGUGUCAUAGAU | 532 |
| | AUAG | | GAC | |
| LPA-3306 | AUCUAUGACACCACACCAGAA | 133 | ACUAUUCUGGUGUGGUGUCAUAGA | 533 |
| | UAGT | | UGA | |
| LPA-3308 | CUAUGACACCACACCAGCAAA | 134 | CGACUUUGCUGGUGUGGUGUCAUA | 534 |
| | GUCG | | GAU | |
| LPA-3329 | GUCGGACCCCAGAAAACUAAC | 135 | UUUGGUUAGUUUUCUGGGGUCCGA | 535 |
| | CAAA | | CUA | |
| LPA-3330 | UCGGACCCCAGAAAACUACAC | 136 | AUUUGUGUAGUUUUCUGGGGUCCG | 536 |
| | AAAT | | ACU | |
| LPA-3340 | GAAAACUACCCAAAUGCUGAC | 137 | UCAGGUCAGCAUUUGGGUAGUUUU | 537 |
| | CUGA | | CUG | |
| LPA-3391 | GCUGAGAUUCGCCCUUGGUAU | 138 | UGUAAUACCAAGGGCGAAUCUCAG | 538 |
| | UACA | | CAU | |
| LPA-3392 | CUGAGAUUCGCCCUUGGUGAU | 139 | GUGUAUCACCAAGGGCGAAUCUCA | 539 |
| | ACAC | | GCA | |
| LPA-3394 | GAGAUUCGCCCUUGGUGUUAC | 140 | UGGUGUAACACCAAGGGCGAAUCU | 540 |
| | ACCA | | CAG | |
| LPA-3395 | AGAUUCGCCCUUGGUGUUAAA | 141 | AUGGUUUAACACCAAGGGCGAAUC | 541 |
| | CCAT | | UCA | |
| LPA-3398 | UUCGCCCUUGGUGUUACACAA | 142 | UCCAUUGUGUAACACCAAGGGCGA | 542 |
| | UGGA | | AUC | |
| LPA-3404 | CUUGGUGUUACACCAUGGAAC | 143 | CUGGGUUCCAUGGUGUAACACCAA | 543 |
| | CCAG | | GGG | |
| LPA-3405 | UUGGUGUUACACCAUGGAUAC | 144 | ACUGGUAUCCAUGGUGUAACACCA | 544 |
| | CAGT | | AGG | |
| LPA-3406 | UGGUGUUACACCAUGGAUCAC | 145 | CACUGUGAUCCAUGGUGUAACACC | 545 |
| | AGTG | | AAG | |
| LPA-3407 | GGUGUUACACCAUGGAUCCAA | 146 | ACACUUGGAUCCAUGGUGUAACAC | 546 |
| | GUGT | | CAA | |
| LPA-3409 | UGUUACACCAUGGAUCCCAAU | 147 | UGACAUUGGGAUCCAUGGUGUAAC | 547 |
| | GUCA | | ACC | |
| LPA-3472 | GAAUCAAGUGUCCUUGCAAAU | 148 | UGAGAUUUGCAAGGACACUUGAUU | 548 |
| | CUCA | | CUG | |
| LPA-3473 | AAUCAAGUGUCCUUGCAACAC | 149 | GUGAGUGUUGCAAGGACACUUGAU | 549 |
| | UCAC | | UCU | |
| LPA-3474 | AUCAAGUGUCCUUGCAACUAU | 150 | CGUGAUAGUUGCAAGGACACUUGA | 550 |
| | CACG | | UUC | |
| LPA-3584 | AUGGACAGAGUUAUCGAGGAU | 151 | AAUGAUCCUCGAUAACUCUGUCCA | 551 |
| | CATT | | UCA | |
| LPA-3585 | UGGACAGAGUUAUCGAGGCAC | 152 | GAAUGUGCCUCGAUAACUCUGUCC | 552 |
| | AUTC | | AUC | |
| LPA-3655 | ACACCACACUGGCAUCAGAAG | 153 | UUGUCUUCUGAUGCCAGUGUGGUG | 553 |
| | ACAA | | UCA | |
| LPA-3747 | UUGGUGUUAUACCAUGGAUAC | 154 | AUUGGUAUCCAUGGUAUAACACCA | 554 |
| | CAAT | | AGG | |
| LPA-3748 | UGGUGUUAUACCAUGGAUCAC | 155 | CAUUGUGAUCCAUGGUAUAACACC | 555 |
| | AATG | | AAG | |
| LPA-3749 | GGUGUUAUACCAUGGAUCCAA | 156 | ACAUUUGGAUCCAUGGUAUAACAC | 556 |
| | AUGT | | CAA | |
| LPA-3750 | GUGUUAUACCAUGGAUCCCAA | 157 | GACAUUGGGAUCCAUGGUAUAACA | 557 |
| | UGTC | | CCA | |
| LPA-3773 | UCAGAUGGGAGUACUGCAAAC | 158 | GUCAGUUUGCAGUACUCCCAUCUG | 558 |
| | UGAC | | ACA | |
| LPA-3776 | GAUGGGAGUACUGCAACCUAA | 159 | UGUGUUAGGUUGCAGUACUCCCAU | 559 |
| | CACA | | CUG | |
| LPA-3777 | AUGGGAGUACUGCAACCUGAC | 160 | UUGUGUCAGGUUGCAGUACUCCCA | 560 |
| | ACAA | | UCU | |
| LPA-3778 | UGGGAGUACUGCAACCUGAAA | 161 | AUUGUUUCAGGUUGCAGUACUCCC | 561 |
| | CAAT | | AUC | |
| LPA-3779 | GGGAGUACUGCAACCUGACAC | 162 | CAUUGUGUCAGGUUGCAGUACUCC | 562 |
| | AATG | | CAU | |
| LPA-3840 | GGCUGUUUCUGAACAAGCAAC | 163 | CGUUGUUGCUUGUUCAGAAACAGC | 563 |
| | AACG | | CGU | |
| LPA-3844 | GUUUCUGAACAAGCACCAAAG | 164 | GCUCCUUUGGUGCUUGUUCAGAAA | 564 |
| | GAGC | | CAG | |
| LPA-3927 | CUCCACCACUGUUACAGGAAG | 165 | UGUCCUUCCUGUAACAGUGGUGGA | 565 |
| | GACA | | GAA | |
| LPA-3928 | UCCACCACUGUUACAGGAAAG | 166 | AUGUCUUUCCUGUAACAGUGGUGG | 566 |
| | ACAT | | AGA | |
| LPA-3929 | CCACCACUGUUACAGGAAGAA | 167 | CAUGUUCUUCCUGUAACAGUGGUG | 567 |
| | CATG | | GAG | |
| LPA-3972 | GACACCACACUGGCAUCAGAG | 168 | GGUUCUCUGAUGCCAGUGUGGUGU | 568 |
| | AACC | | CAU | |
| LPA-3973 | ACACCACACUGGCAUCAGAAA | 169 | UGGUUUUCUGAUGCCAGUGUGGUG | 569 |
| | ACCA | | UCA | |
| LPA-3999 | AGAAUACUACCCAAAUGGUAG | 170 | CAGGCUACCAUUUGGGUAGUAUUC | 570 |
| | CCTG | | UGU | |
| LPA-4000 | GAAUACUACCCAAAUGGUGAC | 171 | UCAGGUCACCAUUUGGGUAGUAUU | 571 |
| | CUGA | | CUG | |
| LPA-4001 | AAUACUACCCAAAUGGUGGAC | 172 | GUCAGUCCACCAUUUGGGUAGUAU | 572 |
| | UGAC | | UCU | |
| LPA-4185 | UCCUUCUGAAGAAGCACCAAC | 173 | UUCAGUUGGUGCUUCUUCAGAAGG | 573 |
| | UGAA | | AAG | |
| LPA-4186 | CCUUCUGAAGAAGCACCAAAU | 174 | UUUCAUUUGGUGCUUCUUCAGAAG | 574 |
| | GAAA | | GAA | |
| LPA-4187 | CUUCUGAAGAAGCACCAACAG | 175 | UUUUCUGUUGGUGCUUCUUCAGAA | 575 |
| | AAAA | | GGA | |
| LPA-4188 | UUCUGAAGAAGCACCAACUAA | 176 | GUUUUUAGUUGGUGCUUCUUCAGA | 576 |
| | AAAC | | AGG | |
| LPA-4189 | UCUGAAGAAGCACCAACUGAA | 177 | UGUUUUCAGUUGGUGCUUCUUCAG | 577 |
| | AACA | | AAG | |
| LPA-4190 | CUGAAGAAGCACCAACUGAAA | 178 | CUGUUUUCAGUUGGUGCUUCUUCA | 578 |
| | ACAG | | GAA | |
| LPA-4191 | UGAAGAAGCACCAACUGAAAA | 179 | GCUGUUUUCAGUUGGUGCUUCUUC | 579 |
| | CAGC | | AGA | |
| LPA-4192 | GAAGAAGCACCAACUGAAAAC | 180 | UGCUGUUUUCAGUUGGUGCUUCUU | 580 |
| | AGCA | | CAG | |
| LPA-4193 | AAGAAGCACCAACUGAAAAAA | 181 | GUGCUUUUUUCAGUUGGUGCUUCU | 581 |
| | GCAC | | UCA | |
| LPA-4194 | AGAAGCACCAACUGAAAACAG | 182 | AGUGCUGUUUUCAGUUGGUGCUUC | 582 |
| | CACT | | UUC | |
| LPA-4195 | GAAGCACCAACUGAAAACAAC | 183 | CAGUGUUGUUUUCAGUUGGUGCUU | 583 |
| | ACTG | | CUU | |
| LPA-4196 | AAGCACCAACUGAAAACAGAA | 184 | CCAGUUCUGUUUUCAGUUGGUGCU | 584 |
| | CUGG | | UCU | |
| LPA-4239 | AGGUGAUGGACAGAGUUAUAG | 185 | GCCUCUAUAACUCUGUCCAUCACC | 585 |
| | AGGC | | UCG | |
| LPA-4269 | CUCCACCACUAUCACAGGAAG | 186 | UGUUCUUCCUGUGAUAGUGGUGGA | 586 |
| | AACA | | GAG | |
| LPA-4270 | UCCACCACUAUCACAGGAAAA | 187 | AUGUUUUUCCUGUGAUAGUGGUGG | 587 |
| | ACAT | | AGA | |
| LPA-4271 | CCACCACUAUCACAGGAAGAA | 188 | CAUGUUCUUCCUGUGAUAGUGGUG | 588 |
| | CATG | | GAG | |
| LPA-4272 | CACCACUAUCACAGGAAGAAC | 189 | ACAUGUUCUUCCUGUGAUAGUGGU | 589 |
| | AUGT | | GGA | |
| LPA-4273 | ACCACUAUCACAGGAAGAAAA | 190 | GACAUUUUCUUCCUGUGAUAGUGG | 590 |
| | UGTC | | UGG | |
| LPA-4274 | CCACUAUCACAGGAAGAACAU | 191 | UGACAUGUUCUUCCUGUGAUAGUG | 591 |
| | GUCA | | GUG | |
| LPA-4275 | CACUAUCACAGGAAGAACAAG | 192 | CUGACUUGUUCUUCCUGUGAUAGU | 592 |
| | UCAG | | GGU | |
| LPA-4276 | ACUAUCACAGGAAGAACAUAU | 193 | ACUGAUAUGUUCUUCCUGUGAUAG | 593 |
| | CAGT | | UGG | |
| LPA-4277 | CUAUCACAGGAAGAACAUGAC | 194 | GACUGUCAUGUUCUUCCUGUGAUA | 594 |
| | AGTC | | GUG | |
| LPA-4278 | UAUCACAGGAAGAACAUGUAA | 195 | AGACUUACAUGUUCUUCCUGUGAU | 595 |
| | GUCT | | AGU | |
| LPA-4279 | AUCACAGGAAGAACAUGUCAG | 196 | AAGACUGACAUGUUCUUCCUGUGA | 596 |
| | UCTT | | UAG | |
| LPA-4280 | UCACAGGAAGAACAUGUCAAU | 197 | CAAGAUUGACAUGUUCUUCCUGUG | 597 |
| | CUTG | | AUA | |
| LPA-4281 | CACAGGAAGAACAUGUCAGAC | 198 | CCAAGUCUGACAUGUUCUUCCUGU | 598 |
| | UUGG | | GAU | |
| LPA-4282 | ACAGGAAGAACAUGUCAGUAU | 199 | ACCAAUACUGACAUGUUCUUCCUG | 599 |
| | UGGT | | UGA | |
| LPA-4285 | GGAAGAACAUGUCAGUCUUAG | 200 | ACGACUAAGACUGACAUGUUCUUC | 600 |
| | UCGT | | CUG | |
| LPA-4286 | GAAGAACAUGUCAGUCUUGAU | 201 | GACGAUCAAGACUGACAUGUUCUU | 601 |
| | CGTC | | CCU | |
| LPA-4287 | AAGAACAUGUCAGUCUUGGAC | 202 | AGACGUCCAAGACUGACAUGUUCU | 602 |
| | GUCT | | UCC | |
| LPA-4288 | AGAACAUGUCAGUCUUGGUAG | 203 | UAGACUACCAAGACUGACAUGUUC | 603 |
| | UCTA | | UUC | |
| LPA-4325 | GGCAUCGGAGGAUCCCAUUAU | 204 | UAGUAUAAUGGGAUCCUCCGAUGC | 604 |
| | ACTA | | CAA | |
| LPA-4346 | ACUAUCCAAAUGCUGGCCUAA | 205 | CUGGUUAGGCCAGCAUUUGGAUAG | 605 |
| | CCAG | | UAU | |
| LPA-4517 | GCACAGAGGCUCCUUCUGAAC | 206 | GCUUGUUCAGAAGGAGCCUCUGUG | 606 |
| | AAGC | | CUU | |
| LPA-4527 | UCCUUCUGAACAAGCACCAAC | 207 | CUCAGUUGGUGCUUGUUCAGAAGG | 607 |
| | UGAG | | AGC | |
| LPA-4528 | CCUUCUGAACAAGCACCACAU | 208 | UCUCAUGUGGUGCUUGUUCAGAAG | 608 |
| | GAGA | | GAG | |
| LPA-4529 | CUUCUGAACAAGCACCACCAG | 209 | UUCUCUGGUGGUGCUUGUUCAGAA | 609 |
| | AGAA | | GGA | |
| LPA-4530 | UUCUGAACAAGCACCACCUAA | 210 | UUUCUUAGGUGGUGCUUGUUCAGA | 610 |
| | GAAA | | AGG | |
| LPA-4531 | UCUGAACAAGCACCACCUGAG | 211 | UUUUCUCAGGUGGUGCUUGUUCAG | 611 |
| | AAAA | | AAG | |
| LPA-4532 | CUGAACAAGCACCACCUGAAA | 212 | CUUUUUUCAGGUGGUGCUUGUUCA | 612 |
| | AAAG | | GAA | |
| LPA-4533 | UGAACAAGCACCACCUGAGAA | 213 | GCUUUUCUCAGGUGGUGCUUGUUC | 613 |
| | AAGC | | AGA | |
| LPA-4534 | GAACAAGCACCACCUGAGAAA | 214 | GGCUUUUCUCAGGUGGUGCUUGUU | 614 |
| | AGCC | | CAG | |
| LPA-4535 | AACAAGCACCACCUGAGAAAA | 215 | GGGCUUUUCUCAGGUGGUGCUUGU | 615 |
| | GCCC | | UCA | |
| LPA-4537 | CAAGCACCACCUGAGAAAAAC | 216 | CAGGGUUUUUCUCAGGUGGUGCUU | 616 |
| | CCTG | | GUU | |
| LPA-4538 | AAGCACCACCUGAGAAAAGAC | 217 | ACAGGUCUUUUCUCAGGUGGUGCU | 617 |
| | CUGT | | UGU | |
| LPA-4539 | AGCACCACCUGAGAAAAGCAC | 218 | CACAGUGCUUUUCUCAGGUGGUGC | 618 |
| | UGTG | | UUG | |
| LPA-4547 | CUGAGAAAAGCCCUGUGGUAC | 219 | UCCUGUACCACAGGGCUUUUCUCA | 619 |
| | AGGA | | GGU | |
| LPA-4556 | GCCCUGUGGUCCAGGAUUGAU | 220 | UGGUAUCAAUCCUGGACCACAGGG | 620 |
| | ACCA | | CUU | |
| LPA-4559 | CUGUGGUCCAGGAUUGCUAAC | 221 | CCAUGUUAGCAAUCCUGGACCACA | 621 |
| | AUGG | | GGG | |
| LPA-4611 | CUCCACCACUGUCACAGGAAG | 222 | GGUCCUUCCUGUGACAGUGGUGGA | 622 |
| | GACC | | GGA | |
| LPA-4612 | UCCACCACUGUCACAGGAAAG | 223 | AGGUCUUUCCUGUGACAGUGGUGG | 623 |
| | ACCT | | AGG | |
| LPA-4642 | UCUUGGUCAUCUAUGAUACAA | 224 | AGUGUUGUAUCAUAGAUGACCAAG | 624 |
| | CACT | | AUU | |
| LPA-4643 | CUUGGUCAUCUAUGAUACCAC | 225 | CAGUGUGGUAUCAUAGAUGACCAA | 625 |
| | ACTG | | GAU | |
| LPA-4644 | UUGGUCAUCUAUGAUACCAAA | 226 | CCAGUUUGGUAUCAUAGAUGACCA | 626 |
| | CUGG | | AGA | |
| LPA-4645 | UGGUCAUCUAUGAUACCACAC | 227 | GCCAGUGUGGUAUCAUAGAUGACC | 627 |
| | UGGC | | AAG | |
| LPA-4646 | GGUCAUCUAUGAUACCACAAU | 228 | UGCCAUUGUGGUAUCAUAGAUGAC | 628 |
| | GGCA | | CAA | |
| LPA-4647 | GUCAUCUAUGAUACCACACAG | 229 | AUGCCUGUGUGGUAUCAUAGAUGA | 629 |
| | GCAT | | CCA | |
| LPA-4648 | UCAUCUAUGAUACCACACUAG | 230 | GAUGCUAGUGUGGUAUCAUAGAUG | 630 |
| | CATC | | ACC | |
| LPA-4649 | CAUCUAUGAUACCACACUGAC | 231 | UGAUGUCAGUGUGGUAUCAUAGAU | 631 |
| | AUCA | | GAC | |
| LPA-4650 | AUCUAUGAUACCACACUGGAA | 232 | CUGAUUCCAGUGUGGUAUCAUAGA | 632 |
| | UCAG | | UGA | |
| LPA-4651 | UCUAUGAUACCACACUGGCAU | 233 | UCUGAUGCCAGUGUGGUAUCAUAG | 633 |
| | CAGA | | AUG | |
| LPA-4652 | CUAUGAUACCACACUGGCAAC | 234 | CUCUGUUGCCAGUGUGGUAUCAUA | 634 |
| | AGAG | | GAU | |
| LPA-4655 | UGAUACCACACUGGCAUCAAA | 235 | GUCCUUUGAUGCCAGUGUGGUAUC | 635 |
| | GGAC | | AUA | |
| LPA-4657 | AUACCACACUGGCAUCAGAAG | 236 | GGGUCUUCUGAUGCCAGUGUGGUA | 636 |
| | ACCC | | UCA | |
| LPA-4673 | AGAGGACCCCAGAAAACUAAC | 237 | UUUGGUUAGUUUUCUGGGGUCCUC | 637 |
| | CAAA | | UGA | |
| LPA-4674 | GAGGACCCCAGAAAACUACAC | 238 | AUUUGUGUAGUUUUCUGGGGUCCU | 638 |
| | AAAT | | CUG | |
| LPA-4712 | AGAACUACUGCAGGAAUCCAG | 239 | GAAUCUGGAUUCCUGCAGUAGUUC | 639 |
| | AUTC | | UCG | |
| LPA-4715 | ACUACUGCAGGAAUCCAGAAU | 240 | CCAGAUUCUGGAUUCCUGCAGUAG | 640 |
| | CUGG | | UUC | |
| LPA-4717 | UACUGCAGGAAUCCAGAUUAU | 241 | UCCCAUAAUCUGGAUUCCUGCAGU | 641 |
| | GGGA | | AGU | |
| LPA-4718 | ACUGCAGGAAUCCAGAUUCAG | 242 | UUCCCUGAAUCUGGAUUCCUGCAG | 642 |
| | GGAA | | UAG | |
| LPA-4719 | CUGCAGGAAUCCAGAUUCUAG | 243 | UUUCCUAGAAUCUGGAUUCCUGCA | 643 |
| | GAAA | | GUA | |
| LPA-4720 | UGCAGGAAUCCAGAUUCUGAG | 244 | GUUUCUCAGAAUCUGGAUUCCUGC | 644 |
| | AAAC | | AGU | |
| LPA-4721 | GCAGGAAUCCAGAUUCUGGAA | 245 | UGUUUUCCAGAAUCUGGAUUCCUG | 645 |
| | AACA | | CAG | |
| LPA-4724 | GGAAUCCAGAUUCUGGGAAAC | 246 | GGUUGUUUCCCAGAAUCUGGAUUC | 646 |
| | AACC | | CUG | |
| LPA-4738 | GGGAAACAACCCUGGUGUUAC | 247 | UUGUGUAACACCAGGGUUGUUUCC | 647 |
| | ACAA | | CAG | |
| LPA-4739 | GGAAACAACCCUGGUGUUAAA | 248 | GUUGUUUAACACCAGGGUUGUUUC | 648 |
| | CAAC | | CCA | |
| LPA-4771 | UGUGUGAGGUGGGAGUACUAC | 249 | GAUUGUAGUACUCCCACCUCACAC | 649 |
| | AATC | | ACG | |
| LPA-4772 | GUGUGAGGUGGGAGUACUGAA | 250 | AGAUUUCAGUACUCCCACCUCACA | 650 |
| | AUCT | | CAC | |
| LPA-4774 | GUGAGGUGGGAGUACUGCAAU | 251 | UCAGAUUGCAGUACUCCCACCUCA | 651 |
| | CUGA | | CAC | |
| LPA-4775 | UGAGGUGGGAGUACUGCAAAC | 252 | GUCAGUUUGCAGUACUCCCACCUC | 652 |
| | UGAC | | ACA | |
| LPA-4795 | CUGACACAAUGCUCAGAAAAA | 253 | AUUCUUUUUCUGAGCAUUGUGUCA | 653 |
| | GAAT | | GAU | |
| LPA-4796 | UGACACAAUGCUCAGAAACAG | 254 | GAUUCUGUUUCUGAGCAUUGUGUC | 654 |
| | AATC | | AGA | |
| LPA-4797 | GACACAAUGCUCAGAAACAAA | 255 | UGAUUUUGUUUCUGAGCAUUGUGU | 655 |
| | AUCA | | CAG | |
| LPA-4798 | ACACAAUGCUCAGAAACAGAA | 256 | CUGAUUCUGUUUCUGAGCAUUGUG | 656 |
| | UCAG | | UCA | |
| LPA-4799 | CACAAUGCUCAGAAACAGAAU | 257 | CCUGAUUCUGUUUCUGAGCAUUGU | 657 |
| | CAGG | | GUC | |
| LPA-4800 | ACAAUGCUCAGAAACAGAAAC | 258 | ACCUGUUUCUGUUUCUGAGCAUUG | 658 |
| | AGGT | | UGU | |
| LPA-4801 | CAAUGCUCAGAAACAGAAUAA | 259 | CACCUUAUUCUGUUUCUGAGCAUU | 659 |
| | GGTG | | GUG | |
| LPA-4802 | AAUGCUCAGAAACAGAAUCAG | 260 | ACACCUGAUUCUGUUUCUGAGCAU | 660 |
| | GUGT | | UGU | |
| LPA-4803 | AUGCUCAGAAACAGAAUCAAG | 261 | GACACUUGAUUCUGUUUCUGAGCA | 661 |
| | UGTC | | UUG | |
| LPA-4804 | UGCUCAGAAACAGAAUCAGAU | 262 | GGACAUCUGAUUCUGUUUCUGAGC | 662 |
| | GUCC | | AUU | |
| LPA-4806 | CUCAGAAACAGAAUCAGGUAU | 263 | UAGGAUACCUGAUUCUGUUUCUGA | 663 |
| | CCTA | | GCA | |
| LPA-4808 | CAGAAACAGAAUCAGGUGUAC | 264 | UCUAGUACACCUGAUUCUGUUUCU | 664 |
| | UAGA | | GAG | |
| LPA-4809 | AGAAACAGAAUCAGGUGUCAU | 265 | CUCUAUGACACCUGAUUCUGUUUC | 665 |
| | AGAG | | UGA | |
| LPA-4810 | GAAACAGAAUCAGGUGUCCAA | 266 | UCUCUUGGACACCUGAUUCUGUUU | 666 |
| | GAGA | | CUG | |
| LPA-4811 | AAACAGAAUCAGGUGUCCUAG | 267 | GUCUCUAGGACACCUGAUUCUGUU | 667 |
| | AGAC | | UCU | |
| LPA-4812 | AACAGAAUCAGGUGUCCUAAA | 268 | AGUCUUUAGGACACCUGAUUCUGU | 668 |
| | GACT | | UUC | |
| LPA-4814 | CAGAAUCAGGUGUCCUAGAAA | 269 | GGAGUUUCUAGGACACCUGAUUCU | 669 |
| | CUCC | | GUU | |
| LPA-4816 | GAAUCAGGUGUCCUAGAGAAU | 270 | UGGGAUUCUCUAGGACACCUGAUU | 670 |
| | CCCA | | CUG | |
| LPA-4818 | AUCAGGUGUCCUAGAGACUAC | 271 | AGUGGUAGUCUCUAGGACACCUGA | 671 |
| | CACT | | UUC | |
| LPA-4822 | GGUGUCCUAGAGACUCCCAAU | 272 | CAACAUUGGGAGUCUCUAGGACAC | 672 |
| | GUTG | | CUG | |
| LPA-4827 | CCUAGAGACUCCCACUGUUAU | 273 | UGGAAUAACAGUGGGAGUCUCUAG | 673 |
| | UCCA | | GAC | |
| LPA-4828 | CUAGAGACUCCCACUGUUGAU | 274 | CUGGAUCAACAGUGGGAGUCUCUA | 674 |
| | CCAG | | GGA | |
| LPA-4829 | UAGAGACUCCCACUGUUGUAC | 275 | ACUGGUACAACAGUGGGAGUCUCU | 675 |
| | CAGT | | AGG | |
| LPA-4830 | AGAGACUCCCACUGUUGUUAC | 276 | AACUGUAACAACAGUGGGAGUCUC | 676 |
| | AGTT | | UAG | |
| LPA-4831 | GAGACUCCCACUGUUGUUCAA | 277 | GAACUUGAACAACAGUGGGAGUCU | 677 |
| | GUTC | | CUA | |
| LPA-4832 | AGACUCCCACUGUUGUUCCAG | 278 | GGAACUGGAACAACAGUGGGAGUC | 678 |
| | UUCC | | UCU | |
| LPA-4867 | GCUCAUUCUGAAGCAGCACAA | 279 | CAGUUUGUGCUGCUUCAGAAUGAG | 679 |
| | ACTG | | CCU | |
| LPA-4868 | CUCAUUCUGAAGCAGCACCAA | 280 | UCAGUUGGUGCUGCUUCAGAAUGA | 680 |
| | CUGA | | GCC | |
| LPA-4869 | UCAUUCUGAAGCAGCACCAAC | 281 | CUCAGUUGGUGCUGCUUCAGAAUG | 681 |
| | UGAG | | AGC | |
| LPA-4870 | CAUUCUGAAGCAGCACCAAAU | 282 | GCUCAUUUGGUGCUGCUUCAGAAU | 682 |
| | GAGC | | GAG | |
| LPA-4871 | AUUCUGAAGCAGCACCAACAG | 283 | UGCUCUGUUGGUGCUGCUUCAGAA | 683 |
| | AGCA | | UGA | |
| LPA-4872 | UUCUGAAGCAGCACCAACUAA | 284 | UUGCUUAGUUGGUGCUGCUUCAGA | 684 |
| | GCAA | | AUG | |
| LPA-4873 | UCUGAAGCAGCACCAACUGAG | 285 | UUUGCUCAGUUGGUGCUGCUUCAG | 685 |
| | CAAA | | AAU | |
| LPA-4874 | CUGAAGCAGCACCAACUGAAC | 286 | GUUUGUUCAGUUGGUGCUGCUUCA | 686 |
| | AAAC | | GAA | |
| LPA-4875 | UGAAGCAGCACCAACUGAGAA | 287 | GGUUUUCUCAGUUGGUGCUGCUUC | 687 |
| | AACC | | AGA | |
| LPA-4876 | GAAGCAGCACCAACUGAGCAA | 288 | GGGUUUGCUCAGUUGGUGCUGCUU | 688 |
| | ACCC | | CAG | |
| LPA-4877 | AAGCAGCACCAACUGAGCAAA | 289 | GGGGUUUGCUCAGUUGGUGCUGCU | 689 |
| | CCCC | | UCA | |
| LPA-4912 | CAGUGCUACCAUGGUAAUGAC | 290 | UCUGGUCAUUACCAUGGUAGCACU | 690 |
| | CAGA | | GCC | |
| LPA-4913 | AGUGCUACCAUGGUAAUGGAC | 291 | CUCUGUCCAUUACCAUGGUAGCAC | 691 |
| | AGAG | | UGC | |
| LPA-4948 | ACAUUCUCCACCACUGUCAAA | 292 | UUCCUUUGACAGUGGUGGAGAAUG | 692 |
| | GGAA | | UGC | |
| LPA-4959 | CACUGUCACAGGAAGGACAAG | 293 | UUGACUUGUCCUUCCUGUGACAGU | 693 |
| | UCAA | | GGU | |
| LPA-4960 | ACUGUCACAGGAAGGACAUAU | 294 | AUUGAUAUGUCCUUCCUGUGACAG | 694 |
| | CAAT | | UGG | |
| LPA-4961 | CUGUCACAGGAAGGACAUGAC | 295 | GAUUGUCAUGUCCUUCCUGUGACA | 695 |
| | AATC | | GUG | |
| LPA-4962 | UGUCACAGGAAGGACAUGUAA | 296 | AGAUUUACAUGUCCUUCCUGUGAC | 696 |
| | AUCT | | AGU | |
| LPA-4963 | GUCACAGGAAGGACAUGUCAA | 297 | AAGAUUGACAUGUCCUUCCUGUGA | 697 |
| | UCTT | | CAG | |
| LPA-4964 | UCACAGGAAGGACAUGUCAAU | 298 | CAAGAUUGACAUGUCCUUCCUGUG | 698 |
| | CUTG | | ACA | |
| LPA-4966 | ACAGGAAGGACAUGUCAAUAU | 299 | ACCAAUAUUGACAUGUCCUUCCUG | 699 |
| | UGGT | | UGA | |
| LPA-4967 | CAGGAAGGACAUGUCAAUCAU | 300 | GACCAUGAUUGACAUGUCCUUCCU | 700 |
| | GGTC | | GUG | |
| LPA-4968 | AGGAAGGACAUGUCAAUCUAG | 301 | UGACCUAGAUUGACAUGUCCUUCC | 701 |
| | GUCA | | UGU | |
| LPA-4969 | GGAAGGACAUGUCAAUCUUAG | 302 | AUGACUAAGAUUGACAUGUCCUUC | 702 |
| | UCAT | | CUG | |
| LPA-4970 | GAAGGACAUGUCAAUCUUGAU | 303 | GAUGAUCAAGAUUGACAUGUCCUU | 703 |
| | CATC | | CCU | |
| LPA-4971 | AAGGACAUGUCAAUCUUGGAC | 304 | GGAUGUCCAAGAUUGACAUGUCCU | 704 |
| | AUCC | | UCC | |
| LPA-4972 | AGGACAUGUCAAUCUUGGUAA | 305 | UGGAUUACCAAGAUUGACAUGUCC | 705 |
| | UCCA | | UUC | |
| LPA-4973 | GGACAUGUCAAUCUUGGUCAU | 306 | AUGGAUGACCAAGAUUGACAUGUC | 706 |
| | CCAT | | CUU | |
| LPA-4974 | GACAUGUCAAUCUUGGUCAAC | 307 | CAUGGUUGACCAAGAUUGACAUGU | 707 |
| | CATG | | CCU | |
| LPA-4975 | ACAUGUCAAUCUUGGUCAUAC | 308 | UCAUGUAUGACCAAGAUUGACAUG | 708 |
| | AUGA | | UCC | |
| LPA-4976 | CAUGUCAAUCUUGGUCAUCAA | 309 | GUCAUUGAUGACCAAGAUUGACAU | 709 |
| | UGAC | | GUC | |
| LPA-4977 | AUGUCAAUCUUGGUCAUCCAU | 310 | UGUCAUGGAUGACCAAGAUUGACA | 710 |
| | GACA | | UGU | |
| LPA-4978 | UGUCAAUCUUGGUCAUCCAAG | 311 | GUGUCUUGGAUGACCAAGAUUGAC | 711 |
| | ACAC | | AUG | |
| LPA-4979 | GUCAAUCUUGGUCAUCCAUAA | 312 | GGUGUUAUGGAUGACCAAGAUUGA | 712 |
| | CACC | | CAU | |
| LPA-4980 | UCAAUCUUGGUCAUCCAUGAC | 313 | UGGUGUCAUGGAUGACCAAGAUUG | 713 |
| | ACCA | | ACA | |
| LPA-4981 | CAAUCUUGGUCAUCCAUGAAA | 314 | GUGGUUUCAUGGAUGACCAAGAUU | 714 |
| | CCAC | | GAC | |
| LPA-4982 | AAUCUUGGUCAUCCAUGACAC | 315 | UGUGGUGUCAUGGAUGACCAAGAU | 715 |
| | CACA | | UGA | |
| LPA-4983 | AUCUUGGUCAUCCAUGACAAC | 316 | GUGUGUUGUCAUGGAUGACCAAGA | 716 |
| | ACAC | | UUG | |
| LPA-5048 | UGACAAUGAACUACUGCAGAA | 317 | GGAUUUCUGCAGUAGUUCAUUGUC | 717 |
| | AUCC | | AGG | |
| LPA-5049 | GACAAUGAACUACUGCAGGAA | 318 | UGGAUUCCUGCAGUAGUUCAUUGU | 718 |
| | UCCA | | CAG | |
| LPA-5050 | ACAAUGAACUACUGCAGGAAU | 319 | CUGGAUUCCUGCAGUAGUUCAUUG | 719 |
| | CCAG | | UCA | |
| LPA-5051 | CAAUGAACUACUGCAGGAAAC | 320 | UCUGGUUUCCUGCAGUAGUUCAUU | 720 |
| | CAGA | | GUC | |
| LPA-5052 | AAUGAACUACUGCAGGAAUAC | 321 | AUCUGUAUUCCUGCAGUAGUUCAU | 721 |
| | AGAT | | UGU | |
| LPA-5053 | AUGAACUACUGCAGGAAUCAA | 322 | CAUCUUGAUUCCUGCAGUAGUUCA | 722 |
| | GATG | | UUG | |
| LPA-5054 | UGAACUACUGCAGGAAUCCAG | 323 | GCAUCUGGAUUCCUGCAGUAGUUC | 723 |
| | AUGC | | AUU | |
| LPA-5058 | CUACUGCAGGAAUCCAGAUAC | 324 | AUCGGUAUCUGGAUUCCUGCAGUA | 724 |
| | CGAT | | GUU | |
| LPA-5084 | CAGGCCCUUGGUGUUUUACAA | 325 | UCCAUUGUAAAACACCAAGGGCCU | 725 |
| | UGGA | | GUA | |
| LPA-5090 | CUUGGUGUUUUACCAUGGAAC | 326 | CUGGGUUCCAUGGUAAAACACCAA | 726 |
| | CCAG | | GGG | |
| LPA-5091 | UUGGUGUUUUACCAUGGACAC | 327 | GCUGGUGUCCAUGGUAAAACACCA | 727 |
| | CAGC | | AGG | |
| LPA-5092 | UGGUGUUUUACCAUGGACCAC | 328 | UGCUGUGGUCCAUGGUAAAACACC | 728 |
| | AGCA | | AAG | |
| LPA-5093 | GGUGUUUUACCAUGGACCCAA | 329 | AUGCUUGGGUCCAUGGUAAAACAC | 729 |
| | GCAT | | CAA | |
| LPA-5094 | GUGUUUUACCAUGGACCCCAG | 330 | GAUGCUGGGGUCCAUGGUAAAACA | 730 |
| | CATC | | CCA | |
| LPA-5096 | GUUUUACCAUGGACCCCAGAA | 331 | CUGAUUCUGGGGUCCAUGGUAAAA | 731 |
| | UCAG | | CAC | |
| LPA-5124 | GGAGUACUGCAACCUGACGAG | 332 | GCAUCUCGUCAGGUUGCAGUACUC | 732 |
| | AUGC | | CCA | |
| LPA-5125 | GAGUACUGCAACCUGACGCAA | 333 | AGCAUUGCGUCAGGUUGCAGUACU | 733 |
| | UGCT | | CCC | |
| LPA-5127 | GUACUGCAACCUGACGCGAAG | 334 | UGAGCUUCGCGUCAGGUUGCAGUA | 734 |
| | CUCA | | CUC | |
| LPA-5128 | UACUGCAACCUGACGCGAUAC | 335 | CUGAGUAUCGCGUCAGGUUGCAGU | 735 |
| | UCAG | | ACU | |
| LPA-5131 | UGCAACCUGACGCGAUGCUAA | 336 | UGUCUUAGCAUCGCGUCAGGUUGC | 736 |
| | GACA | | AGU | |
| LPA-5136 | CCUGACGCGAUGCUCAGACAC | 337 | UUCUGUGUCUGAGCAUCGCGUCAG | 737 |
| | AGAA | | GUU | |
| LPA-5137 | CUGACGCGAUGCUCAGACAAA | 338 | CUUCUUUGUCUGAGCAUCGCGUCA | 738 |
| | GAAG | | GGU | |
| LPA-5144 | GAUGCUCAGACACAGAAGGAA | 339 | ACAGUUCCUUCUGUGUCUGAGCAU | 739 |
| | CUGT | | CGC | |
| LPA-5145 | AUGCUCAGACACAGAAGGGAC | 340 | CACAGUCCCUUCUGUGUCUGAGCA | 740 |
| | UGTG | | UCG | |
| LPA-5151 | AGACACAGAAGGGACUGUGAU | 341 | AGCGAUCACAGUCCCUUCUGUGUC | 741 |
| | CGCT | | UGA | |
| LPA-5467 | GCAUCCUCUUCAUUUGAUUAU | 342 | UCCCAUAAUCAAAUGAAGAGGAUG | 742 |
| | GGGA | | CAC | |
| LPA-5468 | CAUCCUCUUCAUUUGAUUGAG | 343 | UUCCCUCAAUCAAAUGAAGAGGAU | 743 |
| | GGAA | | GCA | |
| LPA-5469 | AUCCUCUUCAUUUGAUUGUAG | 344 | CUUCCUACAAUCAAAUGAAGAGGA | 744 |
| | GAAG | | UGC | |
| LPA-5470 | UCCUCUUCAUUUGAUUGUGAG | 345 | GCUUCUCACAAUCAAAUGAAGAGG | 745 |
| | AAGC | | AUG | |
| LPA-5471 | CCUCUUCAUUUGAUUGUGGAA | 346 | GGCUUUCCACAAUCAAAUGAAGAG | 746 |
| | AGCC | | GAU | |
| LPA-5474 | CUUCAUUUGAUUGUGGGAAAC | 347 | UGAGGUUUCCCACAAUCAAAUGAA | 747 |
| | CUCA | | GAG | |
| LPA-5475 | UUCAUUUGAUUGUGGGAAGAC | 348 | UUGAGUCUUCCCACAAUCAAAUGA | 748 |
| | UCAA | | AGA | |
| LPA-5476 | UCAUUUGAUUGUGGGAAGCAU | 349 | CUUGAUGCUUCCCACAAUCAAAUG | 749 |
| | CAAG | | AAG | |
| LPA-5477 | CAUUUGAUUGUGGGAAGCCAC | 350 | ACUUGUGGCUUCCCACAAUCAAAU | 750 |
| | AAGT | | GAA | |
| LPA-5478 | AUUUGAUUGUGGGAAGCCUAA | 351 | CACUUUAGGCUUCCCACAAUCAAA | 751 |
| | AGTG | | UGA | |
| LPA-5486 | GUGGGAAGCCUCAAGUGGAAC | 352 | UUCGGUUCCACUUGAGGCUUCCCA | 752 |
| | CGAA | | CAA | |
| LPA-5509 | AAGAAAUGUCCUGGAAGCAAU | 353 | CUACAUUGCUUCCAGGACAUUUCU | 753 |
| | GUAG | | UCG | |
| LPA-5510 | AGAAAUGUCCUGGAAGCAUAG | 354 | CCUACUAUGCUUCCAGGACAUUUC | 754 |
| | UAGG | | UUC | |
| LPA-5511 | GAAAUGUCCUGGAAGCAUUAU | 355 | CCCUAUAAUGCUUCCAGGACAUUU | 755 |
| | AGGG | | CUU | |
| LPA-5513 | AAUGUCCUGGAAGCAUUGUAG | 356 | CCCCCUACAAUGCUUCCAGGACAU | 756 |
| | GGGG | | UUC | |
| LPA-5514 | AUGUCCUGGAAGCAUUGUAAG | 357 | CCCCCUUACAAUGCUUCCAGGACA | 757 |
| | GGGG | | UUU | |
| LPA-5581 | AGAACAAGGUUUGGAAAGCAC | 358 | AGAAGUGCUUUCCAAACCUUGUUC | 758 |
| | UUCT | | UGA | |
| LPA-5582 | GAACAAGGUUUGGAAAGCAAU | 359 | CAGAAUUGCUUUCCAAACCUUGUU | 759 |
| | UCTG | | CUG | |
| LPA-5583 | AACAAGGUUUGGAAAGCACAU | 360 | ACAGAUGUGCUUUCCAAACCUUGU | 760 |
| | CUGT | | UCU | |
| LPA-5584 | ACAAGGUUUGGAAAGCACUAC | 361 | CACAGUAGUGCUUUCCAAACCUUG | 761 |
| | UGTG | | UUC | |
| LPA-5585 | CAAGGUUUGGAAAGCACUUAU | 362 | CCACAUAAGUGCUUUCCAAACCUU | 762 |
| | GUGG | | GUU | |
| LPA-5586 | AAGGUUUGGAAAGCACUUCAG | 363 | UCCACUGAAGUGCUUUCCAAACCU | 763 |
| | UGGA | | UGU | |
| LPA-5587 | AGGUUUGGAAAGCACUUCUAU | 364 | CUCCAUAGAAGUGCUUUCCAAACC | 764 |
| | GGAG | | UUG | |
| LPA-5592 | UGGAAAGCACUUCUGUGGAAG | 365 | GGUGCUUCCACAGAAGUGCUUUCC | 765 |
| | CACC | | AAA | |
| LPA-5606 | GUGGAGGCACCUUAAUAUCAC | 366 | UCUGGUGAUAUUAAGGUGCCUCCA | 766 |
| | CAGA | | CAG | |
| LPA-5616 | CUUAAUAUCCCCAGAGUGGAU | 367 | CAGCAUCCACUCUGGGGAUAUUAA | 767 |
| | GCTG | | GGU | |
| LPA-5618 | UAAUAUCCCCAGAGUGGGUAC | 368 | GUCAGUACCCACUCUGGGGAUAUU | 768 |
| | UGAC | | AAG | |
| LPA-5628 | AGAGUGGGUGCUGACUGCUAC | 369 | GUGAGUAGCAGUCAGCACCCACUC | 769 |
| | UCAC | | UGG | |
| LPA-5685 | CAAGGUCAUCCUGGGUGCAAA | 370 | UUGGUUUGCACCCAGGAUGACCUU | 770 |
| | CCAA | | GUA | |
| LPA-5694 | CCUGGGUGCACACCAAGAAAU | 371 | GUUCAUUUCUUGGUGUGCACCCAG | 771 |
| | GAAC | | GAU | |
| LPA-5699 | GUGCACACCAAGAAGUGAAAC | 372 | UCGAGUUUCACUUCUUGGUGUGCA | 772 |
| | UCGA | | CCC | |
| LPA-5775 | AGCAGAUAUUGCCUUGCUAAA | 373 | UAGCUUUAGCAAGGCAAUAUCUGC | 773 |
| | GCTA | | UUG | |
| LPA-5776 | GCAGAUAUUGCCUUGCUAAAG | 374 | UUAGCUUUAGCAAGGCAAUAUCUG | 774 |
| | CUAA | | CUU | |
| LPA-5777 | CAGAUAUUGCCUUGCUAAAAC | 375 | CUUAGUUUUAGCAAGGCAAUAUCU | 775 |
| | UAAG | | GCU | |
| LPA-5778 | AGAUAUUGCCUUGCUAAAGAU | 376 | GCUUAUCUUUAGCAAGGCAAUAUC | 776 |
| | AAGC | | UGC | |
| LPA-5779 | GAUAUUGCCUUGCUAAAGCAA | 377 | UGCUUUGCUUUAGCAAGGCAAUAU | 777 |
| | AGCA | | CUG | |
| LPA-5780 | AUAUUGCCUUGCUAAAGCUAA | 378 | CUGCUUAGCUUUAGCAAGGCAAUA | 778 |
| | GCAG | | UCU | |
| LPA-5781 | UAUUGCCUUGCUAAAGCUAAG | 379 | CCUGCUUAGCUUUAGCAAGGCAAU | 779 |
| | CAGG | | AUC | |
| LPA-5813 | UCAUCACUGACAAAGUAAUAC | 380 | GCUGGUAUUACUUUGUCAGUGAUG | 780 |
| | CAGC | | ACG | |
| LPA-5873 | GGACUGAAUGUUACAUCACAG | 381 | CAGCCUGUGAUGUAACAUUCAGUC | 781 |
| | GCTG | | CUG | |
| LPA-5874 | GACUGAAUGUUACAUCACUAG | 382 | CCAGCUAGUGAUGUAACAUUCAGU | 782 |
| | CUGG | | CCU | |
| LPA-5875 | ACUGAAUGUUACAUCACUGAC | 383 | CCCAGUCAGUGAUGUAACAUUCAG | 783 |
| | UGGG | | UCC | |
| LPA-5876 | CUGAAUGUUACAUCACUGGAU | 384 | CCCCAUCCAGUGAUGUAACAUUCA | 784 |
| | GGGG | | GUC | |
| LPA-5877 | UGAAUGUUACAUCACUGGCAG | 385 | UCCCCUGCCAGUGAUGUAACAUUC | 785 |
| | GGGA | | AGU | |
| LPA-5879 | AAUGUUACAUCACUGGCUGAG | 386 | UCUCCUCAGCCAGUGAUGUAACAU | 786 |
| | GAGA | | UCA | |
| LPA-5902 | GAAACCCAAGGUACCUUUGAG | 387 | CAGUCUCAAAGGUACCUUGGGUUU | 787 |
| | ACTG | | CUC | |
| LPA-0190-M1 | UCCACCACUGUCACAGGAAAG | 388 | UUUCCUGUGACAGUGGUGGAGG | 788 |
| | CAGCCGAAAGGCUGC | | | |
| LPA-0501-M1 | UGGUAAUGGACAGAGUUAUAG | 389 | UAUAACUCUGUCCAUUACCAGG | 789 |
| | CAGCCGAAAGGCUGC | | | |
| LPA-3100-M1 | UACUGCAACCUGACACGAUAG | 390 | UAUCGUGUCAGGUUGCAGUAGG | 790 |
| | CAGCCGAAAGGCUGC | | | |
| LPA-3286-M1 | AGAACUUGCCAAGCUUGGUAG | 391 | UACCAAGCUUGGCAAGUUCUGG | 791 |
| | CAGCCGAAAGGCUGC | | | |
| LPA-3288-M1 | AACUUGCCAAGCUUGGUCAAG | 392 | UUGACCAAGCUUGGCAAGUUGG | 792 |
| | CAGCCGAAAGGCUGC | | | |
| LPA-3291-M1 | UUGCCAAGCUUGGUCAUCUAG | 393 | UAGAUGACCAAGCUUGGCAAGG | 793 |
| | CAGCCGAAAGGCUGC | | | |
| LPA-3584-M1 | AUGGACAGAGUUAUCGAGGAG | 394 | UCCUCGAUAACUCUGUCCAUGG | 794 |
| | CAGCCGAAAGGCUGC | | | |
| LPA-3585-M1 | UGGACAGAGUUAUCGAGGCAG | 395 | UGCCUCGAUAACUCUGUCCAGG | 795 |
| | CAGCCGAAAGGCUGC | | | |
| LPA-4645-M1 | UGGUCAUCUAUGAUACCACAG | 396 | UGUGGUAUCAUAGAUGACCAGG | 796 |
| | CAGCCGAAAGGCUGC | | | |
| LPA-4717-M1 | UACUGCAGGAAUCCAGAUUAG | 397 | UAAUCUGGAUUCCUGCAGUAGG | 797 |
| | CAGCCGAAAGGCUGC | | | |
| LPA-5510-M1 | AGAAAUGUCCUGGAAGCAUAG | 398 | UAUGCUUCCAGGACAUUUCUGG | 798 |
| | CAGCCGAAAGGCUGC | | | |
| LPA-3750-M1 | GACAACAGAAUAUUAUCCAAG | 399 | UUGGAUAAUAUUCUGUUGUCGG | 799 |
| | CAGCCGAAAGGCUGC | | | |
| LPA-2900-M2 | AUGGACAGAGUUAUCAAGGAG | 400 | UCCUUGAUAACUCUGUCCAUGG | 800 |
| | CAGCCGAAAGGCUGC | | | |
| LPA-3675-M2 | GACAACAGAAUAUUAUCCAAG | 401 | UUGGAUAAUAUUCUGUUGUCGG | 801 |
| | CAGCCGAAAGGCUGC | | | |
| LPA-2900-M3 | AUGGACAGAGUUAUCAAGGAG | 402 | UCCUUGAUAACUCUGUCCAUGG | 802 |
| | CAGCCGAAAGGCUGC | | | |
| LPA-3675-M3 | GACAACAGAAUAUUAUCCAAG | 403 | UUGGAUAAUAUUCUGUUGUCGG | 803 |
| | CAGCCGAAAGGCUGC | | | |

Human (Hs): NM_005577.3 (SEQ ID NO: 1)
CTGGGATTGG GACACACTTT CTGGGCACTG CTGGCCAGTC CCAAAATGGA ACATAAGGAA
GTGGTTCTTC TACTTCTTTT ATTTCTGAAA TCAGCAGCAC CTGAGCAAAG CCATGTGGTC
CAGGATTGCT ACCATGGTGA TGGACAGAGT TATCGAGGCA CGTACTCCAC CACTGTCACA
GGAAGGACCT GCCAAGCTTG GTCATCTATG ACACCACATC AACATAATAG GACCACAGAA
AACTACCCAA ATGCTGGCTT GATCATGAAC TACTGCAGGA ATCCAGATGC TGTGGCAGCT
CCTTATTGTT ATACGAGGGA TCCCGGTGTC AGGTGGGAGT ACTGCAACCT GACGCAATGC
TCAGACGCAG AAGGGACTGC CGTCGCGCCT CCGACTGTTA CCCCGGTTCC AAGCCTAGAG
GCTCCTTCCG AACAAGCACC GACTGAGCAA AGGCCTGGGG TGCAGGAGTG CTACCATGGT
AATGGACAGA GTTATCGAGG CACATACTCC ACCACTGTCA CAGGAAGAAC CTGCCAAGCT
TGGTCATCTA TGACACCACA CTCGCATAGT CGGACCCCAG AATACTACCC AAATGCTGGC
TTGATCATGA ACTACTGCAG GAATCCAGAT GCTGTGGCAG CTCCTTATTG TTATACGAGG
GATCCCGGTG TCAGGTGGGA GTACTGCAAC CTGACGCAAT GCTCAGACGC AGAAGGGACT
GCCGTCGCGC CTCCGACTGT TACCCCGGTT CCAAGCCTAG AGGCTCCTTC CGAACAAGCA
CCGACTGAGC AAAGGCCTGG GGTGCAGGAG TGCTACCATG GTAATGGACA GAGTTATCGA
GGCACATACT CCACCACTGT CACAGGAAGA ACCTGCCAAG CTTGGTCATC TATGACACCA
CACTCGCATA GTCGGACCCC AGAATACTAC CCAAATGCTG GCTTGATCAT GAACTACTGC
AGGAATCCAG ATGCTGTGGC AGCTCCTTAT TGTTATACGA GGGATCCCGG TGTCAGGTGG
GAGTACTGCA ACCTGACGCA ATGCTCAGAC GCAGAAGGGA CTGCCGTCGC GCCTCCGACT
GTTACCCCGG TTCCAAGCCT AGAGGCTCCT TCCGAACAAG CACCGACTGA GCAGAGGCCT
GGGGTGCAGG AGTGCTACCA CGGTAATGGA CAGAGTTATC GAGGCACATA CTCCACCACT
GTCACTGGAA GAACCTGCCA AGCTTGGTCA TCTATGACAC CACACTCGCA TAGTCGGACC
CCAGAATACT ACCCAAATGC TGGCTTGATC ATGAACTACT GCAGGAATCC AGATGCTGTG
GCAGCTCCTT ATTGTTATAC GAGGGATCCC GGTGTCAGGT GGGAGTACTG CAACCTGACG
CAATGCTCAG ACGCAGAAGG GACTGCCGTC GCGCCTCCGA CTGTTACCCC GGTTCCAAGC
CTAGAGGCTC CTTCCGAACA AGCACCGACT GAGCAAAGGC CTGGGGTGCA GGAGTGCTAC
CATGGTAATG GACAGAGTTA TCGAGGCACA TACTCCACCA CTGTCACAGG AAGAACCTGC
CAAGCTTGGT CATCTATGAC ACCACACTCG CATAGTCGGA CCCCAGAATA CTACCCAAAT
GCTGGCTTGA TCATGAACTA CTGCAGGAAT CCAGATGCTG TGGCAGCTCC TTATTGTTAT
ACGAGGGATC CCGGTGTCAG GTGGGAGTAC TGCAACCTGA CGCAATGCTC AGACGCAGAA
GGGACTGCCG TCGCGCCTCC GACTGTTACC CCGGTTCCAA GCCTAGAGGC TCCTTCCGAA
CAAGCACCGA CTGAGCAAAG GCCTGGGGTG CAGGAGTGCT ACCATGGTAA TGGACAGAGT
TATCGAGGCA CATACTCCAC CACTGTCACA GGAAGAACCT GCCAAGCTTG GTCATCTATG
ACACCACACT CGCATAGTCG GACCCCAGAA TACTACCCAA ATGCTGGCTT GATCATGAAC
TACTGCAGGA ATCCAGATGC TGTGGCAGCT CCTTATTGTT ATACGAGGGA TCCCGGTGTC
AGGTGGGAGT ACTGCAACCT GACGCAATGC TCAGACGCAG AAGGGACTGC CGTCGCGCCT
CCGACTGTTA CCCCGGTTCC AAGCCTAGAG GCTCCTTCCG AACAAGCACC GACTGAGCAA
AGGCCTGGGG TGCAGGAGTG CTACCATGGT AATGGACAGA GTTATCGAGG CACATACTCC
ACCACTGTCA CAGGAAGAAC CTGCCAAGCT TGGTCATCTA TGACACCACA CTCGCATAGT
CGGACCCCAG AATACTACCC AAATGCTGGC TTGATCATGA ACTACTGCAG GAATCCAGAT
GCTGTGGCAG CTCCTTATTG TTATACGAGG GATCCCGGTG TCAGGTGGGA GTACTGCAAC
CTGACGCAAT GCTCAGACGC AGAAGGGACT GCCGTCGCGC CTCCGACTGT TACCCCGGTT
CCAAGCCTAG AGGCTCCTTC CGAACAAGCA CCGACTGAGC AGAGGCCTGG GGTGCAGGAG
TGCTACCACG GTAATGGACA GAGTTATCGA GGCACATACT CCACCACTGT CACTGGAAGA
ACCTGCCAAG CTTGGTCATC TATGACACCA CACTCGCATA GTCGGACCCC AGAATACTAC
CCAAATGCTG GCTTGATCAT GAACTACTGC AGGAATCCAG ATCCTGTGGC AGCCCCTTAT
TGTTATACGA GGGATCCCAG TGTCAGGTGG GAGTACTGCA ACCTGACACA ATGCTCAGAC
GCAGAAGGGA CTGCCGTCGC GCCTCCAACT ATTACCCCGA TTCCAAGCCT AGAGGCTCCT
TCTGAACAAG CACCAACTGA GCAAAGGCCT GGGGTGCAGG AGTGCTACCA CGGAAATGGA
CAGAGTTATC AAGGCACATA CTTCATTACT GTCACAGGAA GAACCTGCCA AGCTTGGTCA
TCTATGACAC CACACTCGCA TAGTCGGACC CCAGCATACT ACCCAAATGC TGGCTTGATC
AAGAACTACT GCCGAAATCC AGATCCTGTG GCAGCCCCTT GGTGTTATAC AACAGATCCC
AGTGTCAGGT GGGAGTACTG CAACCTGACA CGATGCTCAG ATGCAGAATG GACTGCCTTC
GTCCCTCCGA ATGTTATTCT GGCTCCAAGC CTAGAGGCTT TTTTTGAACA AGCACTGACT
GAGGAAACCC CCGGGGTACA GGACTGCTAC TACCATTATG GACAGAGTTA CCGAGGCACA
TACTCCACCA CTGTCACAGG AAGAACTTGC CAAGCTTGGT CATCTATGAC ACCACACCAG
CATAGTCGGA CCCCAGAAAA CTACCCAAAT GCTGGCCTGA CCAGGAACTA CTGCAGGAAT
CCAGATGCTG AGATTCGCCC TTGGTGTTAC ACCATGGATC CCAGTGTCAG GTGGGAGTAC
TGCAACCTGA CACAATGCCT GGTGACAGAA TCAAGTGTCC TTGCAACTCT CACGGTGGTC
CCAGATCCAA GCACAGAGGC TTCTTCTGAA GAAGCACCAA CGGAGCAAAG CCCCGGGGTC
CAGGATTGCT ACCATGGTGA TGGACAGAGT TATCGAGGCT CATTCTCTAC CACTGTCACA
GGAAGGACAT GTCAGTCTTG GTCCTCTATG ACACCACACT GGCATCAGAG GACAACAGAA
TATTATCCAA ATGGTGGCCT GACCAGGAAC TACTGCAGGA ATCCAGATGC TGAGATTAGT
CCTTGGTGTT ATACCATGGA TCCCAATGTC AGATGGGAGT ACTGCAACCT GACACAATGT
CCAGTGACAG AATCAAGTGT CCTTGCGACG TCCACGGCTG TTTCTGAACA AGCACCAACG
GAGCAAAGCC CCACAGTCCA GGACTGCTAC CATGGTGATG GACAGAGTTA TCGAGGCTCA
TTCTCCACCA CTGTTACAGG AAGGACATGT CAGTCTTGGT CCTCTATGAC ACCACACTGG
CATCAGAGAA CCACAGAATA CTACCCAAAT GGTGGCCTGA CCAGGAACTA CTGCAGGAAT
CCAGATGCTG AGATTCGCCC TTGGTGTTAT ACCATGGATC CCAGTGTCAG ATGGGAGTAC
TGCAACCTGA CGCAATGTCC AGTGATGGAA TCAACTCTCC TCACAACTCC CACGGTGGTC
CCAGTTCCAA GCACAGAGCT TCCTTCTGAA GAAGCACCAA CTGAAAACAG CACTGGGGTC
CAGGACTGCT ACCGAGGTGA TGGACAGAGT TATCGAGGCA CACTCTCCAC CACTATCACA
GGAAGAACAT GTCAGTCTTG GTCGTCTATG ACACCACATT GGCATCGGAG GATCCCATTA
TACTATCCAA ATGCTGGCCT GACCAGGAAC TACTGCAGGA ATCCAGATGC TGAGATTCGC
CCTTGGTGTT ACACCATGGA TCCCAGTGTC AGGTGGGAGT ACTGCAACCT GACACGATGT
CCAGTGACAG AATCGAGTGT CCTCACAACT CCCACAGTGG CCCCGGTTCC AAGCACAGAG
GCTCCTTCTG AACAAGCACC ACCTGAGAAA AGCCCTGTGG TCCAGGATTG CTACCATGGT
GATGGACGGA GTTATCGAGG CATATCCTCC ACCACTGTCA CAGGAAGGAC CTGTCAATCT
TGGTCATCTA TGATACCACA CTGGCATCAG AGGACCCCAG AAAACTACCC AAATGCTGGC
CTGACCGAGA ACTACTGCAG GAATCCAGAT TCTGGGAAAC AACCCTGGTG TTACACAACC
GATCCGTGTG TGAGGTGGGA GTACTGCAAT CTGACACAAT GCTCAGAAAC AGAATCAGGT
GTCCTAGAGA CTCCCACTGT TGTTCCAGTT CCAAGCATGG AGGCTCATTC TGAAGCAGCA
CCAACTGAGC AAACCCCTGT GGTCCGGCAG TGCTACCATG GTAATGGCCA GAGTTATCGA
GGCACATTCT CCACCACTGT CACAGGAAGG ACATGTCAAT CTTGGTCATC CATGACACCA
CACCGGCATC AGAGGACCCC AGAAAACTAC CCAAATGATG GCCTGACAAT GAACTACTGC
AGGAATCCAG ATGCCGATAC AGGCCCTTGG TGTTTTACCA TGGACCCCAG CATCAGGTGG
GAGTACTGCA ACCTGACGCG ATGCTCAGAC ACAGAAGGGA CTGTGGTCGC TCCTCCGACT
GTCATCCAGG TTCCAAGCCT AGGGCCTCCT TCTGAACAAG ACTGTATGTT TGGGAATGGG
AAAGGATACC GGGGCAAGAA GGCAACCACT GTTACTGGGA CGCCATGCCA GGAATGGGCT
GCCCAGGAGC CCCATAGACA CAGCACGTTC ATTCCAGGGA CAAATAAATG GGCAGGTCTG
GAAAAAAATT ACTGCCGTAA CCCTGATGGT GACATCAATG GTCCCTGGTG CTACACAATG
AATCCAAGAA AACTTTTTGA CTACTGTGAT ATCCCTCTCT GTGCATCCTC TTCATTTGAT
TGTGGGAAGC CTCAAGTGGA GCCGAAGAAA TGTCCTGGAA GCATTGTAGG GGGGTGTGTG
GCCCACCCAC ATTCCTGGCC CTGGCAAGTC AGTCTCAGAA CAAGGTTTGG AAAGCACTTC
TGTGGAGGCA CCTTAATATC CCCAGAGTGG GTGCTGACTG CTGCTCACTG CTTGAAGAAG
TCCTCAAGGC CTTCATCCTA CAAGGTCATC CTGGGTGCAC ACCAAGAAGT GAACCTCGAA
TCTCATGTTC AGGAAATAGA AGTGTCTAGG CTGTTCTTGG AGCCCACACA AGCAGATATT
GCCTTGCTAA AGCTAAGCAG GCCTGCCGTC ATCACTGACA AAGTAATGCC AGCTTGTCTG
CCATCCCCAG ACTACATGGT CACCGCCAGG ACTGAATGTT ACATCACTGG CTGGGGAGAA
ACCCAAGGTA CCTTTGGGAC TGGCCTTCTC AAGGAAGCCC AGCTCCTTGT TATTGAGAAT
GAAGTGTGCA ATCACTATAA GTATATTTGT GCTGAGCATT TGGCCAGAGG CACTGACAGT
TGCCAGGGTG ACAGTGGAGG GCCTCTGGTT TGCTTCGAGA AGGACAAATA CATTTTACAA
GGAGTCACTT CTTGGGGTCT TGGCTGTGCA CGCCCCAATA AGCCTGGTGT CTATGCTCGT
GTTTCAAGGT TTGTTACTTG GATTGAGGGA ATGATGAGAA ATAATTAATT GGACGGGAGA
CAGAGTGAAG CATCAACCTA CTTAGAAGCT GAAACGTGGG TAAGGATTTA GCATGCTGGA
AATAATAGAC AGCAATCAAA CGAAGACACT GTTCCCAGCT ACCAGCTATG CCAAACCTTG
GCATTTTTGG TATTTTTGTG TATAAGCTTT TAAGGTCTGA CTGACAAATT CTGTATTAAG
GTGTCATAGC TATGACATTT GTTAAAAATA AACTCTGCAC TTATTTTGAT TTGA
Cynomolgus monkey (Mf): XM_015448517. 1 (SEQ ID NO: 2)
GATGCTGCAT ACTTAATGTC GAAAGGTTGC TTCATCCAAG AGCCTGGAGT TTTCAGAGAC
ACTGTCCTGA AACTATGTCC TGAAACTATG TCATTGAAAC TGAAACATTG TCCTGAAGCT
GGTATTGGGC AATACCAGCG CCTGCAGGCA ACAGCTCGGA TGCACTTAAG ATTTAAATAT
TACCCACAGA AGTTCTGGCT TGTCTGGGAA AACCTTTTGC TAAACAGAAG AGCAACATTT
TTTTTTTTTT CTTTTCTGGA ATTTGTAAAC AGCATTTATT CTCAGCCTTA CCTTCCAAAC
GTTGCACTTG GAACATTGCT GGGCCCCGTG GAAACAGAAG CGAACGTCAG CCAGGCCGGC
AGGGGGCGGC AGACCCCACA CTTCGCCGGG CGCCCTCACC TCCCTGGGAG GGAGTGTGCA
GCTGCCAAAA TCTTCGGCGG GGTTCAGTCC AAGCGACTTC AGCCAGCAGA TGGTCATTCT
CCTGTGACCG TGTGTACTAC AGACTGTTTC AAAACCGGGC AGGCAATTAA CAATGGGAAT
TCTGCCATCA TCGCTGACAA AGTCATCCCA GTTTGTCTGC CATCCCCAAA TTATGTGGTC
GCCAACCAGA CTGAATGTTA TGTCACTGGC TGGGGAGAAA CCCAAGCACT ACCTGAGCAA
AGCCATGTGG TCCAGGATTG CTACCATGGT GATGGACAGA GTTATCAAGG CACATCCTCC
ACCACTGTCA CAGGAAGGAC CTGCCAAGCT TGGTCATCTA TGGAACCACA TCAGCATAAT
AGAACCACAG AAAACTACCC AAATGCTGGC TTGATCAGGA ACTACTGCAG GAATCCAGAT
CCTGTGGCAG CCCCTTATTG TTATACGATG GATCCCAATG TCAGGTGGGA GTACTGCAAC
CTGACACAAT GCTCAGACGC AGAAGGGACT GCCGTCGCAC CTCCGAATGT CACCCCGGTT
CCAAGCCTAG AGGCTCCTTC CGAACAAGCA CCGACTGAGC AAAGGCCTGG GGTGCAGGAG
TGCTACCACG GTAATGGACA GAGTTATCGA GGCACATACT TCACCACTGT GACAGGAAGA
ACCTGCCAAG CTTGGTCATC TATGACACCG CACTCTCATA GTCGGACCCC GGAAAACTAC
CCAAATGGTG GCTTGATCAG GAACTACTGC AGGAATCCAG ATCCTGTGGC AGCCCCTTAT
TGTTATACCA TGGATCCCAA TGTCAGGTGG GAGTACTGCA ACCTGACACA ATGCTCAGAC
GCAGAAGGGA TTGCCGTCAC ACCTCTGACT GTTACCCCGG TTCCAAGCCT AGAGGCTCCT
TCCAAGCAAG CACCAACTGA GCAAAGGCCT GGTGTCCAGG AGTGCTACCA CGGTAATGGA
CAGAGTTATC GAGGCACATA CTTCACCACT GTGACAGGAA GAACCTGCCA AGCTTGGTCA
TCTATGACAC CACATTCTCA TAGTCGTACC CCAGAAAACT ACCCAAATGG TGGCTTGATC
AGGAACTACT GCAGGAATCC AGATCCTGTG GCAGCCCCTT ATTGTTATAC CATGGATCCC
AATGTCAGGT GGGAGTACTG CAACCTGACA CAATGCTCAG ACGCAGAAGG GACTGCCGTC
GCACCTCCGA CTGTCACCCC GGTTCCAAGC CTAGAGGCTC CTTCCGAACA AGCACCGACT
GAGCAAAGGC CTGGGGTGCA GGAGTGCTAC CACGGTAATG GACAGAGTTA TCGAGGCACA
TACTTCACCA CTGTGACAGG AAGAACCTGC CAAGCTTGGT CATCTATGAC ACCGCACTCT
CATAGTCGGA CCCCGGAAAA CTACCCAAAT GGTGGCTTGA TCAGGAACTA CTGCAGGAAT
CCAGATCCTG TGGCAGCCCC TTATTGTTAT ACCATGGATC CCAATGTCAG GTGGGAGTAC
TGCAACCTGA CACAATGCTC AGACGCAGAA GGGACTGCCG TCGCACCTCC GAATGTCACC
CCGGTTCCAA GCCTAGAGGC TCCTTCTGAG CAAGCACCAA CTGAGCAAAG GCTTGGGGTG
CAGGAGTGCT ACCACGGTAA TGGACAGAGT TATCGAGGCA CATACTTCAC CACTGTGACA
GGAAGAACCT GCCAAGCTTG GTCATCTATG ACACCACACT CTCATAGTCG GACCCCAGAA
AACTACCCAA ATGCTGGCTT GGTCAAGAAC TACTGCCGAA ATCCAGATCC TGTGGCAGCC
CCTTGGTGTT ATACAACGGA TCCCAGTGTC AGGTGGGAGT ACTGCAACCT GACACGATGC
TCAGATGCAG AAGGGACTGC TGTTGTGCCT CCAAATATTA TTCCGGTTCC AAGCCTAGAG
GCTTTTCTTG AACAAGAACC GACTGAGGAA ACCCCCGGGG TACAGGAGTG CTACTACCAT
TATGGACAGA GTTATAGAGG CACATACTCC ACCACTGTTA CAGGAAGAAC TTGCCAAGCT
TGGTCATCTA TGACACCACA CCAGCATAGT CGGACCCCAA AAAACTATCC AAATGCTGGC
CTGACCAGGA ACTACTGCAG GAATCCAGAT GCTGAGATTC GCCCTTGGTG TTATACCATG
GATCCCAGTG TCAGGTGGGA GTACTGCAAC CTGACACAAT GTCTGGTGAC AGAATCAAGT
GTCCTTGAAA CTCTCACAGT GGTCCCAGAT CCAAGCACAC AGGCTTCTTC TGAAGAAGCA
CCAACGGAGC AAAGTCCCGA GGTCCAGGAC TGCTACCATG GTGATGGACA GAGTTATCGA
GGCTCATTCT CCACCACTGT CACAGGAAGG ACATGTCAGT CTTGGTCCTC TATGACACCA
CACTGGCATC AGAGGACAAC AGAATATTAT CCAGATGGTG GCCTGACCAG GAACTACTGC
AGGAATCCAG ATGCTGAGAT TCGCCCTTGG TGTTATACCA TGGATCCCAG TGTCAGGTGG
GAGTACTGCA ACCTGACACA ATGTCCAGTG ACAGAATCAA GTGTCCTCGC AACGTCCATG
GCTGTTTCTG AACAAGCACC AATGGAGCAA AGCCCCGGGG TCCAGGACTG CTACCATGGT
GATGGACAGA GTTATCGAGG TTCATTCTCC ACCACTGTCA CAGGAAGGAC ATGTCAGTCT
TGGTCCTCTA TGACACCACA CTGGCATCAG AGGACCATAG AATACTACCC AAATGGTGGC
CTGACCAAGA ACTACTGCAG GAATCCAGAT GCTGAGATTC GCCCTTGGTG TTATACCATG
GATCCCAGAG TCAGATGGGA GTACTGCAAC CTGACACAAT GTGTGGTGAT GGAATCAAGT
GTCCTTGCAA CTCCCATGGT GGTCCCAGTT CCAAGCAGAG AGGTTCCTTC TGAAGAAGCA
CCAACTGAAA ACAGCCCTGG GGTCCAGGAC TGCTACCAAG GTGATGGACA GAGTTATCGA
GGCACATTCT CCACCACTAT CACAGGAAGA ACATGTCAGT CTTGGTTGTC TATGACACCA
CATCGGCATC GGAGGATCCC ATTACGCTAT CCAAATGCTG GCCTGACCAG GAACTATTGC
AGAAATCCAG ATGCTGAGAT TCGCCCTTGG TGTTACACCA TGGATCCCAG TGTCAGGTGG
GAGTACTGCA ACCTGACACA ATGTCCAGTG ACAGAATCAA GTGTCCTCAC AACTCCCACG
GTGGTCCCGG TTCCAAGCAC AGAGGCTCCT TCTGAACAAG CACCACCTGA GAAAAGCCCT
GTGGTCCAGG ATTGCTACCA TGGTGATGGA CAGAGTTATC GAGGCACATC CTCCACCACT
GTCACAGGAA GGAACTGTCA GTCTTGGTCA TCTATGATAC CACACTGGCA TCAGAGGACC
CCAGAAAACT ACCCAAATGC TGGCCTGACC AGGAACTACT GCAGGAATCC AGATTCTGGG
AAACAACCCT GGTGTTACAC GACTGATCCA TGTGTGAGGT GGGAGTACTG CAACCTGACA
CAATGCTCAG AAACAGAATC AGGTGTCCTA GAGACTCCCA CTGTTGTTCC GGTTCCAAGC
ATGGAAGCTC ATTCTGAAGC AGCACCAACT GAGCAAACTC CTGTGGTCCA GCAGTGCTAC
CATGGTAATG GACAGAGTTA TCGAGGCACA TTCTCCACCA CTGTCACAGG AAGGACATGT
CAATCTTGGT CATCCATGAC ACCACACCAG CATAAGAGGA CCCCGGAAAA CCACCCAAAT
GATGACTTGA CAATGAACTA CTGCAGGAAT CCAGATGCTG ACACAGGCCC TTGGTGTTTT
ACCATGGACC CCAGCGTCAG GCGGGAGTAC TGCAACCTGA CGCGATGCTC AGACACAGAA
GGGACTGTGG TCACACCTCC GACTGTTATC CCGGTTCCAA GCCTAGAGGC TCCTTCTGAA
CAAGCATCCT CTTCATTTGA TTGTGGGAAG CCTCAAGTGG AGCCAAAGAA ATGTCCTGGA
AGCATTGTAG GTGGGTGTGT GGCCCACCCA CATTCCTGGC CCTGGCAAGT CAGTCTTAGA
ACAAGGTTTG GAAAGCACTT CTGTGGAGGC ACCTTAATAT CCCCAGAGTG GGTGCTGACT
GCTGCTTGCT GCTTGGAGAC GTTCTCAAGG CCTTCCTTCT ACAAGGTCAT CCTGGGTGCA
CACCAAGAAG TGAATCTCGA ATCTCACGTT CAAGAAATAG AAGTGTCTAG GTTGTTCTTG
GAGCCCATAG GAGCAGATAT TGCCTTGCTA AAGCTAAGCA GGCCTGCCAT CATCACTGAC
AAAGTAATCC CAGCCTGTCT GCCGTCTCCA AATTACGTGA TCACCGTCTG GACTGAATGT
TACATCACTG GCTGGGGAGA AACCCAAGGT ACCTTTGGGG CTGGCCTTCT CAAGGAAGCC
CAGCTTCATG TGATTGAGAA TACAGTGTGC AATCACTACG AGTTTCTGAA TGGAAGAGTC
AAATCCACCG AGCTCTGTGC TGGGCATTTG GCCGGAGGCA CTGACAGATG CCAGGGTGAC
AGTGGAGGGC CTGTGGTTTG CTTCGACAAG GACAAATACA TTTTACGAGG AATAACTTCT
TGGGGTCCTG GCTGTGCATG CCCCAATAAG CCTGGTGTCT ATGTTCGTGT TTCAAGCTTT
GTCACTTGGA TTGAGGGAGT GATGAGAAAT AATTAATTGA ACAAGAGACA GAGTGAAGCA
TTGACTCACC TAGAGGCTAG AATGGGGGTA GGGATTTAGC ACGCTGGAAA TAACGGACAG
TAATCAAACG AAGACACTGT CCCCAGCTAC CAACTATGCC AAACCTCAGC ATTTTTGGTA
TTATTGTGTA TAAGCTTTTC CCGTCTGACT GCTGGGTTCT CCAATAAGGT GACATAGCTA
TGCCATTTGT TAAAAATAAA CTCTGTACTT ATTTTGATTT GAGTAAA
Rhesus monkey: XM_028847001.1 (SEQ ID NO: 3)
AGCCTTGCCT TTGAAATGTT CCAGTTGGAA CATTGCTGGG CAGCGTGCAA ACAGGAGCGA
ACGTCAGCCG GGGCGGCAGG GGGCAGCAGA CCCCACACTT TGTCCATGCC TCAGGTGGGA
GGAAGTGTCC GGCTCCAGAA ACCTGCCGCG GGCTTTATCC CAAGCGACTT CAGCCAGCAG
ACGGTTCATG TCCTGAGGCT GCAAAATACG AGTTCTGCCA TCATCGCTGA CAAAGTCATC
CCAGTTTGTC TGCCATCCCC AAATTATGCG ATCGCCAACC AGACTGAATG TTATGTCACT
GGCTGGGGAG AAACCCAAGC ACTACCTGAG CAAAGCCATG TGGTCCAGGA TTGCTACCAT
GGTGATGGAC AGAGTTATCA AGGCACATCC TCCACCACTG TCACAGGAAG GACCTGCCAA
GCTTGGTCAT CTATGGAACC ACATCAGCAT AATAGAACCA CAGAAAACTA CCCAAATGCT
GGCTTGATCA GGAACTACTG CAGGAATCCA GATCCTGTGG CAGCCCCTTA TTGTTATACG
ATGGATCCCA ATGTCAGGTG GGAGTACTGC AACCTGACAC AATGCTCAGA CGCAGAAGGG
ACTGCCGTCG CACCTCCGAA TGTCACCCCG GTTCCAAGCC TAGAGGCTCT TTCCGAACAA
GCACCGACTG AGCAAAGGCC TGGGGTGCAG GAGTGCTACC ACGGTAATGG ACAGAGTTAT
CGAGGCACAT ACTTCACCAC TGTGACAGGA AGAACCTGCC AAGCTTGGTC ATCTATGACA
CCACATTCTC ATAGTCGTAC CCCAGAAAAC TACCCAAATG GTGGCTTGAT CAGGAACTAC
TGCAGGAATC CAGATCCTGT GGCAGCCCCT TATTGTTATA CCATGGATCC CAATGTCAGG
TGGGAGTACT GCAACCTGAC ACAATGCTCA GACGCAGAAG GGACTGCCGT CGCACCTCCG
AATGTCACCC CGGTTCCAAG CCTAGAGGCT CCTTCCGAAC AAGCACCGAC TGAGCAAAGG
CCTGGGGTGC AGGAGTGCTA CCACGGTAAT GGACAGAGTT ATCGAGGCAC ATACTTCACC
ACTGTGACAG GAAGAACCTG CCAAGCTTGG TCATCTATGA CACCACATTC TCATAGTCGT
ACCCCAGAAA ACTACCCAAA TGGTGGCTTG ATCAGGAACT ACTGCAGGAA TCCAGATCCT
GTGGCAGCCC CTTATTGTTA TACCATGGAT CCCAATGTCA GGTGGGAGTA CTGCAACCTG
ACACAATGCT CAGACGCAGA GGGGACTGCC GTCGCACCTC CGACTGTCAC CCCGGTTCCA
AGCCTAGAGG CTCCTTCTGA GCAAGCACCG ACTGAGCAAA GGCCTGGGGT GCAGGAGTGC
TACCACGGTA ATGGACAGAG TTATCGAGGC ACATACTTCA CCACTGTGAC AGGAAGAACC
TGCCAAGCTT GGTCATCTAT GACACCGCAC TCTCATAGTC GGACCCCGGA AAACTACCCA
AATGGTGGCT TGATCAGGAA CTACTGCAGG AATCCAGATC CTGTGGCAGC CCCTTATTGT
TATACGATGG ATCCCAATGT CAGGTGGGAG TACTGCAACC TGACACAATG CTCAGACGCA
GAAGGGACTG CCGTCGCACC TCCGAATGTC ACCCCGGTTC CAAGCCTAGA GGCTCCTTCC
GAACAAGCAC CGACTGAGCA AAGGCCTGGG GTGCAGGAGT GCTACCACGG TAATGGACAG
AGTTATCGAG GCACATACTT CACCACTGTG ACAGGAAGAA CCTGCCAAGC TTGGTCATCT
ATGACACCGC ACTCTCATAG TCGGACCCCG GAAAACTACC CAAATGGTGG CTTGATCAGG
AACTACTGCA GGAATCCAGA TCCTGTGGCA GCCCCTTATT GTTATACGAT GGATCCCAAT
GTCAGGTGGG AGTACTGCAA CCTGACACAA TGCTCAGACG CAGAAGGGAC TGCCGTCGCA
CCTCCGAATG TCACCCCGGT TCCAAGCCTA GAGGCTCCTT CCGAACAAGC ACCAACTGAG
CAAAGGCCTG GGNTGCAGGA GTGCTACCAT GGTAATGGAC AGAGTTATCG AGGCACATAC
TTCACCACTG TGACAGGAAG AACCTGCCAA GCTTGGTCAT CTATGACACC GCACTCTCAT
AGTCGGACCC CGGAAAACTA CCCAAATGGT GGCTTGATCA GGAACTACTG CAGGAATCCA
GATCCTGTGG CAGCCCCTTA TTGTTATACC ATGGATCCCA ATGTCAGGTG GNAGTACTGC
AACCTGACAC AATGCTCAGA CGCAGAAGGG ACTGCCGTCG CACCTCCGAC TGTCACCCCG
GTTCCAAGCC TAGAGGCTCC TTCGAGCAAG GCACCGACTG AGCAAAGGCC TGGGNTGCAG
GAGTGCTACC ACGGTAATGG ACAGAGTTAT CGAGGCACAT ACTTCACCAC TGTGACAGGA
AGAACCTGCC AAGCTTGGTC ATCTATGACA CCGCACTCTC ATAGTCGGAC CCCGGAAAAC
TACCCAAATG GTGGCTTGAT CAGGAACTAC TGCAGGAATC CAGATCCTGT GGCAGCCCCT
TATTGTTATA CGATGGATCC CAATGTCAGG TGGGAGTACT GCAACCTGAC ACAATGCTCA
GACGCAGAAG GGACTGCCGT CGCACCTCCG AATGTCACCC CGGTTCCAAG CCTAGAGGCT
CCTTCCGAAC AAGCACCGAC TGAGCAAAGG CCTGGGGTGC AGGAGTGCTA CCACGGTAAT
GGACAGAGTT ATCGAGGCAC ATACTTCACC ACTGTGACAG GAAGAACCTG CCAAGCTTGG
TCATCTATGA CACCGCACTC TCATAGTCGG ACCCCGGAAA ACTACCCAAA TGGTGGCTTG
ATCAGGAACT ACTGCAGGAA TCCAGATCCT GTGGCAGCCC CTTATTGTTA TACCATGGAT
CCCAATGTCA GGTGGGAGTA CTGCAACCTG ACACAATGCT CAGACGCAGA AGGGACTGCC
GTCGCACCTC CGAATGTCAC CCCGGTTCCA AGCCTAGAGG CTCCTTCTGA GCAAGCACCA
ACTGAGCAAA GGCTTGGGGT GCAGGAGTGC TACCACAGTA ATGGACAGAG TTATCGAGGC
ACATACTTCA CCACTGTGAC AGGAAGAACC TGCCAAGCTT GGTCATCTAT GACACCACAC
TCTCATAGTC GGACCCCAGA AAACTACCCA AATGCTGGCT TGGTCAAGAA CTACTGCCGA
AATCCAGATC CTGTGGCAGC CCCTTGGTGT TATACAACGG ATCCCAGTGT CAGGTGGGAG
TACTGCAACC TGACACGATG CTCAGATGCA GAAGGGACTG CTGTCATGCC TCCAAATATT
ATTCCGGTTC CAAGCCTAGA GGCTTTTCTT GAACAAGAAC CTACTGAGGA AACCCCCGGG
GTACAGGAGT GCTACTACCA TTATGGACAG AGTTATCGAG GCACATACTC CACCACTGTT
ACAGGAAGAA CTTGCCAAGC TTGGTCATCT ATGACACCAC ACCAGCATAG TCGGACCCCA
AAAAACTATC CAAATGCTGG CCTGACCAGG AACTACTGCA GGAATCCAGA TGCTGAGATT
CGCCCTTGGT GTTATACCAT GGATCCCAGT GTCAGGTGGG AGTACTGCAA CCTGACACAA
TGTCTGGTGA CAGAATCAAG TGTCCTTGAA ACTCTCACAG TGGTCCCAGA TCCAAGCACA
CAGGCTTCTT CTGAAGAAGC ACCAACGGAG CAAAGTCCCG AGGTCCAGGA CTGCTACCAT
GGTGATGGAC AGAGTTATCG AGGCTCATTC TCCACCACTG TCACAGGAAG GACATGTCAG
TCTTGGTCCT CTATGACACC ACACTGGCAT CAGAGGACAA CAGAATATTA TCCAGATGGT
GGCCTGACCA GGAACTACTG CAGGAATCCA GATGCTGAGA TTCGCCCTTG GTGTTATACC
ATGGATCCCA GTGTCAGGTG GGAGTACTGC AACCTGACAC AATGTCCAGT GACAGAATCA
AGTGTCCTCG CAACGTCCAT GGCTGTTTCT GAACAAGCAC CAATGGAGCA AAGCCCCGGG
GTCCAGGACT GCTACCATGG TGATGGACAG AGTTATCGAG GTTCATTCTC CACCACTGTC
ACAGGAAGGA CATGTCAGTC TTGGTCCTCT ATGACACCAC ACTGGCATCA GAGGACCATA
GAATACTACC CAAATGGTGG CCTGACCAAG AACTACTGCA GGAATCCAGA TGCTGAGATT
CGCCCTTGGT GTTATACCAT GGATCCCAGA GTCAGATGGG AGTACTGCAA CCTGACACAA
TGTGTGGTGA TGGAATCAAG TGTCCTTGCA ACTCCCATGG TGGTCCCAGT TCCAAGCAGA
GAGGTTCCTT CTGAAGAAGC ACCAACTGAA AACAGCCCTG GGGTCCAGGA CTGCTACCAA
GGTGATGGAC AGAGTTATCG AGGCACATTC TCCACCACTA TCACAGGAAG AACATGTCAG
TCTTGGTTGT CTATGACACC ACATCGGCAT CGGAGGATCC CATTACGCTA TCCAAATGCT
GGCCTGACCA GGAACTATTG CAGAAATCCA GATGCTGAGA TTCGCCCTTG GTGTTACACC
ATGGATCCCA GTGTCAGGTG GGAGTACTGC AACCTGACAC AATGTCCAGT GACAGAATCA
AGTGTCCTCA CAACTCCCAC GGTGGTCCCG GTTCCAAGCA CAGAGGCTCC TTCTGAACAA
GCACCACCTG AGAAAAGCCC TGTGGTCCAG GATTGCTACC ATGGTGATGG ACAGAGTTAT
CGAGGCACAT CCTCCACCAC TGTCACAGGA AGGAACTGTC AATCTTGGTC ATCTATGATA
CCACACTGGC ATCAGAGGAC CCCAGAAAAC TACCCAAATG CTGGCCTGAC CAGGAACTAC
TGCAGGAATC CAGATTCTGG GAAACAACCC TGGTGTTACA CGACTGATCC ATGTGTGAGG
TGGGAGTACT GCAACCTGAC ACAATGCTCA GAAACAGAAT CAGGTGTCCT AGAGACTCCC
ACTGTTGTTC CGGTTCCAAG CATGGAAGCT CATTCTGAAG CAGCACCAAC TGAGCAAACC
CCTGTGGTCC AGCAGTGCTA CCATGGTAAT GGACAGAGTT ATCGAGGCAC ATTCTCCACC
ACTGTCACAG GAAGGACATG TCAATCTTGG TCATCCATGA CACCACACCA GCATAAGAGG
ACCCCGGAAA ACCACCCAAA TGATGACTTG ACAATGAACT ACTGCAGGAA TCCAGATGCT
GACACAGGCC CTTGGTGTTT TACCATGGAC CCCAGCGTCA GGCGGGAGTA CTGCAACCTG
ACGCGATGCT CAGACACAGA AGGGACTGTG GTCACACCTC CGACTGTTAT CCCGGTTCCA
AGCCTAGAGG CTCCTTCTGA ACAAGCATCC TCTTCATTTG ATTGTGGGAA GCCTCAAGTG
GAGCCAAAGA AATGTCCTGG AAGCATTGTA GGTGGGTGTG TGGCCCACCC ACATTCCTGG
CCCTGGCAAG TCAGTCTTAG AACAAGGTTT GGAAAGCACT TCTGTGGAGG CACCTTAATA
TCCCCAGAGT GGGTGCTGAC TGCTGCTTGC TGCTTGGAGA CGTTCTCAAG GCCTTCCTTC
TACAAGGTCA TCCTGGGTGC ACACCAAGAA GTGAATCTCG AATCTCATGT TCAAGAAATA
GAAGTGTCTA GGTTGTTCTT GGAGCCCATA GGAGCAGATA TTGCCTTGCT AAAGCTAAGC
AGGCCTGCCA TCATCACTGA CAAAGTAATC CCAGCCTGTC TGCCGTCTCC AAATTACGTG
ATCACCGCCT GGACTGAATG TTACATCACT GGCTGGGGAG AAACCCAAGG TACCTTTGGG
GCTGGCCTTC TCAAGGAAGC CCAGCTTCAT GTGATTGAGA ATACAGTGTG CAATCACTAC
GAGTTTCTGA ATGGAAGAGT CAAATCCACT GAGCTCTGTG CTGGGCATTT GGCCGGAGGC
ACTGACAGAT GCCAGGGTGA CAATGGAGGG CCTGTGGTTT GCTTCGACAA GGACAAATAC
ATTTTACGAG GAATAACTTC TTGGGGTCCT GGCTGTGCAT GCCCCAATAA GCCTGGTGTC
TATGTTCGTG TTTCAAGCTT TGTCACTTGG ATTGAGGGAG TGATGAGAAA TAATTAATTG
AACAAGAGAC AGAGTGAAGC ATTGACTCAC CTAGAGGCTA GAATGGGGGT AGGGATTTAG
CACGCTGGAA ATAACGGACA GTAATCAAAC GAAGACACTG TCCCCAGCTA CCAACTATGC
CAAACCTCAG CATTTTTGGT ATTATTGTGT ATAAGCTTTT CCTGTCTGAC TGCTGGGTTC
TCCAATAAGG TGACATAGCT ATGCCATTTG TTAAAAATAA ACTCTGTACT TATTTTGATT
TGAGTAAA

TABLE 6
LPA Oligonucleotide Sequences (modified)

| | | SEQ | | SEQ |
|---|---|---|---|---|
| | Sequence | ID | Sequence | ID |
| Oligonucleotide | (Sense Strand) | NO: | (Antisense Strand) | NO: |
| LPA-0190-M1 | [mUs][mC][mC][mA][mC] | 388 | [Me Phosphonate-4O- | 788 |
| | [mC][mA][fC][fU][fG] | | mUs][fUs][fUs][fC][fC] | |
| | [fU][mC][mA][mC][mA] | | [mU][fG][mU][mG][fA][mC] | |
| | [mG][mG][mA][mA][mA] | | [mA][mG][fU][mG][mG][mU] | |
| | [mG][mC][mA][mG][mC][mC] | | [mG][mG][mAs][mGs][mG] | |
| | [mG][ademA- | | | |
| | GalNAc][ademA- | | | |
| | GalNAc][ademA- | | | |
| | GalNAc][mG][mG][mC] | | | |
| | [mU][mG][mC] | | | |
| LPA-0501-M1 | [mUs][mG][mG][mU][mA] | 389 | [Me Phosphonate-4O- | 789 |
| | [mA][mU][fG][fG][fA] | | mUs][fAs][fUs][fA][fA] | |
| | [fC][mA][mG][mA][mG][mU] | | [mC][fU][mC][mU][fG][mU] | |
| | [mU][mA][mU][mA][mG] | | [mC][mC][fA][mU][mU][mA] | |
| | [mC][mA][mG][mC][mC] | | [mC][mC][mAs][mGs][mG] | |
| | [mG][ademA- | | | |
| | GalNAc][ademA- | | | |
| | GalNAc][ademA- | | | |
| | GalNAc][mG][mG][mC] | | | |
| | [mU][mG][mC] | | | |
| LPA-3100-M1 | [mUs][mA][mC][mU][mG] | 390 | [Me Phosphonate-4O- | 790 |
| | [mC][mA][fA][fC][fC] | | mUs][fAs][fUs][fC][fG] | |
| | [fU][mG][mA][mC][mA][mC] | | [mU][fG][mU][mC][fA][mG] | |
| | [mG][mA][mU][mA][mG] | | [mG][mU][fU][mG][mC][mA] | |
| | [mC][mA][mG][mC][mC] | | [mG][mU][mAs][mGs][mG] | |
| | [mG][ademA- | | | |
| | GalNAc][ademA- | | | |
| | GalNAc][ademA- | | | |
| | GalNAc][mG][mG][mC][mU] | | | |
| | [mG][mC] | | | |
| LPA-3286-M1 | [mAs][mG][mA][mA][mC] | 391 | [Me Phosphonate-4O- | 791 |
| | [mU][mU][fG][fC][fC] | | mUs][fAs][fCs][fC][fA] | |
| | [fA][mA][mG][mC][mU][mU] | | [mA][fG][mC][mU][fU][mG] | |
| | [mG][mG][mU][mA][mG] | | [mG][mC][fA][mA][mG][mU] | |
| | [mC][mA][mG][mC][mC] | | [mU][mC][mUs][mGs][mG] | |
| | [mG][ademA- | | | |
| | GalNAc][ademA- | | | |
| | GalNAc][ademA- | | | |
| | GalNAc][mG][mG][mC][mU] | | | |
| | [mG][mC] | | | |
| LPA-3288-M1 | [mAs][mA][mC][mU][mU] | 392 | [Me Phosphonate-4O- | 792 |
| | [mG][mC][fC][fA][fA] | | mUs][fUs][fGs][fA][fC] | |
| | [fG][mC][mU][mU][mG][mG] | | [mC][FA][mA][mG][fC][mU] | |
| | [mU][mC][mA][mA][mG] | | [mU][mG][fG][mC][mA][mA] | |
| | [mC][mA][mG][mC][mC] | | [mG][mU][mUs][mGs][mG] | |
| | [mG][ademA- | | | |
| | GalNAc][ademA- | | | |
| | GalNAc][ademA- | | | |
| | GalNAc][mG][mG][mC][mU] | | | |
| | [mG][mC] | | | |
| LPA-3291-M1 | [mUs][mU][mG][mC][mC] | 393 | [Me Phosphonate-4O- | 793 |
| | [mA][mA][fG][fC][fU] | | mUs][fAs][fGs][fA][fU] | |
| | [fU][mG][mG][mU][mC][mA] | | [mG][fA][mC][mC][fA][mA] | |
| | [mU][mC][mU][mA][mG] | | [mG][mC][fU][mU][mG][mG] | |
| | [mC][mA][mG][mC][mC] | | [mC][mA][mAs][mGs][mG] | |
| | [mG][ademA- | | | |
| | GalNAc][ademA- | | | |
| | GalNAc][ademA- | | | |
| | GalNAc][mG][mG][mC][mU] | | | |
| | [mG][mC] | | | |
| LPA-3584-M1 | [mAs][mU][mG][mG][mA] | 394 | [Me Phosphonate-4O- | 794 |
| | [mC][mA][fG][fA][fG] | | mUs][fCs][fCs][fU][fC] | |
| | [fU][mU][mA][mU][mC][mG] | | [mG][fA][mU][mA][fA][mC] | |
| | [mA][mG][mG][mA][mG] | | [mU][mC][fU][mG][mU][mC] | |
| | [mC][mA][mG][mC][mC] | | [mC][mA][mUs][mGs][mG] | |
| | [mG][ademA- | | | |
| | GalNAc][ademA- | | | |
| | GalNAc][ademA- | | | |
| | GalNAc][mG][mG][mC][mU] | | | |
| | [mG][mC] | | | |
| LPA-3585-M1 | [mUs][mG][mG][mA][mC] | 395 | [Me Phosphonate-4O- | 795 |
| | [mA][mG][fA][fG][fU] | | mUs][fGs][fCs][fC][fU] | |
| | [fU][mA][mU][mC][mG][mA] | | [mC][fG][mA][mU][fA][mA] | |
| | [mG][mG][mC][mA][mG] | | [mC][mU][fC][mU][mG][mU] | |
| | [mC][mA][mG][mC][mC] | | [mC][mC][mAs][mGs][mG] | |
| | [mG][ademA- | | | |
| | GalNAc][ademA- | | | |
| | GalNAc][ademA- | | | |
| | GalNAc][mG][mG][mC][mU] | | | |
| | [mG][mC] | | | |
| LPA-4645-M1 | [mUs][mG][mG][mU][mC] | 396 | [Me Phosphonate-4O- | 796 |
| | [mA][mU][fC][fU][fA] | | mUs][fGs][fUs][fG][fG] | |
| | [fU][mG][mA][mU][mA][mC] | | [mU][fA][mU][mC][fA][mU] | |
| | [mC][mA][mC][mA][mG] | | [mA][mG][fA][mU][mG][mA] | |
| | [mC][mA][mG][mC][mC] | | [mC][mC][mAs][mGs][mG] | |
| | [mG][ademA- | | | |
| | GalNAc][ademA- | | | |
| | GalNAc][ademA- | | | |
| | GalNAc][mG][mG][mC][mU] | | | |
| | [mG][mC] | | | |
| LPA-4717-M1 | [mUs][mA][mC][mU][mG] | 397 | [Me Phosphonate-4O- | 797 |
| | [mC][mA][fG][fG][fA] | | mUs][fAs][fAs][fU][fC] | |
| | [fA][mU][mC][mC][mA][mG] | | [mU][fG][mG][mA][fU][mU] | |
| | [mA][mU][mU][mA][mG] | | [mC][mC][fU][mG][mC][mA] | |
| | [mC][mA][mG][mC][mC] | | [mG][mU][mAs][mGs][mG] | |
| | [mG][ademA- | | | |
| | GalNAc][ademA- | | | |
| | GalNAc][ademA- | | | |
| | GalNAc][mG][mG][mC][mU] | | | |
| | [mG][mC] | | | |
| LPA-5510-M1 | [mAs][mG][mA][mA][mA] | 398 | [Me Phosphonate-4O- | 798 |
| | [mU][mG][fU][fC][fC] | | mUs][fAs][fUs][fG][fC] | |
| | [fU][mG][mG][mA][mA][mG] | | [mU][fU][mC][mC][fA][mG] | |
| | [mC][mA][mU][mA][mG] | | [mG][mA][fC][mA][mU][mU] | |
| | [mC][mA][mG][mC][mC] | | [mU][mC][mUs][mGs][mG] | |
| | [mG][ademA- | | | |
| | GalNAc][ademA- | | | |
| | GalNAc][ademA- | | | |
| | GalNAc][mG][mG][mC][mU] | | | |
| | [mG][mC] | | | |
| LPA-3750-M1 | [mGs][mA][mC][mA][mA] | 399 | [Me Phosphonate-4O- | 799 |
| | [mC][mA][fG][fA][fA] | | mUs][fUs][fGs][fG][fA] | |
| | [fU][mA][mU][mU][mA][mU] | | [mU][fA][mA][mU][fA][mU] | |
| | [mC][mC][mA][mA][mG] | | [mU][mC][fU][mG][mU][mU] | |
| | [mC][mA][mG][mC][mC] | | [mG][mU][mCs][mGs][mG] | |
| | [mG][ademA- | | | |
| | GalNAc][ademA- | | | |
| | GalNAc][ademA- | | | |
| | GalNAc][mG][mG][mC][mU] | | | |
| | [mG][mC] | | | |
| LPA-2900-M2 | [mAs][mU][mG][mG][mA] | 400 | [Me Phosphonate-4O- | 800 |
| | [mC][mA][fG][fA][fG] | | mUs][fCs][fC][fU][fU][mG] | |
| | [fU][mU][mA][mU][mC][mA] | | [fA][mU][mA][fA][mC][mU] | |
| | [mA][mG][mG][mA][mG] | | [mC][fU][mG][mU][mC][mC] | |
| | [mC][mA][mG][mC][mC] | | [mA][mUs][mGs][mG] | |
| | [mG][ademA- | | | |
| | GalNAc][ademA- | | | |
| | GalNAc][ademA- | | | |
| | GalNAc][mG][mG][mC][mU] | | | |
| | [mG][mC] | | | |
| LPA-3675-M2 | [mGs][mA][mC][mA][mA] | 401 | [Me Phosphonate-4O- | 801 |
| | [mC][mA][fG][fA][fA] | | mUs][fUs][fG][fG][fA][mU] | |
| | [fU][mA][mU][mU][mA][mU] | | [fA][mA][mU][fA][mU][mU] | |
| | [mC][mC][mA][mA][mG] | | [mC][fU][mG][mU][mU][mG] | |
| | [mC][mA][mG][mC][mC] | | [mU][mCs][mGs][mG] | |
| | [mG][ademA- | | | |
| | GalNAc][ademA- | | | |
| | GalNAc][ademA- | | | |
| | GalNAc][mG][mG][mC][mU] | | | |
| | [mG][mC] | | | |
| LPA-2900-M3 | [mAs][mU][mG][mG][mA] | 402 | [Me Phosphonate-4O- | 802 |
| | [mC][mA][fG][fA][fG] | | mUs][fCs][fC][mU][fU][mG] | |
| | [fU][mU][mA][mU][mC][mA] | | [fA][mU][mA][fA][mC][mU] | |
| | [mA][mG][mG][mA][mG] | | [mC][fU][mG][mU][mC][mC] | |
| | [mC][mA][mG][mC][mC] | | [mA][mUs][mGs][mG] | |
| | [mG][ademA- | | | |
| | GalNAc][ademA- | | | |
| | GalNAc][ademA- | | | |
| | GalNAc][mG][mG][mC][mU] | | | |
| | [mG][mC] | | | |
| LPA-3675-M3 | [mGs][mA][mC][mA][mA] | 403 | [Me Phosphonate-4O- | 803 |
| | [mC][mA][fG][fA][fA] | | mUs][fUs][fG][mG][fA][mU] | |
| | [fU][mA][mU][mU][mA][mU] | | [fA][mA][mU][fA][mU][mU] | |
| | [mC][mC][mA][mA][mG] | | [mC][fU][mG][mU][mU][mG] | |
| | [mC][mA][mG][mC][mC] | | [mU][mCs][mGs][mG] | |
| | [mG][ademA- | | | |
| | GalNAc][ademA- | | | |
| | GalNAc][ademA- | | | |
| | GalNAc][mG][mG][mC][mU] | | | |
| | [mG][mC] | | | |

### Modifications in Table 6:

mC, mA, mG, mU=2′-OMe ribonucleosides; fA, fC, fG, fU=2′-F ribonucleosides; s=phosphorothioate; MePhosphonate-4O-mUs=

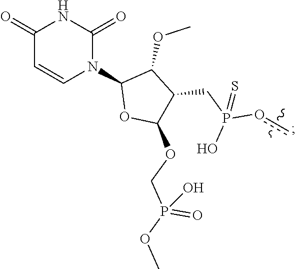

ademA-GalNAc=GalNAc attached to an adenine nucleotide:

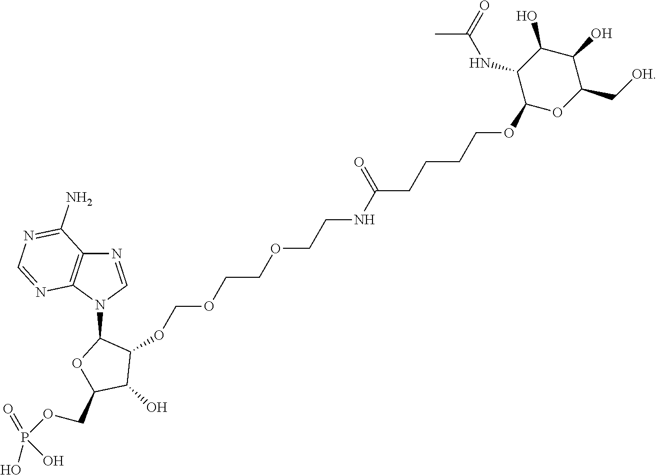 [SMILES: CC(=O)N[C@H]1[C@H](OCCCCC(=O)NCCOCCOCO[C@@H]2[C@H](O)[C@@H](COP(=O)(O)O)O[C@H]2n2cnc3c(N)ncnc32)O[C@H](CO)[C@H](O)[C@@H]1O]

1-92. (canceled)

93. An RNAi oligonucleotide for reducing apolipoprotein (a) (LPA) expression, the oligonucleotide comprising a sense strand and an antisense strand, wherein the sense strand and the antisense strand form a duplex region, wherein the antisense strand is:
5′-[MePhosphonate-4O-mUs] [fAs] [fGs] [fA] [fU] [mG] [fA] [mC] [mC] [fA] [mA] [mG] [mC] [fU] [mU] [mG] [mG] [mC] [mA] [mAs] [mGs] [mG]-3′ (SEQ ID NO: 793), and the sense strand is:
5′-[mUs] [mU] [mG] [mC] [mC] [mA] [mA] [fG] [fC] [fU] [fU] [mG] [mG] [mU] [mC] [mA] [mU] [mC] [mU] [mA] [mG] [mC] [mA] [mG] [mC] [mC] [mG] [ademA-GalNAc] [ademA-GalNAc] [ademA-GalNAc] [mG] [mG] [mC] [mU] [mG] [mC]-3′ (SEQ ID NO: 393),
wherein:
mA represents 2′-OMe adenosine;
mC represents 2′-OMe cytosine;
mG represents 2′-OMe guanosine;
mU represents 2′-OMe uridine;
fA represents 2′-F adenosine,
fC represents 2′-F cytosine;
fG represents 2′-F guanosine;
fU represents 2′-F uridine;
fAs represents 2′-F adenosine with a 3′-phosphorothioate linkage;
fGs represents 2′-F guanosine with a 3′-phosphorothioate linkage;
mAs represents 2′-OMe adenosine with a 3′-phosphorothioate linkage;
mGs represents 2′-OMe guanosine with a 3′-phosphorothioate linkage;
mUs represents 2′-OMe uridine with a 3′-phosphorothioate linkage;
ademA-GalNAc represents 2′-aminodiethoxymethanol-adenine-GalNAc: 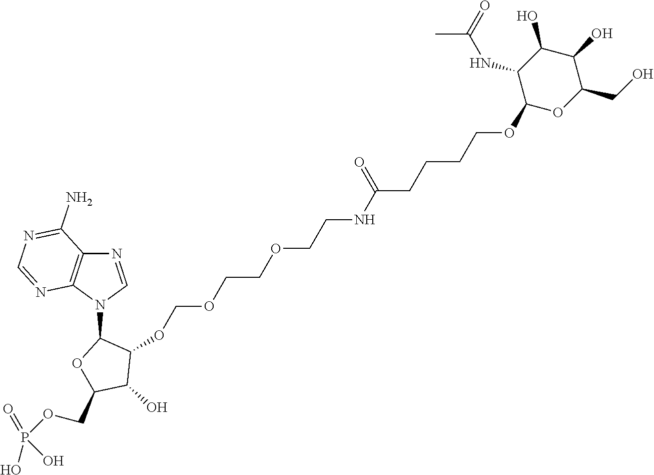 [SMILES: CC(=O)N[C@H]1[C@H](OCCCCC(=O)NCCOCCOCO[C@@H]2[C@H](O)[C@@H](COP(=O)(O)O)O[C@H]2n2cnc3c(N)ncnc32)O[C@H](CO)[C@H](O)[C@@H]1O]
and
MePhosphonate-4O-mUs represents

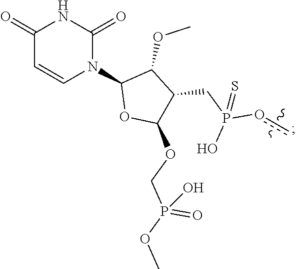

94. A pharmaceutical composition comprising:
(i) the RNAi oligonucleotide of claim 93; and
(ii) a pharmaceutically acceptable carrier, delivery agent or excipient.

95. The pharmaceutical composition of claim 94, wherein the carrier comprises phosphate buffered saline.

96. The pharmaceutical composition of claim 94, wherein the carrier comprises water.

97. A method of treating a subject having a cardiometabolic disease, comprising administering to the subject a therapeutically effective amount of the RNAi oligonucleotide of claim 93, or a pharmaceutical composition thereof.

98. The method of claim 97, wherein the cardiometabolic disease is atherosclerosis, dyslipidemia, nonalcoholic fatty liver disease (NAFLD), or nonalcoholic steatohepatitis (NASH).

99. The method of claim 97, wherein the RNAi oligonucleotide, or a pharmaceutical composition thereof, is administered subcutaneously.
